(19) United States
(12) Patent Application Publication
(10) Pub. No.: US 2023/0365709 A1
(43) Pub. Date: Nov. 16, 2023

(54) TRISPECIFIC BINDERS

(71) Applicant: Affimed GmbH, Heidelberg (DE)

(72) Inventors: Thorsten ROSS, Heidelberg (DE); Joachim KOCH, Heidelberg (DE); Michael TESAR, Heidelberg (DE); Holger DULAT, Heidelberg (DE); Christian BREUNIG, Heidelberg (DE); Eike PETERS, Heidelberg (DE); Kristina ELLWANGER, Heidelberg (DE); Ivica FUCEK, Heidelberg (DE); Uwe REUSCH, Heidelberg (DE); Jens PAHL, Heidelberg (DE)

(21) Appl. No.: 18/030,628

(22) PCT Filed: Oct. 8, 2021
(86) PCT No.: PCT/EP2021/077882

§ 371 Date: Apr. 6, 2023

(30) Foreign Application Priority Data
Oct. 8, 2020 (EP) 20200845.4

(51) Int. Cl. C07K 16/32; C07K 16/28; C07K 16/46; A61P 35/00

(52) U.S. Cl. CPC C07K 16/32; C07K 16/283; C07K 16/468; A61P 35/00; C07K 16/2863; C07K 16/2803; C07K 2317/31; C07K 2317/622; C07K 2317/64; C07K 2317/732; C07K 2317/92; C07K 2317/75

(57) ABSTRACT

The present invention relates to a trispecific antibody construct comprising (i.) a first binding domain (A), which is capable of specifically binding to a first target (A′) that is CD16A on the surface of an immune effector cell; (ii.) a second binding domain (B), which is capable of specifically binding to a second target (B′) that is another antigen on the surface of an immune effector cell, wherein said antigen is selected from the group comprising CD56, NKG2A, NKG2D, NKp30, NKp44, NKp46, NKp80, DNAM-1, SLAMF7, OX40, CD47/SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5 and CD3; and (iii.) a third binding domain (C), which is capable of specifically binding to a third target (C′) that is an antigen on the surface of a target cell. The present invention also relates to related nucleic acid molecules, vectors, host cells, methods of producing the antibody constructs, pharmaceutical compositions, medical uses, and kits.

### FIELD OF THE INVENTION

The present invention relates to a trispecific antibody construct comprising (i.) a first binding domain (A), which is capable of specifically binding to a first target (A′) that is CD16A on the surface of an immune effector cell; (ii.) a second binding domain (B), which is capable of specifically binding to a second target (B′) that is another antigen on the surface of an immune effector cell; and (iii.) a third binding domain (C), which is capable of specifically binding to a third target (C′) that is an antigen on the surface of a target cell. The present invention also relates to related nucleic acid molecules, vectors, host cells, methods of producing the antibody constructs, pharmaceutical compositions, medical uses, and kits.

### BACKGROUND

WO 2006/125668 and Reusch et al, MABS, 2014, 6:3:728-739 describe an antigen-binding protein—a bispecific tandem diabody—for engagement of CD16A and its use for natural killer (NK) cell therapy. WO 2019/198051 and Ellwanger et al., mAbs 2019 describe multispecific antigen-binding proteins for engagement of CD16A (FcγRIIIA) on NK cells through this triggering NK cell cytotoxicity.

Natural killer cells are cytotoxic, IFN-γ and TNF-α producing innate lymphoid cells that are considered the first line of defense against virus-infected cells and cancer cells (Cerwenka and Lanier 2001). The cytotoxic potential of NK cells can be utilized in cancer immunotherapy by redirecting NK cell lysis to tumor cells and stimulating the activating receptor CD16A, also known as FcγRIIIA, expressed on the surface of NK cells. CD16A activation promotes NK cell proliferation and memory-like cytotoxicity against cancer cells (Pahl et al 2018 Cancer Immunol Res; 6(5), 517-27; DOI: 10.1158/2326-6066.CIR-17-0550). The cytotoxic activity of NK cells can be enhanced by increasing the avidity through multivalent binding to CD16A, e.g. using constructs with bivalent binding to CD16A (WO2019/198051 Affimed GmbH).

Directing NK cells for tumor cell lysis using multispecific antibodies is considered a potent immunotherapeutic approach and offers opportunities for increasing specificity, potency, and utilizing novel mechanisms of action. For example, each of the antigen-binding moieties may be selected from the group consisting of a single-chain diabody (scDb), a diabody (db), a single chain Fv (scFv) or a Fab fragment. Bispecific antibodies consisting of one arm which binds CD16A and another which binds a tumor-associated antigen (e.g. CD19) have been developed (Kellner et al 2011 Cancer Lett. 303(2): 128-139).

NK cells are equipped with multiple activating and inhibitory receptors on their surface jointly regulating NK cell activation and triggering of effector functions. Several of these receptors play a pivotal role for NK cell mediated recognition and killing of cancer cells. Bi- or multispecific antibodies or binding proteins cross-linking two different NK cell receptors to recruit and activate NK cells are in development. In one approach a multifunctional binding protein engages NK cells by binding NKp46 and CD16A in addition to an antigen on cancer cells. In another approach, a bispecific antibody has incorporated one antigen-binding site for NKG2D and another one for a tumor-associated antigen. This antibody format contains a Fc domain, which can bind CD16A of NK cells. In a third approach a multispecific NK cell engager targeting NKp30 with one antigen-binding site and a tumor-associated antigen with the second antigen-binding site was used.

The lack of NK cell fratricide is an important feature for high-affinity, at least bivalent and/or multi-specific immune cell engager formats that are characterized by longer cell retention times and that are either to be used for the engagement of endogenous NK cells or that are to be combined with NK cellular therapeutic approaches (WO 2019/198051 Affimed GmbH). Hence, NK cell cross-linking with NK cells or other immune cells is expected to reduce therapeutic efficacy of NK cell-engagement. Most importantly, cross-linking of a NK cell with one or more NK cells or other immune cells through bivalent or multivalent interactions with FcRγ or in combination with a second immune cell antigen (e.g. NKG2D, NKp30, SLAMF7, CD38) can cause immune cell activation. This might lead to induction of target cell-driven fratricide or immune cell killing (e.g. NK-NK cell lysis), ultimately resulting in efficient NK cell depletion in vivo, as previously described for a CD16-directed murine IgG antibody (3G8), the CD38-directed antibody daratumumab and other approaches (Choi et al 2008 Immunology 124 (2) 215-22; DOI: 10.1111/j.1365-2567.2007.02757.x; Yoshida 2010 Front. Microbiol 1:128 DOI: 10.3389/fmicb.2010.00128; Wang et al 2018 Clin Cancer Res, 24(16): 4006-4017; DOI: 10.1158/1078-0432.CCR-17-3117; His et al 2008; Nakamura 2013 PNAS; 110(23) 9421-9426; DOI: 10.1073/pnas.1300140110; Breman et al 2018 Front Immunol, 12(9)2940; DOI: 10.3389/fimmu.2018.02940).

The existing immuno-oncology therapies with multispecific binding proteins inducing NK cell activation by binding to CD16 as well as a second NK cell antigen are only effective to a certain extent for most tumor indications. Further improvement of multispecific binding proteins with sufficient reduction of immune cell fratricide are urgently needed. The present invention addresses this need as indicated herein.

### BRIEF DESCRIPTION OF THE DRAWINGS

FIG. 1: Schematic depiction of antibody constructs 2Fab-scFc-1scDb (left) and 2Fab-scFc-1scFv (right). The first binding domain (A) is specific for CD16A, the second binding domain (B) is specific for another target for on the surface of an immune effector cell (IC), the third binding domain (A) is specific for an antigen on the surface of a target cell (TAA).

FIG. 2: Schematic depiction of antibody constructs 2Fab-1scDb-AFc (left) and 2Fab-1scFv-AFc (right).

FIG. 3: Schematic depiction of antibody construct 1Fab-1scDb-AFc.

FIG. 4: Schematic depiction of antibody constructs 2scDb-AFc (left) and 1scDb-1scFv-AFc (right).

FIG. 5: Schematic depiction of antibody constructs 2tascFv-AFc (left) and 1tascFv-1scFv-1scFv-AFc (right).

FIG. 6: Schematic depiction of antibody constructs 1scDb-2Fab-AFc (left) and 1scDb-1Fab-AFc (right).

FIG. 7: Schematic depiction of antibody construct AIG-2scFv.

FIG. 8: Schematic depiction of antibody construct IG-2scDb.

FIG. 9: Schematic depiction of antibody construct AIG-2scDb.

FIG. 10: Schematic depiction of antibody construct AIG-1scDb.

FIG. 11: Schematic depiction of antibody construct AIG-1scFv.

FIG. 12: Schematic depiction of antibody construct 1Fab-AFc-1Fab.

FIG. 13: Purity of NK cells enriched from PBMC. PBMC were isolated from buffy coats by density gradient centrifugation. NK cells were enriched from PBMC by negative selection. After flow cytometric staining with fluorescent-labeled antibodies, single-cell SSC/FSC-live-gated cells were gated as CD45⁺ PBMC or enriched NK cells. Afterwards, monocytes were gated as CD14⁺ FSC^{high} cells in PBMC or enriched NK cells. Within the CD14⁻ cell population, NK cells were gated as CD56⁺ CD3⁻ cells and T cells (devoid of CD56⁺ NKT cells) as CD3⁺ CD56⁻ cells. Within the CD14⁻ cell population, the overall content of B cells and T cells were determined as CD19⁺ CD3⁻ cells and CD3⁺ CD19⁻ cells, respectively. Within the CD56⁺ CD3⁻ CD14⁻ cell population, the proportion of CD56⁺ CD16⁺ NK cells was additionally determined.

FIG. 14: Representation of CD16a (left) and NKp46 (right). The binding region of CD16a to Fc as well as the location of Y158 are highlighted in the structure of CD16a. The location of epitopes NKp46-1 and NKp46-3 are highlighted in the structure of NKp46.

FIG. 15: Schematic representation of different exemplary antibody constructs and the theoretical distance between the first (CD16a) binding site and the second (here: NKG2D) binding site.

FIG. 16 NK cell fratricide assay with trispecifc HER2/CD16A/NKG2D antibody constructs. HER2/CD16A/NKG2D trispecific constructs AIG-2scFv-7, AIG-2scFv-8, and AIG-2scFv-10, were tested together with control antibody constructs AIG-2scFv-14 (HER2/NKG2D/NKG2D), AIG-2scFv-15 (HER2/NKG2D/RSV), and AIG-1scFv-4 (HER2/NKG2D) at the indicated concentrations in 4 h calcein-release NK cell fratricide assays. Human IgG1 anti-CD38 (IgAb-51, SEQ ID NOs: 429-430) was used as a positive control for induction of NK cell fratricide.

FIG. 17 shows analysis of expressed half-antibodies containing (A) knob- or (B) hole mutations in their Fc, and (C-D) during the heterodimerization by asymmetric assembly. Protein samples were run in SDS-PAGE under non-reducing (nR) or reducing (R) conditions to separate disulfide bridges between heavy chains (HC) and light chains (LC), whereby the intact half antibodies run at the expected mass of 100 kDa under non-reducing conditions, or 77 kDa for the HC and 23 kDa for the LC under reducing conditions. (C) Assembly of the asymmetric antibody AIG-2scFv-8 (SEQ ID NOs: 434-436) takes place fast after mixing and supplementation with reduced L-Glutathione (0d) and is complete after one day (1d) incubation as visualized by formation of the assembled product running at >200 kDa on non-reducing SDS-PAGE. (D) Purity and size of the assembled asymmetric antibody analyzed by SEC/MALS-HPLC revealed the expected sizes of assembled antibody with 89% purity and minor factions of ˜5% higher molecular weight (HMW) and ˜6% lower molecular weight (LMW) forms.

FIG. 18 Concentration-dependent induction of tumor cell lysis by trispecific antibody constructs using primary NK cells as effector cells in 4 h calcein-release cytotoxicity assays. Calcein-labeled CD19⁺ GRANTA-519 target cells (A) or EGFR⁺ A-431 target cells (B) were co-cultured for 4 h with enriched primary human NK cells as effector cells at an E:T ratio of 5:1 in the presence of serial dilutions of the respective antibodies in duplicates. Fc-enhanced anti-CD19 IgG1 (IgAb-67), anti-EGFR IgG1 (IgAb-53), and without (w/o) antibody were used as controls. Mean lysis values and standard deviations (SD) as error bars are plotted. The experiments were carried out in biological duplicates and the graph of one representative experiment is shown.

FIG. 19 Exemplary binding of trispecific molecules CD19/CD16A/NKG2D AIG-2scFv-17 and CD19/CD16A/NKp46 AIG-2scFv-18 to recombinant human CD16A (158F), CD16B (NA1), CD32, CD64, NKG2D, and NKp46 expressed on the surface of CHO cells. CHO cells were incubated with the indicated concentrations of antibodies. Cell bound antibodies were detected via incubation with FITC-labeled secondary antibodies and flow cytometric analysis. The assays were performed in two biological replicates, of which one representative graph is shown. Control (ctrl) antibodies for the respective receptors have been included: mAb anti-CD16 (anti-human CD16A and CD16B), mAb anti-human CD32, and mAb anti-CD64 as well as mAb anti-CD355 (NKp46) and mAb anti-CD314 (NKG2D).

FIG. 20 Exemplary binding of trispecific molecules CD19/CD16A/NKG2D 2tascFv-AFc-2, CD19/CD16A/NKG2D 2Fab-scFc-1scDb-2, CD19/CD16A/NKp46 2Fab-scFc-1scDb-4, CD19/Fc/NKp46 1Fab-AFc-1Fab-1, and CD19/Fc-enhanced Fc/NKp46 1Fab-AFc-1Fab-6 to recombinant human CD16A (158F), CD16B (NA1), CD32, CD64, NKG2D, and NKp46 expressed on the surface of CHO cells. CHO cells were incubated with the indicated concentrations of antibodies. Cell bound antibodies were detected via incubation with FITC-labeled secondary antibodies and flow cytometric analysis. The assays were performed in two biological replicates, of which one representative graph is shown. Control (ctrl) antibodies for the respective receptors have been included: mAb anti-CD16 (anti-human CD16A and CD16B), mAb anti-human CD32, and mAb anti-CD64 as well as mAb anti-CD355 (NKp46) and mAb anti-CD314 (NKG2D).

FIG. 21 4 h calcein-release cytotoxicity assays with calcein-labeled NK cells as target cells and autologous NK cells as effector cells to assess concentration-dependent NK cell fratricide induced by trispecific antibody constructs. IG-scDb, 1Fab-1scDb-AFc, AIG-2scFv, and scFv-IgAb (A), 1scDb-1scFv-AFc, 2Fab-scFc-1scDb, and 1Fab-AFc-1Fab (B), 2Fab-1scFv-AFc, 2Fab-1scDb-AFc, AIG-1scDb-AFc, and AIG-1scDb (C), 1tascFv-1scFv-AFc, 2scDb-AFc, 2Fab-scFc-1scFv, and AIG-1scFv (D). Anti-CD38 IgG1 (IgAb-51) was used as a positive control in all assays. Mean and SD of duplicate lysis values are plotted.

FIG. 22 4 h calcein-release cytotoxicity assays with calcein-labeled THP-1 target cells and enriched primary human NK cells as effector cells at an E:T ratio of 5:1 in the presence of serial dilutions of 2Fab-1scDb-AFc (A), 2scDb-AFc, 1scDb-1scFv-AFc, 2tascFv-AFc, and 2Fab-scFc-1scDb (B), AIG-2scFv and AIG-2scDb (C), 2Fab-scFc-1scFv and 1Fab-AFc-1Fab (D), 2Fab-1scDb-AFc, 2Fab-1scFv-AFc, and 1Fab-1scDb-AFc (E), and IG-scDb and scFv-IgAb (F). Anti-CD16A IgG1 (IgAb-50) was used as a positive control in all assays. As a negative control (ctrl) target cells were incubated with NK cells without (w/o) antibodies on each plate. Mean and SD of duplicate lysis values are plotted.

FIG. 23 Concentration-dependent induction of tumor cell lysis by trispecific antibody constructs using PBMCs as effector cells in 4 h calcein-release cytotoxicity assays. Calcein-labeled CD19⁺ GRANTA-519 target cells were incubated with human PBMC as effector cells at an E:T ratio of 50:1 in the presence of serial dilutions of the respective antibodies in duplicates. Fc-enhanced anti-CD19 IgG1 (IgAb-67) was used as a positive control, and target and effector cells without (w/o) antibodies were used as a negative control (ctrl). Mean lysis values and error bars indicate the standard deviation (SD). The experiments were carried out in biological duplicates and one representative resulting diagram is shown.

FIG. 24 Size-related heterogeneities analyzed under native conditions by SE-HPLC. (A) 2Fab-1Fab-1scDb-AFc-1; (B) 2tascFv-AFc-2; (C) AIG-2scFv-18; (D) IG-scDb-1; (E) 2Fab-scFc-1scDb-1; (F) 1Fab-AFc-1Fab-1 (ComparatorA_1)

FIG. 25 Size-related heterogeneities analyzed under denaturing, non-reducing (nR) or reducing (R) conditions by SDS-PAGE. (A) 2Fab-1Fab-1scDb-AFc-1; (B) 2tascFv-AFc-2; (C) AIG-2scFv-18; D IG-scDb-1; (E) 2Fab-scFc-1scDb-1; (F) 1Fab-AFc-1Fab-1 (ComparatorA_1)

FIG. 26 Induction of ADCP by HER2/CD16A/CD89 trispecific antibody constructs. CMFDA-labeled SK-BR-3 target cells were co-cultured for 4 h with macrophages at an E:T ratio of 1:1 in the presence of serial dilutions of trispecific HER2/CD16A/CD89 constructs AIG-2scFv-28 and AIG-1scDb-1scFv-5 or bispecific HER2/CD16A constructs AIG-1scFv-2 and AIG-1scDb-9, or without (w/o) antibody in two independent experiments (Experiment 1: A, C; Experiment 2: B, D). After incubation CD11b⁺/CMFDA⁺ phagocytic events and remaining viable CD11b⁻/CMFDA⁺ target cells were quantified by flow cytometry, and fold change in phagocytosis (A, B) and target cell depletion (C, D) were calculated using samples without (w/o) antibodies as a reference (=1). Mean and SD of duplicate values are plotted.

FIG. 27 4 h cytotoxicity assays with HER2⁺ SK-BR-3 target cells and neutrophils as effector cells in the presence of HER2/CD16A/CD89 trispecific antibody constructs. Calcein-labeled SK-BR-3 target cells were co-cultured with primary human neutrophils at the indicated E:T ratios in the presence of 3 μg/mL HER2/CD16A/CD89 constructs AIG-2scFv-28 and AIG-1scDb-1scFv-5 or HER2/CD16A constructs AIG-1scFv-2 and AIG-1scDb-9, or without (w/o) antibody. Mean and SD of duplicate lysis values are plotted.

FIG. 28 Structure information and description of trispecific molecules.

### DEFINITIONS

The term “binding domain” characterizes in connection with the present invention a domain which is capable of specifically binding to/interacting with/recognizing a given target epitope or a given target site on the target molecules (antigens), e.g. CD16A, e.g. another antigen on the surface of an immune effector cell, and/or e.g. a target cell surface antigen, respectively. The structure and/or function of the first binding domain (recognizing e.g. CD16A), the structure and/or function of the second binding domain (recognizing e.g. another antigen on the surface of an immune effector cell), and also the structure and/or function of the third binding domain (recognizing the target cell surface antigen), is/are preferably based on the structure and/or function of an antibody, e.g. of a full-length or whole immunoglobulin molecule and/or is/are drawn from the variable heavy chain (VH) and/or variable light chain (VL) domains of an antibody or fragment thereof.

The term “specifically binding”, as used herein means that the binding domain preferentially binds or recognizes the target even when the binding partner is present in a mixture of other molecules or other structures. The binding may be mediated by covalent or non-covalent interactions or a combination of both. In preferred embodiments, “simultaneous binding to a target cell and a immune effector cell” comprises the physical interaction between the binding domains and their targets on the cells, but preferably also includes the induction of an action mediated by the simultaneous binding of the two cells. Such an action may be an immune effector function of the immune effector cell, such as a cytotoxic effect.

The term “antibody construct” refers to a molecule in which the structure and/or function is/are based on the structure and/or function of an antibody, e.g., of a full-length or whole immunoglobulin molecule and/or is/are drawn from the variable heavy chain (VH) and/or variable light chain (VL) domains of an antibody or fragment thereof. An antibody construct is hence capable of binding to its specific target or antigen. Furthermore, the binding region of an antibody construct defined in the context of the invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to a specific antibody competing with the epitope of the defined antibody. The antibodies on which the constructs defined in the context of the invention are based include for example monoclonal, recombinant, chimeric, deimmunized, humanized and human antibodies.

The binding region of an antibody construct defined in the context of the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding region. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge domain; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFv-library).

An antibody construct as defined in the context of the invention may comprise a fragment of a full-length antibody, such as VH, VHH, VL, (s)dAb, Fv, Fd, Fab, Fab′, F(ab′)₂ or “r IgG” (“half antibody”). Antibody constructs as defined in the context of the invention may also comprise modified fragments of antibodies, also called antibody variants, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. A preferred linker for this purpose is a glycine serine linker, which preferably comprises from about 15 to about 30 amino acids. Preferred glycine serine linkers may have one or more repeats of GGS, GGGS (SEQ ID NO: 451), or GGGGS (SEQ ID NO: 84). Such linker preferably comprises 5, 6, 7, 8, 9 and/or 10 repeats of GGS, preferably (GGS)₆ (SEQ ID NO 82) (which are preferably used for scFvs having the arrangement VH-VL), or preferably (GGS)₇ (SEQ ID NO: 83) (which are preferably used for scFvs having the arrangement VL-VH). Single chain antibodies are discussed in detail by Plueckthun in The Pharmacology of Monoclonal Antibodies, vol. 1 13, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic. The term “bi-scFv” or “ta-scFv” (tandem scFv) as used herein refers to two scFv that are fused together. Such a bi-scFv or ta-scFv may comprise a linker between the two scFv moieties. Generally, the arrangement of the VH and VL domains on the polypeptide chain within each of the scFv may be in any order. This means that the “bi-scFv” of “ta-scFv” can be arranged in the order VH(1)-VL(1)-VH(2)-VL(2), VL(1)-VH(1)-VH(2)-VL(2), VH(1)-VL(1)-VL(2)-VH(2), or VL(1)-VH(1)-VL(2)-VH(2), where (1) and (2) stand for the first and second scFv, respectively.

The term “double Fab” as used herein refers to two Fab fragments that are fused together, which are preferably staggered. Here, a first chain of a first Fab is N-terminally fused to a first chain of a second Fab, or a second chain of a first Fab is N-terminally fused to a second chain of a second Fab, or both, the first chain of a first Fab and the second chain of a first Fab are fused to first and second chains of a second Fab, respectively. A linker may be present between the fused chains of the first and second Fab. The first and second chains of the first and second Fab can be individually selected from a light chain-derived chain of a Fab (VL-CL), a heavy chain derived chain of a Fab (VH-CH1), as long as each Fab contains a VH, a VL, a CH1, and a CL. As an illustrative example, the light chain-derived chain of the first Fab can be fused to the light chain derived-chain of the second Fab. As another illustrative example, the heavy chain-derived chain of the first Fab can be fused to the heavy chain derived-chain of the second Fab. As a further illustrative example, the heavy chain-derived chain of the first Fab can be fused to the light chain derived-chain of the second Fab. In some double Fabs, both chains of the two Fabs are fused together. For example, the light chain-derived chain of the first Fab can be fused to the light chain derived-chain of the second Fab while the heavy chain-derived chain of the first Fab can be fused to the heavy chain derived-chain of the second Fab. Alternatively, the light chain-derived chain of the first Fab can be fused to the heavy chain derived-chain of the second Fab while the heavy chain-derived chain of the first Fab can be fused to the light chain derived-chain of the second Fab. A fusion of two Fab chains may optionally comprise a linker. Suitable and preferred linkers comprise the upper hinge sequence (SEQ ID NO: 89) or glycine serine linkers with about up to 20 amino acids, preferably up to 10 amino acids, or most preferably 10 amino acids, e.g. two repeats of GGGGS (SEQ ID NO: 84). Glycine serine linkers comprised in a double Fab may have one or more repeats of GGS, GGGS (SEQ ID NO: 451), or GGGGS (SEQ ID NO: 84), such as one, two, three, or four repeats.

As used herein, a “diabody” or “db” refers to an antibody construct comprising two binding domains, which may be constructed using heavy and light chains disclosed herein, as well as by using individual CDR regions disclosed herein. Typically, a diabody comprise a heavy chain variable domain (VH) connected to a light chain variable domain (VL) by a linker which is too short to allow pairing between the two domains on the same chain. Preferred linkers for this purpose include glycine serine linkers with about up to 12 amino acids, preferably up to about 10 amino acids. Preferred glycine serine linkers may have one or more repeats of GGS, GGGS (SEQ ID NO: 451), or GGGGS (SEQ ID NO: 84). A preferred linker is (GGS)₂ SEQ ID NO: (80). Another preferred linker is (GGS)₃ SEQ ID NO: (81). Accordingly, the VH and VL domains of one fragment are forced to pair with the complementary VH and VL domains of another fragment, thereby forming two antigen-binding sites. A diabody can be formed by two separate polypeptide chains, each comprising a VH and a VL. Alternatively, all four variable domains can be comprised in one single polypeptide chain comprising two VH and two VL domains. In such a case, the diabody can also be termed “single chain diabody” or “scDb”. Typically, a scDb comprises the two chains of a non-single chain diabody that are fused together, preferably via a linker. A preferred linker for this purpose is a glycine serine linker, which preferably comprises from about 15 to about 30 amino acids. Preferred glycine serine linkers may have one or more repeats of GGS, GGGS (SEQ ID NO: 451), or GGGGS (SEQ ID NO: 84). Such linker preferably comprises 5, 6, 7, 8, 9, and/or 10 repeats of GGS, preferably (GGS)₆, (SEQ ID NO 82) or preferably (GGS)₇ (SEQ ID NO: 83). On the polypeptide chain, the variable domains of a scDb can be arranged (from N to C terminus) in a VL-VH-VL-VH or VH-VL-VH-VL order. Similarly, the spatial arrangement of the four domains in the tertiary/quaternary structure can be in a VL-VH-VL-VH or VH-VL-VH-VL order. The term diabody does not exclude the fusion of further binding domains to the diabody.

Furthermore, the definition of the term “antibody construct” includes monovalent, bivalent and polyvalent/multivalent constructs and, thus, bispecific constructs, specifically binding to only two antigenic structure, as well as polyspecific/multispecific constructs, which specifically bind more than two antigenic structures, e.g. three, four or more, through distinct binding domains. Moreover, the definition of the term “antibody construct” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The term “valent” denotes the presence of a determined number of antigen-binding domains in the antigen-binding protein. A natural IgG has two antigen-binding domains and is bivalent. The antigen-binding proteins as defined in the context of the invention are at least trivalent. Examples of tetra-, penta- and hexavalent antigen-binding proteins are described herein.

The term “trispecific” as used herein refers to an antibody construct which is “at least trispecific”, i.e., it comprises at least a first binding domain, a second binding domain, and a third binding domain, wherein the first binding domain binds to one antigen or target (here: CD16a), the second binding domain binds to another antigen or target (here: an antigen on the surface of an immune effector cell) which is not CD16A, and the third binding domain binds to another antigen or target (here: the target cell surface antigen) which is not CD16A. Accordingly, antibody constructs as defined in the context of the invention comprise specificities for at least three different antigens or targets. For example, the first binding domain does preferably bind to an extracellular epitope of an NK cell receptor of one or more of the species selected from human, Macaca spec. and rodent species.

“CD16A” or “CD16a” refers to the activating receptor CD16A, also known as FcγRIIIA, expressed on the cell surface of NK cells. CD16A is an activating receptor triggering the cytotoxic activity of NK cells. The amino acid sequence of human CD16A is given in UniProt entry P08637 (version 212 of 12 Aug. 2020) as well as in SEQ ID NO: 449. The affinity of antibodies for CD16A directly correlates with their ability to trigger NK cell activation, thus higher affinity towards CD16A reduces the antibody dose required for activation. The antigen-binding site of the antigen-binding protein binds to CD16A, but preferably not to CD16B. For example, an antigen-binding site comprising heavy (VH) and light (VL) chain variable domains binding to CD16A, but not binding to CD16B, may be provided by an antigen-binding site which specifically binds to an epitope of CD16A which comprises amino acid residues of the C-terminal sequence SFFPPGYQ (positions 201-208 of SEQ ID NO:449) and/or residues G147 and/or Y158 of CD16A which are not present in CD16B.

“CD16B” refers to receptor CD16B, also known as FcγRIIIB, expressed on neutrophils and eosinophils. The receptor is glycosylphosphatidyl inositol (GPI) anchored and is understood to not trigger any kind of cytotoxic activity of CD16B positives immune cells.

The term “target cell” describes a cell or a group of cells, which is/are the target of the mode of action applied by the antibody construct of the invention. This cell/group of cells comprise e.g. pathological cells, which are eliminated or inhibited by engaging these cells with the effector cell via the antibody construct of the invention. A preferred target cell is a cancer cell.

The term “target cell surface antigen” refers to an antigenic structure expressed by a cell and which is present at the cell surface such that it is accessible for an antibody construct as described herein. It may be a protein, preferably the extracellular portion of a protein, a peptide that is presented on the cell surface in an MHC context (including HLA-A2, HLA-All, HLA-A24, HLA-B44, HLA-C4) or a carbohydrate structure, preferably a carbohydrate structure of a protein, such as a glycoprotein. It is preferably a tumor associated or tumor restricted antigen. It is envisaged that CD16A is not a target cell surface antigen of the present invention.

The term “antibody construct” of the invention is at least trispecific but may encompass further specificities resulting in multispecific antibody constructs such as tetraspecific antibody constructs, the latter ones including four or more binding domains, or constructs having more than four (e.g. five, six . . . ) specificities. It is however envisaged, that also in these multispecific constructs it is only the first binding domain, which is CD16A specific. Examples for tri- or multispecific antibody constructs are provided e.g. in WO 2015/158636, WO 2017/064221, WO/2019/198051, and Ellwanger et a. (MAbs. 2019 July; 11(5):899-918).

Given that the antibody constructs as defined in the context of the invention are (at least) trispecific, they do not occur naturally and they are markedly different from naturally occurring products. A “trispecific” antibody construct is hence an artificial hybrid antibody having at least three distinct binding sides with different specificities. Trispecific antibody constructs can be produced by a variety of methods including fusion of hybridomas or linking of Fab′ fragments. See, e.g., Songsivilai & Lachmann, Clin. Exp. Immunol. 79:315-321 (1990).

The binding domains and the variable domains (VH/VL) of the antibody construct of the present invention may or may not comprise peptide linkers (spacer peptides). The term “peptide linker” comprises in accordance with the present invention an amino acid sequence by which the amino acid sequences of one (variable and/or binding) domain and another (variable and/or binding) domain of the antibody construct defined herein are linked with each other. The peptide linkers can also be used to fuse one domain to another domain of the antibody construct defined herein. In such cases, the peptide linker may also be referred to as a “connector”. Such a connector is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or even less. The length of the linker is preferably determined as described by Rossmalen et al Biochemistry 2017, 56, 6565-6574, which also describes suitable linkers that are well known to the skilled person. An example for a connector is a glycine serine linker or a serine linker, which preferably comprise no more than about 75 amino acids, preferably not more than about 50 amino acids. In illustrative examples, a suitable linker comprises one or more (e.g. 1, 2, 3, 4, 5, 6, 7, or 8) GGGGS sequences (SEQ ID NO: 84), such as (GGGGS)₂ (SEQ ID NO: 85), (GGGGS)₄ (SEQ ID NO: 86), or preferably (GGGGS)₆ (SEQ ID NO: 87). Other illustrative examples for linkers are shown in SEQ ID NOs: 80-83. A preferred technical feature of such peptide linker is that it does not comprise any polymerization activity.

The antibody constructs as defined in the context of the invention are preferably “in vitro generated antibody constructs”. This term refers to an antibody construct according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target cell surface antigen, (Schier, Human Antibodies Hybridomas 7 (1996), 97-105; Malmborg, J. Immunol. Methods 183 (1995), 7-13). Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et ai, Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antibody constructs involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human target cell surface antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antibody constructs of the present disclosure specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antibody construct, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antibody constructs, variants or fragments thereof (such as Fv, Fab, Fab′, F(ab′)₂ or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor ei a/., Immunology Today, 4: 7279, 1983; Olsson et al., Meth. Enzymol., 92: 3-16, 1982, and EP 239 400).

The term “human antibody”, “human antibody construct” and “human binding domain” includes antibodies, antibody constructs and binding domains having antibody regions such as variable and constant regions or domains which correspond substantially to human germline immunoglobulin sequences known in the art, including, for example, those described by Kabat et al. (1991) (loc. cit.). The human antibodies, antibody constructs or binding domains as defined in the context of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antibody constructs defined herein are “isolated” or “substantially pure” antibody constructs. “Isolated” or “substantially pure”, when used to describe the antibody constructs disclosed herein, means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antibody constructs may e.g constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antibody construct in a wide variety of organisms and/or host cells that are known in the art. In preferred embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody construct will be prepared by at least one purification step.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” or “polypeptide chain” as used herein describes a group of molecules, which usually consist of more than 30 amino acids. The terms “peptide”, “polypeptide” and “protein” also refer to naturally modified peptides/polypeptides/proteins wherein the modification is affected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below. The above modifications (glycosylation, pegylation etc.) also apply to the antibody constructs of the invention.

Preferably the binding domain which binds to CD16A, the binding domain which binds to another antigen on the surface of an immune effector cell, and/or the binding domain which binds to the target cell surface antigen is/are human binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs are expected to minimize the immunogenic and allergic responses intrinsic to mouse or mouse-derivatized mAbs and thus to increase the efficacy and safety of the administered antibodies/antibody constructs. The use of fully human antibodies or antibody constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antibody constructs comprising a human binding domain against the target cell surface antigen and a human binding domain against CD16 in order to vitiate concerns and/or effects of HAMA or HACA response.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction site”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically, a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

The interaction between the binding domain and the epitope or the region comprising the epitope implies that a binding domain exhibits appreciable affinity for the epitope/the region comprising the epitope on a particular protein or antigen (here: e.g. CD16a, another antigen on the surface of an immune effector cell, and/or the target cell surface antigen, respectively) and, generally, does not exhibit significant reactivity with proteins or antigens other than e.g. CD16a, the other antigen on the surface of an immune effector cell, and/or the target cell surface antigen. “Appreciable affinity” includes binding with an affinity of about 10⁻⁶ M (KD) or stronger. Preferably, binding is considered specific when the binding affinity is about 10⁻² to 10⁻¹ M, 10⁻¹ to 10⁻⁹ M, 10⁻¹ to 10⁻¹ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than e.g. the CD16a, the another antigen on the surface of an immune effector cell, and/or the target cell surface antigen.

The term “does not essentially/substantially bind” or “is not capable of binding” means that a binding domain of the present invention does not bind a protein or antigen other e.g. the CD16a, the other antigen on the surface of an immune effector cell, and/or the target cell surface antigen, i.e., does not show reactivity of more than 30%, preferably not more than 20%, more preferably not more than 10%, particularly preferably not more than 9%, 8%, 7%, 6% or 5% with proteins or antigens other than e.g. the CD16a, the other antigen on the surface of an immune effector cell, and/or the target cell surface antigen, whereby binding to e.g. the CD16a, the other antigen on the surface of an immune effector cell, and/or the target cell surface antigen, respectively, is set to be 100%.

Specific binding is believed to be affected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding side.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a p-sheet configuration, connected by three hypervariable regions, which form loops connecting, and in some cases forming part of, the j-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit; Chothia et al., J. Mol. Biol, 1987, 196: 901-917; and MacCallum et al., J. Mol. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. A global reference in immunoinformatics is the three-dimensional (3D) structure database of IMGT (international ImMunoGenetics information system) (Ehrenmann et al., 2010, Nucleic Acids Res., 38, D301-307). The IMGT/3Dstructure-DB structural data are extracted from the Protein Data Bank (PDB) and annotated according to the IMGT concepts of classification, using internal tools. Thus, IMGT/3Dstructure-DB provides the closest genes and alleles that are expressed in the amino acid sequences of the 3D structures, by aligning these sequences with the IMGT domain reference directory. This directory contains, for the antigen receptors, amino acid sequences of the domains encoded by the constant genes and the translation of the germline variable and joining genes. The CDR regions of our amino acid sequences were preferably determined by using the IMGT/3Dstructure database.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2nd ed., eds. Jonio et al., Academic Press, San Diego, C A, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The antibody construct defined in the context of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. Strepll-tag) and His-tag. All herein disclosed antibody constructs characterized by the identified CDRs may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antibody construct, preferably it is located at the C-terminus. Most preferably, a hexa-histidine tag is linked via peptide bond to the C-terminus of the antibody construct according to the invention. Additionally, a conjugate system of PLGA-PEG-PLGA may be combined with a poly-histidine tag for sustained release application and improved pharmacokinetic profile.

Amino acid sequence modifications of the antibody constructs described herein are also contemplated. For example, it may be desirable to improve the binding affinity and/or other biological properties of the antibody construct. Amino acid sequence variants of the antibody constructs are prepared by introducing appropriate nucleotide changes into the antibody constructs nucleic acid, or by peptide synthesis. All of the below described amino acid sequence modifications should result in an antibody construct which still retains the desired biological activity (e.g. binding to CD16a, the other antigen on the surface of an immune effector cell, and/or the target cell surface antigen) of the unmodified parental molecule.

The term “amino acid” or “amino acid residue” typically refers to an amino acid having its art recognized definition such as an amino acid selected from the group consisting of alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, Ile, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within a third binding domain of the antibody construct defined in the context of the invention. An insertional variant of the antibody construct defined in the context of the invention includes the fusion to the N-terminus or to the C-terminus of the antibody construct of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antibody constructs that are preferred locations for mutagenesis is called “alanine scanning mutagenesis” as described by Cunningham and Wells in Science, 244: 1081-1085 (1989). Here, a residue or group of target residues within the antibody construct is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as for the binding to e.g. CD16a, the other antigen on the surface of an immune effector cell, and/or the target cell surface antigen binding.

Generally, if amino acids are substituted in one or more or all of the CDRs of the heavy and/or light chain, it is preferred that the then-obtained “substituted” sequence is at least 60% or at least 65%, more preferably at least 70% or at least 75%, even more preferably at least 80% or at least 85%, and particularly preferably at least 90% or at least 95% identical to the “original” CDR sequence. This means that it is dependent of the length of the CDR to which degree it is identical to the “substituted” sequence. For example, a CDR having 5 amino acids is preferably at least 80% identical to its substituted sequence in order to have at least one amino acid substituted. Accordingly, the CDRs of the antibody construct may have different degrees of identity to their substituted sequences, e.g., CDRL1 may have at least 80%, while CDRL3 may have at least 90%.

Preferred substitutions (or replacements) are conservative substitutions. However, any substitution (including non-conservative substitution) is envisaged as long as the antibody construct retains its capability to bind to e.g. the CD16a via the first binding domain, to the other antigen on the surface of an immune effector cell via the second binding domain, and/or to the target cell surface antigen via the third binding domain and/or its CDRs have an identity to the then substituted sequence (at least 60% or at least 65%, more preferably at least 70% or at least 75%, even more preferably at least 80% or at least 85%, and particularly preferably at least 90% or at least 95% identical to the “original” CDR sequence).

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened for a desired characteristic.

TABLE 1
Amino acid substitutions

| Original | Exemplary Substitutions | Preferred Substitutions |
|---|---|---|
| Ala (A) | val, leu, ile | val |
| Arg (R) | lys, gln, asn | lys |
| Asn (N) | gln, his, asp, lys, arg | gln |
| Asp (D) | glu, asn | glu |
| Cys (C) | ser, ala | ser |
| Gln (Q) | asn, glu | asn |
| Glu (E) | asp, gln | asp |
| Gly (G) | ala | ala |
| His (H) | asn, gln, lys, arg | arg |
| Ile (I) | leu, val, met, ala, phe | leu |
| Leu (L) | norleucine, ile, val, met, ala | lie |
| Lys (K) | arg, gln, asn | arg |
| Met (M) | leu, phe, ile | leu |
| Phe (F) | leu, val, ile, ala, tyr | tyr |
| Pro (P) | ala | ala |
| Ser (S) | thr | thr |
| Thr (T) | ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=ll. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antibody construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL defined in the context of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antibody constructs according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antibody constructs leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antibody constructs. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

The term “EGFR” refers to the epidermal growth factor receptor (EGFR; ErbB-1; HER1 in humans, including all isoforms or variants described with activation, mutations and implicated in pathophysiological processes. The EGFR antigen-binding site recognizes an epitope in the extracellular domain of the EGFR. In certain embodiments the antigen-binding site specifically binds to human and cynomolgus EGFR. The epidermal growth factor receptor (EGFR) is a member of the HER family of receptor tyrosine kinases and consists of four members: EGFR (ErbB1/HER1), HER2/neu (ErbB2), HER3 (ErbB3) and HER4 (ErbB4). Stimulation of the receptor through ligand binding (e.g. EGF, TGFa, HB-EGF, neuregulins, betacellulin, amphiregulin) activates the intrinsic receptor tyrosine kinase in the intracellular domain through tyrosine phosphorylation and promotes receptor homo- or heterodimerization with HER family members. These intracellular phospho-tyrosines serve as docking sites for various adaptor proteins or enzymes including SHC, GRB2, PLCg and PI(3)K/Akt, which simultaneously initiate many signaling cascades that influence cell proliferation, angiogenesis, apoptosis resistance, invasion and metastasis.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukaemia, chronic lymphocyte leukaemia and non-Hodgkin lymphoma. It is also an early marker of B cell progenitors. See, e.g., Nicholson et al. Mol. Immun. 34 (16-17): 1157-1165 (1997).

The term “immune effector cell” as used herein may refer to any leukocyte or precursor involved e.g. in defending the body against cancer, diseases induced by infectious agents, foreign materials or autoimmune reactions. For example, the immune effector cells comprise B lymphocytes (B cells), T lymphocytes (T cells, including CD4+ and CD8+ T cells), NK cells, NKT cells, monocytes, macrophages, dendritic cells, mast cells, granulocytes such as neutrophils, basophils and eosinophils, innate lymphoid cells (ILCs, which comprise ILC-1, ILC-2 and ILC-3) or any combinations thereof. Preferably, the term immune effector cell refers to an NK cell, an ILC-1 cell, a NKT cell, a macrophage, a monocyte, and/or a T cell, such as a CD8+ T cell or a γδ T cell.

Natural killer (NK) cells are CD56+CD3− large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+ T lymphocytes, NK cells launch cytotoxicity against tumor cells without the requirement for prior sensitization and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

Monocytes are produced by the bone marrow from haematopoietic stem cell precursors called monoblasts. Monocytes circulate in the bloodstream for about one to three days and then typically move into tissues throughout the body. They constitute between three to eight percent of the leukocytes in the blood. In the tissue monocytes mature into different types of macrophages at different anatomical locations. Monocytes have two main functions in the immune system: (1) replenish resident macrophages and dendritic cells under normal states, and (2) in response to inflammation signals, monocytes can move quickly (approx. 8-12 hours) to sites of infection in the tissues and divide/differentiate into macrophages and dendritic cells to elicit an immune response. Monocytes are usually identified in stained smears by their large bilobate nucleus.

Macrophages are potent effectors of the innate immune system and are capable of at least three distinct anti-tumor functions: phagocytosis, cellular cytotoxicity, and antigen presentation to orchestrate an adaptive immune response. While T cells require antigen-dependent activation via the T cell receptor or the chimeric immunoreceptor, macrophages can be activated in a variety of ways. Direct macrophage activation is antigen-independent, relying on mechanisms such as pathogen associated molecular pattern recognition by Toll-like receptors (TLRs). Immune-complex mediated activation is antigen dependent but requires the presence of antigen-specific antibodies and absence of the inhibitory CD47-SIRPa interaction.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, TH9, or TFH, which secrete different cytokines to facilitate a different type of immune response.

Cytotoxic T cells (TC cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described-naturally occurring Treg cells and adaptive Treg cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

As used herein, the term “half-life extensions domain” relates to a moiety that prolongs serum half-life of the antibody construct. The half-life extension domain may comprise a portion of an antibody, such as an Fc part of an immunoglobulin, a hinge domain, a CH2 domain, a CH3 domain, and/or a CH4 domain. Although less preferred, a half-life extension domain can also comprise elements that are not comprised in an antibody, such as an albumin binding peptide, an albumin binding protein, or transferrin to name only a few. A half-life extension domain preferably does not have an immune-modulatory function. If a half-life extension domain comprises a hinge, CH2 and/or CH3 domain, the half-life extension domain preferably does not essentially bind to an Fc receptor. This can e.g. be achieved through “silencing” of the Fcγ receptor binding domain.

As used herein, “silencing” of the Fc or Fcγ receptor binding domain refers to any modification that reduces binding of a CH2 domain to an Fc receptor, in particular an Fcγ receptor. Such modification can be done by replacement and/or deletion of one or more amino acids that are involved in Fc(γ) receptor-binding. Such mutations are well known in the art and have e.g. been described by Saunders (2019, Front. Immunol. 10:1296). For example, a mutation can be located at any one of the positions 233, 234, 235, 236, 237, 239, 263, 265, 267, 273, 297, 329, and 331. Examples for such mutations are: deletion of Glu 233->Pro, Glu 233, Leu 234->Phe, Leu 234->Ala, Leu 234->Gly, Leu 234->Glu, Leu 234->Val, deletion of Leu 234, Leu 235->Glu, Leu 235->Ala, Leu 235->Arg, Leu 235->Phe, deletion of Leu 235, deletion of Gly 236, Gly 237->Ala, Ser 239->Lys, Val 263->Leu, Asp 265->Ala, Ser 267->Lys, Val 273->Glu, Asn 297->Gly, Asn 297->Ala, Lys 332->Ala, Pro 329->Gly, Pro 331->Ser and combinations thereof. Preferably, such a modification comprises one or both of Leu 234->Ala and Leu 235->Ala (also known as “LALA” mutation). Preferably, such a modification further comprises a Pro 329->Gly mutation, also known as “LALA-PG” mutation (Leu 234->Ala, Leu 235->Ala, and Pro 329->Gly). Preferably, such a modification comprises 1, 2, or 3 of the mutations Leu 234->Phe, Leu 235->Glu, and Asp 265->Ala, more preferably all three of these mutations. The combination Leu 234->Phe, Leu 235->Glu, and Asp 265->Ala, which is a preferred modification in the context of the present invention, is also known as “FEA” mutation. Preferably, such a modification further comprises Asn 297->Gly. Such a preferred modification comprises the mutations Leu 234->Phe, Leu 235->Glu, Asp 265->Ala, and Asn 297->Gly.

The term “fratricide” describes in the context of the invention the reduction of effector cells by cytotoxic kill and, thereby the reduction of the available effector cell population/compartment. Fratricide can be caused by cross-linking of two immune cells. As an illustrative example, cross-linking of NK cells can cause the killing of either one or both of the NK cells. Since the antibody construct in some embodiments recruits two different types of effector cells, e.g. NK cells and macrophages or NK cells and T cells also the elimination of one type of effector cells by the other type of effector cells is understood as fratricide in the context of the invention. Fratricide can be e.g. measured in an assay as essentially described in Example 12 or 13.

### DETAILED DESCRIPTION

Innate immune effector cells (e.g. natural killer (NK) cells, macrophages) are activated by a complex mechanism of several different signaling pathways. NK cells and macrophages can be harnessed in cancer immunotherapy by redirecting NK cell lysis or macrophage-induced phagocytosis to tumor cells through stimulation of the activating antigen CD16A (FcγRIIIA) expressed on their cell surface. CD16A is associated with the signaling adaptor CD3ζ chain containing an immunoreceptor tyrosine-based activation motif (ITAM), initiating signaling cascades that ultimately mediate ADCC and ADCP in NK cells and macrophages, respectively.

Signaling via CD16A has been reported sufficient to activate the cytotoxic activity of NK cells. However, in circumstances of e.g. an immunosuppressive tumor microenvironment stimulation via CD16A may be suboptimal or insufficient for maximal anti-tumor activity. Therefore, targeting of an additional surface antigen on NK cells, macrophages, or other immune cell types such as, but not limited to, CD8+ αβ T cells or γδ T cells may improve or maximize anti-tumor activity.

However, since cross-linking of two immune effector cell may result in fratricide of immune effector cells, the present invention aims at providing an antibody construct that is capable of simultaneously binding an immune effector cell via either the first binding domain (A) or the second binding domain (B) and a target cell via the third binding domain (C), while the capacity of the antibody construct to simultaneously bind to two different immune effector cells, e.g. two different NK cells or an NK cell and a macrophage or T cell, is reduced or preferably even absent. This may be achieved by adjusting the distance of binding sites of the first binding domain (A) and the second binding domain (B). This may also be achieved by adjusting the spatial orientation of the first binding domain (A) and the second binding domain (B) relative to each other. Accordingly, the antibody construct of the present invention preferably binds to a target cell and one immune effector cell simultaneously. In this context, “one” is preferably to be understood as “only one” or “not more than one”.

The present invention thus envisions an antibody construct comprising a first binding domain (A), which is capable of specifically binding to a first target (A′) that is CD16A, a second binding domain (B), which is capable of specifically binding to a second target (B′) that is an antigen on the surface of an immune effector cell, which is not CD16A, and a third binding domain (C), which is capable of specifically binding a third target (C′) that is an antigen on the surface of a target cell. Accordingly, the antibody construct of the invention is at least trispecific.

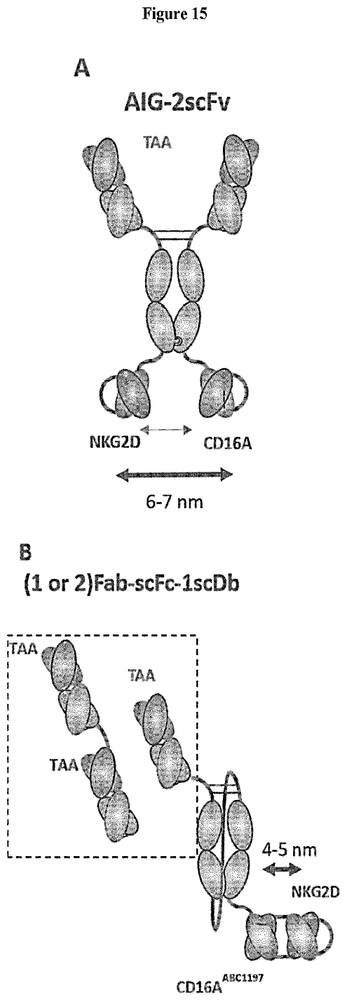

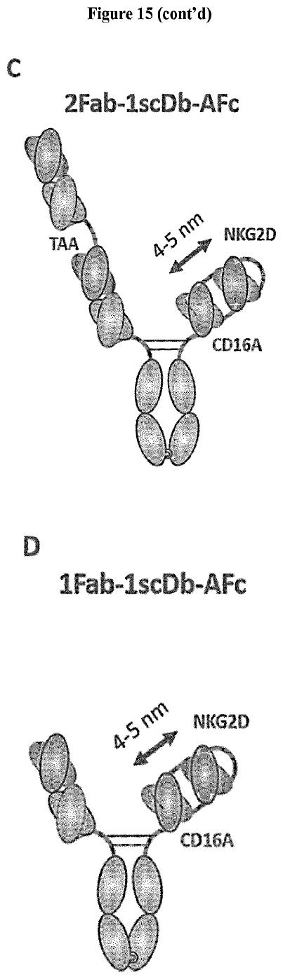

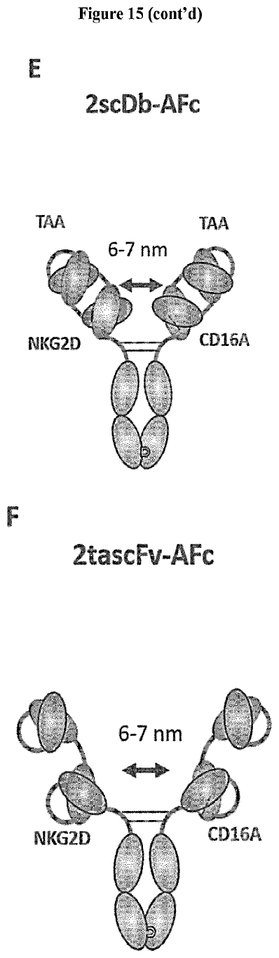

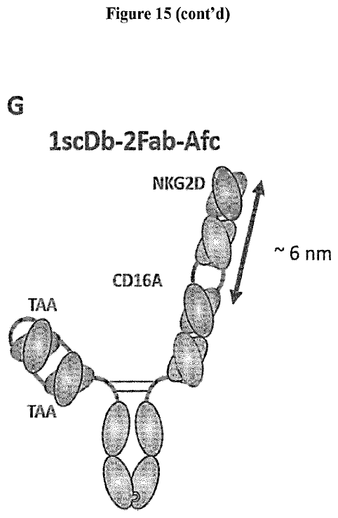

Without wishing to be bound by theory, the inventors of the present application believe that the binding sites of the first binding domain (A) and the second binding domain (B) must have at least a certain distance to each other to have the capacity of simultaneous binding of two immune effector cells. This is based on the assumption that there is a minimum possible distance between two neighboring cells. It is assumed that this minimum possible distance is in the range of about 10-30 nm (i.e. ≥10 nm), which corresponds to the size of the immunological synapse (sometimes also denoted synaptic cleft) between an immune effector cell (e.g. NK cell) and its target cell (cf. Mace et al., Immunol Cell Biol. 2014 March; 92(3): 245-255; McCann et al., J Immunol. 2003 Mar. 15; 170(6):2862-70). In line with this consideration, it is further believed that there is a transition range above the theoretical minimum distance, where an antibody construct's capacity to simultaneously engage two different immune effector cells is reduced due to emerging steric hindrance at shorter distances between the binding sites of the first binding domain (A) and the second binding domain (B). It is thus believed that if the distance between the first binding domain (A) and the second binding domain (B) is small in an antibody construct comprising a first binding domain (A) that is specific for CD16A and a second binding domain (B) that is specific for another target on an immune effector cell (e.g. NKp46), the antibody construct's capacity of simultaneously binding to two immune effector cells will be significantly reduced. This is because antibody constructs having short distances between both engager domains are less accessible for the second immune effector cells, which results in a lower likelihood of binding of the second immune effector cell. At even smaller distances, at which the distance is too short to bridge the minimum possible distance between two immune effector cells, it is assumed that the antibody construct's capacity of simultaneously binding to two immune effector cells is essentially absent. A reduced or impaired simultaneous binding of two different immune effector cells is believed to reduce or impair fratricide. A distance between the engager domains and preferably between the antigen binding sites of the engager domains at which the antibody construct's capacity to simultaneously bind to two different immune effector cells is reduced is preferably about 25 nm or less (illustrated exemplarily in FIG. 15). However, even shorter distances are more preferred because it is believed that the shorter the distance between the engager domains and preferably the antigen-binding sites of the two engager domains is, the stronger will be the reduction of the antibody construct's capacity of simultaneously binding two immune effector cells. Thus, a more preferred distance between the engager domains and preferably the antigen-binding sites of the two engager domains (first binding domain (A) and second binding domain (B)) is about 20 nm or less, even more preferred is a distance of about 15 nm or less, even more preferred is a distance of about 10 nm or less. At distances below about 10 nm in an antibody construct of the invention, in particular for antibody constructs in which the first binding domain (A) is specific for CD16A and the second binding domain (B) is specific for NKG2D or NKp46, it is believed that simultaneous binding to different immune effector cells via these two binding domains is essentially absent.

The antibody construct of the invention is characterized by inducing a low degree of fratricide, which is also referred to as a “sufficiently reduced” degree of fratricide. The degree of fratricide can be measured in a cytotoxicity assay, such as an assay as essentially described in Example 8. Such an assay is preferably conducted as follows. For calcein-release cytotoxicity assays to assess NK-NK cell lysis, half of the enriched, non-activated NK cells were washed with RPMI 1640 medium without FCS and labeled with 10 μM calcein AM (Invitrogen/Molecular Probes, cat.: C3100MP) for 30 min in RPMI 1640 medium without FCS at 37° C. After gentle washing, the labeled cells were resuspended in complete RPMI medium (RPMI 1640 medium supplemented with 10% heat-inactivated FCS, 4 mM L-glutamine, 100 U/mL penicillin G sodium, 100 μg/mL streptomycin sulfate) to a density of 5×10⁵/mL. 5×10⁴ calcein-labeled NK cells (E) were then seeded together with 5×10⁴ non-labeled NK cells (T) from the same donor at an E:T ratio of 1:1 in the presence of increasing concentrations of the indicated antibodies, preferentially in the range between 10 ng/mL and 100 μg/mL, in individual wells of a round-bottom 96-well microplate in a total volume of 200 μL/well in duplicates. Human IgG1 anti-CD38 (IgAb_51, SEQ ID NOs: 429 and 430 can be used as a positive control). Spontaneous release, maximal release and killing of targets by effectors (E) in the absence of antibodies are determined in quadruplicate on each plate. For induction of maximal calcein-release Triton X-100 was added to the respective wells at a final concentration of 1%. After centrifugation for 2 min at 200×g the assay was incubated for 4 h at 37° C. in a humidified atmosphere with 5% CO₂. After an additional centrifugation for 5 min at 500×g 100 μL cell culture supernatant were harvested from each well, transferred to a black flat-bottom microplate, and the fluorescence of the released calcein was measured at 520 nm using a fluorescence plate reader (EnSight, Perkin Elmer). On the basis of the measured fluorescence counts, the specific cell lysis was calculated according to the following formula: [fluorescence (sample)−fluorescence (spontaneous)]/[fluorescence (maximum)−fluorescence (spontaneous)]×100%. Fluorescence (spontaneous) represents the fluorescent counts from calcein-labeled NK cells (T) in the absence of non-labeled NK cells and antibodies and fluorescence (maximum) represents the total cell lysis induced by the addition of Triton X-100 (1% final concentration). The degree of fratricide is preferably determined at a concentration of 100 μg/mL of the test antibody and/or the control.

The afore-mentioned assay is preferably used for determining NK-NK cell lysis. However, if the second binding site (B) binds to a second target (B′) that is expressed on the surface of another immune effector cell, such as a T cell, the assay can be adapted to measure NK cell-mediated lysis (fratricide) of the other immune effector cell, such as NK-T cell lysis. For this purpose, the population of cells, of which the lysis should be measured, can be labeled with calcein AM (instead of using the calcein-labeled NK cells as described above). For example, if NK-T cell lysis is to be measured, the calcein-labeled NK cells as described above should be replaced with calcein-labeled T cells. The remaining steps of the assay are essentially the same. In preferred embodiments, “fratricide” relates to NK cell-mediated lysis of a given immune effector cell. This means that the population of cells, of which lysis should be measured should be a population that expresses the second target (B′) on its surface.

In some embodiments, a “low degree of fratricide” means that the degree of fratricide of a test molecule, such as an antibody construct of the invention, is about 25% or lower. The degree of fratricide of an antibody construct of the invention is preferably about 22% or lower, more preferably about 20% or lower, more preferably about 19% or lower, more preferably about 18% or lower, more preferably about 17% or lower, more preferably about 16% or lower, more preferably about 15% or lower, more preferably about 14% or lower, more preferably about 13% or lower, more preferably about 12% or lower, more preferably about 11% or lower, more preferably about 10% or lower, preferably determined at a concentration of 100 μg/mL. In some even more preferred embodiments, the degree of fratricide of an antibody of the invention is even lower, such as preferably about 9% or lower, more preferably about 8% or lower, more preferably about 7% or lower, more preferably about 6% or lower, more preferably about 5% or lower, more preferably about 4% or lower, more preferably about 3% or lower, more preferably about 2% or lower, or more preferably about 1% or lower, or most preferably non-detectable with an assay essentially described herein, preferably as defined supra, preferably determined at a concentration of 100 μg/mL.

In some embodiments, an antibody construct of the invention induces a degree of fratricide that is lower as compared to the anti-CD38 antibody shown in SEQ ID NOs: 429-430, preferably determined at a concentration of 100 μg/mL of the test antibody and the control.

In some embodiments, an antibody construct of the invention induces a degree of fratricide that that is lower as compared to a control antibody as show in SEQ ID NOs: 393-395, preferably determined at a concentration of 100 μg/mL of the test antibody and the control. In some embodiments, an antibody construct of the invention induces a degree of fratricide that is lower as compared to a control antibody as show in SEQ ID NOs: 396-398, preferably determined at a concentration of 100 μg/mL of the test antibody and the control. In some embodiments, an antibody construct of the invention induces a degree of fratricide that that is lower as compared to a control antibody as show in SEQ ID NOs: 399-401, preferably determined at a concentration of 100 μg/mL of the test antibody and the control. In some embodiments, an antibody construct of the invention induces a degree of fratricide that is lower as compared to a control antibody as show in SEQ ID NOs: 402-404, preferably determined at a concentration of 100 μg/mL of the test antibody and the control. In some embodiments, an antibody construct of the invention induces a degree of fratricide that is lower as compared to a control antibody as show in SEQ ID NOs: 405-407, preferably determined at a concentration of 100 μg/mL of the test antibody and the control. In some embodiments, an antibody construct of the invention induces a degree of fratricide that is lower as compared to a control antibody as show in SEQ ID NOs: 408-410, preferably determined at a concentration of 100 μg/mL of the test antibody and the control. In some embodiments, an antibody construct of the invention induces a degree of fratricide that is lower as compared to a control antibody as show in SEQ ID NOs: 411-413, preferably determined at a concentration of 100 μg/mL of the test antibody and the control. In some embodiments, an antibody construct of the invention induces a degree of fratricide that is lower as compared to a control antibody as show in SEQ ID NOs: 414-416, preferably determined at a concentration of 100 μg/mL of the test antibody and the control. In some embodiments, an antibody construct of the invention induces a degree of fratricide that is lower as compared to a control antibody as show in SEQ ID NOs: 417-419, preferably determined at a concentration of 100 μg/mL of the test antibody and the control. In some embodiments, an antibody construct of the invention induces a degree of fratricide that is lower as compared to a control antibody as show in SEQ ID NOs: 420-422, preferably determined at a concentration of 100 μg/mL of the test antibody and the control. In some embodiments, an antibody construct of the invention induces a degree of fratricide that is lower as compared to a control antibody as show in SEQ ID NOs: 423-425, preferably determined at a concentration of 100 μg/mL of the test antibody and the control. In some embodiments, an antibody construct of the invention induces a degree of fratricide that is lower as compared to a control antibody as show in SEQ ID NOs: 426-428, preferably determined at a concentration of 100 μg/mL of the test antibody and the control.

In some embodiments, an antibody construct of the invention induces a degree of fratricide that is lower as compared to a control antibody construct that has a format as essentially shown in FIG. 11, wherein the third binding domains (C) of the control antibody construct and the antibody construct of the invention have the same CDR sequences, or preferably the same VH and VL regions, and wherein the second binding domains (B) of the control antibody construct and the antibody construct of the invention have the same CDR sequences, or preferably the same VH and VL regions, and wherein the control antibody construct comprises a CH2 domain in which the Fcγ receptor binding domain has not been silenced, preferably determined at a concentration of 100 μg/mL of the test antibody and the control.

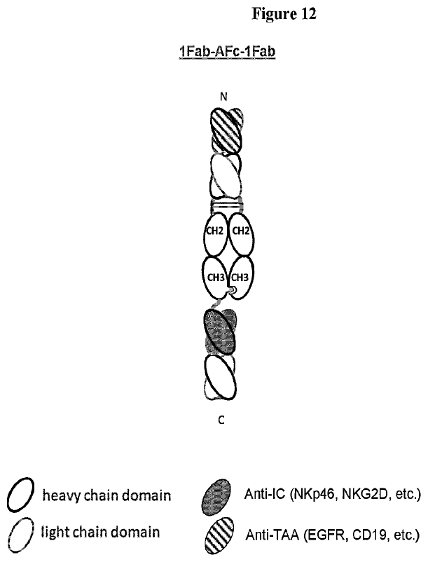

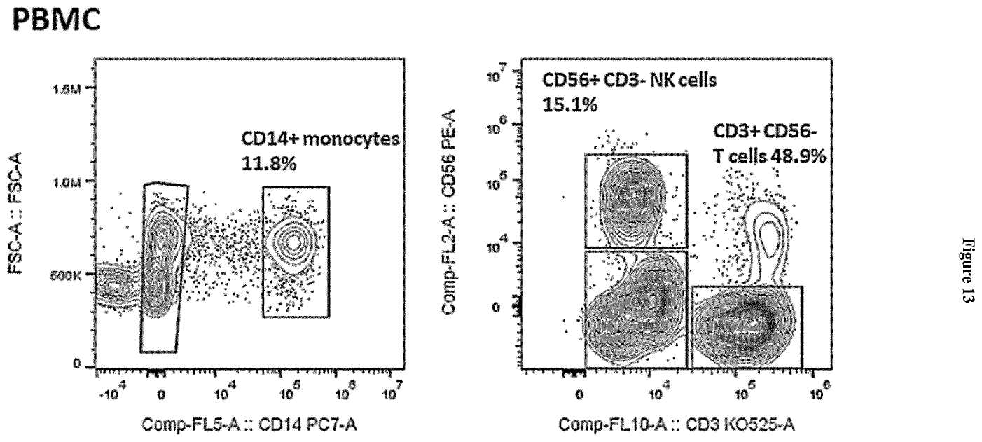

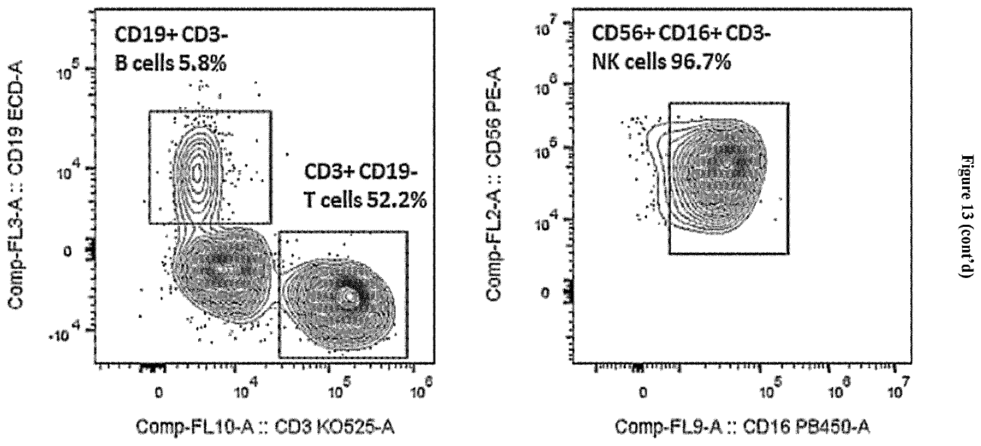

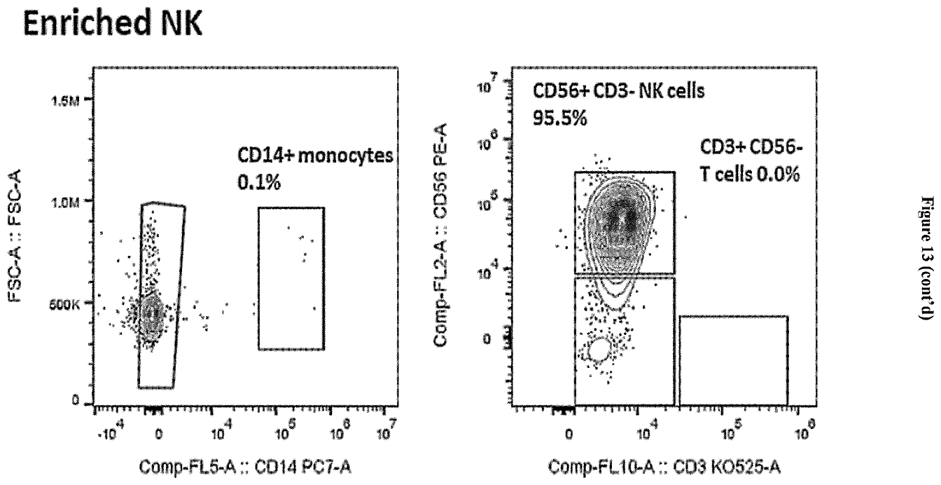

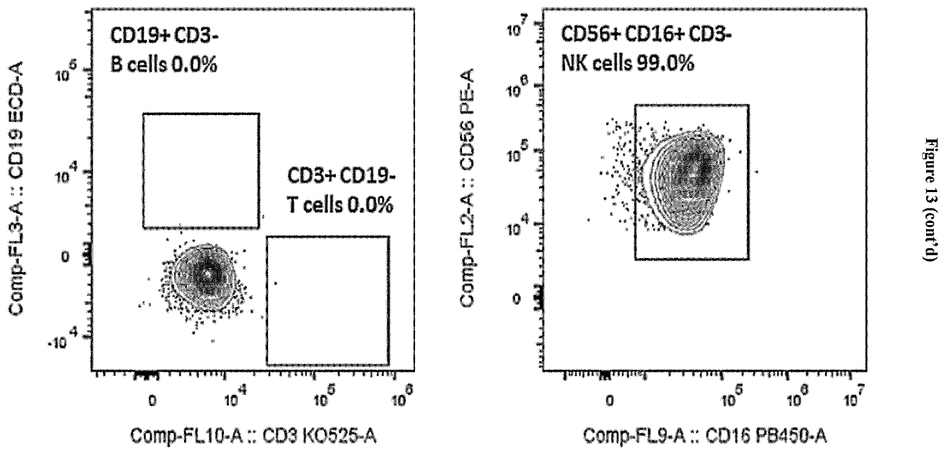

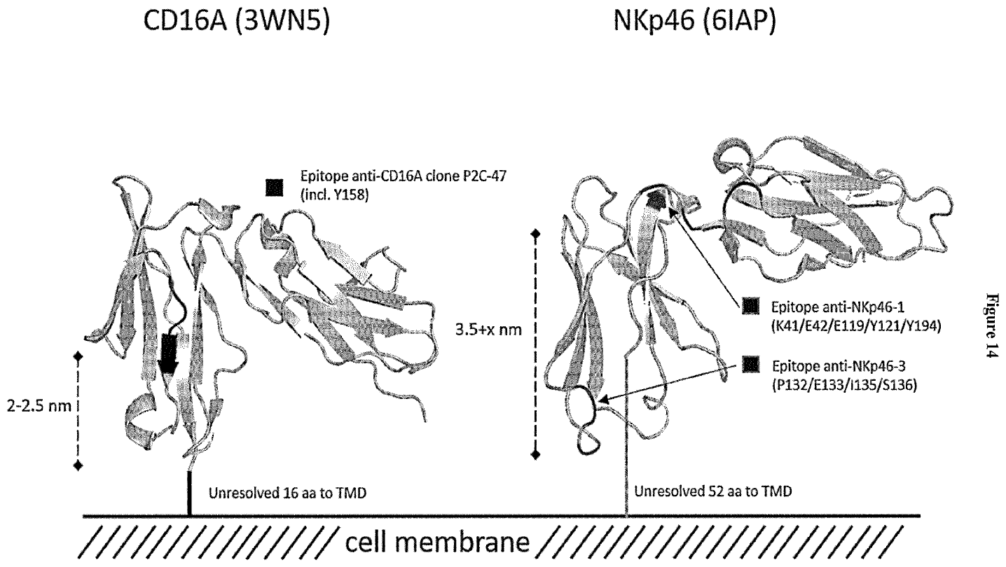

In some embodiments, an antibody construct of the invention induces a degree of fratricide that is lower as compared a control antibody construct that has a format as essentially shown in FIG. 12, wherein the third binding domains (C) of the control antibody construct and the antibody construct of the invention have the same CDR sequences, or preferably the same VH and VL regions, and wherein the second binding domains (B) of the control antibody construct and the antibody construct of the invention have the same CDR sequences, or preferably the same VH and VL regions, and wherein the control antibody construct comprises a CH2 domain in which the Fcγ receptor binding domain has not been silenced, preferably determined at a concentration of 100 μg/mL of the test antibody and the control.

The antibody construct of the disclosure may comprise a fourth domain (D), which comprises a half-life extension domain as described herein. The half-life extension domain may comprise a CH2 domain, in which the Fcγ receptor binding domain of the CH2 domain is silenced. The half-life extension domain may comprise two such CH2 domains. Whenever a half-life extension domain comprises a CH2 domain, the Fcγ receptor binding domain of the CH2 domain is silenced. The half-life extension domain may comprise a CH3 domain. The half-life extension domain may comprise two CH3 domains. The half-life extension domain may comprise a hinge domain. The half-life extension domain may comprise two hinge domains. The half-life extension domain may comprise a CH2 domain and a CH3 domain. In such a case, the CH2 domain and CH3 domain are preferably fused to each other, preferably in the (amino to carboxyl) order CH2 domain-CH3 domain. Non-limiting examples for such fusions are shown in SEQ ID NOs: 97-105. The half-life extension domain may comprise a hinge domain and a CH2 domain. In such a case, the hinge domain and the CH2 domain are preferably fused to each other, preferably in the (amino to carboxyl) order hinge domain-CH2 domain. The half-life extension domain may comprise a hinge domain, a CH2 domain, and a CH3 domain. In such a case, the hinge domain, the CH2 domain, and CH3 domain are preferably fused to each other, preferably in the (amino to carboxyl) order hinge domain-CH2 domain-CH3 domain. The half-life extending domain may comprise two hinge domain-CH2 domain elements, two CH2 domain-CH3 domain elements, or two hinge domain-CH2 domain-CH3 domain elements. In such a case the two fusions may be located on two different polypeptide strands. Alternatively, the fusions can be located on the same polypeptide strand. An illustrative example for two hinge domain-CH2 domain-CH3 domain elements that are located on the same polypeptide strand is the “single chain Fc” or “scFc” format. Here, both hinge-CH2-CH3 subunits are fused together via a linker that allows assembly of a Fc domain. A preferred linker for this purpose is a glycine serine linker, which preferably comprises from about 20 to about 40 amino acids. Preferred glycine serine linkers may have one or more repeats of GGS, GGGS (SEQ ID NO: 451), or GGGGS (SEQ ID NO: 84). Such linker preferably comprises 4-8 repeats (e.g. 4, 5, 6, 7, or 8 repeats) of GGGGS. Such a linker is preferably (GGGGS)₆, (SEQ ID NO 87). Illustrative examples for such scFc domains are shown in SEQ ID NOs 106-107. Further scFc constant domains are known in the art and inter alia described in WO 2017/134140.

The first binding domain (A) is preferably derived from an antibody. The first binding domain (A) preferably comprises a VH and a VL domain of an antibody. Preferred structures for the first binding domain (A) include a Fv, a scFv, a Fab, or a VL and VH pair which may be comprised in a diabody (db), scDb or a double Fab.

The second binding domain (B) is also preferably derived from an antibody. The second binding domain (B) preferably comprises a VH and a VL domain of an antibody. Preferred structures for the second binding domain (B) include a Fv, a scFv, a Fab, or a VL and VH pair which may be comprised in a diabody (db), scDb or a double Fab.

The third binding domain (C) is also preferably derived from an antibody. The third binding domain (C) preferably comprises a VH and a VL domain of an antibody. Preferred structures for the third binding domain (C) include a Fv, a scFv, a Fab, or a VL and VH pair which may be comprised in a diabody (db), scDb or a double Fab.

In order to provide a short distance between the first binding domain (A) and the second binding domain (B) both domains may be fused to adjacent positions or fused to each other. For example, the first binding domain (A) and the second binding domain (B) can be fused to a pair (e.g. a dimer) of two constant domains of an antibody, such as a pair of two CH3 domains, a pair of two CH2 domains, or a pair of a CH1 domain and a CL domain. In such a case, it is preferred that both the first binding domain (A) and the second binding domain (B) are fused to the C termini of the pair of the two constant domains or that both the first binding domain (A) and the second binding domain (B) are fused to the N termini of the pair of the two constant domains. In a preferred embodiment, the first binding domain (A) is fused to the C terminus of a first CH3 domain and the second binding domain (B) is fused to the C terminus of a second CH3 domain. The third binding domain (C) can be located at any suitable position of the antibody construct.

Generally, the antibody constructs of the disclosure can be monovalent, bivalent, trivalent, or have an even higher valency for any one of the first target (A′), the second target (B′), and/or the third target (C′). The antibody constructs of the disclosure may thus comprise one, two, three, or even more of any one of the first binding domain (A), the second binding domain (B), or the third binding domain (C). It is preferred for the antibody construct of the invention that it is at least bivalent for the first target (A′) and the second target (B′). It is further preferred for the antibody construct of the invention that it is monovalent for the first target (A′) and at least bivalent for the second target (B′). More preferably, the antibody construct of the invention is monovalent for the first target (A′) and the second target (B′). It is preferred for the antibody construct of the invention that it comprises at least two first binding domains (A) and at least two second binding domains (B). It is further preferred for the antibody construct of the invention that it comprises one first binding domain (A) and at least two second binding domains (B). More preferably, the antibody construct of the invention comprises one first binding domain (A) and one second binding domain (B). It also preferred that the antibody construct of the invention is monovalent for the third target (C′). It is also preferred that the antibody construct of the invention is at least trivalent for the third target (C′). It is more preferred that the antibody construct of the invention is bivalent for the third target (C′). It is also preferred for the antibody construct of the invention that it comprises one third binding domain (C). It is also preferred for the antibody construct of the invention that it comprises at least three third binding domains (C). It is more preferred for the antibody construct of the invention that it comprises two third binding domains (C).

It is preferred for the antibody construct of the invention that it is at least bivalent for CD16A and the antigen on the surface of an effector cell, which is not CD16A. It is further preferred for that antibody construct, that it is monovalent for CD16A and at least bivalent for the antigen on the surface of an effector cell, which is not CD16A. More preferably, the antibody construct of the invention is monovalent for CD16A and the antigen on the surface of an effector cell, which is not CD16A.

In a preferred embodiment, the first binding domain (A) and second binding domain (B) are fused to two C termini of a Fc region. Such a fusion format is illustratively shown in FIG. 7. The first binding domain (A) and/or second binding domain (B) may be fused to a constant domain of an antibody via a linker. Such a linker is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or even less. The length of the linker is preferably determined as described by Rossmalen et al Biochemistry 2017, 56, 6565-6574, which also describes suitable linkers that are well known to the skilled person. An example for a suitable linker is a glycine serine linker or a serine linker, which preferably comprise no more than about 75 amino acids, preferably not more than about 50 amino acids. In illustrative example, a suitable linker comprises one or more (e.g. 1, 2, 3, 4, 5, 6, 7, or 8) GGGGS sequences (SEQ ID NO: 84), such as (GGGGS)₂ (SEQ ID NO: 85), (GGGGS)₄ (SEQ ID NO: 86), or preferably (GGGGS)₆ (SEQ ID NO: 87). Other illustrative examples for linkers are shown in SEQ ID NOs: 80-83. The first binding domain (A) and/or the second binding domain (B) are preferably scFv fragments that are fused to two C termini of a Fc domain, preferably via the VL domain of the scFv. Accordingly, the arrangement of the polypeptide chain (from N to C) is preferably . . . -CH2-CH3-VL-VH, optionally with a linker between the Fc and the scFv. The third binding domain can be located at any suitable position of the antibody construct. Where the antibody construct comprises a Fc region, the third binding domain (C) can be located N terminal of the Fc region, either directly or linked via at least a part of a hinge domain. Other linkers disclosed herein can also be used to link the third binding domain to the Fc domain. A hinge domain is however preferred for this purpose. The third binding domain (C) can be any suitable structure disclosed herein, while a Fab structure is preferred.

An antibody construct of the invention is preferably in a format as essentially shown in FIG. 7 and which is also referred to as “AIG-2scFv”. Such an antibody construct comprises an immunoglobulin that has two scFv fragments fused to the C termini of the heavy chains, optionally via a linker, which is preferably a connector, disclosed herein. One of the two scFv forms the first binding domain (A), while the other scFv forms the second binding domain (B). Two third binding domains (C) are formed by the binding sites of the immunoglobulin. The AIG-2scFv format may comprise four polypeptide chains, two light chains in the arrangement VL(C)-CL, one heavy chain fused to a scFv in the arrangement VH(C)-CH1-hinge-CH2-CH3-VL(A)-VH(A) (or less preferred VH(C)-CH1-hinge-CH2-CH3-VH(A)-VL(A)), and one heavy chain fused to a scFv in the arrangement VH(C)-CH1-hinge-CH2-CH3-VL(B)-VH(B) (or less preferred VH(C)-CH1-hinge-CH2-CH3-VH(B)-VL(B)). The letters in parenthesis stand for first binding domain (A), second binding domain (B), or third binding domain (C), respectively. For example, VL(A) stands for a VL domain of a first binding domain (A), while VH(B) stands for a VH domain of a second binding domain (B). Illustrative examples for such antibody constructs are shown in SEQ ID NOs: 329-331; 332-334; 335-337; 338-340, 490-492, and 493-495.

In a preferred embodiment, two first binding domains (A) and two second binding domains (B) are fused to two C termini of a Fc region. Such a fusion format is illustratively shown in FIG. 9. The two first binding domains (A) are preferably fused together in form of a diabody or single chain diabody, preferably via a VL domain of a first binding domain (A). Likewise, the two second binding domains (B) are preferably fused together in form of a diabody or single chain diabody, preferably via a VL domain of a second binding domain. The first binding domains (A) and/or second binding domains (B) may be fused to a constant domain of an antibody via a linker. Such a linker is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or preferably even less. The length of the linker is preferably determined as described by Rossmalen et al Biochemistry 2017, 56, 6565-6574, which also describes suitable linkers that are well known to the skilled person. An example for a suitable linker is a glycine serine linker or a serine linker, which preferably comprises not more than about 75 amino acids, preferably not more than about 50 amino acids. In illustrative examples, a suitable linker comprises one or more GGGGS sequences (SEQ ID NO: 84), such as (GGGGS)₂ (SEQ ID NO: 85), (GGGGS)₄ (SEQ ID NO: 86), or preferably (GGGGS)₆ (SEQ ID NO: 87). Other illustrative examples for linkers are shown in SEQ ID NOs: 80-83. The first binding domains (A) and/or the second binding domains (B) are preferably scDb fragments that are fused to two C termini of a Fc domain, preferably via a VL domain of the scDb. Accordingly, the arrangement of on the polypeptide chain (from N to C) is preferably . . . -CH2-CH3-VL-VH-VL-VH, optionally with a linker between the Fc and the scDb. The third binding domain can be located at any suitable position of the antibody construct. Where the antibody construct comprises a Fc region, the third binding domain (C) can be located N terminal of the Fc region, either directly or linked via at least a part of a hinge domain. Other linkers disclosed herein can also be used to link the third binding domain to the Fc domain. A hinge domain is however preferred for this purpose. The third binding domain (C) can be any suitable structure disclosed herein, while a Fab structure is preferred.

An antibody construct of the invention is preferably in a format as essentially shown in FIG. 9 and which is also referred to as “AIG-2scDb”. Such an antibody construct comprises an immunoglobulin that has two scDb fragments fused to the C-termini of the heavy chains, optionally via a linker, which is preferably a connector, disclosed herein. One of the two scDb comprises two first binding domains (A), while the other scDb comprises two second binding domains (B). Two third binding domains (C) are formed by the binding sites of the immunoglobulin. The AIG-2scDb format may comprise four polypeptide chains, two light chains in the arrangement VL(C)-CL, one heavy chain fused to a scDb in the arrangement VH(C)-CH1-hinge-CH2-CH3-VL(A)-VH(A)-VL(A)-VH(A) (or less preferred VH(C)-CH1-hinge-CH2-CH3-VH(A)-VL(A)-VH(A)-VL(A)), and one heavy chain fused to an scDb in the arrangement VH(C)-CH1-hinge-CH2-CH3-VL(B)-VH(B)-VL(B)-VH(B) (or less preferred VH(C)-CH1-hinge-CH2-CH3-VH(B)-VL(B)-VH(B)-VL(B)). Illustrative examples for such antibody constructs are shown in SEQ ID NOs: 369-371; 372-374; 375-377; 378-380; 431-433; 434-436; and 437-439.

An antibody construct of the invention can also be a combination of a half-molecule of the “AIG-2scFv” and a half-molecule of the “AIG-2scDb” format. Such an antibody construct is also referred to as “AIG-1scDb-1scFv” format. Such an antibody construct comprises an immunoglobulin that has one scDb fragment fused the C-termini of one of the heavy chains, optionally via a linker, which is preferably a connector, disclosed herein. Such an antibody construct further comprises an immunoglobulin that has one scFv fragment fused the C-terminus of another one of the heavy chains, optionally via a linker, which is preferably a connector, disclosed herein. The scDb can comprise two first binding domains (A), while the scFv comprises one second binding domain (B). Alternatively, the scDb can comprise two second binding domains (B), while the scFv comprises one first binding domain (A). Two third binding domains (C) are formed by the binding sites of the immunoglobulin. The AIG-1scDb-1scFv format may comprise four polypeptide chains, two light chains in the arrangement VL(C)-CL, one heavy chain fused to a scDb in the arrangement VH(C)-CH1-hinge-CH2-CH3-VL(A)-VH(A)-VL(A)-VH(A) (or less preferred VH(C)-CH1-hinge-CH2-CH3-VH(A)-VL(A)-VH(A)-VL(A)), and one heavy chain fused to a scFv in the arrangement VH(C)-CH1-hinge-CH2-CH3-VL(B)-VH(B) (or less preferred VH(C)-CH1-hinge-CH2-CH3-VH(B)-VL(B)). Alternatively, the AIG-1scDb-1scFv format may comprise four polypeptide chains, two light chains in the arrangement VL(C)-CL, one heavy chain fused to an scDb in the arrangement VH(C)-CH1-hinge-CH2-CH3-VL(B)-VH(B)-VL(B)-VH(B) (or less preferred VH(C)-CH1-hinge-CH2-CH3-VH(B)-VL(B)-VH(B)-VL(B)), and one heavy chain fused to an scFv in the arrangement VH(C)-CH1-hinge-CH2-CH3-VL(A)-VH(A) (or less preferred VH(C)-CH1-hinge-CH2-CH3-VH(A)-VL(A)). An illustrative example for such an antibody construct is shown in SEQ ID NOs: 500-502.

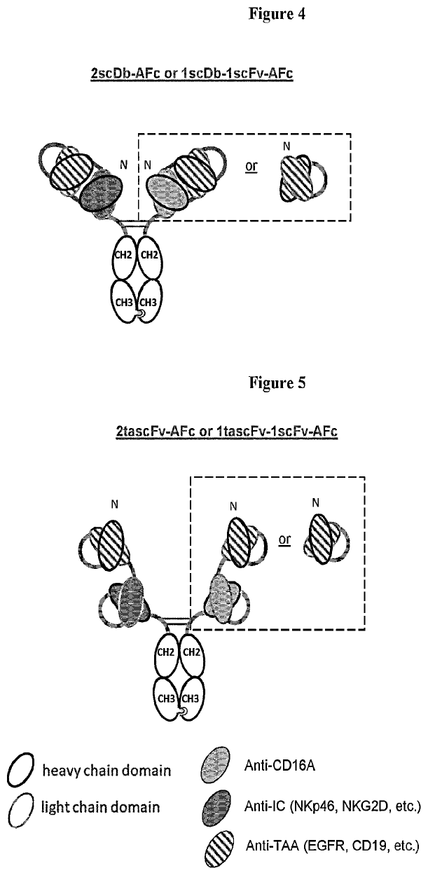

For providing a short distance between the first binding domain (A) and the second binding domain (B), the first binding domain (A) and the second binding domain (B) can also be fused to the N-termini of a pair (e.g. a dimer) of two constant domains of an antibody, such as a pair of two CH3 domains, a pair of two CH2 domains, or a pair of a CH1 domain and a CL domain. In a preferred embodiment, the first binding domain (A) is fused to the N-terminus of a CH2 domain and the second binding domain (B) is fused to the N-terminus of another CH2 domain. In a preferred embodiment, the first binding domain (A) and second binding domain (B) are fused to two N-termini of a Fc region. It is preferred for the antibody constructs of the invention that a first binding domain (A) is fused to the N-terminus of a first hinge domain and the second binding domain (B) is fused to the N-terminus of a second hinge domain. Such a fusion format is illustratively shown in FIG. 4 or FIG. 5. The first binding domain (A) and/or second binding domain (B) may be fused to a constant domain of an antibody via a linker disclosed herein (such as a connector disclosed herein) or a hinge domain, with a hinge domain being preferred.

Generally, a hinge domain comprised in an antibody construct of the disclosure may comprise a full length hinge domain, such as a hinge domain shown in SEQ ID NO: 88. The hinge domain may also comprise a shortened and/or modified hinge domain. A shortened hinge domain may comprise the upper hinge domain as e.g. shown in SEQ ID NO: 89 or the middle hinge domain as e.g. shown in SEQ ID NO: 90, but not the entire hinge domain, with the latter being preferred. Preferred hinge domains in the context of the invention show modulated flexibility relative to an antibody construct having the wild type hinge domain as described in Dall'Acqua et al (J Immunol. 2006 Jul. 15; 177(2):1129-38) or in WO 2009/006520. A hinge domain showing reduced flexibility is preferred for some antibody constructs of the disclosure, in particular if the first binding domain (A) and/or second binding domain (B) are fused to the hinge domain. Moreover, preferred hinge domains are characterized to consist of less than 25 aa residues. More preferably, the length of the hinge is 10 to 20 aa residues. A hinge domain comprised in an antibody construct of the disclosure may also comprise or consists of the IgG2 subtype hinge sequence ERKCCVECPPCP (SEQ ID NO: 452), the IgG3 subtype hinge sequence ELKTPLDTTHTCPRCP (SEQ ID NO: 453) or ELKTPLGDTTHTCPRCP (SEQ ID NO: 454), and/or the IgG4 subtype hinge sequence ESKYGPPCPSCP (SEQ ID NO: 455). Further hinge domains that can be used in the context of the present invention are known to the skilled person and are e.g. described in WO 2017/134140.

When the first binding domain (A) is fused to the N-terminus of a CH2 domain and the second binding domain (B) is fused to the N-terminus of another CH2 domain, such as the first binding domain (A) and second binding domain (B) are fused to two N termini of a Fc region or hinge domains, with the hinge domains being preferred, the third binding domain (C) can be fused to the N-terminus or C terminus of either one of the two polypeptide chains. In preferred embodiments, two third binding domains (C) are fused to the two chains. Preferably, one third binding domain (C) is fused N-terminal to the first binding domain (A) and one third binding domain is fused to the N-terminus of the second binding domain (B). In a preferred antibody construct, the first binding domain (A), the second binding domain (B), and the third binding domain(s) (C) are scFvs. In such an antibody construct both polypeptide strand may have the arrangement (from N to C) of scFv of the third domain (C)-scFv of the first/second binding domain (A)/(B)-hinge-CH2-CH3. In the scFv moieties, the VL and VH domain can be arranged in any order. However, the arrangement VH-VL is preferred for the third binding domain(s), while the arrangement VL-VH is preferred for the first binding domain (A) and/or the second binding domain (B).

An antibody construct of the invention is preferably in a format as essentially shown in FIG. 5 and which is also referred to as “2tascFv-AFc”. Such an antibody construct comprises two polypeptide chains, in which a third binding domain (C) in form of an scFv fused to the N-terminus of a first/second binding domain (A)/(B) in form of a scFv, optionally via a linker disclosed herein, such as a connector discloses herein. The first/second binding domain (A)/(B) is further fused to the N-terminus of a hinge domain that is connected to a CH2-CH3 domain. The 2tascFv-AFc format may comprise two polypeptide chains, one polypeptide chain in the arrangement VH(C)-VL(C)-VL(A)-VH(A)-hinge-CH2-CH3, and one polypeptide chain in the arrangement VH(C)-VL(C)-VL(B)-VH(B)-hinge-CH2-CH3. Illustrative examples for such antibody constructs are shown in SEQ ID NOs: 269-270; 271-272; 273-274; 275-276; 277-278; 279-280; 281-282; and 283-284.

When the first binding domain (A) is fused to the N-terminus of a CH2 domain and the second binding domain (B) is fused to the N-terminus of another CH2 domain, such as when the first binding domain (A) and second binding domain (B) are fused to two N-termini of a Fc region, the third binding domain (C) can be fused to first binding domain (A) and/or the second binding domain (B) in form of a diabody or a single chain diabody. The first binding domain (A) and/or second binding domain (B) may be fused to the CH2 domain or Fc domain via a linker disclosed herein (such as a connector discloses herein) or a hinge domain, with a hinge domain being preferred. In the spatial arrangement of the diabody, the first binding domain (A) and/or the second binding domain (B) should be adjacent to the hinge or CH2 domain while the third binding domain (C) is remote from the hinge or CH2 domain. This is e.g. achieved by fusing a VL or VH of the first or second binding domain (A) or (B) to the hinge or CH2 domain. For diabodies, this means that the arrangement on one of the “heavy chain” of the antibody construct is VL(C)-VH(A)-hinge/CH2- . . . or VH(C)-VL(A)-hinge/CH2- . . . or VL(C)-VH(B)-hinge/CH2- . . . or VH(C)-VL(B)-hinge/CH2- . . . , while the arrangement on the “light chain” is VL(A)-VH(C) or VH(A)-VL(C) or VL(B)-VH(C) or VH(B)-VL(C), respectively. For single chain diabodies, the arrangement of the domains on the polypeptide chains may be VL(A)-VH(C)-VL(C)-VH(A)-hinge/CH2- . . . or VH(A)-VL(C)-VH(C)-VL(A)-hinge/CH2- . . . or VL(B)-VH(C)-VL(C)-VH(B)-hinge/CH2- . . . or VH(B)-VL(C)-VH(C)-VL(B)-hinge/CH2- . . . , with the latter one being preferred.

An antibody construct of the invention is preferably in a format as essentially shown in FIG. 4 and which is also referred to as “2scDb-AFc”. Such an antibody construct comprises two polypeptide chains. In the first polypeptide chain, a third binding domain (C) and a first binding domain (A) are fused to each other in form of a scDb, which is fused to a hinge-CH2-CH3 domain via a variable domain of the first binding domain (A). In the second polypeptide chain, a third binding domain (C) and a second binding domain (B) are fused to each other in form of a scDb, which is fused to a hinge-CH2-CH3 domain via a variable domain of the first binding domain (A). The first polypeptide chain preferably has the arrangement VH(A)-VL(C)-VH(C)-VL(A)-hinge-CH2-CH3. The second polypeptide chain preferably has the arrangement VH(B)-VL(C)-VH(C)-VL(B)-hinge-CH2-CH3. Illustrative examples for such antibody constructs are shown in SEQ ID NOs: 237-238, 239-240, 241-242, 243-244, 245-246, 247-248, 249-250, and 251-252.

In order to provide a short distance between the first binding domain (A) and the second binding domain (B) both domains may also be fused to each other. There are several possibilities of fusing the first binding domain (A) and the second binding domain (B). In some embodiments, the C-terminus of the VL of the first binding domain (A) is fused to the N-terminus of the VH of the second binding domain (B) while the C-terminus of the VL of the second binding domain (B) is fused to the N-terminus of the VH of the first binding domain (A). The two VH and two VL can either be comprised in one single polypeptide chain or into separate polypeptide chains. In some embodiments, the N-terminus of the VL of the first binding domain (A) is fused to the C-terminus of the VH of the second binding domain (B) while the N-terminus of the VL of the second binding domain (B) is fused to the C-terminus of the VH of the first binding domain (A). The two VH and two VL can either be comprised in one single polypeptide chain or into separate polypeptide chains. In some embodiments, the C-terminus of the VL of the first binding domain (A) is fused to the N-terminus of the VL of the second binding domain (B) while the C-terminus of the VH of the first binding domain (B) is fused to the N-terminus of the VH of the second binding domain (A). The two VH and two VL can either be comprised in one single polypeptide chain or in two separate polypeptide chains. In some embodiments, the C-terminus of the VL of the second binding domain (A) is fused to the N-terminus of the VL of the first binding domain (B) while the C-terminus of the VH of the second binding domain (B) is fused to the N-terminus of the VH of the first binding domain (A). The two VH and two VL can either be comprised in one single polypeptide chain or in two separate polypeptide chains. It is also preferred that the first and the second binding domain are fused to each other in form of a ta-scFv, a double Fab, a db or scDb, wherein a db or scDb is preferred, with the scDb being most preferred. The spatial arrangement of the variable domains of a db or a scDb is preferably in a VL-VH-VL-VH order.

Generally, if the first binding domain (A) and the second binding domain (B) are fused to each other, the fusion of first binding domain (A) and second binding domain (B) can be N-terminally fused to a hinge domain. In such a case, it is preferred that the first binding domain (A) is N-terminally fused to the hinge domain and the second binding domain (B) is N-terminally fused to the first binding domain (A). In this context, N-terminally fused may be understood in terms of the interconnection of the subunits, but it may also be understood as the spatial orientation of the subunits to each other, depending on the context.

Generally, if the first binding domain (A) and the second binding domain (B) are fused to each other, the fusion of first binding domain (A) and second binding domain (B) can be C-terminally to a CH3 domain. In such a case, it is preferred that the first binding domain (A) is C-terminally fused to the CH3 domain and the second binding domain (B) is C-terminally fused to the first binding domain (A). In this context, C-terminally fused may be understood in terms of the interconnection of the subunits, but it may also be understood as the spatial orientation of the subunits to each other, depending on the context.

Some preferred antibody constructs of the invention comprise a first binding domain (A) and a second binding domain (B) that are fused together in form of a db or scDb. In such a scDb, the domains on the polypeptide on the polypeptide chain are preferably arranged in the (N to C) order VL-VH-VL-VH. The preferred arrangements are VL(A)-VH(B)-VL(B)-VH(A) and VL(B)-VH(A)-VL(A)-VH(B) with VL(A)-VH(B)-VL(B)-VH(A) being more preferred. In a preferred version of a db, one polypeptide chain comprises two variable domains in the arrangement VL(B)-VH(A) and another polypeptide chain comprises two variable domains in the arrangement VL(A)-VH(B). In a more preferred version of a db, one polypeptide chain comprises two variable domains in the arrangement VL(A)-VH(B) and another polypeptide chain comprises two variable domains in the arrangement VL(B)-VH(A). The db or scDb are preferably fused to the antibody construct via the N-terminus of VL(A) or the C-terminus of VH(A). As an illustrative example, if such a db or more preferably a scDb is fused to the C-terminus of a CH3 domain, it is preferably fused via the N-terminus of the VL domain of the first binding domain (A). As another illustrative example, if such a db or more preferably a scDb is fused to the N-terminus of a CH3 domain, it is preferably fused via the C-terminus of the VH domain of the first binding domain (A).

In an antibody construct of the invention that comprises a first binding domain (A) that is fused to a second binding domain (B), the fusion of the first binding domain (A) and the second binding domain (B) can be fused to the third binding domain (C) in any order. It can be directly fused to the third binding domain (C). However, it is preferred that both, the fusion of the first and second binding domain (A) and (B) and the third binding domain (D) are fused to a fourth domain (D). If the fourth domain (D) consists of one single polypeptide chain, the fusion of the first and second binding domain (A) and (B) can either be fused to the N- or C-terminus of the fourth domain (D) while the third binding domain can be fused to the other terminus (either C- or N-terminus) of the fourth domain (D). If the fourth domain (D) comprises of two polypeptide chains, the fusion of the first and second binding domain (A) and (B) can either be fused to the N- or C-terminus of the fourth domain (D) while the third binding domain can be fused to any other “free” terminus (either C- or N-terminus) of the fourth domain (D).

In preferred antibody constructs of the invention, the antibody construct comprises two hinge-CH2-CH3 elements. These two hinge-CH2-CH3 can be located on one single polypeptide chain, e.g. in form of a scFc. It is however more preferred that these two hinge-CH2-CH3 are located on two separate polypeptide chains.

Some preferred formats for antibody constructs of the invention comprise (i) a first binding domain (A) and a second binding domain (B) that are fused together as described herein and (ii) a fourth domain (D) that comprise two hinge-CH2-CH3 elements.

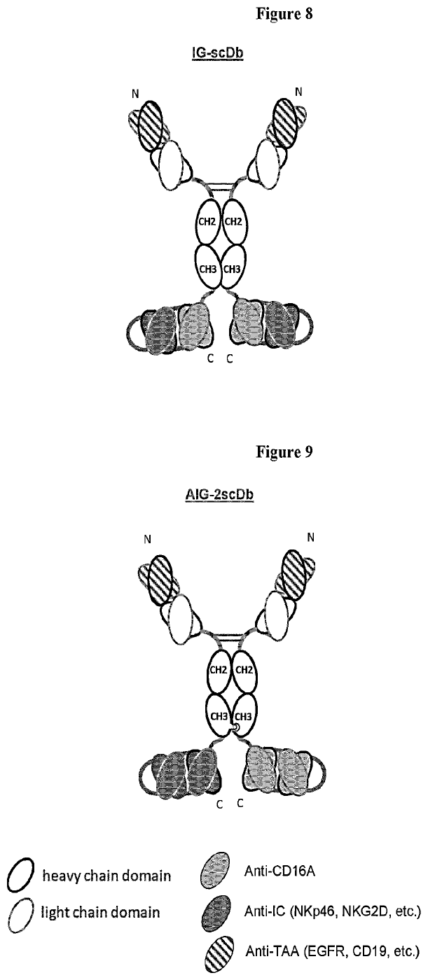

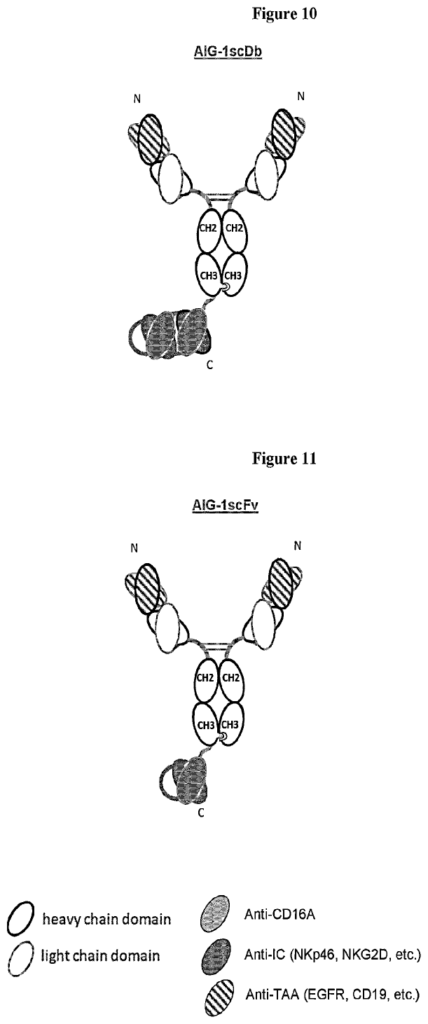

In a preferred embodiment, two fusions of a first binding domain (A) and a second binding domain (B), preferably in form of a scDb, are fused to two C termini of a Fc region, preferably via the N-terminus of the VL of the first binding domain (A). Such a fusion format is illustratively shown in FIG. 8. The scDb may be fused to a constant domain of an antibody via a linker. Such a linker is preferably a short linker, which preferably has a length of about 10 nm or less, preferably about 9 nm or less, preferably about 8 nm or less, preferably about 7 nm or less, preferably about 6 nm or less, preferably about 5 nm or less, preferably about 4 nm or less, or even less. The length of the linker is preferably determined as described by Rossmalen et al Biochemistry 2017, 56, 6565-6574, which also describes suitable linkers that are are well known to the skilled person. An example for a suitable linker is a glycine serine linker or a serine linker, which preferably comprises not more than about 75 amino acids, preferably not more than about 50 amino acids. In illustrative examples a suitable linker comprises one or more GGGGS sequences (SEQ ID NO: 84), such as (GGGGS)₂ (SEQ ID NO: 85), (GGGGS)₄ (SEQ ID NO: 86), or preferably (GGGGS)₆ (SEQ ID NO: 87). Other illustrative examples for linkers are shown in SEQ ID NOs: 80-83. The third binding domain can be located at any suitable position of the antibody construct. It is however preferred that the third binding domain (C) is located N-terminal of the Fc region, either directly or linked via at least a part of a hinge domain. Other linkers disclosed herein can also be used to link the third binding domain to the Fc domain. A hinge domain is however preferred for this purpose. The third binding domain (C) can be any suitable structure disclosed herein, while a Fab structure is preferred.

An antibody construct of the invention is preferably in a format as essentially shown in FIG. 8 and which is also referred to as “IG-scDb”. Such an antibody construct comprises an immunoglobulin that has two scDb fragments fused to the C termini of the heavy chains, optionally via a linker disclosed herein, such as a connector disclosed herein. The two scDb each comprises a first binding domain (A) and a second binding domain (B). Two third binding domains (C) are formed by the binding sites of the immunoglobulin. The IG-scDb format may comprise four polypeptide chains, two light chains in the arrangement VL(C)-CL, and two heavy chain fused to a scDb in the arrangement VH(C)-CH1-hinge-CH2-CH3-VL(A)-VH(B)-VL(B)-VH(A) (or less preferred VH(C)-CH1-hinge-CH2-CH3-VH(A)-VL(B)-VH(B)-VL(A), VH(C)-CH1-hinge-CH2-CH3-VH(B)-VL(A)-VH(A)-VL(B), VH(C)-CH1-hinge-CH2-CH3-VL(B)-VH(A)-VL(A)-VH(B)). Illustrative examples for such antibody constructs are shown in SEQ ID NOs: 353-354; 355-356; 357-358; and 359-360.

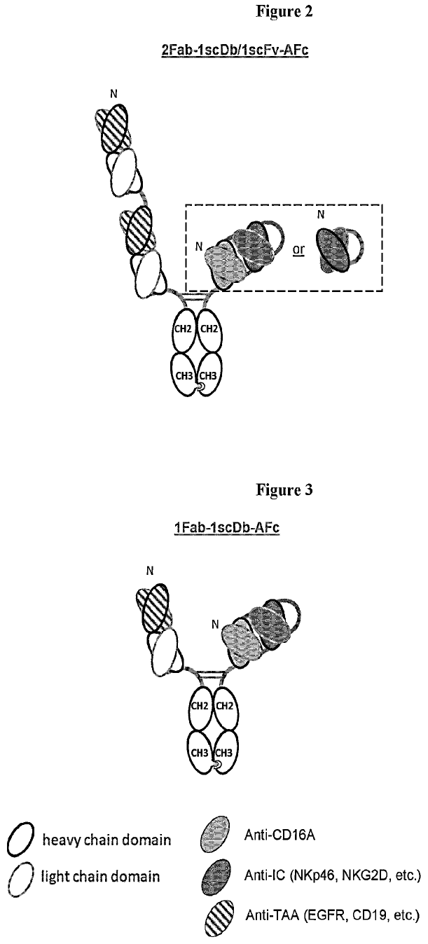

When a fusion of a first binding domain (A) and a second binding domain (B) is fused to the N-terminus of a CH2 domain, a third binding domain (C) can be fused to the N-terminus of another CH2 domain. For example, the fusion of a first binding domain (A) and a second binding domain (B) and the third binding domain (C) can be fused to two N-termini of a Fc region, as illustratively shown in FIG. 2, 3, or 6. The fusion of a first binding domain (A) and a second binding domain (B) is preferably in the form of db, a double Fab or, more preferably, in form of a scDb. The third binding domain (C) is preferably in the form of a Fab. In some preferred embodiments, the antibody construct comprises two third binding domains (C), which are preferably in the form of two Fabs that are fused together, or a diabody. The fusion of a first binding domain (A) and a second binding domain (B) may be fused to the CH2 domain or Fc domain via a linker disclosed herein (such as a connector disclosed herein) or a hinge domain, with a hinge domain being preferred. In the spatial arrangement of the diabody, the first binding domain (A) is preferably adjacent to the hinge or CH2 domain while the second binding domain (B) is remote from the hinge or CH2 domain. This is e.g. achieved by fusing a VL or VH of the first binding domain (A) to the hinge or CH2 domain. For diabodies, this means that the arrangement on one of the “heavy chain” of the antibody construct is VL(B)-VH(A)-hinge/CH2- . . . or VH(B)-VL(A)-hinge/CH2- . . . , while the arrangement on the “light chain” is VL(A)-VH(B) or VH(A)-VL(B), respectively. For single chain diabodies, the arrangement of the domains on the polypeptide chains may be VL(A)-VH(B)-VL(B)-VH(A)-hinge/CH2- . . . or VH(A)-VL(B)-VH(B)-VL(A)-hinge/CH2- . . . , with the latter one being preferred.

An antibody construct of the invention is preferably in a format as essentially shown in shown in FIG. 3 and which is also referred to as “1Fab-1scDb-AFc”. Such an antibody construct comprises three polypeptide chains. The first polypeptide chain comprises a heavy chain of an antibody that binds to the third target (C′), i.e. that comprises a variable domain of a third binding domain (C). The first polypeptide chain preferably has the arrangement VH(C)-CH1-hinge-CH2-CH3. The second polypeptide chain comprises a light chain of an antibody that binds to the third target (C′), i.e. that comprise the variable domain of a third binding domain. The second polypeptide chain preferably has the arrangement VL(C)-CL. The third polypeptide comprises a scDb comprising the first binding domain (A) and the second binding domain (B), which is fused to the N-terminus of a hinge-CH2-CH3 domain. The scDb is preferably fused to the hinge-CH2-CH3 domain via a variable region of the first binding domain (A), more preferably via the C-terminus of the VH domain of the first binding domain (A). The third polypeptide preferably comprises the arrangement VL(A)-VH(B)-VL(B)-VH(A)-hinge-CH2-CH3. Illustrative examples for such antibody constructs are shown in SEQ ID NOs: 225-227; 228-230; 231-233; 234-236.

An antibody construct of the invention is preferably in a format as essentially shown in FIG. 2 and which is also referred to as “2Fab-1scDb-AFc”. Such an antibody construct comprises four or three polypeptide chains. One polypeptide chain comprises a heavy chain of an antibody that binds to the third target (C′), i.e. that comprises a variable domain of a third binding domain (C), which further comprises a polypeptide chain of a Fab fused to its N-terminus. The Fab fused to the N-terminus also binds the third target (C′). The first polypeptide chain preferably has the arrangement VH(C)-CH1-VH(C)-CH1-hinge-CH2-CH3, while other arrangement such as VL(C)-CL-VH(C)-CH1-hinge-CH2-CH3 are also possible but less preferred. Another polypeptide chain of the 2Fab-1scDb-AFc construct comprises light chain of an antibody that binds to the third target (C′), i.e. that comprise the variable domain of a third binding domain (C). The further polypeptide chain comprises a variable and the constant region that forms the second polypeptide chain of the Fab that is fused to the N-terminus of the heavy chain. Depending on which chain of the Fab is fused to the N-terminus of the heavy chain, the further polypeptide chain may have the arrangement VH(C)-CH1 or VL(C)-CL, with VL(C)-CL being preferred. Optionally, although not preferred, the two “light chains” that form the two third binding domains (C) can be fused together, optionally via a linker, optionally a linker disclosed herein. A further polypeptide comprises a diabody comprising the first binding domain (A) and the second binding domain (B), which is fused to the N-terminus of a hinge-CH2-CH3 domain. The scDb is preferably fused to the hinge-CH2—CH3 domain via a variable region of the first binding domain (A), more preferably via the C-terminus of the VH domain of the first binding domain (A). The further polypeptide preferably comprises the arrangement VL(A)-VH(B)-VL(B)-VH(A)-hinge-CH2-CH3. Illustrative examples for such antibody constructs are shown in SEQ ID NOs: 177-179; 180-182; 183-185; 186-188; 189-191; 192-194; 195-197; and 198-200.

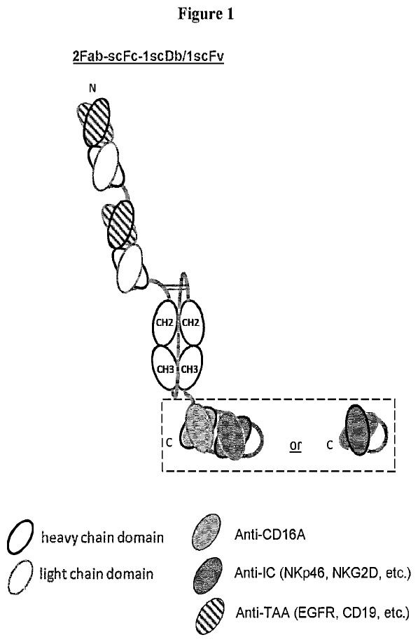

An antibody construct of the invention is preferably in a format as essentially shown in FIG. 1 and which is also referred to as “2Fab-1scFc-1scDb”. Such an antibody construct comprises three or two polypeptide chains. One polypeptide chain comprises two chains of a Fab that binds to the third target (C′) fused to the N-terminus of a scFc, which is further fused via its C-terminus to a diabody comprising the first binding domain (A) and the second binding domain (B). While any two chains of the two Fabs that bind to the third target (C′) can be fused to the scFc domain, two VH-CH1 elements are preferred. Similarly, the diabody can be fused to the scFc via any one of its variable domains. However, it is preferred that a variable domain of the first binding domain (A) is fused to the scFc element. It is even more preferred that the VL of the first binding domain (A) is fused to the scFc domain. A preferred arrangement for this polypeptide chain is VH(C)-CH1-VH(C)-CH1-hinge-CH2-CH3-hinge-CH2-CH3-VL(A)-VH(B)-VL(B)-VH(A). The two other polypeptide chains of the 2Fab-1scFc-1scDb construct each comprise a variable and a constant region that form the second polypeptide chain of the two Fabs that are fused to the N-terminus of the scFc. Depending on which chain of the Fabs fused to the N-terminus of the scFc, the further polypeptide chains may have the arrangement VH(C)-CH1 or VL(C)-CL, with VL(C)-CL being preferred. Optionally, although not preferred, the two “light chains” that form the two third binding domains (C) can be fused together, optionally via a linker, optionally a linker disclosed herein. Illustrative examples for such antibody constructs are shown in SEQ ID NOs: 161-162; 163-164; 165-166; and 167-168.

An antibody construct of the invention is preferably in a format as referred to as “1scFv-1scFc-1scDb”. Such an antibody construct comprises one polypeptide chain. The polypeptide chain comprises an scFv that binds to the third target (C′) fused to the N-terminus of a scFc, which is further fused via its C-terminus to a diabody comprising the first binding domain (A) and the second binding domain (B). Any chain of the scFv that binds to the third target (C′) can be fused to the scFc domain. Accordingly, either the VL domain or the VH domain of the scFv can be fused to the scFc domain, with a VH domain being preferred. Similarly, the diabody can be fused to the scFc via any one of its variable domains. However, it is preferred that a variable domain of the first binding domain (A) is fused to the scFc element. It is even more preferred that the VL of the first binding domain (A) is fused to the scFc domain. A preferred arrangement for this polypeptide chain is VL(C)-VH(C)-hinge-CH2-CH3-hinge-CH2-CH3-VL(A)-VH(B)-VL(B)-VH(A). Another preferred arrangement for this polypeptide chain is VH(C)-VL(C)-hinge-CH2-CH3-hinge-CH2-CH3-VL(A)-VH(B)-VL(B)-VH(A).

An antibody construct of the invention is preferably in a format as referred to as “1tascFv-1scFc-1scDb”. Such an antibody construct comprises one polypeptide chain. The polypeptide chain comprises an ta-scFv in which both scFv bind to the third target (C′). The two scFv comprised in the ta-scFv are optionally fused to each other via a linker disclosed herein. The ta-scFv is fused to the N-terminus of a scFc, which is further fused via its C-terminus to a diabody comprising the first binding domain (A) and the second binding domain (B). Any arrangement of the ta-scFv can be used. Accordingly, the ta-scFv moiety can have the arrangement VL(C)-VH(C)-VL(C)-VH(C)- . . . , VH(C)-VL(C)-VH(C)-VL(C)- . . . , VL(C)-VH(C)-VH(C)-VL(C)- . . . , or VH(C)-VL(C)-VL(C)-VH(C)- . . . , with VH(C)-VL(C)-VL(C)-VH(C)- . . . being preferred. Accordingly, either a VL domain or a VH domain of the ta-scFv can be fused to the scFc domain, with a VH domain being preferred. Similarly, the diabody can be fused to the scFc via any one of its variable domains. However, it is preferred that a variable domain of the first binding domain (A) is fused to the scFc element. It is even more preferred that the VL of the first binding domain (A) is fused to the scFc domain. A preferred arrangement for this polypeptide chain is VL(C)-VH(C)-VL(C)-VH(C)-hinge-CH2-CH3-hinge-CH2-CH3-VL(A)-VH(B)-VL(B)-VH(A). Another preferred arrangement for this polypeptide chain is VH(C)-VL(C)-VH(C)-VL(C)-hinge-CH2-CH3-hinge-CH2-CH3-VL(A)-VH(B)-VL(B)-VH(A). Another preferred arrangement for this polypeptide chain is VL(C)-VH(C)-VH(C)-VL(C)-hinge-CH2-CH3-hinge-CH2-CH3-VL(A)-VH(B)-VL(B)-VH(A). Another preferred arrangement for this polypeptide chain is VH(C)-VL(C)-VL(C)-VH(C)-hinge-CH2-CH3-hinge-CH2-CH3-VL(A)-VH(B)-VL(B)-VH(A).

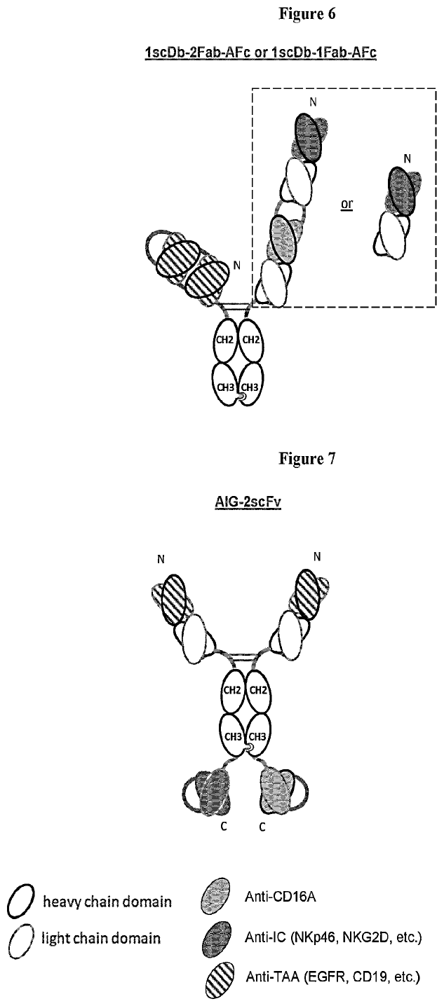

An antibody construct of the invention is preferably in a format as essentially shown in FIG. 6 and which is also referred to as “1scDb-2Fab-AFc”. Such an antibody construct comprises three polypeptide chains. A first polypeptide comprises a diabody comprising two third binding domains (C), which is fused to the N-terminus of a hinge-CH2-CH3 domain. The diabody can be fused to the hinge-CH2-CH3 domain via any variable region of the diabody. However, a fusion via the C-terminus of a VL domain is preferred. This polypeptide chain preferably comprises the arrangement VH(C)-VL(C)-VH(C)-VL(C)-hinge-CH2-CH3. A second polypeptide chain comprises a chain of a Fab specific for the first target (A′) fused to a chain of a Fab specific for the second target (B′) fused together, which is further fused via their C-terminus to a hinge-CH2-CH3 region. The arrangements of the Fab chains to each other can be in any order, i.e. the Fab chain specific for the first target (A′) can be fused to the Fab chain specific for the second target (B′) either N-terminally or C-terminally. However, it is preferred that the Fab chain specific for the second target (B′) is N-terminal of the Fab chain specific for the first target (A′). While any two chains of the two Fabs that bind to the first target (A′) and the second target (B′) can be fused to the hinge-CH2-CH3 domain, two VH-CH1 elements are preferred. This polypeptide chain preferably comprises the arrangement VH(B)-CH1-VH(A)-CH1-hinge-CH2-CH3. The third polypeptide chain comprises the other two Fab chains that bind to the first target (A′) and the second target (B′). Depending on which chains are fused to the hinge-CH2-CH3 region, the Fab chains comprised in the third polypeptide may comprise VL-CL or VH-CH1, with VL-CL being preferred. The arrangement of the two Fab chains to each other also depend on the arrangement of the Fab chains fused to the hinge-CH2-CH3 region. If the Fab chain specific for the second target (B′) is N-terminal to the Fab chain specific for the first target (A′) on the second polypeptide chain, the Fab chain specific for the second target (B′) should also be N-terminal to the Fab chain specific for the first target (A′) on the third polypeptide chain, and vice versa. The third polypeptide chain preferably comprises the arrangement VL(B)-CL(B)-VL(A)-CL(A). Illustrative examples for such antibody constructs are shown in SEQ ID NOs: 293-295; 296-298; 299-301; 302-304; 305-307; 308-310; 311-313; and 314-316.

Ideally, the distance between the binding site of the first binding domain (A) and the second binding domain (B) is short. It is thus preferred that the two binding domains are within the distance of about 25 nm or less, more preferably about 22 nm or less, more preferably about 20 nm or less, more preferably about 19 nm or less, more preferably about 18 nm or less, more preferably about 17 nm or less, more preferably about 16 nm or less, more preferably about 15 nm or less, more preferably about 14 nm or less, more preferably about 13 nm or less, more preferably about 12 nm or less, more preferably about 11 nm or less, more preferably about 10 nm or less, more preferably about 9 nm or less, more preferably about 8 nm or less, more preferably about 7 nm or less, more preferably about 6 nm or less, more preferably about 5 nm or less. The distance is preferably determined from the center of the binding site. If the antibody construct comprises more than one first binding domain (A) and/or second binding domain (B), the distance between the domains are preferably measured between the first binding domain (A) and the second binding domain (B) that have the largest distance to each other. For determining the distance between two binding domains, crystal structures are preferred. Where crystal structures are not available, structural considerations according Rossmalen et al Biochemistry 2017, 56, 6565-6574, are preferably applied, in particular with regard to linkers.

Apart from the distance between the first binding domain (A) and a second binding domain (B), also the orientation of their binding sites can contribute to avoiding simultaneous binding to two different immune effector cells, or at least reducing its likelihood. Without wishing to be bound by theory, it is believed that the more both binding domains face the same direction the less likely it becomes that the two binding domains simultaneously bind to two different immune effector cells. It is further believed that binding sites facing more or less the same direction allow for a longer distance between the two binding domains (A) and (B) without mediating the simultaneous binding of two immune effector cells. The spatial orientation of the binding domain can also be modulated by the domain via it is fused to another element of the antibody construct it is fused to. For example, the antibody construct comprises a Fc domain having two (hinge)-CH2-CH3 elements, and if the first binding domain (A) and the second binding domain (B) are fused to different chains of this Fc domain it is preferred to fuse the light chain of the two binding domains (A) and (B) to the Fc domain, since such an arrangement is believed to provide binding sites of the two binding domains which face a more similar direction. Also, in a diabody comprising the two binding domains (A) and (B), it is preferred to have the arrangement VL-VH-VL-VH, since this also provides binding sites that face a more similar direction.

Accordingly, it is preferred for the antibody constructs of the invention that the binding sites of the first binding domain (A) and the binding site of the second binding domain (B) are in cis-orientation. In this context, cis-orientation means that the binding sites of the two binding domains point into directions which form an angle of about 1200 or less, preferably about 900 or less, which preferably favors binding of both domains to the same effector cell.

However, in order to facilitate simultaneous binding of an effector cell and a target cell, the third binding site (C) may point to an opposite direction as at least one, preferably both of the binding sites of the first binding domain (A) and/or second binding domain (B) which is referred to as trans-orientation. Therefore, it is preferred for the antibody constructs of the invention that the binding site of the first binding domain (A) and the binding site of the third binding domain (C) are in trans-orientation. Further, it is also preferred for the antibody construct of the invention that the binding site of the second binding domain (B) and the binding site of the third binding domain (C) are in trans-orientation. It is even more preferred for the antibody constructs of the invention that the binding site of both the first binding domain (A) and the second binding domain are in trans-orientation to the binding site of the third binding domain (C). In this context, trans-orientation means that the two binding sites face directions which are in an angle of about 1200 or more, preferably about 1350 or more.

Where an antibody construct of the invention comprises CH3 regions, modifications to the CH3 region can be introduced to improve heterodimeric pairing of the polypeptides comprising the CH3 regions. The CH3 regions can be altered by the “knob-into-holes” technology which is described in detail with several examples in e.g. WO 96/027011, Ridgway, J., B., et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M., et al., Nature Biotech 16 (1998) 677-681; Atwell, S., et al., J. Mol. Biol. 270 (1997) 26-35) and increases the yield.

Thus the antibody constructs of the disclosure may be further characterized in that the CH3 domain of one polypeptide chain and the CH3 domain of another polypeptide chain each meet at an interface which comprises an original interface between the antibody CH3 domains; wherein the interface is altered to promote the formation of the antibody construct. An alteration may be characterized in that: a) the CH3 domain of one polypeptide chain is altered, so that within the original interface the CH3 domain of one polypeptide chain that meets the original interface of the CH3 domain of the other polypeptide chain within the antibody construct, an amino acid residue is replaced with an amino acid residue having a larger side chain volume, thereby generating a protuberance within the interface of the CH3 domain of one polypeptide chain which is positionable in a cavity within the interface of the CH3 domain of the other polypeptide chain and b) the CH3 domain of the other polypeptide chain is altered, so that within the original interface of the second CH3 domain that meets the original interface of the first CH3 domain within the antibody construct an amino acid residue is replaced with an amino acid residue having a smaller side chain volume, thereby generating a cavity within the interface of the second CH3 domain within which a protuberance within the interface of the first CH3 domain is positionable.

Preferably the amino acid residue having a larger side chain volume is selected from the group consisting of arginine (R), phenylalanine (F), tyrosine (Y), tryptophan (W). Preferably the amino acid residue having a smaller side chain volume is selected from the group consisting of alanine (A), serine (S), threonine (T), valine (V).

Both CH3 domains further be altered by the introduction of cysteine (C) as amino acid in the corresponding positions of each CH3 domain such that a disulfide bridge between both CH3 domains can be formed.

In a preferred embodiment, the antibody construct comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M, et al., Nature Biotech 16 (1998) 677-681) e.g. by introducing a Y349C mutation into the CH3 domain of the “knobs chain” and a E356C mutation or a S354C mutation into the CH3 domain of the “hole chain”. Alternatively, the antibody construct may comprise a T366Y in the CH3 domain of the “knobs chain” and a Y407T mutation in the “hole chain”. Other knobs-in-holes technologies that can also be used are described in Labrijn A F, Janmaat M L, Reichert J M, Parren P. Bispecific antibodies: a mechanistic review of the pipeline. Nat Rev Drug Discov 2019; 18:585-608. Preferred versions of knob chain CH2-CH3 heavy chain constant domains are shown in SEQ ID NOs: 101 and 103. Preferred versions of hole chain CH2-CH3 heavy chain constant domains are shown in SEQ ID NOs: 100 and 102.

The present invention preferably relates to a trispecific antibody construct, which binds to a target cell and one immune effector cell simultaneously, said antibody construct comprising (i.) a first binding domain (A), which is capable of specifically binding to a first target (A′) that is CD16A which is preferably on the surface of an immune effector cell; (ii.) a second binding domain (B), which is capable of specifically binding to a second target (B′) that is another antigen which is on the surface of an immune effector cell, with the exception of CD16A wherein it is preferred that said antigen is selected from the group comprising CD56, NKG2A, NKG2D, NKp30, NKp44, NKp46, NKp80, DNAM-1, SLAMF7, OX40, CD47/SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5 and CD3; and (iii.) a third binding domain (C), which is capable of specifically binding to a third target (C′) that is an antigen which is preferably on the surface of a target cell.

As already described herein, the first binding domain (A) is capable of specifically binding CD16A, which preferably includes the capacity to discriminate between CD16A and CD16B. With other words, the first binding domain (A) preferably binds CD16A with higher affinity than CD16B, which may be at least about 10-fold higher, at least about 100-fold higher, or at least about 1000-fold higher. More preferably, the first binding domain does not essentially bind CD16B. It is thus understood that the first binding domain is preferably not a non-silenced CH2 domain, i.e. a CH2 domain that is capable of binding both CD16A and CD16B.

Accordingly the first binding domain preferably binds to an epitope of CD16A which comprises amino acid residues of the C-terminal sequence SFFPPGYQ (positions 201-209 of SEQ ID NO: 449), and/or residue G147 and/or residue Y158 of CD16A, which are not present in CD16B. It is preferred in the context of the invention that the first binding domain, which binds CD16A on the surface of an effector cell binds to an epitope on CD16A, which is membrane proximal relative to the physiological Fcγ receptor binding domain of CD16A. A binding domain that specifically binds to an epitope comprising Y158 is preferred, because this epitope is proximal to the cell membrane and thus further contributes to reducing the likelihood of simultaneously binding a second immune effector cell. Examples for respective binding domains are characterized e.g. by the following groups of CDRs: CDR-H1 as depicted in SEQ ID NO: 26, a CDR-H2 as depicted in SEQ ID NO: 27, a CDR-H3 as depicted in SEQ ID NO: 28, a CDR-L1 as depicted in SEQ ID NO: 29, a CDR-L2 as depicted in SEQ ID NO: 30, a CDR-L3 as depicted in SEQ ID NO: 31 and binding domains which bind to the same epitope. Preferred CD16A binding domains are characterized by the following groups of CDRs: CDR-H1 as depicted in SEQ ID NO: 32, a CDR-H2 as depicted in SEQ ID NO: 33, a CDR-H3 as depicted in SEQ ID NO: 34, a CDR-L1 as depicted in SEQ ID NO: 35, a CDR-L2 as depicted in SEQ ID NO: 36, a CDR-L3 as depicted in SEQ ID NO: 37 and binding domains which bind to the same epitope. Examples for such CD16A binder are also described in WO2020043670.

In some embodiments, the first binding domain comprises (i) a VL region comprising CDR-L1, CDR-L2 and CDR-L3 selected from: (a) CDR-L1 as depicted in SEQ ID NO: 29, a CDR-L2 as depicted in SEQ ID NO: 30, a CDR-L3 as depicted in SEQ ID NO: 31; and (b) CDR-L1 as depicted in SEQ ID NO: 35, a CDR-L2 as depicted in SEQ ID NO: 36, a CDR-L3 as depicted in SEQ ID NO: 37 and (ii) a VH region comprising CDR-H1, CDR-H2 and CDR-H3 selected from: (a) CDR-H1 as depicted in SEQ ID NO: 26, a CDR-H2 as depicted in SEQ ID NO: 27, a CDR-H3 as depicted in SEQ ID NO: 28; and a CDR-L1 as depicted in SEQ ID NO: 29, a CDR-L2 as depicted in SEQ ID NO: 30, a CDR-L3 as depicted in SEQ ID NO: 31.

In some preferred embodiments, the first binding domain (A) comprises a VH domain comprising the three heavy chain CDRs and a VL domain comprising the three light chain CDRs selected form the group consisting of:
(a) a CDR-H1 as depicted in SEQ ID NO: 26, a CDR-H2 as depicted in SEQ ID NO: 27, a CDR-H3 as depicted in SEQ ID NO: 28, a CDR-L1 as depicted in SEQ ID NO: 29, a CDR-L2 as depicted in SEQ ID NO: 30, a CDR-L3 as depicted in SEQ ID NO: 31; and
(b) a CDR-H1 as depicted in SEQ ID NO: 32, a CDR-H2 as depicted in SEQ ID NO: 33, a CDR-H3 as depicted in SEQ ID NO: 34, a CDR-L1 as depicted in SEQ ID NO: 35, a CDR-L2 as depicted in SEQ ID NO: 36, a CDR-L3 as depicted in SEQ ID NO: 37.

In some preferred embodiments, the first binding domain (A) comprises a pair of VH- and VL-chains having a sequence as depicted in the pairs of sequences selected form the group consisting of SEQ ID NOs: 1 and 5; SEQ ID NOs: 2 and 7, SEQ ID NOs: 3 and 6; and SEQ ID NOs: 4 and 7.

In some embodiments, the first binding domain (A) comprises a VH domain comprising the following three heavy chain CDRs and a VL domain comprising the following three light chain CDRs: a CDR-H1 as depicted in SEQ ID NO: 38, a CDR-H2 as depicted in SEQ ID NO: 39, a CDR-H3 as depicted in SEQ ID NO: 40, a CDR-L1 as depicted in SEQ ID NO: 41, a CDR-L2 as depicted in SEQ ID NO: 42, a CDR-L3 as depicted in SEQ ID NO: 43.

In some embodiments, the first binding domain (A) comprises a pair of VH- and VL-chains having a sequence as depicted in the pairs of sequences selected form the group consisting of SEQ ID NOs: 8 and 9.

Several different antigens can be chosen as the second target (B′) for the selection of the second binding domain (B) of the antibody construct of the disclosure. On the one hand, binding of this second binding domain might boost the functionality of immune effector cells by inducing activation signals or blocking inhibitory signals on e.g. NK cells, macrophages, monocytes, CD8+ T cells through engagement of antigens such as, but not limited to, NKG2D, NKp30, NKp44, NKp46, NKp80, DNAM-1, SLAMF7, OX40, CD137, CD89, CD160, killer-cell immunoglobulin-like receptors (e.g. KIR2DS1-5), CD3, CD96, TIGIT, PD-1, PD-L1, LAG-3, CTLA-4 and TIM-3. Moreover, antigens for the second binding domain can be grouped into different categories depending on the mechanism of action: (1) Antigens inducing an activation in synergy with CD16A such as, but not limited to, NKG2D, NKp30, NKp44, NKp46, NKp80, DNAM-1, SLAMF7, OX40, CD137, CD89, CD160, killer-cell immunoglobulin-like receptors. (2) Antigens inducing activation of effector cells independent of CD16A including such as, but not limited, to NKG2D, NKp30, NKp44, NKp46, NKp80, DNAM-1, SLAMF7, OX40, CD137, CD160 and CD3. (3) Blockage of inhibitory antigens on effector cells comprising e.g. NKG2A, TIGIT, PD-1, PD-L1, CD47, SIRPα, LAG-3, CTLA-4, CD96, TIM-3, CD137, KIR2DL1-5 and KIR3DL1-3 to counteract inhibition and/or functional exhaustion. On the other hand, the second binding domain might reduce the inhibitory functionality of e.g. immunosuppressive cells such as, but not limited to, tumor-associated macrophages, regulatory T cells, myeloid-derived suppressor cells and cancer cells through engagement of antigens such as, but not limited to, CD47, PD-L1 and nectin 4.

The antigens inducing activation of the effector cells can be additionally classified in groups according the signaling cascade in comparison to CD16A: (1) CD3ζ-dependent/CD16A-associated signaling such as NKp46, NKp30 and (2) CD3ζ-independent signaling such as, but not limited to, NKG2D, NKp44, NKp80, DNAM-1, SLAMF7 and killer-cell immunoglobulin-like receptors (e.g. KIR2DS1).

Depending on the selection of the antigen for the second binding domain, different cell types will be potentially targeted/activated such as, but not limited to, NK cells with antigens comprising e.g. NKG2D, NKp30, NKp44, NKp46, NKp80, DNAM-1, SLAMF7, OX40, CD137, CD160, KIR2DS1-5, NKG2A, TIGIT, PD-1, PD-L1, CD47, LAG-3, CTLA-4, CD96, TIM-3, CD137, KIR2DL1-5 and KIR3DL1-3; monocytes and macrophages with e.g. CD89, SLAMF7, SIRPα, CD47; T cells with antigens comprising such as CD3, NKG2D, NKp30, NKp44, NKp46, CD160, OX40, CD137, PD-1, PD-L1, LAG-3, CTLA-4, TIM-3 and killer-cell immunoglobulin-like receptors. Moreover, dependent on the antigen different subpopulations (e.g. CD56^{dim}CD16^{bright} NK cells, CD56^{bright}CD16^{negative} NK cells, peripheral or tissue resident NK cells, M1 or M2 macrophages, tumor-associated macrophages, CD16^{pos} or CD16^{neg} monocytes, CD4+ or CD8+ αβT cells, γδ T cells, regulatory T cells and myeloid-derived suppressor cells) can be addressed in combination with CD16A or independent of CD16A.

In some embodiments, the second binding domain (B) is specific for a CD antigen, with the exception of CD16A. In some embodiments, the second binding domain (B) is capable of specifically binding to a second target (B′) that is selected from the group consisting of CD56, NKG2A, NKG2D, NKp30, NKp44, NKp46, NKp80, DNAM-1, SLAMF7, OX40, CD47/SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5 and CD3.

Antibodies against such targets are well known in the art. Antibodies against CD56 are e.g. described in WO2012138537 and WO2017023780. Antibodies against NKG2A are e.g. described in WO2008009545, WO2009092805, WO2016032334, WO2020094071, WO2020102501. Antibodies against NKG2D are e.g. described in WO2009077483, WO2018148447, WO2019157366. Antibodies against NKp30 are e.g. described in WO2020172605. Antibodies against NKp46 are e.g. described in WO2011086179 and WO2016209021. Antibodies against DNAM-1 are e.g. described in WO2013140787. Antibodies against SLAMF7 are e.g. described in US2018208653. Antibodies against OX40 are e.g. described in WO2007062245, US2010136030, US2019100596, WO2013008171, WO2013028231. Antibodies against CD47/SIRPα are e.g. described in WO9727873, WO2005044857, US2014161799. Antibodies against CD89 are e.g. described in WO02064634, WO2020084056. Antibodies against CD96 are e.g. described in WO2019091449. Antibodies against CD137 are e.g. described in WO2005035584, WO2006088464, US2006188439. Antibodies against CD160 are e.g. described in US2012003224, US2013122006. Antibodies against TIGIT are e.g. described in US2020040082 and WO2019062832. Antibodies against nectin-4 are e.g. described in WO2018158398. Antibodies against PD-1 are e.g. described in WO2009014708, US2012237522, US2013095098, and US2011229461. Antibodies against PD-L1 are e.g. described in US2012237522, WO2014022758, WO2014055897, and WO2014195852. Antibodies against LAG-3 are e.g. described in WO2008132601, US2016176965, and WO2010019570. Antibodies against CTLA-4 are e.g. described in WO2005092380, US2009252741, and WO2006066568. Antibodies against TIM-3 are e.g. described in US2014134639, WO2011155607, and WO2015117002. Antibodies against KIR2DS1-5 and are e.g. described in WO2016031936. Antibodies against CD3 are e.g. described in U.S. Pat. No. 6,750,325, WO9304187, and WO9516037.

In some preferred embodiments, the second binding domain (B) is specific for NKG2D and preferably comprises three heavy chain CDRs and three light chain CDRs selected form the group consisting of (a) a CDR-H1 as depicted in SEQ ID NO: 56, a CDR-H2 as depicted in SEQ ID NO: 57, a CDR-H3 as depicted in SEQ ID NO: 58, a CDR-L1 as depicted in SEQ ID NO: 59, a CDR-L2 as depicted in SEQ ID NO: 60, a CDR-L3 as depicted in SEQ ID NO: 61; and (b) a CDR-H1 as depicted in SEQ ID NO: 62, a CDR-H2 as depicted in SEQ ID NO: 63, a CDR-H3 as depicted in SEQ ID NO: 64, a CDR-L1 as depicted in SEQ ID NO: 65, a CDR-L2 as depicted in SEQ ID NO: 66, a CDR-L3 as depicted in SEQ ID NO: 67.

In some preferred embodiments, the second binding domain (B) comprises a pair of VH- and VL-chains having a sequence as depicted in the pairs of sequences selected form the group consisting of SEQ ID NOs: 15 and 17, SEQ ID NOs: 16 and 17, SEQ ID NOs: 18 and 20, SEQ ID NOs: 19 and 20.

In some preferred embodiments, the second binding domain (B) is specific for Nkp46 and preferably comprises a VH domain comprising the three heavy chain CDRs and a VL domain comprising the three light chain CDRs selected form the group consisting of (a) a CDR-H1 as depicted in SEQ ID NO: 68, a CDR-H2 as depicted in SEQ ID NO: 69, a CDR-H3 as depicted in SEQ ID NO: 70, a CDR-L1 as depicted in SEQ ID NO: 71, a CDR-L2 as depicted in SEQ ID NO: 72, a CDR-L3 as depicted in SEQ ID NO: 73; and (b) a CDR-H1 as depicted in SEQ ID NO: 74, a CDR-H2 as depicted in SEQ ID NO: 75, a CDR-H3 as depicted in SEQ ID NO: 76, a CDR-L1 as depicted in SEQ ID NO: 77, a CDR-L2 as depicted in SEQ ID NO: 78, a CDR-L3 as depicted in SEQ ID NO: 79.

In some preferred embodiments, the second binding domain (B) comprises a pair of VH- and VL-chains having a sequence as depicted in the pairs of sequences selected form the group consisting of SEQ ID NOs: 21 and 23, SEQ ID NOs: 22 and 23, and SEQ ID NOs: 24 and 25.

In some preferred embodiments, the second binding domain (B) is specific for CD89 and preferably comprises a VH domain comprising the three heavy chain CDRs and a VL domain comprising the three light chain CDRs selected form the group consisting of (a) a CDR-H1 as depicted in SEQ ID NO: 460, a CDR-H2 as depicted in SEQ ID NO: 461, a CDR-H3 as depicted in SEQ ID NO: 462, a CDR-L1 as depicted in SEQ ID NO: 463, a CDR-L2 as depicted in SEQ ID NO: 464, a CDR-L3 as depicted in SEQ ID NO: 465; and (b) a CDR-H1 as depicted in SEQ ID NO: 466, a CDR-H2 as depicted in SEQ ID NO: 467, a CDR-H3 as depicted in SEQ ID NO: 468, a CDR-L1 as depicted in SEQ ID NO: 469, a CDR-L2 as depicted in SEQ ID NO: 470, a CDR-L3 as depicted in SEQ ID NO: 471.

In some preferred embodiments, the second binding domain (B) comprises a pair of VH- and VL-chains having a sequence as depicted in the pairs of sequences selected form the group consisting of SEQ ID NOs: 456 and 457 and SEQ ID NOs: 458 and 459.

In some embodiments, the third binding domain (C) is specific for a third target (C′) that is a tumor associated antigen. The third target (C′) is preferably selected from the group consisting of CD19, CD20, CD22, CD30, CD33, CD52, CD70, CD74, CD79b, CD123, CLL1, BCMA, FCRH5, EGFR, EGFRvlll, HER2, GD2.

These cell surface antigens on the surface of target cells are connected with specific disease entities. CD30 is a cell surface antigen characteristic for malignant cells in Hodgkin lymphoma. CD19, CD20, CD22, CD70, CD74 and CD79b are cell surface antigens characteristic for malignant cells in Non-Hodgkin lymphomas (Diffuse large B-cell lymphoma (DLBCL), Mantle cell lymphoma (MCL), Follicular lymphoma (FL), T-cell lymphomas (both peripheral and cutaneous, including transformed mycosis fungoides/Sezary syndrome TMF/SS and Anaplastic large-cell lymphoma (ALCL)). CD52, CD33, CD123, CLL1 are cell surface antigens characteristic for malignant cells in Leukemias (Chronic lymphocytic leukemia (CLL), Acute lymphoblastic leukemia (ALL), Acute myeloid leukemia (AML)). BCMA, FCRH5 are cell surface antigens characteristic for malignant cells in Multiple Myeloma. EGFR, HER2, GD2 are cell surface antigens characteristic for solid cancers (Triple-negative breast cancer (TNBC), breast cancer BC, Colorectal cancer (CRC), Non-small-cell lung carcinoma (NSCLC), Small-cell carcinoma (SCLC also known as “small-cell lung cancer”, or “oat-cell carcinoma”), Prostate cancer (PC), Glioblastoma (also known as glioblastoma multiforme (GBM)).

Antibodies against such targets are well known in the art. Antibodies against CD19 are e.g. described in WO2018002031, WO2015157286, and WO2016112855. Antibodies against CD20 are e.g. described in WO2017185949, US2009197330, and WO2019164821. Antibodies against CD22 are e.g. described in WO2020014482, WO2013163519, U.S. Ser. No. 10/590,197. Antibodies against CD30 are e.g. described in WO2007044616, WO2014164067, and WO2020135426. Antibodies against CD33 are e.g. described in WO2019006280, WO2018200562, and WO2016201389. Antibodies against CD52 are e.g. described in WO2005042581, WO2011109662, and US2003124127. Antibodies against CD70 are e.g. described in US2012294863, WO2014158821, and WO2006113909. Antibodies against CD74 are e.g. described in WO03074567, US2014030273, and WO2017132617. Antibodies against CD79b are e.g. described in US2009028856, US2010215669, and WO2020088587. Antibodies against CD123 are e.g. described in US2017183413, WO2016116626, and U.S. Ser. No. 10/100,118. Antibodies against CLL1 are e.g. described in WO2020083406. Antibodies against BCMA are e.g. described in WO02066516, U.S. Ser. No. 10/745,486, and US2019112382. Antibodies against FCRH5 are e.g. described in US2013089497. Antibodies against EGFR are e.g. described in WO9520045, WO9525167, and WO02066058. Antibodies against EGFRvlll are e.g. described in WO2017125831. Antibodies against HER2 are e.g. described in US2011189168, WO0105425, and US2002076695. Antibodies against GD2 are e.g. described in WO8600909, WO8802006, and U.S. Pat. No. 5,977,316.

In some preferred embodiments, the third binding domain (C) is specific for EGFR and preferably comprises a VH domain comprising the following three heavy chain CDRs and a VL domain comprising the following three light chain CDRs: a CDR-H1 as depicted in SEQ ID NO: 44, a CDR-H2 as depicted in SEQ ID NO: 45, a CDR-H3 as depicted in SEQ ID NO: 46, a CDR-L1 as depicted in SEQ ID NO: 47, a CDR-L2 as depicted in SEQ ID NO: 48, a CDR-L3 as depicted in SEQ ID NO: 49.

In some preferred embodiments, the third binding domain (C) comprises a pair of VH- and VL-chains having a sequence as depicted in the pairs of sequences selected form the group consisting of SEQ ID NOs: 10 and 12 and SEQ ID NOs: 11 and 12.

In some preferred embodiments, the third binding domain (C) is specific for CD19 and preferably comprises a VH domain comprising the following three heavy chain CDRs and a VH domain comprising the following three light chain CDRs: a CDR-H1 as depicted in SEQ ID NO: 50, a CDR-H2 as depicted in SEQ ID NO: 51, a CDR-H3 as depicted in SEQ ID NO: 52, a CDR-L1 as depicted in SEQ ID NO: 53, a CDR-L2 as depicted in SEQ ID NO: 54, a CDR-L3 as depicted in SEQ ID NO: 55.

In some preferred embodiments, the third binding domain (C) comprises a pair of VH- and VL-chains having a sequence as depicted in SEQ ID NOs: 13 and 14.

An antibody construct of the invention is preferably an antibody construct selected from the group consisting of SEQ ID NOs: 161-162; 163-164; 165-166; 167-168; 177-179; 180-182; 183-185; 186-188; 189-191; 192-194; 195-197; 198-200; 225-227; 228-230; 231-233; 234-236 237-238, 239-240, 241-242, 243-244, 245-246, 247-248, 249-250, 251-252; 269-270; 271-272; 273-274; 275-276; 277-278; 279-280; 281-282; 283-284; 293-295; 296-298; 299-301; 302-304; 305-307; 308-310; 311-313; 314-316; 329-331; 332-334; 335-337; 338-340; 353-354; 355-356; 357-358; 359-360; 369-371; 372-374; 375-377; 378-380; 431-433; 434-436; 437-439, 490-492, 493-495, and 500-502.

An antibody construct of the invention is preferably an variant of an antibody construct selected from the group consisting of SEQ ID NOs: 161-162; 163-164; 165-166; 167-168; 177-179; 180-182; 183-185; 186-188; 189-191; 192-194; 195-197; 198-200; 225-227; 228-230; 231-233; 234-236 237-238, 239-240, 241-242, 243-244, 245-246, 247-248, 249-250, 251-252; 269-270; 271-272; 273-274; 275-276; 277-278; 279-280; 281-282; 283-284; 293-295; 296-298; 299-301; 302-304; 305-307; 308-310; 311-313; 314-316; 329-331; 332-334; 335-337; 338-340; 353-354; 355-356; 357-358; 359-360; 369-371; 372-374; 375-377; 378-380; 431-433; 434-436; 437-439, 490-492, 493-495, and 500-502, wherein the variant has at least 90%, preferably at least 95%, more preferably at least 98%, even more preferably at least 99% sequence identity to any one of these aforementioned antibody constructs, preferably provided that the CDR sequences comprised in these antibody constructs are not altered.

The present invention also relates to a nucleic acid molecule (DNA and RNA) that includes nucleotide sequences encoding an antibody construct disclosed herein. The present disclosure also encompasses a vector comprising a nucleic acid molecule of the invention. The present invention also encompasses a host cell containing said nucleic acid molecule or said vector. Since the degeneracy of the genetic code permits substitutions of certain codons by other codons specifying the same amino acid, the disclosure is not limited to a specific nucleic acid molecule encoding a antibody construct as described herein but encompasses all nucleic acid molecules that include nucleotide sequences encoding a functional polypeptide. In this regard, the present disclosure also relates to nucleotide sequences encoding the antibody constructs of the disclosure.

A nucleic acid molecule disclosed in this application may be “operably linked” to a regulatory sequence (or regulatory sequences) to allow expression of this nucleic acid molecule.

A nucleic acid molecule, such as DNA, is referred to as “capable of expressing a nucleic acid molecule” or capable “to allow expression of a nucleotide sequence” if it includes sequence elements which contain information regarding to transcriptional and/or translational regulation, and such sequences are “operably linked” to the nucleotide sequence encoding the polypeptide. An operable linkage is a linkage in which the regulatory sequence elements and the sequence to be expressed are connected in a way that enables gene expression. The precise nature of the regulatory regions necessary for gene expression may vary among species, but in general these regions include a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the disclosure can include a regulatory sequence, such as a promoter sequence. In some embodiments a nucleic acid molecule of the disclosure includes a promoter sequence and a transcriptional termination sequence. Examples of promoters useful for expression in eukaryotic cells are the SV40 promoter or the CMV promoter.

The nucleic acid molecules of the disclosure can also be part of a vector or any other kind of cloning vehicle, such as a plasmid, a phagemid, a phage, a baculovirus, a cosmid or an artificial chromosome.

Such cloning vehicles can include, aside from the regulatory sequences described above and a nucleic acid sequence encoding a antibody construct as described herein, replication and control sequences derived from a species compatible with the host cell that is used for expression as well as selection markers conferring a selectable phenotype on transformed or transfected cells. Large numbers of suitable cloning vectors are known in the art, and are commercially available.

The disclosure also relates to a method for the production of an antibody construct of the disclosure, wherein the antibody construct is produced starting from the nucleic acid coding for the antibody construct or any subunit therein. The method can be carried out in vivo, the polypeptide can, for example, be produced in a bacterial or eukaryotic host organism and then isolated from this host organism or its culture. It is also possible to produce an antibody construct of the disclosure in vitro, for example by use of an in vitro translation system.

When producing the antibody construct in vivo, a nucleic acid encoding such polypeptide is introduced into a suitable bacterial or eukaryotic host organism by means of recombinant DNA technology. For this purpose, the host cell may be transformed with a cloning vector that includes a nucleic acid molecule encoding an antibody construct as described herein using established standard methods. The host cell may then be cultured under conditions, which allow expression of the heterologous DNA and thus the synthesis of the corresponding polypeptide or antibody construct. Subsequently, the polypeptide or antibody construct is recovered either from the cell or from the cultivation medium.

Suitable host cells can eukaryotic, such as immortalized mammalian cell lines (e.g., HeLa cells or CHO cells) or primary mammalian cells.

An antibody construct of the disclosure as described herein may be not necessarily generated or produced only by use of genetic engineering. Rather, such polypeptide can also be obtained by chemical synthesis such as Merrifield solid phase polypeptide synthesis or by in vitro transcription and translation. Methods for the solid phase and/or solution phase synthesis of proteins are well known in the art (see e.g. Bruckdorfer, T. et al. (2004) Curr. Pharm. Biotechnol. 5, 29-43).

An antibody construct of the disclosure may be produced by in vitro transcription/translation employing well-established methods known to those skilled in the art.

The invention also provides a composition, preferably a pharmaceutical composition comprising an antibody construct of the invention.

Certain embodiments provide pharmaceutical compositions comprising the antibody construct defined in the context of the invention and further one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of one aspect of the invention to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18″ Edition, (A.R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine
antimicrobials such as antibacterial and antifungal agents
antioxidants such as ascorbic acid, methionine, sodium sulfite or sodium hydrogen-sulfite;
buffers, buffer systems and buffering agents which are used to maintain the composition at physiological pH or at a slightly lower pH; examples of buffers are borate, bicarbonate,
Tris-HCl, citrates, phosphates or other organic acids, succinate, phosphate, and histidine; for example Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetraacetic acid (EDTA);
isotonic and absorption delaying agents;
complexing agents such as caffeine, polyvinylpyrrolidone, beta-cyclodextrin or hydroxypropyl-beta-cyclodextrin)
fillers;
monosaccharides; disaccharides; and other carbohydrates (such as glucose, mannose or dextrins); carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as glycerin, propylene glycol or polyethylene glycol);
sugars and sugar alcohols, such as trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

It is evident to those skilled in the art that the different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles.

In one embodiment of the pharmaceutical composition according to one aspect of the invention the composition is administered to a patient intravenously.

Methods and protocols for the intravenous (iv) administration of pharmaceutical compositions described herein are well known in the art.

The antibody construct of the invention and/or pharmaceutical composition of the invention is preferably used in the prevention, treatment or amelioration of a disease selected from a proliferative disease, a tumorous disease, a viral disease or an immunological disorder. Preferably, said tumorous disease is a malignant disease, preferably cancer.

In one embodiment of the pharmaceutical composition of the invention the identified malignant disease is selected from the group consisting of Hodgkin lymphoma, Non-Hodgkin lymphoma, leukemia, multiple myeloma and solid tumors.

The present invention also provides a method for the treatment or amelioration of a disease, the method comprising the step of administering to a subject in need thereof an antibody construct according to the invention.

In one embodiment of said method for the treatment or amelioration of a disease the subject suffers from a proliferative disease, a tumorous disease, an infectious disease such as a viral disease, or an immunological disorder. It is preferred that said tumorous disease is a malignant disease, preferably cancer.

In one embodiment of said method for the treatment or amelioration of a disease said malignant disease is selected from the group consisting of Hodgkin lymphoma, Non-Hodgkin lymphoma, leukemia, multiple myeloma and solid tumors.

The present invention also relates to a method of simultaneously binding a target cell and an immune effector cell, comprising administering to a subject the antibody construct of the invention, wherein the antibody construct binds the tumor cell and a first immune effector cell but does not essentially bind a further immune effector cell. Such a method preferably for the treatment or amelioration of a disease defined herein. Simultaneously binding of a target cell and an immune effector cell preferably comprises target cell specific activation of the immune effector cell. In some embodiments, the first binding domain and the second binding domain preferably bind to a first target (A′) and a second target (B′) that are on the same first immune effector cell. In some embodiments, only one of the first binding domain (A) and the second binding domain (B) binds to an immune effector cell, in particular if the first target (A′) and the second target (B′) are expressed on two different immune effector cells.

The present invention also relates to a kit comprising an antibody construct of the invention, a nucleic acid molecule of the invention, a vector of the invention or a host cell of the invention. The kit of the invention will typically comprise a container comprising the antibody construct of the invention, the nucleic acid molecule of the invention, the vector of the invention, or the host cell of the invention, and optionally one or more other containers comprising materials desirable from a commercial and user standpoint, including buffers, diluents, filters, needles, syringes, and package inserts with instructions for use.

The invention is further characterized by the following items.

Item 1. A trispecific antibody construct comprising (i) a first binding domain (A), which is capable of specifically binding to a first target (A′) that is CD16A on the surface of an immune effector cell; (ii) a second binding domain (B), which is capable of specifically binding to a second target (B′) that is another antigen on the surface of an immune effector cell, wherein said antigen is selected from the group comprising CD56, NKG2A, NKG2D, NKp30, NKp44, NKp46, NKp80, DNAM-1, SLAMF7, OX40, CD47/SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5 and CD3; and (iii) a third binding domain (C), which is capable of specifically binding to a third target (C′) that is an antigen on the surface of a target cell.

Item 2. The antibody construct of item 1, wherein the first binding domain (A) and the second binding domain (B) are positioned to each other in a way that simultaneous binding of two immune effector cells is reduced or preferably prevented.

Item 3. The antibody construct of item 1 or 2, wherein the antibody construct binds to a target cell and one immune effector cell simultaneously.

Item 4. The antibody construct of the preceding items, further comprising a fourth domain (D) comprising a half-life extension domain.

Item 5. The antibody construct of item 4, wherein said half-life extension domain comprises a CH2 domain, wherein the Fcγ receptor binding domain is silenced.

Item 6. The antibody construct of item 4 or 5, wherein said half-life extension domain comprises a CH3 domain.

Item 7. The antibody construct of any one of items 4 to 6, wherein the antibody construct comprise at least one hinge domain and CH3 domain fused to a CH2 domain in an amino to carboxyl order in the order hinge domain-CH2 domain-CH3 domain.

Item 8. The antibody construct of any one of items 4 to 7, wherein the antibody construct comprises at least two of the hinge domain-CH2 domain-CH3 domain elements.

Item 9. The antibody construct of any one of the preceding items, wherein the third binding domain (C) comprises an VH and a VL domain of an antibody.

Item 10. The antibody construct of any one of the preceding items, wherein the third binding domain (C) binds to an antigen on the surface of a target cell, which antigen is selected from the group consisting of CD19, CD20, CD22, CD30, CD33, CD52, CD70, CD74, CD79b, CD123, CLL1, BCMA, FCRH5, EGFR, EGFRvlll, HER2, and GD2.

Item 11. The antibody construct of any one of the preceding items, wherein the second binding domain (B) comprises an VH and a VL domain of an antibody.

Item 12. The antibody construct of any one of the preceding items, wherein the first binding domain (A) comprises an VH and a VL domain of an antibody.

Item 13. The antibody construct of any one of the preceding items, wherein the first binding domain (A) binds to an epitope on CD16A which is C-terminal to the physiological Fcγ receptor binding domain, said epitope preferably comprises Y158 of SEQ ID NO: 449.

Item 14. The antibody construct of any one of the preceding items, wherein the first binding domain (A) is fused to the C terminus of a first CH3 domain and the second binding domain (B) is fused to the C terminus of a second CH3 domain.

Item 15. The antibody construct of item 14, wherein the antibody construct is monovalent for the first binding domain (A) and monovalent for the second binding domain (B).

Item 16. The antibody construct of any one of items 1 to 13, wherein the first binding domain (A) is fused to the N-terminus of a first hinge and the second binding domain (B) is fused to the N-terminus of a second hinge.

Item 17. The antibody construct of any one of items 1 to 13, wherein the first binding domain (A) and the second binding domain (B) are fused to each other.

Item 18. The antibody construct of item 17, wherein the antibody construct is monovalent for the first binding domain (A) and monovalent for the second binding domain (B).

Item 19. The antibody construct of item 17, wherein the antibody construct is bivalent for the first binding domain (A) and bivalent for the second binding domain (B), wherein each of the first binding domains (A) is fused to a second binding domain (B).

Item 20. The antibody construct of any one of items 17 to 19, wherein the C terminus of the VL of the first binding domain (A) is fused to the N terminus of the VH of the second binding domain (B) and the C terminus of the VL of the second binding domain (B) is fused to the N terminus of the VH of the first binding domain (A).

Item 21. The antibody construct of any one of items 17 to 19, wherein the N terminus of the VL of the first binding domain (A) is fused to the C terminus of the VH of the second binding domain (B) and the N terminus of the VL of the second binding domain (B) is fused to the C terminus of the VH of the first binding domain (A).

Item 22. The antibody construct of any one of items 17 to 19, wherein the C terminus of the VL of the first binding domain (A) is fused to the N terminus of the VL of the second binding domain (B) and the C terminus of the VH of the first binding domain (A) is fused to the N terminus of the VH of the second binding domain (B).

Item 23. The antibody construct of any one of items 17 to 19, wherein the C terminus of the VL of the second binding domain (B) is fused to the N terminus of the VL of the first binding domain (A) and the C terminus of the VH of the second binding domain (B) is fused to the N terminus of the VH of the first binding domain (A).

Item 24. The antibody construct of any one of items 17 to 19, wherein the first binding domain (A) and the second binding domain (B) are fused to each other in form of a bi-scFv, double Fab, db or scDb.

Item 25. The antibody construct of item 24, wherein the first binding domain (A) and the second binding domain (B) are fused to each other in form of a db or scDb.

Item 26. The antibody construct of item 25, wherein the variable domains of the db or scDb are arranged in V_{L}-V_{H}-V_{L}-V_{H} order.

Item 27. The antibody construct of any one of items 16 to 26, wherein (a) the first binding domain (A) is fused N-terminally to a hinge domain and the second binding domain (B) is fused N-terminally to the first binding domain (A); or (b) the first binding domain (A) is fused C-terminally to a CH3 domain and the second binding domain (B) is fused C-terminally to the first binding domain.

Item 28. The antibody construct of any one of items 16 to 27, wherein the first binding domain (A) is fused N-terminally to a hinge domain and the second binding domain (B) is fused N-terminally to the first binding domain (A).

Item 29. The antibody construct of any one of the preceding items, wherein the binding site of the first binding domain (A) and the binding site of the second binding domain (B) are within a distance of about 25 nm or less, preferably about 20 nm or less, preferably about 15 nm or less, preferably about 10 nm or less.

Item 30. The antibody construct of any one of the preceding items, wherein the binding site of the first binding domain (A) and the binding site of the second binding domain (B) are in cis orientation.

Item 31. The antibody construct of any one of the preceding items, wherein the binding site of the first binding domain (A) and the binding site of the third binding domain (C) are in trans orientation.

Item 32. The antibody construct of any one of the preceding items, wherein the binding site of the second binding domain (B) and the binding site of the third binding domain (C) are in trans orientation.

Item 33. The antibody construct of any one of the preceding items, wherein the first binding domain (A) comprises:
(i) a VL region comprising CDR-L1, CDR-L2 and CDR-L3 selected from:
   (a) CDR-L1 as depicted in SEQ ID NO: 29, a CDR-L2 as depicted in SEQ ID NO: 30, a CDR-L3 as depicted in SEQ ID NO: 31; and
   (b) CDR-L1 as depicted in SEQ ID NO: 35, a CDR-L2 as depicted in SEQ ID NO: 36, a CDR-L3 as depicted in SEQ ID NO: 37;
(ii) a VH region comprising CDR-H1, CDR-H2 and CDR-H3 selected from:
   (a) CDR-H1 as depicted in SEQ ID NO: 26, a CDR-H2 as depicted in SEQ ID NO: 27, a CDR-H3 as depicted in SEQ ID NO: 28; and
   (b) a CDR-L1 as depicted in SEQ ID NO: 29, a CDR-L2 as depicted in SEQ ID NO: 30, a CDR-L3 as depicted in SEQ ID NO: 31.

Item 34. The antibody construct of any one of the preceding items, having an amino acid sequence selected from the group consisting of SEQ ID NOs: 161-162; 163-164; 165-166; 167-168; 177-179; 180-182; 183-185; 186-188; 189-191; 192-194; 195-197; 198-200; 225-227; 228-230; 231-233; 234-236 237-238, 239-240, 241-242, 243-244, 245-246, 247-248, 249-250, 251-252; 269-270; 271-272; 273-274; 275-276; 277-278; 279-280; 281-282; 283-284; 293-295; 296-298; 299-301; 302-304; 305-307; 308-310; 311-313; 314-316; 329-331; 332-334; 335-337; 338-340; 353-354; 355-356; 357-358; 359-360; 369-371; 372-374; 375-377; 378-380; 431-433; 434-436; 437-439, 490-492, 493-495, and 500-502.

Item 35. The antibody construct of and one of the preceding items, wherein the antibody construct induces a lower degree of fratricide as compared to a control construct selected from the group consisting of SEQ ID NOs: 393-395; 396-398; 399-401; 402-404; 405-407; 408-410; 411-413; 414-416; 417-419; 420-422; 423-425; and 426-428.

Item 36. The antibody construct of any one of the preceding items, wherein the antibody construct induces a lower degree of fratricide as compared to the anti-CD38 antibody of SEQ ID NOs: 429 and 430.

Item 37. The antibody construct of any one of the preceding items, wherein the antibody construct induces about 25% or less NK cell fratricide in a cytotoxicity assay.

Item 38. A nucleic acid molecule comprising a sequence encoding an antibody construct of any one of items 1 to 37.

Item 39. A vector comprising a nucleic acid molecule of item 38.

Item 40. A host cell comprising a nucleic acid molecule of item 38 or a vector of item 39.

Item 41. A method of producing an antibody construct of any one of items 1 to 37, said method comprising culturing a host cell of item 40 under conditions allowing the expression of the antibody construct of any one of items 1 to 37 and recovering the produced antibody construct from the culture.

Item 42. A pharmaceutical composition comprising an antibody construct of any one of items 1 to 37, or produced of the method of item 41.

Item 43. An antibody construct of any one of items 1 to 37 for use in therapy.

Item 44. The antibody construct of any one of items 1 to 37, or produced of the method of item 41, for use in the prevention, treatment or amelioration of a disease selected from a proliferative disease, a tumorous disease, a viral disease or an immunological disorder.

Item 45. A method of treatment or amelioration of a proliferative disease, a tumorous disease, a viral disease or an immunological disorder, comprising the step of administering to a subject in need thereof the antibody construct of any one of items 1 to 37, or produced of the process of item 41.

Item 46. A kit comprising an antibody construct of any one of items 1 to 37, or produced of the method of item 41, a nucleic acid molecule of item 38, a vector of item 39, and/or a host cell of item 40.

Item 47. A method of simultaneously binding a target cell and an immune effector cell, comprising administering to a subject the antibody construct of any one of items 1 to 37, wherein the antibody construct binds the tumor cell and a first immune effector cell but does not essentially bind a further immune effector cell.

Item 48. The method of item 47, wherein the first binding domain and the second binding domain bind to a first target (A′) and a second target (B′) that are on the same first immune effector cell.

Item 49. The method of item 47 or 48, wherein the method comprises target cell specific activation of the first immune effector cell.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 10%, preferably within 5%, more preferably within 2%, even more preferably within 1% of a given value or range (plus (+) or minus (−)). It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms. For example, the disclosure of the term “comprising” includes the disclosure of the terms “consisting essentially of” as well as the disclosure of the term “consisting of”.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended to limit the scope of the present invention in any way.

### EXAMPLES

### Example 1: Culture of Transfected CHO Cells

Stably transfected CHO cells expressing recombinant cell surface anchored CD16A, CD16B, CD32, CD64, NKp46, NKG2D, or other innate cell receptors, or EGFR, CD19, HER2, CD30, CD33, or other tumor target antigens were cultured in HyClone CDM4 CHO (Cytiva Lifesciences, cat. SH30557.02) supplemented with 2 mM L-Glutamine (Life Technologies, cat. 25030-024) and 0.5× HT supplement (Life Technologies, cat. 41065-012). To maintain stable recombinant antigen expression, culture media was supplemented with selection antibiotics, e.g. 7 μg/mL Puromycin (Fisher Scientific, cat. A1113803) or 500 μg/mL Hygromycin B (Fisher Scientific, cat. 10687010). Suspension cultures were seeded at a density of 3×10⁵ viable cells/mL for a subsequent 3-day passage, or 6×10⁵ viable cells/mL for a subsequent 2-day passage.

### Example 2: Culture of Cell Lines

EGFR⁺ tumor cells, e.g. A-431 (DSMZ; cat.: ACC 91) or SW-982 (ATCC; cat.: HTB-93) and CD19⁺ GRANTA-519 cells (DSMZ; cat.: ACC 342) were cultured under standard conditions in DMEM medium supplemented with 10% heat-inactivated FCS, 2 mM L-glutamine and 100 IU/mL penicillin G sodium and 100 μg/mL streptomycin sulfate (all components from Invitrogen) as recommended by the supplier. CD32⁺/CD64⁺ tumor cells, e.g. THP-1 (DSMZ; ACC 16), and CD19⁺ tumor cells, e.g. Raji (DSMZ, cat.: ACC 319) were cultured under standard conditions in RPMI 1640 medium supplemented with 10% heat-inactivated FCS, 2 mM L-glutamine and 100 IU/mL penicillin G sodium and 100 μg/mL streptomycin sulfate (all components from Invitrogen). The HER2⁺ SK-BR-3 cell line was purchased from DSMZ (cat.: ACC 736) and cultured in McCoy's medium (ATCC, cat.: ATCC30-2007) supplemented with 20% heat-inactivated FCS, 2 mM L-glutamine and 100 IU/mL penicillin G sodium and 100 μg/mL streptomycin sulfate (all components from Invitrogen). All cell lines were cultured at 37° C. in a humidified atmosphere with 5% CO₂.

### Example 3: Isolation of PBMC from Buffy Coats

Peripheral blood mononuclear cells (PBMC) were isolated from buffy coats (German Red Cross, Mannheim, Germany) by density gradient centrifugation. The buffy coat samples were diluted with a two-to-threefold volume of PBS (Invitrogen, cat.: 14190-169), layered on a cushion of Lymphoprep (Stem Cell Technologies, cat.: 07861) SepMate™-50 (IVD) tubes (Stem Cell Technologies, cat.: 85460), and centrifuged at 800×g for 25 min at room temperature w/o brake. PBMC located in the interface were collected and washed three times with PBS before use. Where indicated, PBMC were cultured overnight without stimulation in complete RPMI 1640 medium (RPMI 1640 medium supplemented 10% heat-inactivated FCS, 2 mM L-glutamine and 100 IU/mL penicillin G sodium and 100 μg/mL streptomycin sulfate (all components from Invitrogen).

### Example 4: Enrichment of Human NK Cells or T Cells and Depletion of B Cells from PBMC

For the immunomagnetic enrichment of untouched primary human NK or T cells, PBMC were harvested from overnight cultures and used for one or two rounds of negative selection using the EasySep™ Human NK Cell Enrichment Kit (Stem Cell Technologies, cat.: 17055) or the EasySep™ Human T Cell Enrichment Kit (Stem Cell Technologies, cat.: 19051) with the Big Easy EasySep™ Magnet (Stem Cell Technologies, cat.: 18001) according to the manufacturer's instructions.

For the depletion of CD19⁺ cells from PBMC, PBMC were subjected to one or two rounds of B cell depletion using the B cell EasySep™ Human CD19 Positive Selection Kit (Stem Cell Technologies, cat: 18054) according to manufacturer's instructions.

### Example 5: Assessment of Purity of Enriched NK Cells by Flow Cytometry

Aliquots of e.g. 1×10⁶ enriched human NK cells were washed in FACS buffer (PBS (Invitrogen, cat.: 14190-169) containing 2% heat-inactivated FCS (Invitrogen, cat.: 10270-106), and 0.1% sodium azide (Roth, Karlsruhe, Germany, cat.: A1430.0100)), and were then resuspended in FACS/hIgG buffer (FACS buffer (PBS containing 2% heat-inactivated FCS, and 0.1% sodium azide) supplemented with 1 mg/mL polyclonal human IgG (hIgG, e.g. Cutaquig, Octapharma)) containing the antibody panel for immunophenotyping presented in Table 2.

TABLE 2
Antibody panel for immunophenotyping

| Reagent | Supplier | Cat. |
|---|---|---|
| Viability dye eFluor780 | Thermo Fisher | 65-0865-14 |
| CD3-BV510 (UCHT-1) | Biolegend | 300448 |
| CD19-ECD (J3-119) | Beckman Coulter | A07770 |
| CD56-PE (N901) | Beckman Coulter | A07788 |
| CD66b-Alexa647 (G10F5) | Biolegend | 305110 |
| CD16-BV421 (3G8) | BioLegend | 302038 |
| CD45-BV605 (HIT30) | Biolegend | 304042 |
| CD14-PC7 (RMO52) | Beckman Coulter | A22331 |
| CD16-158V-FITC (MEM-154) | Thermo Fisher | MA1-19563 |
| CD8-BV785 (SK1) | Biolegend | 344740 |

After incubation for 30 min on ice in the dark, cells were washed twice in FACS buffer, and were then resuspended in PBS. Cells were then analyzed using a standardized 10-color protocol using a CytoFlex 3L flow cytometer (Beckman Colter) to determine the purity of enriched NK cells and the relative amount of other cell subsets. The purity of enriched NK cells after one round of negative selection was typically >80% CD16⁺/CD56⁺ cells of total cells. An exemplary dot plot from an NK cell enrichment experiment is shown in FIG. 13.

### Example 6: Cell Binding Assays and Flow Cytometric Analysis

Aliquots of 1×10⁵ to 1×10⁶ of the indicated cells were incubated with 100 μL of the indicated antibody constructs at the indicated concentrations, e.g. 10 μg/mL or 100 μg/mL, in FACS buffer (PBS (Invitrogen, cat.: 14190-169) containing 2% heat-inactivated FCS (Invitrogen, cat.: 10270-106), and 0.1% sodium azide (Roth, Karlsruhe, Germany, cat.: A1430.0100)) for 45 min at 37° C. After repeated washing with FACS buffer, cell-bound antibodies were detected with fluorescence-labeled secondary reagents, e.g. 15 μg/mL FITC-conjugated goat anti-human IgG Fc (Dianova, cat.: 109-095-098). Fluorescence-labeled mAbs specific for CD16 (clone 3G8, Biolegend), CD32 (clone FLI8.26, BD Biosciences), CD64 (clone 10.1, Biolegend), NKp46 (clone 9E2, BD Bioscience), and NKG2D (clone 1D11, Biolegend) were used as controls. After the last staining step, the cells were washed again and resuspended in 0.2 mL of FACS buffer. The median fluorescence intensity (MFI) of 0.5-5×10⁴ cells was measured using a Beckman Coulter CytoFLEX or CytoFLEX S flow cytometer using CytExpert software (Beckman Coulter, Krefeld, Germany). The MFI of the cell samples were calculated using CytExpert software (Beckman Coulter). Binding histograms of the antibodies to cells were plotted using FlowJo software (version 10.7 for Windows, FlowJo LLC, Ashland, OR, USA). In case of staining of the cells with serial dilutions, the fluorescence intensity values of the cells stained with the secondary reagents alone were subtracted, and the values were used for non-linear regression analysis and plotting dose-response curves using the GraphPad Prism software (version 7.04 for Windows, GraphPad Software, San Diego, CA, USA).

### Example 7: 4 h Calcein-Release Cytotoxicity Assays on Tumor Cell Lines as Target Cells

For calcein-release cytotoxicity assays the indicated target cells were harvested from cultures, washed with RPMI 1640 medium without FCS, and labeled with 10 μM calcein AM (Invitrogen/Molecular Probes, cat.: C3100MP) for 30 min in RPMI 1640 medium without FCS at 37° C. After gently washing, the labeled cells were resuspended in complete RPMI 1640 medium (RPMI 1640 medium supplemented with 10% heat-inactivated FCS, 4 mM L-glutamine, 100 U/mL penicillin G sodium, 100 μg/mL streptomycin sulfate) to a density of 1×10⁵/mL. 1×10⁴ target cells were then seeded together with enriched primary human NK cells at an E:T ratio of 5:1 or unfractionated human PBMC at an E:T ratio of 50:1 in the presence of serial dilutions of the indicated antibodies, preferentially in the range between 1 ng/mL and 30 μg/mL, in individual wells of a round-bottom 96-well microplate in a total volume of 200 μL/well in duplicates. Spontaneous release, maximal release and killing of targets by effectors in the absence of antibodies were determined in quadruplicate on each plate. For induction of maximal calcein-release Triton X-100 was added to the respective wells at a final concentration of 1%.

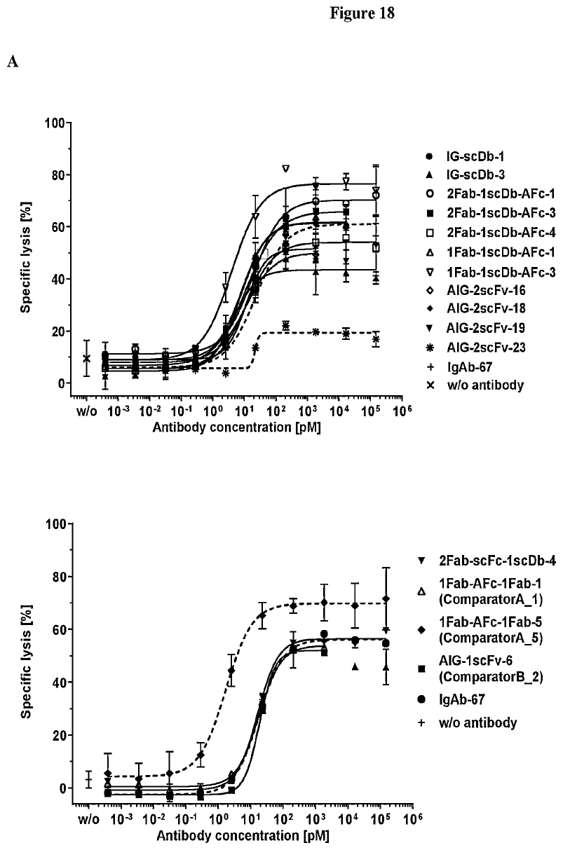

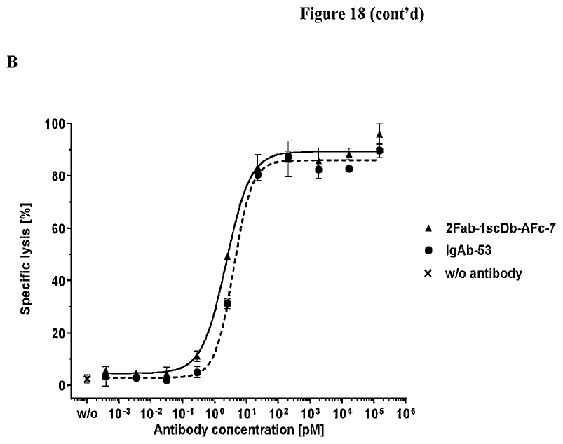

After centrifugation for 2 min at 200×g the assay was incubated for 4 h at 37° C. in a humidified atmosphere with 5% CO₂. 100 μL cell culture supernatant were harvested from each well after an additional centrifugation for 5 min at 500×g, transferred to a black flat-bottom microplate, and the fluorescence of the released calcein was measured at 520 nm using a fluorescence plate reader (EnSight, Perkin Elmer, Waltham, MA, USA). On the basis of the measured counts, the specific cell lysis was calculated according to the following formula: [fluorescence (sample)−fluorescence (spontaneous)]/[fluorescence (maximum)−fluorescence (spontaneous)]×100%. Fluorescence (spontaneous) represents the fluorescent counts from target cells in the absence of effector cells and antibodies and fluorescence (maximum) represents the total cell lysis induced by the addition of Triton X-100. Sigmoidal dose response curves and EC₅₀ values were calculated by non-linear regression/4-parameter logistic fit using the GraphPad Prism software and plotted. The graph of one representative experiment is shown in FIG. 18.

TABLE 3
Potency (EC₅₀) and efficacy (Eₘₐₓ) values determined for trispecific constructs
in 4 h calcein release assays where primary human NK cells were incubated with
calcein-labeled CD19⁺ GRANTA-519 or EGFR⁺ A-431 tumor cells at an
effector:target cell ratio of 5:1 in presence of a serial dilution of the
indicated antibodies. Experiments were performed in duplicates and the resulting
mean and SD values are depicted in the table (n.a. = not applicable)

| | | Effector | Effector | EC50 [pM] | | Eₘₐₓ [%] | |
|---|---|---|---|---|---|---|---|
| Construct | Target | domain 1 | domain 2 | mean | SD | mean | SD |
| IG-scDb-1 | CD19 | P2C47var50 | KYK-2.0 | 14.2 | 10.1 | 45.4 | 6.5 |
| IG-scDb-2 | CD19 | P2C47var50 | ADI-27743 | 12.8 | 5.5 | 24.1 | 6.9 |
| IG-scDb-3 | CD19 | P2C47var50 | NKp46-1 | 5.6 | 1.8 | 40.2 | 1.8 |
| 2Fab-1scDb-AFc-1 | CD19 | ABC1197var8 | KYK-2.0 | 21.4 | 4.0 | 54.8 | 6.1 |
| 2Fab-1scDb-AFc-3 | CD19 | ABC1197var8 | NKp46-1 | 11.2 | 1.6 | 55.4 | 1.6 |
| 2Fab-1scDb-AFc-4 | CD19 | ABC1197var8 | NKp46-3 | 16.1 | 6.8 | 52.1 | 4.8 |
| 1Fab-1scDb-AFc-1 | CD19 | ABC1197var8 | KYK-2.0 | 7.6 | 0.9 | 55.1 | 1.9 |
| 1Fab-1scDb-AFc-3 | CD19 | ABC1197var8 | NKp46-1 | 5.6 | 2.3 | 70.5 | 4.1 |
| AIG-2scFv-16 | CD19 | ABC1197var8 | KYK-2.0 | 10.9 | 4.0 | 32.9 | 5.2 |
| AIG-2scFv-17 | CD19 | ABC1197var8 | ADI-27743 | 6.4 | 2.7 | 33.4 | 8.2 |
| AIG-2scFv-18 | CD19 | ABC1197var8 | NKp46-1 | 7.4 | 0.9 | 32.8 | 6.8 |
| AIG-2scFv-19 | CD19 | ABC1197var8 | NKp46-3 | 12.1 | 3.8 | 37.3 | 6.3 |
| AIG-2scFv-23 | CD19 | 3G8 | NKp46-3 | 7.1 | n.a. | 10.1 | 5.0 |
| AIG-2scDb-4 | CD19 | P2C47var50 | NKp46-3 | 5.7 | 0.9 | 35.8 | 4.1 |
| 2scDb-AFc-3 | CD19 | ABC1197var6 | NKp46-1 | 9.3 | 4.0 | 65.7 | 0.0 |
| 2tascFv-AFc-2 | CD19 | ABC1197var8 | ADI-27743 | 12.5 | 1.7 | 65.6 | 3.4 |
| 2Fab-scFc-1scDb-1 | CD19 | ABC1197var8 | KYK-2.0 | 34.7 | 4.3 | 41.3 | 10.9 |
| 2Fab-scFc-1scDb-2 | CD19 | ABC1197var8 | ADI-27743 | 57.0 | 8.5 | 48.4 | 7.9 |
| 2Fab-scFc-1scDb-3 | CD19 | ABC1197var8 | NKp46-1 | 21.5 | 2.2 | 56.4 | 3.6 |
| 2Fab-scFc-1scDb-4 | CD19 | ABC1197var8 | NKp46-3 | 20.1 | 5.4 | 56.2 | 1.5 |
| 1Fab-AFc-1Fab-1 | CD19 | wt Fc | NKp46-1 | 18.2 | 0.8 | 48.3 | 7.2 |
| (ComparatorA_1) | | | | | | | |
| 1Fab-AFc-1Fab-2 | CD19 | wt Fc | NKp46-3 | 16.7 | 1.8 | 46.2 | 10.3 |
| (ComparatorA_2) | | | | | | | |
| 1Fab-AFc-1Fab-5 | CD19 | Fc enhanced | NKp46-1 | 1.9 | 0.3 | 55.2 | 14.6 |
| (ComparatorA_5) | | | | | | | |
| 1Fab-AFc-1Fab-6 | CD19 | Fc enhanced | NKp46-3 | 2.2 | 0.3 | 47.6 | 15.3 |
| (ComparatorA_6) | | | | | | | |
| AIG-1scFv-6 | CD19 | wt Fc | ADI-27743 | 19.0 | 0.1 | 46.3 | 11.8 |
| (ComparatorB_2) | | | | | | | |
| IgAb_67 | CD19 | Fc enhanced | −/− | 25.7 | 5.9 | 51.9 | 9.3 |
| 2Fab-1scDb-AFc-7 | EGFR | ABC1197var8 | NKp46-1 | 2.1 | 0.3 | 81.2 | 4.9 |
| IgAb_53 | EGFR | Fc enhanced | −/− | 3.4 | 0.7 | 79.9 | 4.5 |

Anti-CD19 trispecifics of the formats IG-scDb, 2Fab-1scDb-AFc, 1Fab-scDb-AFc, AIG-2scFv, 2scDb-AFc, 2tascFv-AFc, 2Fab-scFc1scDb as well as all comparator molecules of formats 1Fab-AFc-1Fab with wt Fe or enhanced Fc domain and the AIG-1scFv molecule and the IgAb-67 antibody lyse target cells with a one to two digit picomolar potency. Efficacies are in the range of 24.1 to 70.5%, except for construct AIG-2scFv-23 which contains a CD16 domain of the 3G8 variant, which performs poorly with only 10.1% efficacy. The IgAb-67 antibody for comparison lyses cells with 25.7 μM potency and an efficacy of 51.9%.

The anti-EGFR/NKp46/CD16 construct 2Fab-1scDb-AFc-7 also exhibits potent ADCC activity (2.1 μM) with an efficacy of 81.2%. The control antibody IgAb-53 for comparison has a potency of 3.4 μM and an efficacy of 79.9% in this assay.

### Example 8: NK Cell Fratricide Assay

For calcein-release cytotoxicity assays to assess NK-NK cell lysis, half of the enriched, non-activated NK cells were washed with RPMI 1640 medium without FCS and labeled with 10 μM calcein AM (Invitrogen/Molecular Probes, cat.: C3100MP) for 30 min in RPMI 1640 medium without FCS at 37° C. After gentle washing, the labeled cells were resuspended in complete RPMI medium (RPMI 1640 medium supplemented with 10% heat-inactivated FCS, 4 mM L-glutamine, 100 U/mL penicillin G sodium, 100 μg/mL streptomycin sulfate) to a density of 5×10⁵/mL. 5×10⁴ calcein-labeled NK cells (T) were then seeded together with 5×10⁴ non-labeled NK cells (E) from the same donor at an E:T ratio of 1:1 in the presence of increasing concentrations of the indicated antibodies, preferentially in the range between 10 ng/mL and 100 μg/mL, in individual wells of a round-bottom 96-well microplate in a total volume of 200 μL/well in duplicates. Human IgG1 anti-CD38 (IgAb_51 as described in WO2020/043670 was used as a positive control). Spontaneous release, maximal release and killing of calcein-labeled NK cells (T) by non-labeled NK cells (E) in the absence of antibodies were determined in quadruplicate on each plate. For induction of maximal calcein-release Triton X-100 was added to the respective wells at a final concentration of 1%. After centrifugation for 2 min at 200×g the assay was incubated for 4 h at 37° C. in a humidified atmosphere with 5% CO₂. After an additional centrifugation for 5 min at 500×g 100 μL cell culture supernatant were harvested from each well, transferred to a black flat-bottom microplate, and the fluorescence of the released calcein was measured at 520 nm using a fluorescence plate reader (EnSight, Perkin Elmer). On the basis of the measured fluorescence counts, the specific cell lysis was calculated according to the following formula: [fluorescence (sample)−fluorescence (spontaneous)]/[fluorescence (maximum)−fluorescence (spontaneous)]×100%. Fluorescence (spontaneous) represents the fluorescent counts from calcein-labeled NK cells (T) in the absence of non-labeled NK cells (E) and antibodies and fluorescence (maximum) represents the total cell lysis induced by the addition of Triton X-100 (1% final concentration). Sigmoidal dose response curves were calculated by non-linear regression/4-parameter logistic fit using the GraphPad Prism software and plotted.

### Example 9: Assessment of NK and T Cell Activation in Cultures of PBMC in the Presence or Absence of Target Cells

For the evaluation of effector cell activation and depletion of target cells by EGFR-targeting antibody constructs, 5×10⁵ unfractionated human PBMCs were seeded in individual wells of a round-bottom 96-well microplate in the presence or absence of 1×10⁴ EGFR⁺ tumor cells, e.g. SW-982 cells, leading to an E:T ratio of 50:1. Before seeding, SW-982 cells were labeled with 0.5 μM CMFDA (Invitrogen, cat.: C7025) for 30 min at 37° C. in serum-free RPMI 1640 medium, and washed twice in serum-free medium.

For the assessment of cell activation and depletion by CD19-targeting antibody constructs 5×10⁵ unfractionated human PBMCs or B cell-depleted PBMC were used.

Cells were cultured in complete RPMI medium (RPMI 1640 medium supplemented with 10% heat-inactivated FCS, 2 mM L-glutamine, 100 U/mL penicillin G sodium, and 100 μg/mL streptomycin sulfate) in the presence of the indicated antibody concentrations, preferentially in the range between 1 ng/mL and 30 μg/mL. After 20 h-24 h incubation at 37° C. with 5% CO₂ in a humidified atmosphere, cells were harvested, washed in FACS buffer (PBS (Invitrogen, cat.: 14190-169) containing 2% heat-inactivated FCS (Invitrogen, cat.: 10270-106), and 0.1% sodium azide (Roth, Karlsruhe, Germany, cat.: A1430.0100)), and were then resuspended in FACS/hIgG buffer (FACS buffer (PBS containing 2% heat-inactivated FCS, and 0.1% sodium azide) supplemented with 1 mg/mL polyclonal human IgG (e.g. Cutaquig, Octapharma)). Cells were then stained with an T cell-specific marker, e.g. CD3-BV510 (Biolegend, cat.: 300448), CD4-PE (Biolegend 317410), or CD8-BV785 (Biolgend, cat.: 344740), B cell-specific marker, e.g. CD20-BV605 (Biolgend, cat.: 302333, NK cell-specific marker, e.g. CD56-PE-Cy7 (Biolegend, cat.: 362510), and activation and inhibitory markers, e.g. CD69-APC (Biolgend, cat.: 310910), or CD25-PE/Dazzle 594 (Biolegend, cat.: 302646), CD137-BV605 (Biolegend, cat.: 309822) or CD154-BV421 (Biolegend, cat.: 310824), OX40-PE (Biolegend, cat.: 350004), PD-1-PE (Miltenyi Biotech, cat.: 130-117-384) or TIGIT-BV421 (Biolegend, cat.: 372710), and a viability dye, e.g. Fixable Viability Dye eFluor™ 780 (Invitrogen, cat.: 65-0865-14), in FACS/hIgG buffer for 15 min on ice in the dark with antibody concentrations recommended by the supplier. After repeated washing with FACS buffer, a defined volume of each cell suspension or cell count, e.g. 1×10⁴ cells, was analyzed by flow cytometry using a CytoFlex or CytoFlex S flow cytometer (Beckman Coulter). For the assessment of antibody-induced effector cell activation the percentage of activated, e.g. CD69⁺ cells of NK cells and the percentage of activated, e.g. CD69⁺ cells of T cells were quantified for each sample. Depletion of EGFR⁺ target cells by anti-EGFR antibody constructs and CD19⁺ target cells by CD19-targeting antibodies was determined by quantification of absolute counts of viable, CMFDA-labeled EGFR⁺ target cells, e.g. SW-982, and viable CD20⁺ B cells, respectively, in a defined volume by flow cytometry, or after acquisition relative to counting beads.

### Example 10: Specific Binding of Trispecific Antibody Constructs to Tumor Antigens on Cells

The specificity of trispecific antibody constructs (e.g. CD19/CD16A/NKG2D, CD19/CD16A/NKp46, EGFR/CD16A/NKG2D, and EGFR/CD16A/NKp46) for the respective tumor cell surface antigens CD19 and EGFR was assessed by incubation of CD19⁺/EGFR⁻ tumor cell lines (e.g. Raji) and CD19⁻/EGFR⁺ tumor cell lines (e.g. SW-982) with the trispecific antibody constructs and control constructs followed by flow cytometric detection with secondary FITC-conjugated goat anti-human IgG Fc antibody. Trispecific constructs comprising anti-CD19 Fv domains specifically bound to CD19⁺/EGFR⁻ tumor cells relative to secondary antibody only, whereas there was no binding detectable to CD19⁻/EGFR⁺ tumor cells. Likewise, antibody constructs comprising anti-EGFR Fv domains exhibited specific binding to CD19⁻/EGFR⁺ tumor cells, but not to CD19⁺/EGFR⁻ tumor cells.

### Example 11: Specific Binding of Trispecifc Antibody Constructs to NK Receptors, e.g. CD16A, CD16B, CD32, CD64, NKG2D, and NKp46, on Cells

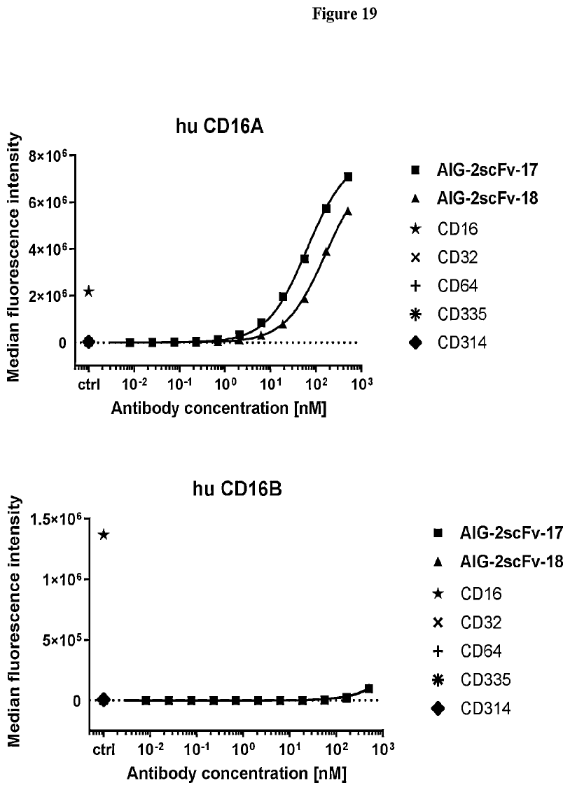

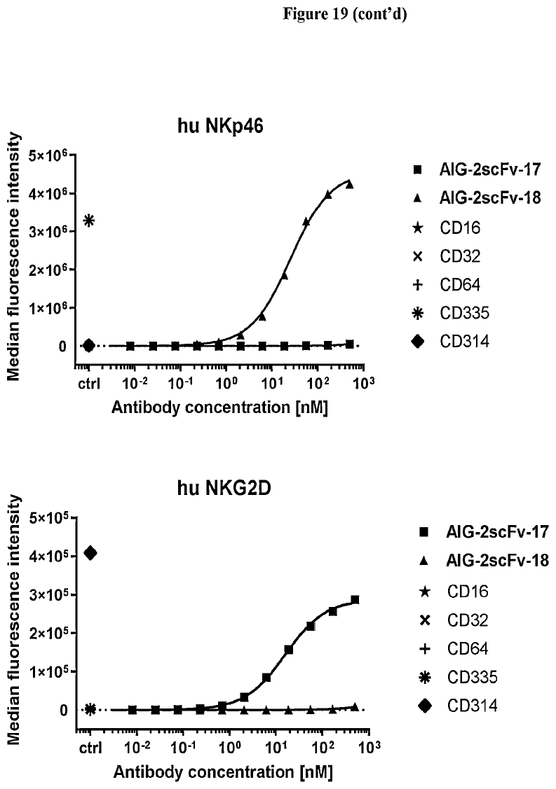

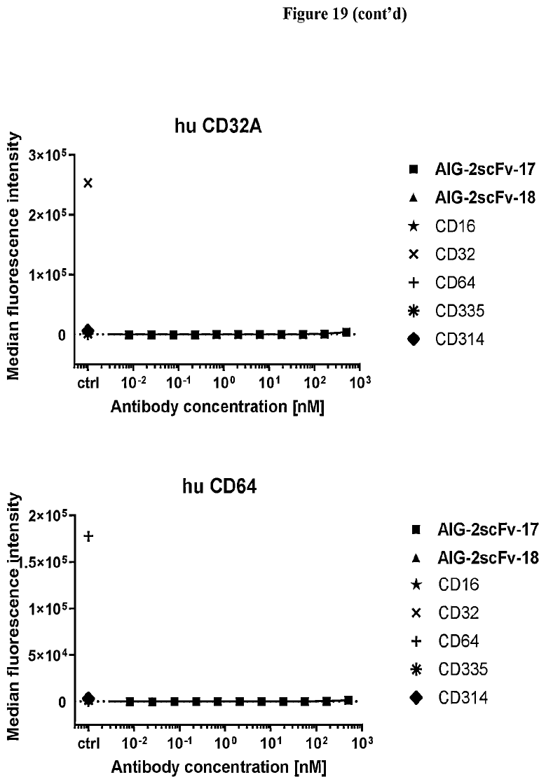

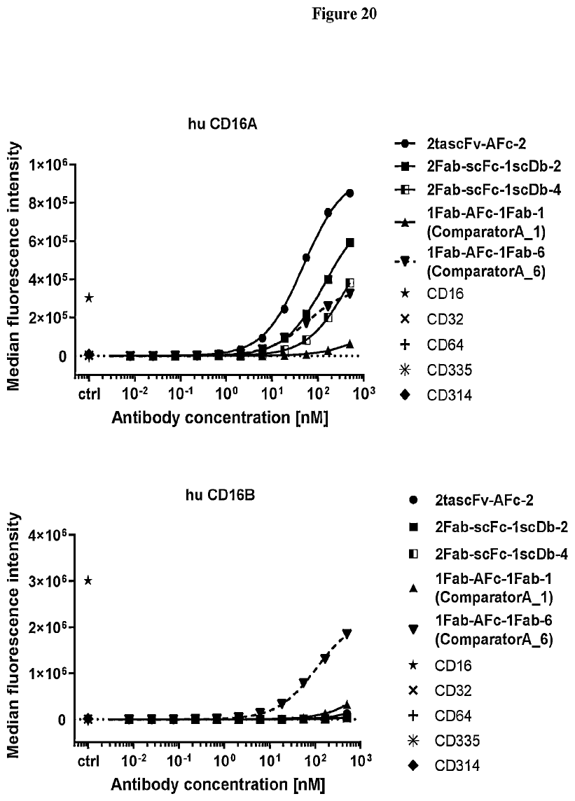

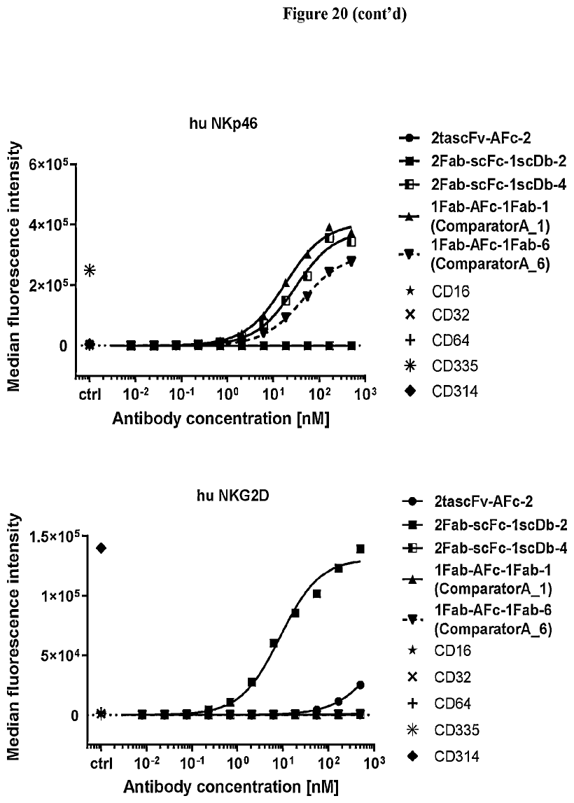

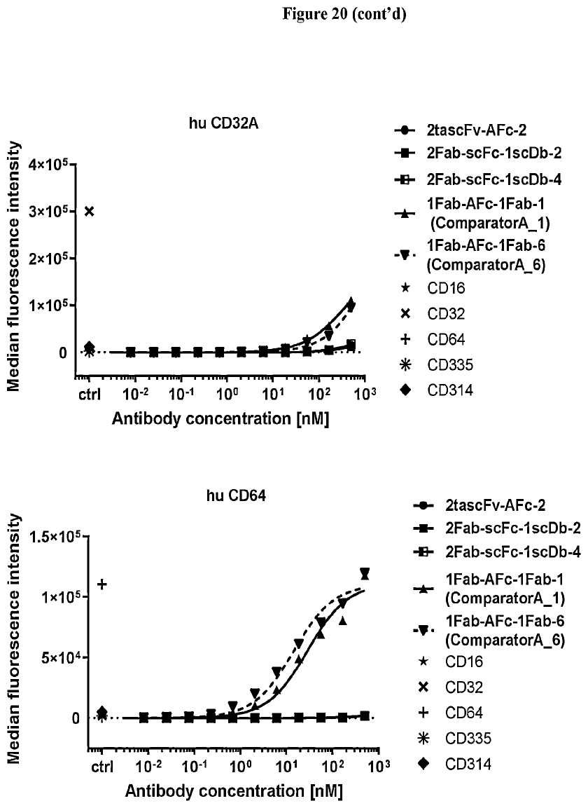

To assess binding specificity of trispecific antibody constructs (e.g. CD19/CD16A/NKG2D, CD19/CD16A/NKp46, EGFR/CD16A/NKG2D, and EGFR/CD16A/NKp46) to their cognate cell surface-bound innate cell receptors, CHO cells transduced with individual recombinant human receptors (e.g. CD16A, CD16B, CD32, CD64, NKG2D, NKp46) and non-transduced control CHO cells were incubated with the trispecific constructs and control constructs followed by flow cytometric detection by e.g. FITC-conjugated goat anti-human IgG Fc secondary antibodies. The results of the cell binding experiments using CHO cell lines expressing recombinant receptors demonstrate specific binding of constructs comprising anti-CD16A Fv domains (e.g. CD19/CD16A/NKG2D, CD19/CD16A/NKp46, EGFR/CD16A/NKG2D, and EGFR/CD16A/NKp46 in tested formats IG-scDb, 2Fab-1scDb-AFc, 2Fab-1scFv-AFc, 1Fab-1scDb-AFc, AIG-2scFv, 2tascFv-AFc and 2Fab-scFc-1scDb) to cells expressing recombinant human CD16A, but no or only background binding to cells expressing other Fcg receptors, e.g. CD16B, CD32, or CD64. Likewise, only constructs comprising anti-NKG2D Fv domains (e.g. CD19/CD16A/NKG2D, or EGFR/CD16A/NKG2D) exhibited binding signals on cells expressing recombinant human NKGD, whereas constructs comprising anti-NKp46 Fv domains displayed binding to recombinant cells expressing NKp46 (Table 4 and FIGS. 19 and 20). None of constructs showed substantial binding signals on CHO cells devoid of recombinant receptors. In contrast, constructs with active Fc domains, such as scFv-IgAb and all 2-Fab-1scFv-AFc constructs (−1, −2, −3, and −4) do not or only weakly bind CD16 but exhibit high affinity to CD64 and moderate affinity to CD32. Constructs with wt or enhanced Fc domains of the formats 1Fab-AFc-1Fab and AIG-1scFv (comparator molecules) also bind to CD64 with high affinity and moderately to CD32. These constructs exhibit CD16A binding and for the Fc enhanced molecules also high affinity CD16B binding (Table 5 and FIGS. 19 and 20).

In addition, after incubation of enriched primary human NK cells, expressing endogenous receptors, e.g. CD16A, NKG2D, or NKp46, with the trispecific constructs and control constructs, all constructs comprising anti-CD16A and/or anti-NKG2D, and/or anti-NKp46 Fv domains elicited specific binding to primary human NK cells. Moreover, constructs comprising anti-NKG2D Fv domains showed binding to the NKG2D⁺ subpopulation of enriched primary human T cells.

TABLE 4
Apparent affinities (K_{D}) determined for binding of trispecific molecules to recombinant
human receptors (e.g. CD16A (48R/158F) and CD16B (NA1) expressed on the surface of CHO
cells. The CHO cells were incubated with serial dilutions of the indicated trispecific
constructs and control constructs at 37° C., and cell surface bound antibodies were
detected by FITC-conjugated goat anti-human IgG Fc and flow cytometric analysis. Measured
median fluorescence intensity values were used to calculate apparent affinities (K_{D}) by
non-linear regression. Mean and SD of two independent experiments are depicted.

| | | | | huCD16A | | huCD16B | |
|---|---|---|---|---|---|---|---|
| | | Effector | Effector | K_{D} [nM] | | K_{D} [nM] | |
| Construct | Target | domain 1 | domain 2 | mean | SD | mean | SD |
| IG-scDb-1 | CD19 | P2C47var50 | KYK-2.0 | 1705.0 | 690.1 | n.a. | n.a. |
| IG-scDb-2 | CD19 | P2C47var50 | ADI- | 194.6 | 6.9 | 9506.0 | n.a. |
| | | | 27743 | | | | |
| IG-scDb-3 | CD19 | P2C47var50 | NKp46-1 | 1068.5 | 50.2 | n.a. | n.a. |
| 2Fab-1scDb-AFc-1 | CD19 | ABC1197var8 | KYK-2.0 | 95.8 | 15.3 | n.a. | n.a. |
| 2Fab-1scDb-AFc-3 | CD19 | ABC1197var8 | NKp46-1 | 95.0 | 12.0 | n.a. | n.a. |
| 2Fab-1scDb-AFc-4 | CD19 | ABC1197var8 | NKp46-3 | 115.3 | 20.8 | n.a. | n.a. |
| 2Fab-1scDb-AFc-7 | EGFR | ABC1197var8 | NKp46-1 | 75.5 | 9.3 | n.a. | n.a. |
| 2Fab-1scFv-AFc-3 | CD19 | wt Fc | NKp46-1 | n.a. | na | n.a. | n.a. |
| 1Fab-1scDb-AFc-1 | CD19 | ABC1197var8 | KYK-2.0 | 67.5 | 28.3 | n.a. | n.a. |
| 1Fab-1scDb-AFc-3 | CD19 | ABC1197var8 | NKp46-1 | 58.4 | 18.9 | n.a. | n.a. |
| AIG-2scFv-16 | CD19 | ABC1197var8 | KYK-2.0 | 90.8 | 65.9 | n.a. | n.a. |
| AIG-2scFv-17 | CD19 | ABC1197var8 | ADI- | 66.0 | 5.6 | n.a. | n.a. |
| | | | 27743 | | | | |
| AIG-2scFv-18 | CD19 | ABC1197var8 | NKp46-1 | 158.9 | 5.0 | n.a. | n.a. |
| AIG-2scFv-19 | CD19 | ABC1197var8 | NKp46-3 | 69.8 | 26.9 | n.a. | n.a. |
| 2scDb-AFc-3 | CD19 | ABC1197var6 | NKp46-1 | 527.8 | 62.5 | n.a. | n.a. |
| 2tascFv-AFc-2 | CD19 | ABC1197var8 | ADI- | 52.6 | 4.8 | 1166.0 | n.a. |
| | | | 27743 | | | | |
| 2Fab-scFc-1scDb-1 | CD19 | ABC1197var8 | KYK-2.0 | 617.8 | 15.1 | 1773.0 | n.a. |
| 2Fab-scFc-1scDb-2 | CD19 | ABC1197var8 | ADI- | 147.1 | 10.6 | 5083.0 | n.a. |
| | | | 27743 | | | | |
| 2Fab-scFc-1scDb-3 | CD19 | ABC1197var8 | NKp46-1 | 425.4 | 98.3 | n.a. | n.a. |
| 2Fab-scFc-1scDb-4 | CD19 | ABC1197var8 | NKp46-3 | 379.7 | 41.2 | n.a. | n.a. |
| 1Fab-AFc-1Fab_1 | CD19 | wt FC | NKp46-1 | 767.1 | 133.4 | 1283.5 | 337.3 |
| (ComparatorA_1) | | | | | | | |
| 1Fab-AFc-1Fab_2 | CD19 | wt Fc | NKp46-3 | 2231.0 | 2373.1 | n.a. | n.a. |
| (ComparatorA_2) | | | | | | | |
| 1Fab-AFc-1Fab_5 | CD19 | Fc-enhanced | NKp46-1 | 96.7 | 30.6 | 111.3 | 51.4 |
| (ComparatorA_5) | | | | | | | |
| 1Fab-AFc-1Fab_6 | CD19 | Fc-enhanced | NKp46-3 | 55.8 | 4.4 | 85.8 | 31.2 |
| (ComparatorA_6) | | | | | | | |
| AIG-1scFv_5 | CD19 | wt Fc | KYK-2.0 | 1215.0 | n.a. | n.a. | n.a. |
| (ComparatorB_1) | | | | | | | |
| AIG-1scFv_6 | CD19 | wt Fc | ADI- | 1003.0 | n.a. | n.a. | n.a. |
| (ComparatorB_2) | | | 27743 | | | | |
| scFv-IgAb-393 | CD19 | wt Fc | KYK-2.0 | n.a. | n.a. | n.a. | n.a. |
| scFv-IgAb-394 | CD19 | wt Fc | ADI- | 553.3 | 386.2 | 531.5 | n.a. |
| | | | 27743 | | | | |
| scFv-IgAb-395 | CD19 | wt Fc | NKp46-1 | n.a. | n.a. | n.a. | n.a. |
| scFv-IgAb-396 | CD19 | wt Fc | NKp46-3 | 5187.0 | n.a. | n.a. | n.a. |

TABLE 5
Apparent affinities (K_{D}) determined for binding of trispecific molecules to recombinant human receptors
CD32A, CD64, NKG2D, and NKp46 expressed on the surface of CHO cells. The CHO cells were incubated
with serial dilutions of the indicated trispecific constructs and control constructs at 37° C.,
and cell surface bound antibodies were detected by FITC-conjugated goat anti-human IgG Fc and flow cytometric
analysis. Measured median fluorescence intensity values were used to calculate apparent affinities
(K_{D}) by non-linear regression. Mean and SD of two independent experiments are depicted.

| | | | | huNKp46 | | huNKG2D | | huCD32A | | huCD64 | |
|---|---|---|---|---|---|---|---|---|---|---|---|
| | | Effector | Effector | K_{D} [nM] | | K_{D} [nM] | | K_{D} [nM] | | K_{D} [nM] | |
| Construct | Target | domain 1 | domain 2 | mean | SD | mean | SD | mean | SD | mean | SD |
| IG-scDb-1 | CD19 | P2C47var50 | KYK-2.0 | 964.3 | 1272.4 | 1.3 | 0.5 | 4094.0 | n.a. | n.a. | n.a. |
| IG-scDb-2 | CD19 | P2C47var50 | ADI- | n.a. | n.a. | 1.4 | 0.5 | n.a. | n.a. | n.a. | n.a. |
| | | | 27743 | | | | | | | | |
| IG-scDb-3 | CD19 | P2C47var50 | NKp46-1 | 4.9 | 1.6 | 2888.0 | 429.9 | n.a. | n.a. | n.a. | n.a. |
| 2Fab-1scDb-AFc-1 | CD19 | ABC1197var8 | KYK-2.0 | n.a. | n.a. | 10.4 | 11.2 | n.a. | n.a. | n.a. | n.a. |
| 2Fab-1scDb-AFc-3 | CD19 | ABC1197var8 | NKp46-1 | 11.1 | 2.8 | n.a. | n.a. | n.a. | n.a. | n.a. | n.a. |
| 2Fab-1scDb-AFc-4 | CD19 | ABC1197var8 | NKp46-3 | 64.0 | 6.4 | n.a. | n.a. | n.a. | n.a. | n.a. | n.a. |
| 2Fab-1scDb-AFc-7 | EGFR | ABC1197var8 | NKp46-1 | 11.5 | 2.4 | 4225.0 | n.a. | n.a. | n.a. | n.a. | n.a. |
| 2Fab-1scFv-AFc-3 | CD19 | wt Fc | NKp46-1 | 9.7 | 2.5 | n.a. | n.a. | 682.2 | 719.6 | 38.1 | 13.7 |
| 1Fab-1scDb-AFc-1 | CD19 | ABC1197var8 | KYK-2.0 | 911.5 | n.a. | 5.7 | 1.3 | n.a. | n.a. | n.a. | n.a. |
| 1Fab-1scDb-AFc-3 | CD19 | ABC1197var8 | NKp46-1 | 8.9 | 1.5 | n.a. | n.a. | n.a. | n.a. | 1169.0 | 1.2. |
| AIG-2scFv-16 | CD19 | ABC1197var8 | KYK-2.0 | n.a. | n.a. | 13.0 | 0.2 | n.a. | n.a. | n.a. | n.a. |
| AIG-2scFv-17 | CD19 | ABC1197var8 | ADI- | n.a. | n.a. | 13.7 | 3.1 | n.a. | n.a. | n.a. | n.a. |
| | | | 27743 | | | | | | | | |
| AIG-2scFv-18 | CD19 | ABC1197var8 | NKp46-1 | 31.0 | 7.5 | n.a. | n.a. | n.a. | n.a. | n.a. | n.a. |
| AIG-2scFv-19 | CD19 | ABC1197var8 | NKp46-3 | 63.6 | 6.7 | n.a. | n.a. | n.a. | n.a. | n.a. | n.a. |
| 2scDb-AFc-3 | CD19 | ABC1197var6 | NKp46-1 | 11.5 | 0.2 | n.a. | n.a. | n.a. | n.a. | n.a. | n.a. |
| 2tascFv-AFc-2 | CD19 | ABC1197var8 | ADI- | n.a. | n.a. | 665.8 | 170.9 | n.a. | n.a. | n.a. | n.a. |
| | | | 27743 | | | | | | | | |
| 2Fab-scFc-1scDb-1 | CD19 | ABC1197var8 | KYK-2.0 | n.a. | n.a. | 8.6 | 1.4 | n.a. | n.a. | n.a. | n.a. |
| 2Fab-scFc-1scDb-2 | CD19 | ABC1197var8 | ADI- | n.a. | 1.2. | 7.0 | 2.4 | n.a. | n.a. | n.a. | n.a. |
| | | | 27743 | | | | | | | | |
| 2Fab-scFc-1scDb-3 | CD19 | ABC1197var8 | NKp46-1 | 15.4 | 2.2 | n.a. | n.a. | n.a. | n.a. | n.a. | n.a. |
| 2Fab-scFc-1scDb-4 | CD19 | ABC1197var8 | NKp46-3 | 32.2 | 3.7 | n.a. | n.a. | n.a. | n.a. | n.a. | n.a. |
| 1Fab-AFc-1Fab-1 | CD19 | wt Fc | NKp46-1 | 19.8 | 2.6 | n.a. | n.a. | 286.2 | 118.9 | 22.7 | 8.7 |
| (ComparatorA_1) | | | | | | | | | | | |
| 1Fab-AFc-1Fab-2 | CD19 | wt Fc | NKp46-3 | 48.0 | 6.2 | n.a. | n.a. | 1147.0 | n.a. | 23.9 | 7.0 |
| (ComparatorA_2) | | | | | | | | | | | |
| 1Fab-AFc-1Fab-5 | CD19 | Fc- | NKp46-1 | 19.4 | 2.4 | n.a. | n.a. | 1054.6 | 236.8 | 15.9 | 3.4 |
| (ComparatorA_5) | | enhanced | | | | | | | | | |
| 1Fab-AFc-1Fab-6 | CD19 | Fc- | NKp46-3 | 45.8 | 2.8 | n.a. | n.a. | 1012.3 | 405.5 | 12.6 | 2.7 |
| (ComparatorA_6) | | enhanced | | | | | | | | | |
| AIG-1scFv-5 | CD19 | wt Fc | KYK-2.0 | n.a. | n.a. | 28.5 | 16.2 | 2470.0 | n.a. | 27.4 | 7.6 |
| (ComparatorB 1) | | | | | | | | | | | |
| AIG-1scFv-6 | CD19 | wt Fc | ADI- | n.a. | n.a. | 22.3 | 6.8 | 2534.0 | n.a. | 22.8 | 7.7 |
| (ComparatorB 2) | | | 27743 | | | | | | | | |
| scFv-IgAb-393 | CD19 | wt Fc | KYK-2.0 | n.a. | n.a. | 1.9 | 0.9 | 3134.0 | n.a. | 36.7 | 1.4 |
| scFv-IgAb-394 | CD19 | wt Fc | ADI- | n.a. | n.a. | 4.0 | 1.4 | 164.4 | 34.0 | 31.8 | 2.8 |
| | | | 27743 | | | | | | | | |
| scFv-IgAb-395 | CD19 | wt Fc | NKp46-1 | 5.7 | 1.9 | n.a. | n.a. | 5634.0 | 2169.4 | 42.3 | 5.8 |
| scFv-IgAb-396 | CD19 | wt Fc | NKp46-3 | 9.8 | 3.7 | n.a. | n.a. | 373.9 | 64.3 | 34.1 | 6.6 |

### Example 12: Assessment of NK Cell Fratricide Induced by Trispecific Antibody Constructs

Antibody constructs that comprise two specificities for NK cell receptors (e.g. anti-CD16A/anti-CD16A, anti-CD16A/anti-NKG2D, anti-CD16A/anti-NKp46, Fc/anti-CD16A, Fc/anti-NKG2D, or Fc/anti-NKp46), in addition to one tumor antigen specificity (e.g. CD19 or EGFR), might mediate crosslinking of NK cells, leading to activation of the individual NK cells and potential NK cell-NK cell killing (i.e. NK cell fratricide). Accordingly, to assess whether trispecific constructs (e.g. CD19/CD16A/NKG2D, CD19/CD16A/NKp46, EGFR/CD16A/NKG2D, or EGFR/CD16A/NKp46) have the potential to induce NK cell fratricide, 4 h calcein-release assays with calcein-labeled NK cells as indicator for NK cell lysis and autologous non-labeled NK cells as effector cells (i.e. both NK cell preparations from the identical donor) were performed in the presence of serial dilutions of the trispecific constructs and control constructs comprising wt Fc domains instead of anti-CD16A Fv domains.

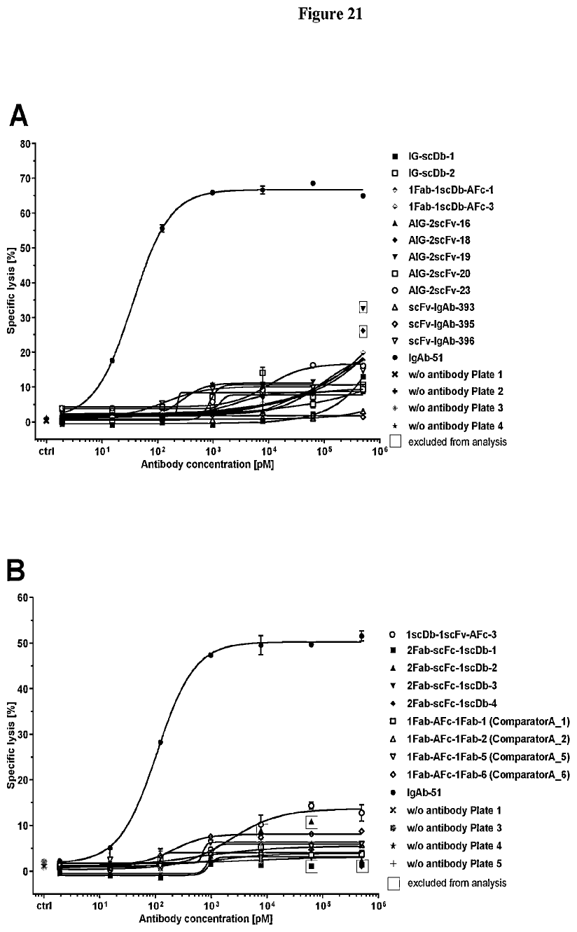

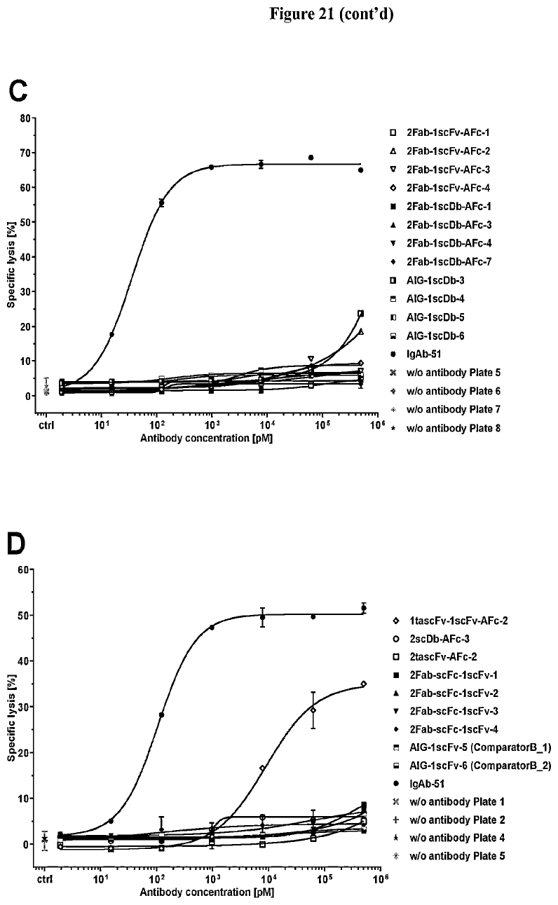

The NK cell fratricide assays resulted in no or low (below 20%) concentration-dependent lysis of NK cell by autologous NK cells in the presence of constructs having anti-CD16A Fv domains e.g. CD19/CD16A/NKG2D, CD19/CD16A/NKp46, EGFR/CD16A/NKG2D, or EGFR/CD16A/NKp46 in 2Fab-scFc-1scDb, 2Fab-1scDb-AFc, 1Fab-1scDb-AFc, 2scDb-AFc, 2tascFv-AFc, AIG-2scFv, IG-scDb, and AIG-1scDb tested formats (Table 6, FIG. 21). In contrast, lysis of NK cell was substantially induced in the presence of control constructs including active Fc domains, e.g. CD19/Fc/NKG2D, CD19/Fc/NKp46, EGFR/Fc/NKG2D, or EGFR/Fc/NKp46. The positive control anti-CD38 IgG1 (IgAb-51) induced strong concentration-dependent NK cell lysis with efficacies of more than 50% lysis. Several constructs comprising an active Fc domain induced stronger NK cell fratricide with higher efficacy than constructs without an active Fc domain but with anti-CD16A Fv domains. CD19/Fc/NKG2D trispecific constructs, e.g. 1scDb-1scFv-AFc-21tascFv-1scFv-AFc-2 induced NK cell fratricide with more than 20% efficacy.

TABLE 6
Potency (EC₅₀) and efficacy (Eₘₐₓ) values determined for trispecific constructs determined in 4 h
calcein-release assays with calcein-labeled NK cells as target cells and autologous NK cells as
effector cells at an E:T ratio of 1:1. Mean and SD of two independent experiments are presented.

| | | Effector | Effector | EC₅₀ [pM] | | Eₘₐₓ [%] | |
|---|---|---|---|---|---|---|---|
| Construct | Target | domain 1 | domain 2 | mean | SD | mean | SD |
| 1Fab-1scDb-AFc-1 | CD19 | ABC1197var8 | KYK-2.0 | 91829.0 | n.a. | 14.2 | 5.4 |
| 1Fab-1scDb-AFc-3 | CD19 | ABC1197var8 | NKp46-1 | 13464.0 | n.a. | 14.3 | 5.1 |
| 1Fab-AFc-1Fab-1 | CD19 | wt Fc | NKp46-1 | 14797.3 | 20754.6 | 2.5 | 1.0 |
| (ComparatorA_1) | | | | | | | |
| 1Fab-AFc-1Fab-2 | CD19 | wt Fc | NKp46-3 | 370.4 | 167.5 | 2.8 | 3.2 |
| (ComparatorA_2) | | | | | | | |
| 1Fab-AFc-1Fab-5 | CD19 | Fc-enhanced Fc | NKp46-1 | 73346.8 | 102728.8 | 2.7 | 2.8 |
| (ComparatorA_5) | | | | | | | |
| 1Fab-AFc-1Fab-6 | CD19 | Fc-enhanced Fc | NKp46-3 | 211.1 | n.a. | 4.8 | 2.9 |
| (ComparatorA_6) | | | | | | | |
| 1scDb-1scFv-AFc-2 | CD19 | wt Fc | ADI-27743 | 171514 | 200359 | 36.7 | 7.0 |
| 1scDb-1scFv-AFc-3 | CD19 | wt Fc | NKp46-1 | 1679.5 | 785.6 | 9.1 | 6.0 |
| 1tascFv-1scFv-AFc-2 | CD19 | wt Fc | ADI-27743 | 34858.0 | 36686.1 | 36.1 | 0.2 |
| 2Fab-1scDb-AFc-1 | CD19 | ABC1197var8 | KYK-2.0 | n.a. | n.a. | 1.8 | 2.5 |
| 2Fab-1scDb-AFc-3 | CD19 | ABC1197var8 | NKp46-1 | n.a. | n.a. | 8.2 | 0.0 |
| 2Fab-1scDb-AFc-4 | CD19 | ABC1197var8 | NKp46-3 | 8666.5 | 430.6 | 10.3 | 1.8 |
| 2Fab-1scDb-AFc-7 | EGFR | ABC1197var8 | NKp46-1 | 141.1 | n.a. | 4.7 | 3.2 |
| 2Fab-1scFv-AFc-1 | CD19 | wt Fc | KYK-2.0 | 136.7 | n.a. | 1.5 | 2.1 |
| 2Fab-1scFv-AFc-2 | CD19 | wt Fc | ADI-27743 | n.a. | n.a. | 12.4 | 6.1 |
| 2Fab-1scFv-AFc-3 | CD19 | wt Fc | NKp46-1 | 2643.0 | n.a. | 3.3 | 4.7 |
| 2Fab-1scFv-AFc-4 | CD19 | wt Fc | NKp46-3 | 8497.0 | 10381.7 | 9.0 | 2.6 |
| 2Fab-scFc-1scDb-1 | CD19 | ABC1197var8 | KYK-2.0 | 898.8 | n.a. | 2.0 | 2.8 |
| 2Fab-scFc-1scDb-2 | CD19 | ABC1197var8 | ADI-27743 | 1079.9 | 216.5 | 6.4 | 0.1 |
| 2Fab-scFc-1scDb-3 | CD19 | ABC1197var8 | NKp46-1 | 5231.0 | n.a. | 0.6 | 0.8 |
| 2Fab-scFc-1scDb-4 | CD19 | ABC1197var8 | NKp46-3 | 1345.0 | n.a. | 1.5 | 2.1 |
| 2Fab-scFc-1scFv-1 | CD19 | wt Fc | KYK-2.0 | 318319.0 | n.a. | 5.9 | 8.4 |
| 2Fab-scFc-1scFv-2 | CD19 | wt Fc | ADI-27743 | 25498.0 | n.a. | 0.9 | 1.2 |
| 2Fab-scFc-1scFv-3 | CD19 | wt Fc | NKp46-1 | 58040.0 | n.a. | 3.3 | 4.7 |
| 2Fab-scFc-1scFv-4 | CD19 | wt Fc | NKp46-3 | 48.4 | 61.8 | 3.2 | 1.3 |
| 2scDb-AFc-3 | CD19 | ABC1197var6 | NKp46-1 | 652.0 | 526.1 | 6.1 | 1.5 |
| 2tascFv-AFc-2 | CD19 | ABC1197var8 | ADI-27743 | 64925.0 | n.a. | 4.9 | 1.6 |
| AIG-1scDb-3 | CD19 | wt Fc | KYK-2.0 | n.a. | n.a. | 19.4 | 0.5 |
| AIG-1scDb-4 | CD19 | wt Fc | ADI-27743 | 5661.5 | 2629.7 | 1.7 | 1.1 |
| AIG-1scDb-5 | CD19 | wt Fc | NKp46-1 | 322.1 | n.a. | 1.0 | 1.4 |
| AIG-1scDb-6 | CD19 | wt Fc | NKp46-3 | 172.4 | n.a. | 1.5 | 2.1 |
| AIG-1scFv-5 | CD19 | wt Fc | KYK-2.0 | n.a | n.a. | 4.2 | 2.1 |
| (ComparatorB_1) | | | | | | | |
| AIG-1scFv-6 | CD19 | wt Fc | ADI-27743 | 27003.0 | n.a. | 0.9 | 1.2 |
| (ComparatorB_2) | | | | | | | |
| AIG-2scFv-16 | CD19 | ABC1197var8 | KYK-2.0 | n.a. | n.a. | 6.5 | 1.6 |
| AIG-2scFv-18 | CD19 | ABC1197var8 | NKp46-1 | 307.9 | 109.0 | 5.5 | 1.6 |
| AIG-2scFv-19 | CD19 | ABC1197var8 | NKp46-3 | 346.7 | 103.4 | 8.8 | 1.5 |
| AIG-2scFv_20 | CD19 | 3G8 | KYK-2.0 | n.a. | n.a. | 0.0 | n.a. |
| AIG-2scFv-23 | CD19 | 3G8 | NKp46-3 | 9787.0 | n.a. | 6.2 | 8.8 |
| IG-scDb-1 | CD19 | P2C-47var50 | KYK-2.0 | n.a. | n.a. | 10.0 | 5.0 |
| IG-scDb-2 | CD19 | P2C-47var50 | ADI-27743 | 886.6 | 31.2 | 7.6 | 3.6 |
| IG-scDb-3 | CD19 | P2C-47var50 | NKp46-1 | 24782.0 | 22927.2 | 13.7 | 3.6 |
| scFv-IgAb-393 | CD19 | wt Fc | KYK-2.0 | n.a. | n.a. | 4.2 | 2.7 |
| scFv-IgAb-395 | CD19 | wt Fc | NKp46-1 | n.a. | n.a. | 0.0 | n.a. |
| scFv-IgAb-396 | CD19 | wt Fc | NKp46-3 | 6812.4 | 9443.6 | 11.1 | 2.8 |

n.a., not applicable.

### Example 13: Assessment of NK Cell Fratricide by Trispecific HER2/CD16A/NKG2D Antibody Constructs

To test whether trispecific HER2/CD16A/NKG2D antibody constructs with one anti-CD16A Fv domain, one anti-NKG2D domain, and two Fab specific for HER2, e.g. AIG-2scFv-7 (SEQ ID NOs: 431-433), AIG-2scFv-8 (SEQ ID NOs: 434-436), or AIG-2scFv-10 (SEQ ID NOs: 437-439), induce NK cell fratricide, 4 h calcein-release assays with enriched primary human NK cells as indicator for NK cell lysis and autologous NK cells as effector cells were performed in the presence of 10 serial 1:5 dilutions of the indicated antibody constructs starting at 100 μg/mL. Control antibody constructs with identical HER2-targeting domains but different effector cell recruiting domains, e.g. AIG-2scFv-14 (SEQ ID NOs: 440-442) with two Fv domains for NKG2D but without an anti-CD16A domain, or AIG-2scFv-15 (SEQ ID NOs: 443-445) with one anti-NKG2D domain and one anti-RSV domain, or AIG-1scFv-4 (SEQ ID NOs: 446-448) with only one anti-NKG2D domain, were used as controls. As a positive control for the induction of NK cell fratricide, the human anti-CD38 IgG1 (IgAb_51, SEQ ID NOs: 429 and 430) was included.

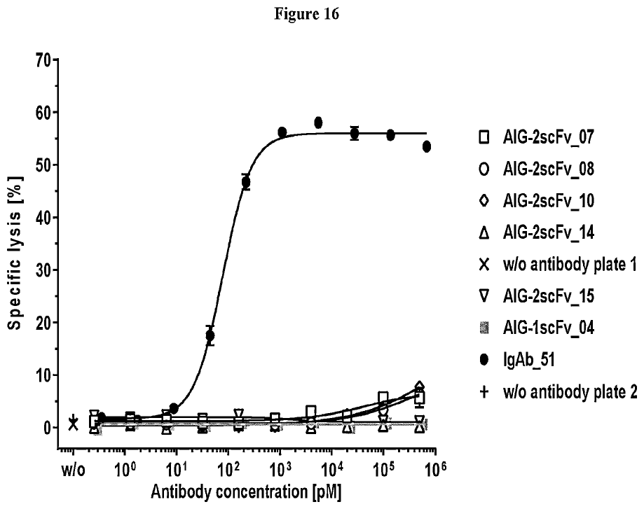

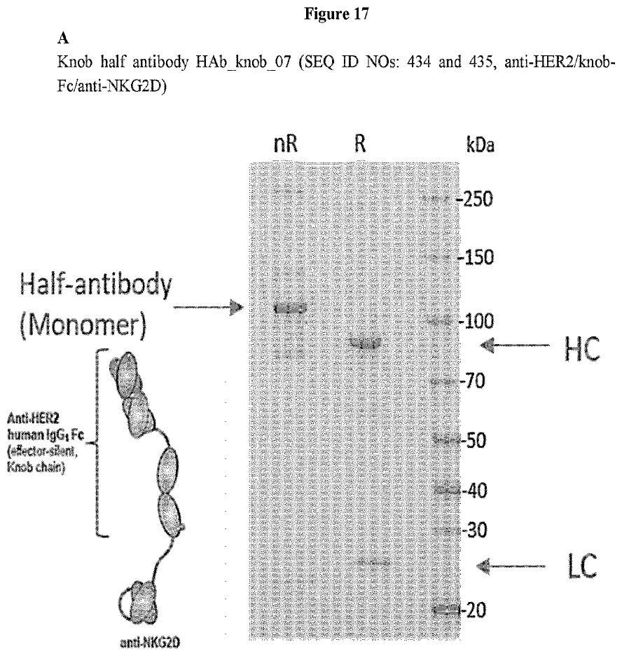

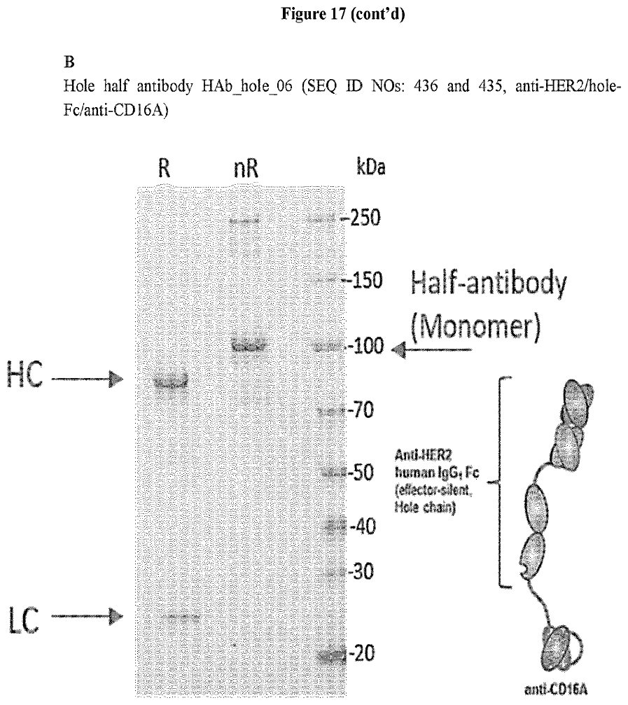

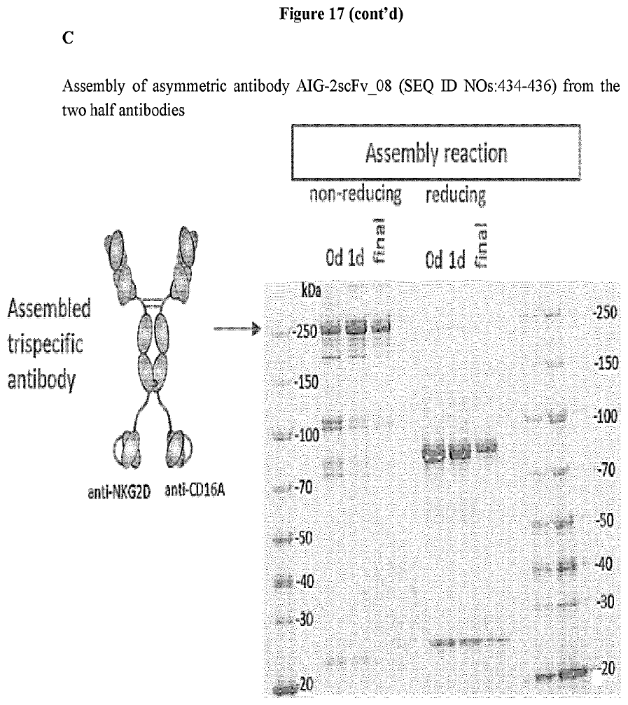

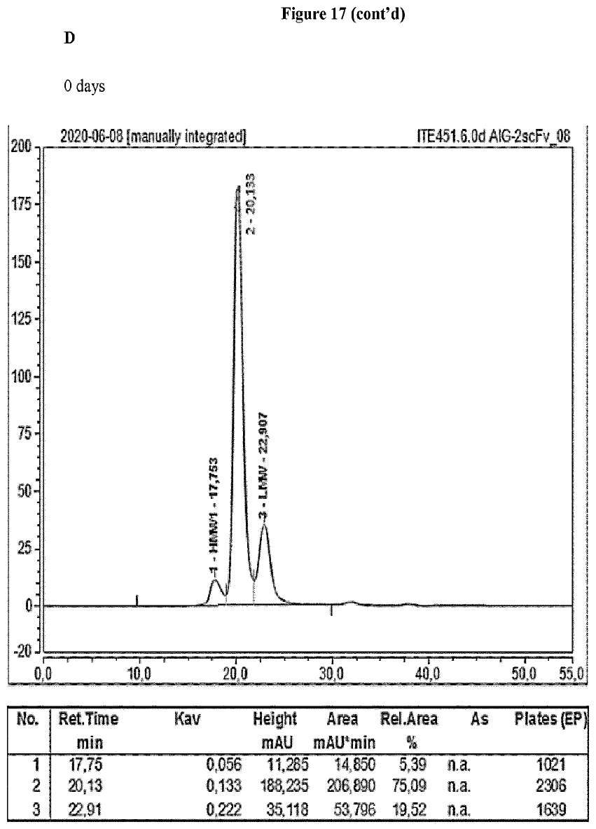

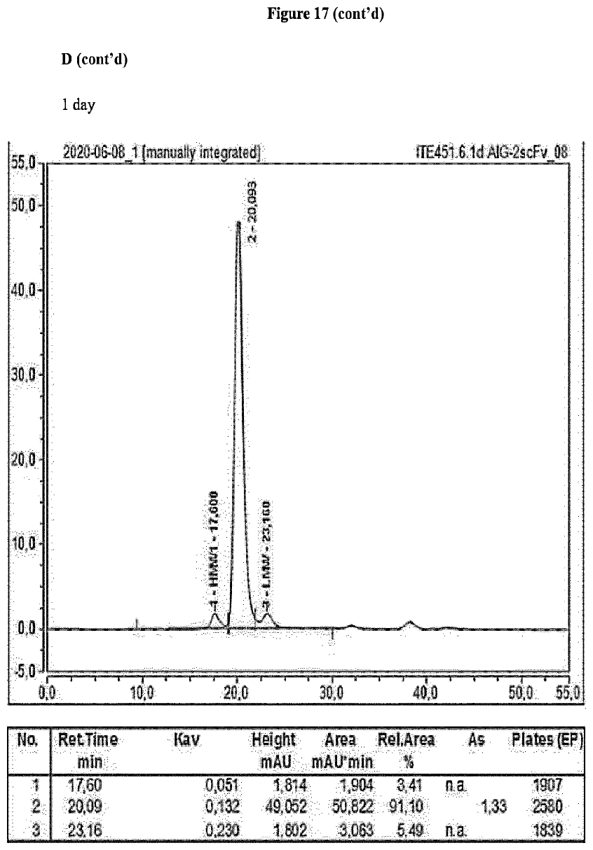

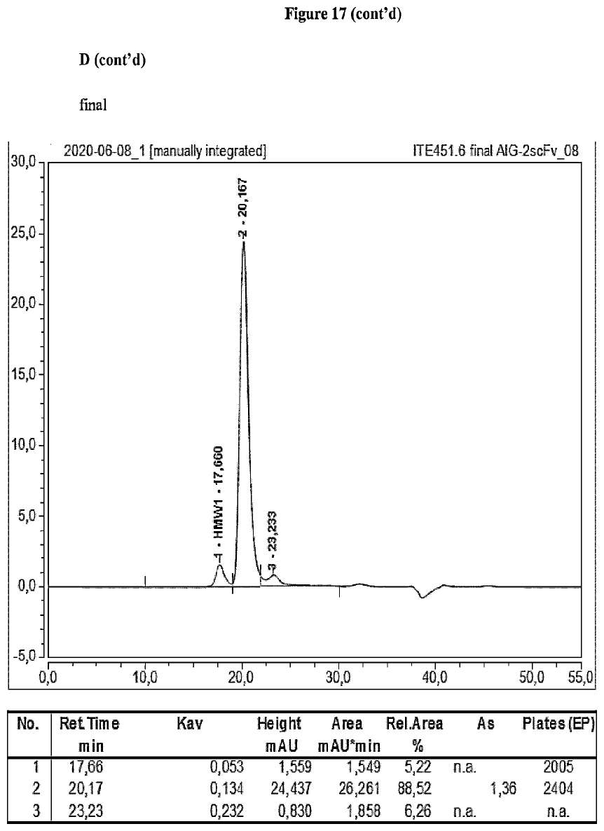

The results of the 4 h cytotoxicity assay in FIG. 16 demonstrate that the trispecific HER2/CD16A/NKG2D antibody constructs induced no or only minimal NK cell fratricide with lysis values below 10% even at the highest antibody concentration of 100 μg/mL. In contrast, the positive control anti-CD38 IgG1 induced strong concentration-dependent NK cell lysis with reached efficacies of more than 50% lysis.

### Example 14: Assessment of Lysis of CD32⁺/CD64⁺ Target Cells by NK Cells Induced by Trispecific Antibody Constructs

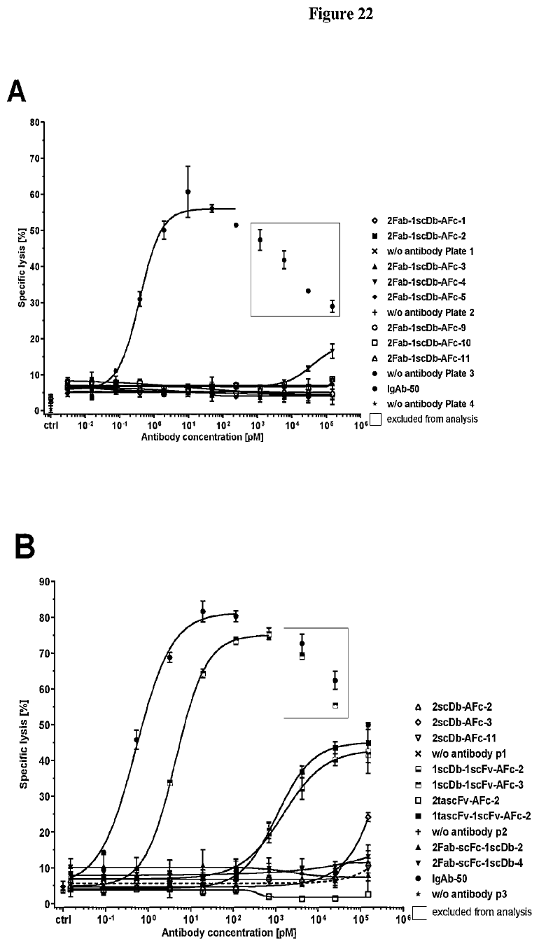

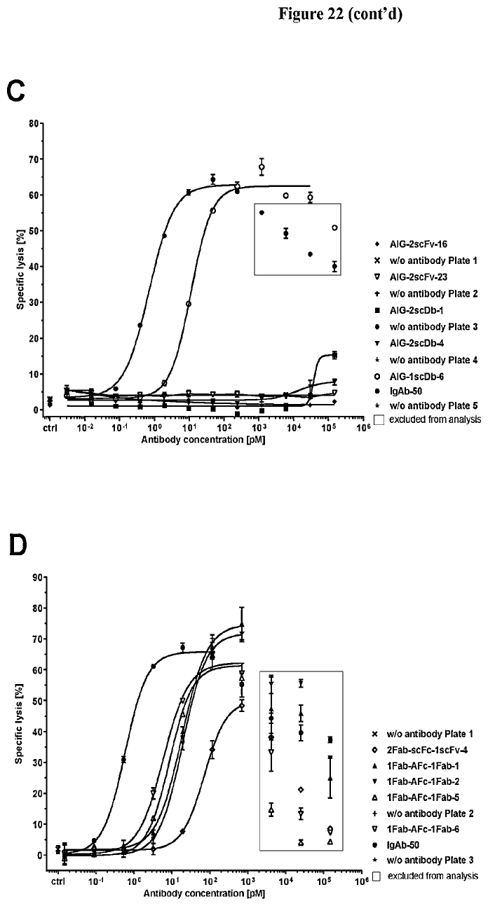

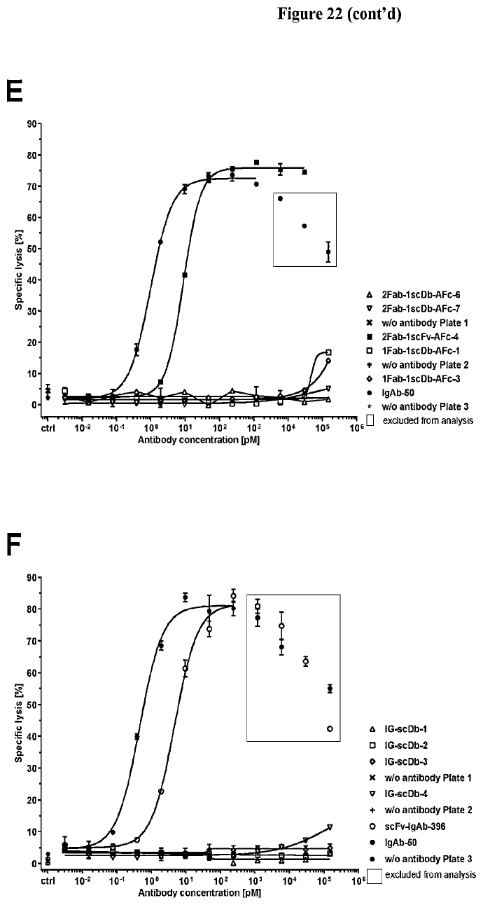

It was tested whether trispecific antibody constructs comprising a silenced Fc domain, and Fv domains specific for a tumor antigen, e.g. CD19 or EGFR, and NK receptors, e.g. CD16A, NKG2D, or NKp46, induced lysis of tumor antigen-negative cells that express Fcg receptors CD32 and/or CD64. In 4 h calcein-release cytotoxicity assays serial dilutions of trispecific antibody constructs comprising a silenced Fc domain, e.g. CD19/CD16A/NKG2D, CD19/CD16A/NKp46, EGFR/CD16A/NKG2D, and EGFR/CD16A/NKp46, exerted a low potential to induce lysis of CD32+/CD64+ EGFR−/CD19− THP-1 target cells by enriched primary human NK cells. However, control trispecific antibody constructs comprising wt Fc or Fc-enhanced Fc domains, e.g. CD19/Fc/NKG2D, CD19/Fc/NKp46, EGFR/Fc/NKG2D, or EGFR/Fc/NKp46, induced significant lysis of CD32+/CD64+ EGFR−/CD19− THP-1 target cells in a concentration-dependent manner. The results of the cytotoxicity assays summarized in Table 7 and the exemplary graphs presented in FIG. 22 clearly demonstrate that the anti-CD16A IgG1 (IgAb-50), used as a positive control, and Fc-containing constructs in various formats such as 1Fab-AFc-1Fab-1, 1Fab-AFc-1Fab-2, 1Fab-AFc-1Fab-5, 1Fab-AFc-1Fab-6, 1scDb-1scFv-AFc-2, 1scDb-1scFv-AFc-3, 1tascFv-1scFv-AFc-2, 2Fab-scFc-1scFv-4, AIG-1scDb-6, and scFv-IgAb-396 induced potent and efficacious lysis of CD32⁺/CD64⁺ THP-1 target cells with Eₘₐₓ values>20%. In contrast, trispecific constructs in various formats without an active Fc domain, e.g. CD19/CD16A/NKG2D AIG-2scFv-16, e.g. CD19/CD16A/NKp46 2tascFv-AFc-2, e.g. EGFR/CD16A/NKG2D Fab-1scDb-AFc-5, or EGFR/CD16A/NKp46 2Fab-1scDb-AFc-7 induced no or only minimal lysis (Eₘₐₓ<20%).

TABLE 7
Potency (EC₅₀) and efficacy (Eₘₐₓ) values determined for trispecific constructs
determined in 4 h calcein-release assays with calcein-labeled THP-1 as target
cells and enriched primary human NK cells as effector cells at an E:T ratio
of 5:1. Mean and SD of two independent experiments are presented.

| | | Effector | Effector | EC₅₀ [pM] | | Eₘₐₓ [%] | |
|---|---|---|---|---|---|---|---|
| Construct | Target | domain 1 | domain 2 | mean | SD | mean | SD |
| 1Fab-1scDb-AFc-1 | CD19 | ABC1197var8 | KYK-2.0 | n.a. | n.a. | 7.5 | 10.7 |
| 1Fab-1scDb-AFc-3 | CD19 | ABC1197var8 | NKp46-1 | n.a. | n.a. | 9.8 | 3.8 |
| 1Fab-AFc-1Fab-1 | CD19 | wt Fc | NKp46-1 | 14.6 | 5.3 | 74.1 | 1.6 |
| (ComparatorA_1) | | | | | | | |
| 1Fab-AFc-1Fab-2 | CD19 | wt Fc | NKp46-3 | 16.1 | 4.4 | 76.9 | 7.3 |
| (ComparatorA_2) | | | | | | | |
| 1Fab-AFc-1Fab-5 | CD19 | Fc-enhanced Fc | NKp46-1 | 6.4 | 3.3 | 67.6 | 8.6 |
| (ComparatorA_5) | | | | | | | |
| 1Fab-AFc-1Fab-6 | CD19 | Fc-enhanced Fc | NKp46-3 | 4.4 | 2.2 | 68.9 | 9.9 |
| (ComparatorA_6) | | | | | | | |
| 1scDb-1scFv-AFc-2 | CD19 | wt Fc | ADI-27743 | 1157.9 | 503.7 | 41.0 | 7.1 |
| 1scDb-1scFv-AFc-3 | CD19 | wt Fc | NKp46-1 | 4.3 | 0.1 | 72.3 | 2.9 |
| 1tascFv-1scFv-AFc-2 | CD19 | wt Fc | ADI-27743 | 782.6 | 409.3 | 43.2 | 3.2 |
| 2Fab-1scDb-AFc-1 | CD19 | ABC1197var8 | KYK-2.0 | n.a. | n.a. | 0.0 | n.a. |
| 2Fab-1scDb-AFc-10 | CD19 | 3G8 | ADI-27743 | n.a. | n.a. | 0.0 | n.a. |
| 2Fab-1scDb-AFc-11 | CD19 | 3G8 | NKp46-1 | n.a. | n.a. | 0.0 | n.a. |
| 2Fab-1scDb-AFc-2 | CD19 | ABC1197var8 | ADI-27743 | n.a. | n.a. | 0.0 | n.a. |
| 2Fab-1scDb-AFc-3 | CD19 | ABC1197var8 | NKp46-1 | n.a. | n.a. | 0.0 | n.a. |
| 2Fab-1scDb-AFc-4 | CD19 | ABC1197var8 | NKp46-3 | 26347.5 | 18297.8 | 10.5 | 2.1 |
| 2Fab-1scDb-AFc-5 | EGFR | ABC1197var8 | KYK-2.0 | n.a. | n.a. | 0.0 | n.a. |
| 2Fab-1scDb-AFc-6 | EGFR | ABC1197var8 | ADI-27743 | n.a. | n.a. | 0.0 | n.a. |
| 2Fab-1scDb-AFc-7 | EGFR | ABC1197var8 | NKp46-1 | n.a. | n.a. | 2.4 | 3.3 |
| 2Fab-1scDb-AFc-9 | CD19 | 3G8 | KYK-2.0 | n.a. | n.a. | 2.1 | 3.0 |
| 2Fab-1scFv-AFc-1 | CD19 | wt Fc | KYK-2.0 | n.a. | n.a. | 1.7 | 2.4 |
| 2Fab-1scFv-AFc-2 | CD19 | wt Fc | ADI-27743 | n.a. | n.a. | 0.0 | n.a. |
| 2Fab-1scFv-AFc-4 | CD19 | wt Fc | NKp46-3 | 9.4 | 0.7 | 70.4 | 4.3 |
| 2Fab-scFc-1scDb-1 | CD19 | ABC1197var8 | KYK-2.0 | n.a. | n.a. | 0.0 | n.a. |
| 2Fab-scFc-1scDb-2 | CD19 | ABC1197var8 | ADI-27743 | n.a. | n.a. | 0.0 | n.a. |
| 2Fab-scFc-1scDb-3 | CD19 | ABC1197var8 | NKp46-1 | n.a. | n.a. | 0.0 | n.a. |
| 2Fab-scFc-1scDb-4 | CD19 | ABC1197var8 | NKp46-3 | n.a. | n.a. | 0.0 | n.a. |
| 2Fab-scFc-1scFv-4 | CD19 | wt Fc | NKp46-3 | 51.1 | 28.4 | 50.3 | 2.6 |
| 2scDb-AFc-10 | CD19 | 3G8 | ADI-27743 | n.a. | n.a. | 0.0 | n.a. |
| 2scDb-AFc-11 | CD19 | 3G8 | NKp46-1 | n.a. | n.a. | 2.1 | 3.0 |
| 2scDb-AFc-2 | CD19 | ABC1197var6 | ADI-27743 | n.a. | n.a. | 0.0 | n.a. |
| 2scDb-AFc-3 | CD19 | ABC1197var6 | NKp46-1 | n.a. | n.a. | 19.0 | 0.8 |
| 2tascFv-AFc-2 | CD19 | ABC1197var8 | ADI-27743 | n.a. | n.a. | 0.0 | 0.0 |
| AIG-1scDb-6 | CD19 | wt Fc | NKp46-3 | 8.3 | 4.1 | 49.0 | 14.5 |
| AIG-2scDb-1 | CD19 | P2C-47var50 | KYK-2.0 | n.a. | n.a. | 7.2 | 10.1 |
| AIG-2scDb-2 | CD19 | P2C-47var50 | ADI-27743 | n.a. | n.a. | 8.3 | 11.7 |
| AIG-2scDb-3 | CD19 | P2C-47var50 | NKp46-1 | n.a. | n.a. | 0.0 | n.a. |
| AIG-2scDb-4 | CD19 | P2C-47var50 | NKp46-3 | n.a. | n.a. | 0.0 | n.a. |
| AIG-2scFv-16 | CD19 | ABC1197var8 | KYK-2.0 | n.a. | n.a. | 0.0 | n.a. |
| AIG-2scFv-17 | CD19 | ABC1197var8 | ADI-27743 | n.a. | n.a. | 0.0 | n.a. |
| AIG-2scFv-18 | CD19 | ABC1197var8 | NKp46-1 | n.a. | n.a. | 0.0 | n.a. |
| AIG-2scFv-19 | CD19 | ABC1197var8 | NKp46-3 | n.a. | n.a. | 0.0 | n.a. |
| AIG-2scFv-20 | CD19 | 3G8 | KYK-2.0 | n.a. | n.a. | 0.0 | n.a. |
| AIG-2scFv-23 | CD19 | 3G8 | NKp46-3 | n.a. | n.a. | 0.0 | n.a. |
| IG-scDb-1 | CD19 | P2C-47var50 | KYK-2.0 | n.a. | n.a. | 0.0 | n.a. |
| IG-scDb-2 | CD19 | P2C-47var50 | ADI-27743 | n.a. | n.a. | 0.0 | n.a. |
| IG-scDb-3 | CD19 | P2C-47var50 | NKp46-1 | n.a. | n.a. | 0.0 | n.a. |
| IG-scDb-4 | CD19 | P2C-47var50 | NKp46-3 | n.a. | n.a. | 0.0 | n.a. |
| scFv-IgAb-396 | CD19 | wt Fc | NKp46-3 | 5.1 | 0.6 | 70.2 | 9.1 |

n.a., not applicable.

### Example 15: Induction of Tumor Cell Lysis by Trispecific Antibody Constructs Using PBMC as Effector Cells in 4 h Calcein-Release Cytotoxicity Assays

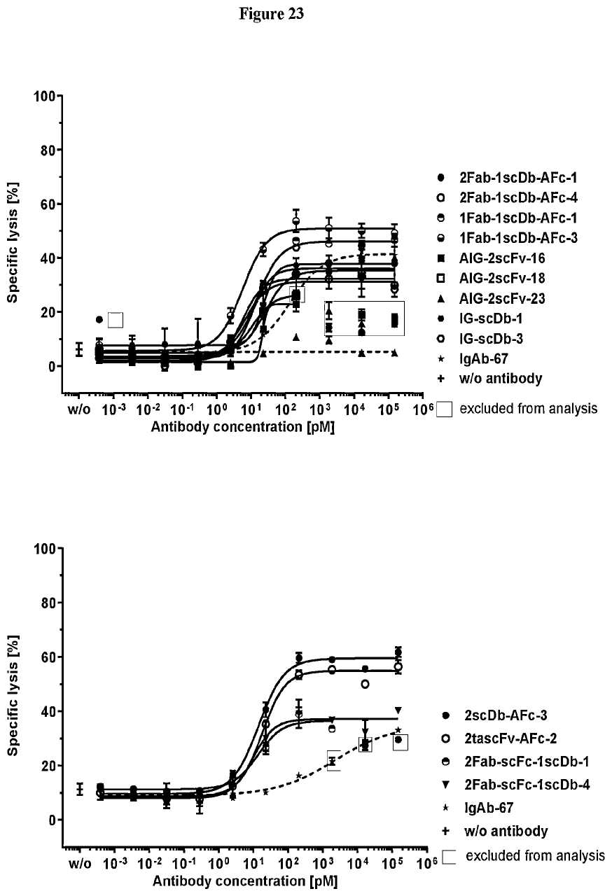

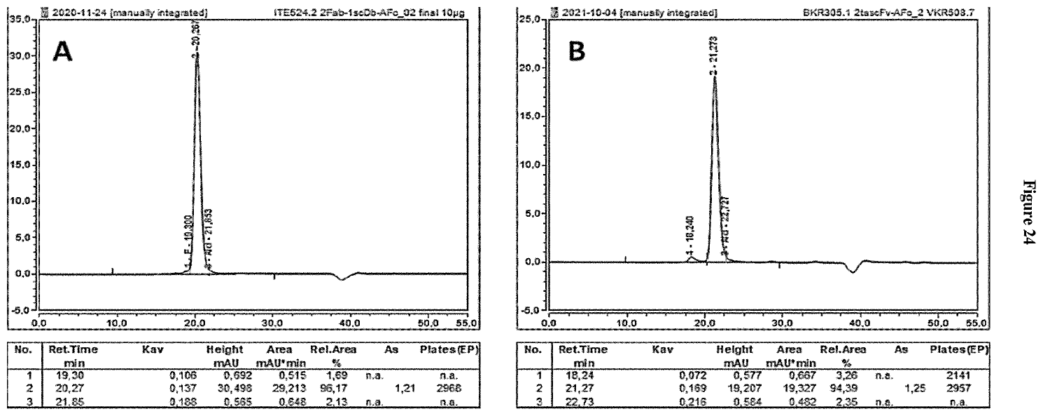

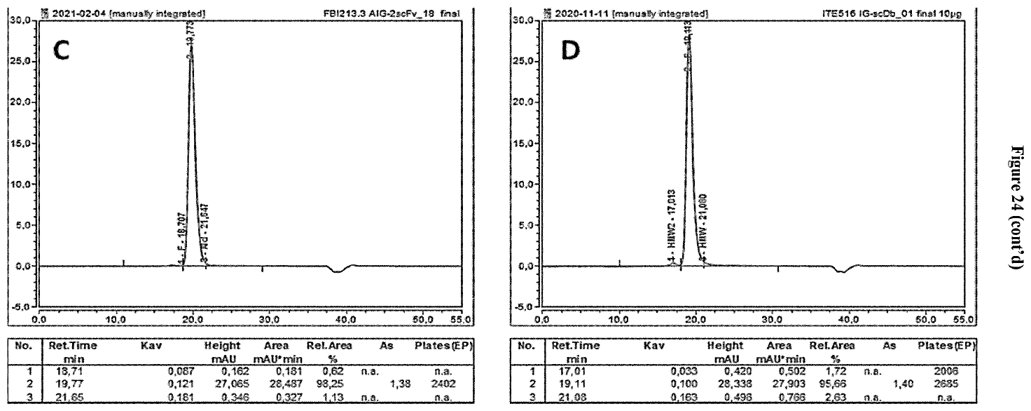

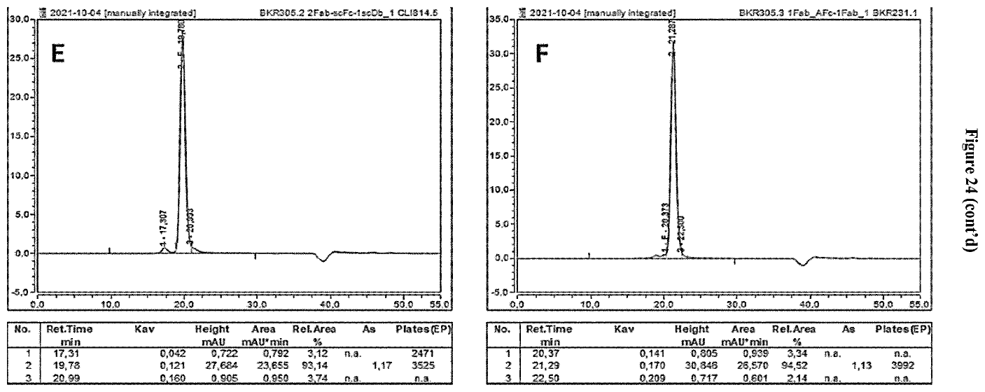

To assess the ADCC activity of CD19-targeting and EGFR-targeting trispecific antibody constructs (e.g. CD19/CD16A/NKG2D, CD19/CD16A/NKp46, EGFR/CD16A/NKG2D, EGFR/CD16A/NKp46), 4 h calcein-release cytotoxicity assays were performed with calcein-labeled CD19⁺ target cells (e.g. Raji or GRANTA-519 cells) or EGFR⁺ target cells (e.g. SW-982 or A-431 cells) and human PBMC as effector cells at an E:T ratio of 50:1 in the presence of serial dilutions of trispecific antibody constructs and control constructs. In the presence of CD19-targeting trispecific antibody constructs, specific lysis of CD19⁺ Raji or GRANTA-519 cells was induced by trispecific formats IG-scDb, 2Fab-1scDb-AFc, 1Fab-1scDb-AFc, AIG-2scFv, 2scDb-AFc, 2tascFv-AFc, 2Fab-scFc-1scDb (Table 8 and FIG. 23). In contrast, no lysis of CD19⁻/EGFR⁺ A-431 cells was observed, indicating specific lysis of target antigen-positive cells by CD19-targeting trispecific antibody constructs. Analogous, EGFR-targeting trispecific antibody constructs of the format 2Fab-1scDb-AFc induced the lysis of EGFR⁺ A-431 or SW-982 cells, whereas EGFR⁻/CD19⁺ Raji or GRANTA-19 cells were spared, indicating target antigen-positive cells by EGFR-targeting trispecific antibody constructs.

These results demonstrate that the trispecific antibody constructs not only bind to their respective recruiting receptors, e.g. CD16A, NKG2D, or NKp46, and target antigens, e.g. CD19 or EGFR, but also trigger specific lysis by human PBMC of target cell expressing the corresponding target antigen.

TABLE 8
Potency (EC₅₀) and efficacy (Eₘₐₓ) values determined for trispecific onstructs in 4 h
calcein release assays where freshly isolated human PBMC were incubated with calcein labeled
CD19⁺ or EGFR⁺ tumor cells at an effector:target ratio of 50:1 in presence of
a serial dilution of the indicated antibodies. Experiments were performed in duplicates
and the resulting mean and SD values are depicted in the table (n.a. = not applicable).

| | | Effector | Effector | EC₅₀ [pM] | | Eₘₐₓ [%] | |
|---|---|---|---|---|---|---|---|
| Construct | Target | domain 1 | domain 2 | mean | SD | mean | SD |
| IG-scDb-1 | CD19 | P2C-47var50 | KYK-2.0 | 17.6 | 7.6 | 30.6 | 0.8 |
| IG-scDb-2 | CD19 | P2C-47var50 | ADI-27743 | 10.3 | 14.1 | 29.6 | 15.5 |
| IG-scDb-3 | CD19 | P2C-47var50 | NKp46-1 | 7.3 | 4.7 | 32.1 | 1.8 |
| 2Fab-1scDb-AFc-1 | CD19 | ABC1197var8 | KYK-2.0 | 18.4 | 11.1 | 28.2 | 3.2 |
| 2Fab-1scDb-AFc-3 | CD19 | ABC1197var8 | NKp46-1 | 6.7 | 0.2 | 28.3 | 0.4 |
| 2Fab-1scDb-AFc-4 | CD19 | ABC1197var8 | NKp46-3 | 4.8 | 1.2 | 29.2 | 1.7 |
| 2Fab-1scDb-AFc-7 | EGFR | ABC1197var8 | NKp46-1 | 1.5 | 0.4 | 38.3 | 14.6 |
| 1Fab-1scDb-AFc-1 | CD19 | ABC1197var8 | KYK-2.0 | 11.9 | 4.0 | 39.9 | 3.7 |
| 1Fab-1scDb-AFc-3 | CD19 | ABC1197var8 | NKp46-1 | 4.5 | 1.7 | 42.8 | 3.5 |
| AIG-2scFv-16 | CD19 | ABC1197var8 | KYK-2.0 | 19.5 | 3.4 | 18.3 | 4.4 |
| AIG-2scFv-17 | CD19 | ABC1197var8 | ADI-27743 | 3.8 | 3.6 | 15.0 | 0.0 |
| AIG-2scFv-18 | CD19 | ABC1197var8 | NKp46-1 | 5.9 | 7.5 | 19.0 | 5.4 |
| AIG-2scFv-19 | CD19 | ABC1197var8 | NKp46-3 | 6.5 | 0.5 | 15.4 | 1.4 |
| AIG-2scFv-23 | CD19 | 3G8 | NKp46-3 | n.a. | n.a. | 0.0 | 0.0 |
| 2scDb-AFc-3 | CD19 | ABC1197var6 | NKp46-1 | 14.7 | 1.0 | 57.2 | 10.3 |
| 2tascFv-AFc-2 | CD19 | ABC1197var8 | ADI-27743 | 16.5 | 0.8 | 64.8 | 31.8 |
| 2Fab-scFc-1scDb-1 | CD19 | ABC1197var8 | KYK-2.0 | 30.7 | 31.5 | 40.5 | 27.6 |
| 2Fab-scFc-1scDb-2 | CD19 | ABC1197var8 | ADI-27743 | 48.3 | 1.0 | 36.1 | 18.7 |
| 2Fab-scFc-1scDb-3 | CD19 | ABC1197var8 | NKp46-1 | 13.3 | 10.8 | 41.0 | 25.3 |
| 2Fab-scFc-1scDb-4 | CD19 | ABC1197var8 | NKp46-3 | 15.0 | 7.8 | 44.8 | 22.3 |
| IgAb-67 | CD19 | Fc-enhanced Fc | −/− | 2332.7 | 3205.0 | 43.1 | 15.1 |
| IgAb-53 | EGFR | Fc-enhanced Fc | −/− | 27.8 | 25.1 | 58.1 | 34.0 |

### Example 16: Assessment of NK and T Cell Activation Induced by Trispecific Constructs in 24 h Cultures of PBMC in the Presence and Absence of Target Cells

To demonstrate target antigen-specific activation of NK cells and T cells by CD19-targeting trispecific antibody constructs (e.g. CD19/CD16A/NKG2D and CD19/CD16A/NKp46) and control constructs, unfractionated PBMC or CD19⁺ B cell-depleted PBMC were incubated for 24 h with trispecific antibody constructs followed by flow cytometric analysis of activation markers on NK cells and T cells. In unfractionated PBMC, CD19-targeting trispecific antibody constructs of the formats IG-scDb, 2Fab-1scDb-AFc, 1Fab-1scDb-AFc, AIG-2scFv, 2scDb-AFc, 2tascFv-AFc, 2Fab-scFc-1scDb and 2Fab-scFc-1scFv induce upregulation of activation markers, e.g. CD25, CD69, or CD137 on NK cells (table 9, columns “in presence of target cells”). Moreover, CD19-targeting trispecific antibody constructs of the formats IG-scDb, 2Fab-1scDb-AFc, 1Fab-1scDb-AFc, AIG-2scFv, 2scDb-AFc, 2tascFv-AFc, 2Fab-scFc-1scDb and 2Fab-scFc-1scFv result in the upregulation of activation marker e.g. CD25, CD69 or CD137 on T cell subsets (table 10, columns “in presence of target cells”). Conversely, NK cell and T cell activation was observed to a much lesser extent for most of the constructs named above using B cell-depleted PBMC, demonstrating target antigen-specific NK cell and T cell activation by CD16A/NKG2D and CD16A/NKp46 engaging trispecific antibody constructs (tables example 16 A and B, columns “without target cells”. In contrast, CD19-targeting trispecific antibody constructs comprising active Fc domains (e.g. CD19/Fc/NKp46) induced significant NK cell and T cell activation in both PBMC and B cell-depleted PBMC (e.g. 1Fab-AFc-1Fab-1 and -2 with wt Fc and effector domains NKp46-1 and NKp46-3, respectively as well as 1Fab-AFc-1Fab-5 and -6, with Fc-enhanced Fc domains and effector domains NKp46-1 and NKp46-3; tables 9 and 10).

Similarly, incubation of PBMC with EGFR-recruiting trispecific antibody constructs of the format 2Fab-1scDb-AFc (e.g. EGFR/CD16A/NKG2D and EGFR/CD16A/NKp46) resulted in the upregulation of activation marker, CD25, CD69, or CD137 on NK cell and T cell subsets only in the presence of supplemented EGFR⁺ target cells (e.g. SW-982 or A-431) but not or to a much lesser extent in the absence of EGFR⁺ target cells (table 9). On CD8⁺ T cells, we observe a moderate upregulation of activation markers (e.g. CD25, CD69, or CD137) for the same trispecific format 2Fab-1scDb-AFc both, in presence and in absence of EGFR⁺ target cells (table 10). However, EGFR-recruiting trispecific antibody constructs comprising active Fc domains (e.g. EGFR/Fc/NKG2D and EGFR/Fc/NKp46) mediated activation of NK cells and T cells in PBMC irrespective of the presence of supplemented EGFR⁺ target cells.

Tables 9 and 10: Induction of NK cell (table 9) and CD8⁺ T cell (table 10) activation by trispecific molecules in presence or absence of target cells. For CD19-targeting trispecifics (see column “target”) or the Fc-enhanced anti-CD19 IgG1 control antibody IgAb-67, unfractionated PBMC or CD19⁺ B cell-depleted PBMC were incubated for 24 h with the indicated concentrations of antibody constructs followed by flow cytometric determination of the percentage of CD69-positive NK cells and T cells. Alternatively, PBMC tested with EGFR targeting trispecific molecules or the Fc-enhanced anti-EGFR IgG1 control antibody IgAb-53 (see column “target”) were incubated with or without supplemented EGFR⁺ CMFDA-labeled A-431 target cells.

### Example 17: Assessment of Target Cell Depletion Induced by Trispecific Constructs in 24 h Cultures of PBMC

To demonstrate depletion of CD19⁺ B cells by CD19-targeting trispecific antibody constructs (e.g. CD19/CD16A/NKG2D and CD19/CD16A/NKp46), unfractionated PBMC were incubated for 24 h with trispecific antibody constructs and control constructs followed by flow cytometric analysis of absolute viable CD20⁺ B cell counts. CD19-targeting trispecific antibody constructs (see column “target”) of the formats IG-scDb, 2Fab-1scDb-AFc, 1Fab-1scDb-AFc, AIG-2scFv, 2scDb-AFc, 2tascFv-AFc, 2Fab-scFc-1scDb and 2Fab-scFc-1scFv resulted in concentration-dependent reduction in autologous B cells (table 11). In most cases these formats induced a higher percentage of reduction of autologous B-cells than the comparator constructs of the formats 1Fab-AFc-1Fab and AIG-1scFv with wt Fe or enhanced Fe domains.

To show depletion of EGFR⁺ target cells by EGFR-targeting trispecific antibody constructs (e.g. EGFR/CD16A/NKG2D and EGFR/CD16A/NKp46) PBMC were co-cultured for 24 h with CMFDA-labelled EGFR⁺ target cells (e.g. SW-982 or A-431) in the presence of trispecific antibody construct 2Fab-1scDb-AFc-7 or control antibody IgAb-53 followed by flow cytometric analysis of absolute viable EGFR⁺ cell counts. The presence of the EGFR-targeting trispecific antibody construct resulted in a substantial reduction in EGFR⁺ target cells (up to 50.7% at 208 ng/mL).

Hence, CD19-targeting and EGFR-targeting trispecific antibody constructs result in target antigen-specific activation of NK cells and T cells but also elicit specific depletion of target cells upon 24 h co-culture with PBMC.

### Example 18: Expression and Purification of Trispecific Innate-Cell Engager Formats

Asymmetric antibody formats were generated by assembly from two separately expressed half-antibodies containing knob- (T366W) or hole- (T366S, L368A, Y407V) mutations in their Fc portions, respectively.

Expression plasmids were generated by standard molecular biology techniques. CHO codon-optimized DNA fragments were gene-synthesized by GeneArt or amplified via PCR from available expression vectors and subcloned into a modified bicistronic mammalian expression vector pcDNA5/FRT (Life Technologies) containing two CMV promoter-controlled expression cassettes and a gene mediating Puromycin resistance.

For asymmetric IgG-scFv fusion formats, tumor targeting variable heavy and light chain domain sequences with specificities for e.g. HER2, EGFR, CD19 or others, were fused at their C-terminus to sequences of CH1 and CL of effector-silent (e.g. L234F/L235E/D265A) human IgG1 containing Knob-into-Hole mutations (Knob chain->T366W, and Hole chain->T366S, L368A, Y407V), respectively. Variable heavy and light chain domain sequences of CD16-specific antibody clones, NKG2D-specific clones or NKp46-specific clones, were fused in scFv format using a (GGGGS)₆ connector to the C-terminus of CH3 of the knob- and hole mutated Fc, respectively. To mediate protein secretion, signal peptides were added to the N-terminus of both (heavy and light) antibody chains. Sequences of all constructs were confirmed by DNA sequencing (Eurofins GATC Biotech, Cologne, Germany).

Recombinant half antibodies were expressed in CHO cells as previously described (Ellwanger et al., MAbs 2019:1-20). An alternative to the stable expression is the co-expression of asymmetric antibody comprising chains using transient transfection and expression (e.g. using ExpiCHO system, Fisher Scientific, Cat. A29133).

Fc containing antibodies were purified from clarified cell culture supernatants (CCS) using Protein A affinity chromatography (MabSelect SuRe 5 mL). Protein A elution fractions containing target protein were formulated in 10 mM Na-Acetate+4.5% Sorbitol pH 5.0 and analyzed via UV-Spectroscopy, SDS-PAGE (non-reducing (nR) or reducing (R)), analytical SE-HPLC and MALS-dRI and revealed the expected sizes of monomeric half-antibodies with a minor proportion of associated dimers.

For the assembly, separately expressed Knob- and Hole-half antibodies were mixed at an equimolar concentration, titrated to pH 8.5 using 100 mM Tris-Arginine pH 9.0 and supplemented with 200× molar excess of freshly prepared reduced L-Glutathione and incubated over night at 32° C. Control samples were drawn initially after mixing (0d) and after one day (1 d) of incubation. Finally, buffer was exchanged to 10 mM Na-Acetate+4.5% Sorbitol pH 5.0 and product was analyzed by analytical SE-HPLC, MALS-dRI and revealed the expected sizes of assembled antibody with 89% purity.

Antibody preparations not showing sufficient purity were further polished via preparative size-exclusion chromatography (SEC) and analyzed using SEC/MALS-HPLC (multi-angle light scattering), SDS-PAGE and UV-Vis spectroscopy (FIG. 24).

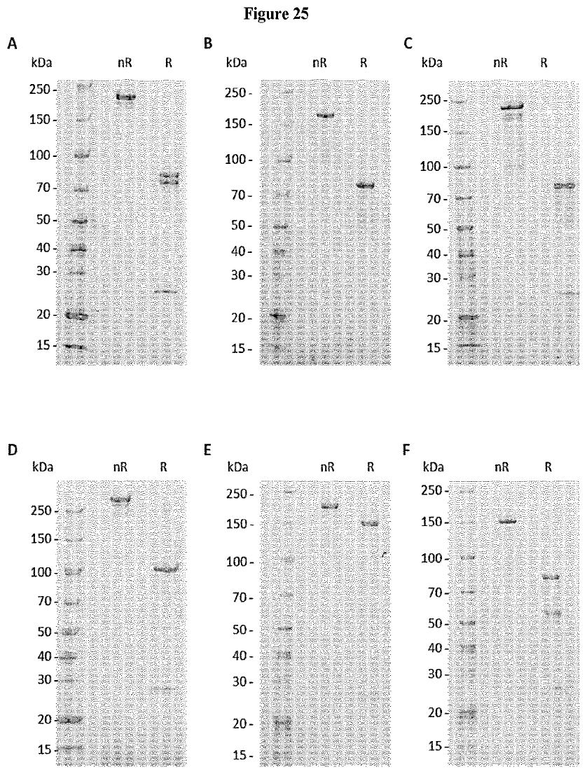

All molecules were able to be expressed and purified or assembled and purified and obtained product purities in the range of 64.42-100% (evaluated by SE-HPLC, table 12). In SDS-PAGE, molecules showed expected apparent molecular weights under non-reducing conditions and presence of expected fragments after reduction (FIG. 25).

TABLE 12
Purities of trispecific molecules as evaluated by SE-HPLC

| | effector | effector | purity |
|---|---|---|---|
| construct | domain 1 | domain 2 | SE-HPLC [%] |
| IG-scDb-1 | CD16A(P2C47var50) | NKG2D(KYK-2.0) | 95.66 |
| IG-scDb-2 | CD16A(P2C47var50) | NKG2D(ADI-27743) | 96.01 |
| IG-scDb-3 | CD16A(P2C47var50) | NKp46(NKp46-1) | 98.03 |
| IG-scDb-4 | CD16A(P2C47var50) | NKp46(NKp46-3) | 95.98 |
| 2Fab-1scDb-AFc-1 | CD16A(ABC1197var8) | NKG2D(KYK-2.0) | 98.96 |
| 2Fab-1scDb-AFc-2 | CD16A(ABC1197var8) | NKG2D(ADI-27743) | 96.17 |
| 2Fab-1scDb-AFc-3 | CD16A(ABC1197var8) | NKp46(NKp46-1) | 97.56 |
| 2Fab-1scDb-AFc-4 | CD16A(ABC1197var8) | NKp46(NKp46-3) | 95.58 |
| 2Fab-1scDb-AFc-5 | CD16A(ABC1197var8) | NKG2D(KYK-2.0) | 96.27 |
| 2Fab-1scDb-AFc-6 | CD16A(ABC1197var8) | NKG2D(ADI-27743) | 94.36 |
| 2Fab-1scDb-AFc-7 | CD16A(ABC1197var8) | NKp46(NKp46-1) | 95.34 |
| 2Fab-1scDb-AFc-9 | CD16A(3G8) | NKG2D(KYK-2.0) | 93.72 |
| 2Fab-1scDb-AFc_10 | CD16A(3G8) | NKG2D(ADI-27743) | 96.70 |
| 2Fab-1scDb-AFc_11 | CD16A(3G8) | NKp46(NKp46-1) | 92.49 |
| 1Fab-1scDb-AFc-1 | CD16A(ABC1197var8) | NKG2D(KYK-2.0) | 96.46 |
| 1Fab-1scDb-AFc-2 | CD16A(ABC1197var8) | NKG2D(ADI-27743) | 95.87 |
| 1Fab-1scDb-AFc-3 | CD16A(ABC1197var8) | NKp46(NKp46-1) | 94.37 |
| AIG-2scFv-16 | CD16A(ABC1197var8) | NKG2D(KYK-2.0) | 98.59 |
| AIG-2scFv-17 | CD16A(ABC1197var8) | NKG2D(ADI-27743) | 95.98 |
| AIG-2scFv-18 | CD16A(ABC1197var8) | NKp46(NKp46-1) | 98.25 |
| AIG-2scFv-19 | CD16A(ABC1197var8) | NKp46(NKp46-3) | 95.67 |
| AIG-2scFv-20 | CD16A(3G8) | NKG2D(KYK-2.0) | 96.94 |
| AIG-2scFv-23 | CD16A(3G8) | NKp46(NKp46-3) | 95.35 |
| 2Fab-1scFv-AFc-1 | wt Fc | NKG2D(KYK-2.0) | 64.42 |
| 2Fab-1scFv-AFc-2 | wt Fc | NKG2D(ADI-27743) | 88.92 |
| 2Fab-1scFv-AFc-3 | wt Fc | NKp46(NKp46-1) | 97.44 |
| 2Fab-1scFv-AFc-4 | wt Fc | NKp46(NKp46-3) | 95.64 |
| AIG-1scDb-3 | wt Fc | NKG2D(KYK-2.0) | 93.80 |
| AIG-1scDb-4 | wt Fc | NKG2D(ADI-27743) | 85.24 |
| AIG-1scDb-5 | wt Fc | NKp46(NKp46-1) | 92.44 |
| AIG-1scDb-6 | wt Fc | NKp46(NKp46-3) | 92.48 |
| 1scDb-1scFv-AFc-2 | wt Fc | NKG2D(ADI-27743) | 95.50 |
| 1scDb-1scFv-AFc-3 | wt Fc | NKp46(NKp46-1) | 99.60 |
| 2scDb-AFc-2 | CD16A(ABC1197var6) | NKG2D(ADI-27743) | 95.40 |
| 2scDb-AFc-3 | CD16A(ABC1197var6) | NKp46(NKp46-1) | 99.80 |
| 2scDb-AFc-10 | CD16A(3G8) | NKG2D(ADI-27743) | 87.30 |
| 2scDb-AFc-11 | CD16A(3G8) | NKp46(NKp46-1) | 97.90 |
| 2tascFv-AFc-2 | CD16A(ABC1197var8) | NKG2D(ADI-27743) | 96.10 |
| 1tascFv-1scFv-AFc-2 | wt Fc | NKG2D(ADI-27743) | 76.20 |
| 2Fab-scFc-1scDb-1 | CD16A(ABC1197var8) | NKG2D(KYK-2.0) | 97.30 |
| 2Fab-scFc-1scDb-2 | CD16A(ABC1197var8) | NKG2D(ADI-27743) | 98.30 |
| 2Fab-scFc-1scDb-3 | CD16A(ABC1197var8) | NKp46(NKp46-1) | 97.90 |
| 2Fab-scFc-1scDb-4 | CD16A(ABC1197var8) | NKp46(NKp46-3) | 97.20 |
| 2Fab-scFc-1scFv-1 | wt Fc | NKG2D(KYK-2.0) | 98.60 |
| 2Fab-scFc-1scFv-2 | wt Fc | NKG2D(ADI-27743) | 98.20 |
| 2Fab-scFc-1scFv-3 | wt Fc | NKp46(NKp46-1) | 99.20 |
| 2Fab-scFc-1scFv-4 | wt Fc | NKp46(NKp46-3) | 99.40 |
| 1Fab-AFc-1Fab-1 | wt Fc | NKp46(NKp46-1) | 97.10 |
| (ComparatorA_1) | | | |
| 1Fab-AFc-1Fab-2 | wt Fc | NKp46(NKp46-3) | 100.00 |
| (ComparatorA_2) | | | |
| 1Fab-AFc-1Fab-5 | Fc-enhanced | NKp46(NKp46-1) | 99.40 |
| (ComparatorA_5) | | | |
| 1Fab-AFc-1Fab_6 | Fc-enhanced | NKp46(NKp46-3) | 98.50 |
| (ComparatorA_6) | | | |
| AIG-1scFv-5 | wt Fc | NKG2D(KYK-2.0) | 97.30 |
| (ComparatorB_1) | | | |
| AIG-1scFv-6 | wt Fc | NKG2D(ADI-27743) | 99.3 |
| (ComparatorB_2) | | | |
| scFv-IgAb-393 | wt Fc | NKG2D(KYK-2.0) | 98.61 |
| scFv-IgAb-394 | wt Fc | NKG2D(ADI-27743) | 97.83 |
| scFv-IgAb-395 | wt Fc | NKp46(NKp46-1) | 97.77 |
| scFv-IgAb-396 | wt Fc | NKp46(NKp46-3) | 96.82 |
| IgAb-67 | Fc-enhanced | −/− | 96.26 |

### Example 19: Monovalent Binding Interaction of Trispecific Antibodies in SPR

To assess functionality of all binding specificities of HER2/CD16A/CD89 trispecific antibody constructs, monovalent interaction kinetics to human CD16A^{158V}, human CD16A^{158F} and human CD89 were analyzed at 37° C. using a Biacore T200 instrument (GE Healthcare) equipped with a research-grade Sensor Chip CAP (Biotin CAPture Kit, GE Healthcare) pre-equilibrated in HIBS-P+ running buffer. For monovalent interaction analysis, trispecific antibody constructs were captured (FC2, FC4) on immobilized biotinylated human HTER2-mFc.silenced/Avi-tag to a density of 50-80 RU, before recombinant monomeric human CD16A^{158V}, human CD16A^{158F} or human CD89 (concentration: 0-240 nM) was injected for 180 s at a flow rate of 40 μL/min and complex was left to dissociate for 300 s at the same flow rate. After each cycle, chip surfaces were regenerated with 6 M guanidine-HCl, 0.25 M NaOH and reloaded with Biotin Capture reagent. Interaction kinetics were determined by fitting data from multi-cycle kinetics experiments to a simple 1:1 interaction model using the local data analysis option (Rmax and RI) available within Biacore T200 Evaluation Software (v3.1). Referencing was done against a flow cell without captured ligand (Fc2-Fc1, Fc4-Fc3).

To show selectivity and to assess functionality of all binding specificities of trispecific antibody constructs, monovalent binding to recombinant human CD16A and CD89 was analyzed by SPR interaction analysis at 37° C. using recombinant monomeric antigen as analyte. Binding affinity (K_{D}) of human CD16A to trispecific antibody constructs was determined to be 31.2 nM to 32.4 nM (human CD16A 158V) and 60.4 nM to 62.9 nM (human CD16A 158F) for molecules comprising only one anti-CD16A binding domain (AIG-2scFv-28, AIG-2scFv-29). The apparent affinity of human CD16A to the bispecific tetravalent control antibody scFv-IgAb-356 was 19.8 nM for CD16A 158V and 37.2 nM for human CD16A 158F. Binding affinity of human CD89 to trispecific constructs with only one anti-CD89 Fv domain (AIG-2scFv-28, AIG-2scFv-29), bispecific constructs with two anti-CD89 Fv domains (scFv-IgAb-441, scFv-IgAb_442), or bispecific constructs with only one anti-CD89 Fv domain (AIG-1scFv-6, AIG-1scFv-7) showed similar and very high, affinities with K_{D} values in the range 0.076 nM to 0.089 nM for constructs with anti-CD89 domain 14.1 and K_{D} values between 0.40 nM and 0.51 nM for constructs with anti-CD89 domain A77.

As antibodies were captured on immobilizes human HER2 prior to analysis for monovalent interaction with human CD16A or human CD89, data from this study suggesting selective binding for all three specificities. Although the objective of this study was to investigate specific interaction kinetics of all binding specificities, data let further suggest, that molecules are able to at least bind two different antigens simultaneously (HER2/CD16A; HER2/CD89).

TABLE 13
Trispecific and bispecific antibody binding to human CD16A (158V), CD16A (158F), and CD89 was measured
in SPR using a monovalent multi-cycle kinetic set-up at 37° C. Trispecific or bispecific constructs
were captured on a HER2-biotin-capture chip and recombinant CD16A (158V), CD16A (158F), and human
CD89 were used as analytes. Affinity and kinetic parameters were evaluated using 1:1 binding model
in three independent experiments; arithmetic mean ± standard deviation is reported here.

| | | | on-rate ka | | off-rate kd | | K_{D} | |
|---|---|---|---|---|---|---|---|---|
| | | | aritmethic | | aritmethic | | aritmethic | |
| | | Binding | mean | SD | mean | SD | mean | SD |
| Sample | Description | partner | (M⁻¹s⁻¹) | (M⁻¹s⁻¹) | (s⁻¹) | (s⁻¹) | (M) | (M) |
| AIG-1scFv-6 | HER2 (4D5) × | human | no binding | | | | | |
| | CD89 (14.1) | CD16A | | | | | | |
| AIG-1scFv-7 | HER2 (4D5) × | 158V | no binding | | | | | |
| | CD89 (A77) | | | | | | | |
| AIG-2scFv-28 | HER2 (4D5) × | | 7.46E+05 | 1.57E+05 | 2.40E−02 | 4.22E−03 | 3.24E−08 | 1.11E−09 |
| | CD89 (14.1) × | | | | | | | |
| | CD16A (P2C- | | | | | | | |
| | 47var50) | | | | | | | |
| AIG-2scFv-29 | HER2 (4D5) × | | 8.85E+05 | 1.51E+05 | 2.77E−02 | 5.08E−03 | 3.12E−08 | 1.07E−09 |
| | CD89 (A47) × | | | | | | | |
| | CD16A (P2C- | | | | | | | |
| | 47var50) | | | | | | | |
| scFv-IgAb-356 | HER2 (4D5) × | | 1.48E+06 | 3.61E+04 | 2.92E−02 | 2.08E−04 | 1.98E−08 | 3.46E−10 |
| | CD16A (P2C- | | | | | | | |
| | 47var50) | | | | | | | |
| scFv-IgAb-441 | HER2 (4D5) × | | no binding | | | | | |
| | CD89 (14.1) | | | | | | | |
| scFv-IgAb-442 | HER2 (4D5) × | | no binding | | | | | |
| | CD89 (A77) | | | | | | | |
| AIG-1scFv-6 | HER2 (4D5) × | human | no binding | | | | | |
| | CD89 (14.1) | CD16A | | | | | | |
| AIG-1scFv-7 | HER2 (4D5) × | 158F | no binding | | | | | |
| | CD89 (A77) | | | | | | | |
| AIG-2scFv-28 | HER2 (4D5) × | | 5.63E+05 | 6.65E+04 | 3.39E−02 | 3.23E−03 | 6.04E−08 | 2.80E−09 |
| | CD89 (14.1) × | | | | | | | |
| | CD16A (P2C- | | | | | | | |
| | 47var50) | | | | | | | |
| AIG-2scFv-29 | HER2 (4D5) × | | 6.54E+05 | 1.66E+05 | 4.05E−02 | 7.00E−03 | 6.29E−08 | 7.04E−09 |
| | CD89 (A47) × | | | | | | | |
| | CD16A (P2C- | | | | | | | |
| | 47var50) | | | | | | | |
| scFv-IgAb-356 | HER2 (4D5) × | | 9.15E+05 | 2.34E+05 | 3.41E−02 | 8.27E−03 | 3.72E−08 | 5.66E−10 |
| | CD16A (P2C- | | | | | | | |
| | 47var50) | | | | | | | |
| scFv-IgAb-441 | HER2 (4D5) × | | no binding | | | | | |
| | CD89 (14.1) | | | | | | | |
| scFv-IgAb-442 | HER2 (4D5) × | | no binding | | | | | |
| | CD89 (A77) | | | | | | | |
| AIG-1scFv-6 | HER2 (4D5) × | human | 3.49E+06 | 7.98E+05 | 2.98E−04 | 3.37E−05 | 8.94E−11 | 2.72E−11 |
| | CD89 (14.1) | FCAR/ | | | | | | |
| AIG-1scFv-7 | HER2 (4D5) × | CD89 | 6.08E+05 | 6.16E+04 | 2.42E−04 | 3.06E−05 | 4.05E−10 | 9.27E−11 |
| | CD89 (A77) | | | | | | | |
| AIG-2scFv-28 | HER2 (4D5) × | | 3.13E+06 | 4.71E+05 | 2.33E−04 | 3.13E−05 | 7.62E−11 | 2.00E−11 |
| | CD89 (14.1) × | | | | | | | |
| | CD16A (P2C- | | | | | | | |
| | 47var50) | | | | | | | |
| AIG-2scFv-29 | HER2 (4D5) × | | 4.78E+05 | 7.97E+04 | 2.34E−04 | 5.63E−05 | 5.08E−10 | 1.83E−10 |
| | CD89 (A47) × | | | | | | | |
| | CD16A (P2C- | | | | | | | |
| | 47var50) | | | | | | | |
| scFv-IgAb-356 | HER2 (4D5) × | | no binding | | | | | |
| | CD16A (P2C- | | | | | | | |
| | 47var50) | | | | | | | |
| scFv-IgAb-441 | HER2 (4D5) × | | 2.63E+06 | 4.86E+05 | 2.22E−04 | 7.51E−06 | 8.63E−11 | 1.53E−11 |
| | CD89 (14.1) | | | | | | | |
| scFv-IgAb-442 | HER2 (4D5) × | | 4.84E+05 | 6.65E+04 | 2.25E−04 | 1.94E−05 | 4.67E−10 | 2.50E−11 |
| | CD89 (A77) | | | | | | | |

### Example 20: Induction of ADCP by Trispecific Antibody Constructs

To assess the ADCP activity of HER2/CD16A/CD89 trispecific constructs relative to the activity of HER2/CD16A bispecific constructs a 4 h ADCP assay on SK-BR-3 target cells was established. PBMC were isolated from buffy coats as described in Example 3. CD14⁺ monocytes were enriched from PBMC by positive immunomagnetic bead selection using the EasySep™ Human CD14 Positive Selection KIT II (Stem Cell Technologies, cat.: 17858) with the Big Easy EasySep™ Magnet (Stem Cell Technologies, cat.: 18001) according to the manufacturer's instructions. Enriched monocytes were cultured for 5 days in complete RPMI 1640 medium (RPMI 1640 medium supplemented with 10% heat-inactivated FCS, 4 mM L-glutamine, 100 U/mL penicillin G sodium, 100 μg/mL streptomycin sulfate) supplemented with 50 ng/mL M-CSF (Thermo Fisher Scientific, cat.: PHC9501), and after medium exchange including M-CSF cultured for additional 2 days. Macrophages were harvested and aliquots of 3×10⁴ macrophages were seeded in individual wells of 96-well UpCell plates (Thermo Fisher Scientific, cat.: 174897) and cultured O/N. Target cells were labeled with 0.5 μM CellTracker™ Green CMFDA Dye (Thermo Fisher Scientific, cat.: C2925) at 37° C. for 30 min, washed, and cultured O/N. Target cells were then seeded on top of the adherent macrophages at an E:T ratio of 1:1 in the presence of serial dilutions of the indicated antibodies in duplicates. After 4 hours incubation, cells were detached from the culture plate by incubation on ice and stained with A700-labeled anti-CD11b (M1/70; BioLegend, cat.: 101222) and fixable viability dye eF780 (Thermo Fisher Scientific, cat.: 65-0865-14) for 30 min at 4° C. Phagocytosis of labeled target cells was quantified by analyzing CD11b⁺/CMFDA⁺ cells in % of viable cells and depletion of target cell was measured by quantification of CD11b⁻/CMFDA⁺ cells by flow cytometry. ADCP in absence of antibodies was assessed in duplicates. Phagocytosis and depletion of target cells in the presence of antibody constructs was normalized to samples incubated in the absence of antibodies.

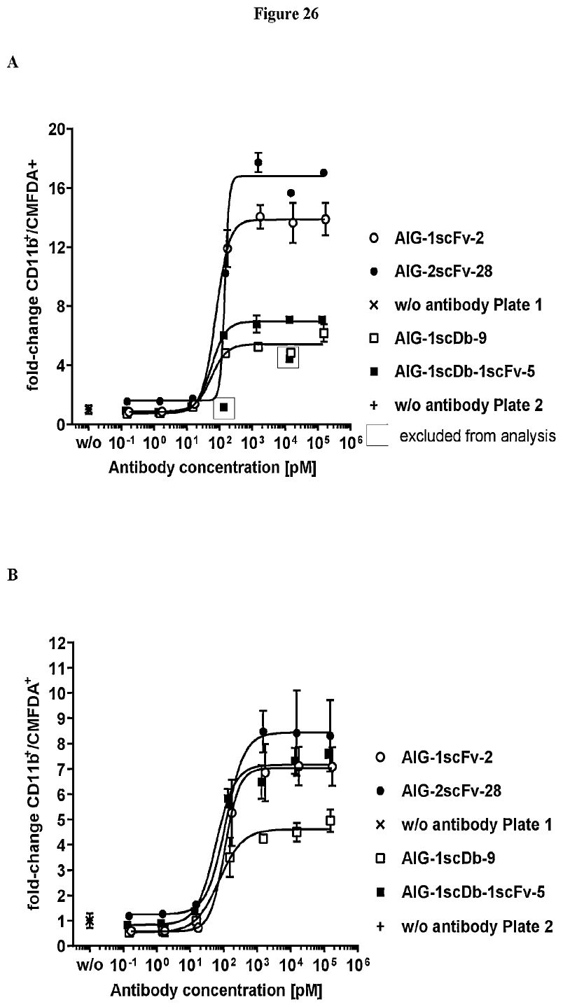

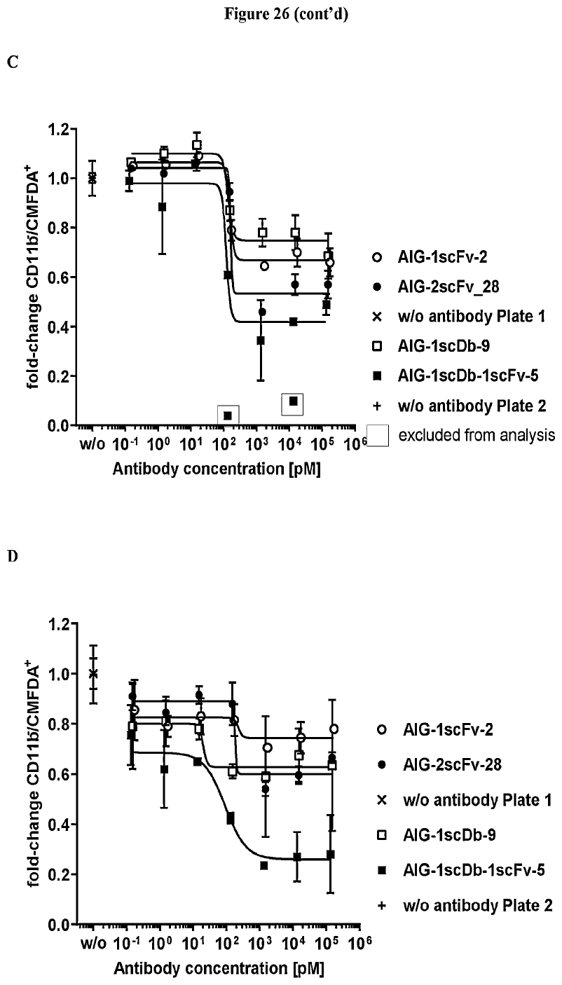

The results of two independent ADCP assays (FIG. 26) demonstrate substantial stronger induction of phagocytosis and target cell depletion by trispecific HER2/CD16A/CD89 constructs AIG-2scFv-28 and AIG-1scDb-1scFv-5 when compared with the corresponding HER2/CD16A bispecific constructs AIG-1scFv-2 and AIG-1scDb-9, respectively.

### Example 21: Induction of Neutrophil-Mediated ADCC by Trispecific Antibodies

For the isolation of neutrophils buffy coat samples were diluted with a two-to-threefold volume of PBS (Invitrogen, cat.: 14190-169), layered on a cushion of Lymphoprep (Stem Cell Technologies, cat.: 07861) in SepMate™-50 (IVD) tubes (Stem Cell Technologies, cat.: 85460), and centrifuged at 800×g for 25 min at room temperature w/o brake. After centrifugation, diluted plasma, PBMC interface, and density gradient medium were discarded, and pellets containing red blood cells and polymorphonuclear cells were pooled. One volume of the pellet was mixed with 9 volumes of ammonium chloride solution (Stem Cell Technologies, cat.: 07800) and incubated for 15 min on ice. After centrifugation for 10 min at 500×g, supernatant was discarded and cell pellet was resuspended in RoboSep buffer (Stem Cell Technologies, cat.: 20104). Neutrophils were then enriched by negative selection using EasySep™ Human Neutrophil Isolation Kit (Stem Cell Technologies, cat.: 17957) according to the manufacturer's instructions and used as effector cells in 4 h calcein-release cytotoxicity assays. The indicated target cells were harvested from cultures, washed with RPMI 1640 medium without FCS, and labeled with 10 μM calcein AM (Invitrogen/Molecular Probes, cat.: C3100MP) for 30 min in RPMI 1640 medium without FCS at 37° C. After gently washing, the labeled cells were resuspended in complete RPMI 1640 medium (RPMI 1640 medium supplemented with 10% heat-inactivated FCS, 4 mM L-glutamine, 100 U/mL penicillin G sodium, 100 μg/mL streptomycin sulfate) to a density of 1×10⁵/mL. 1×10⁴ target cells were then seeded together with neutrophils at the indicated E:T ratios in the presence of 3 μg/mL of the indicated antibody constructs in individual wells of a round-bottom 96-well microplate in a total volume of 200 μL/well in duplicates. Spontaneous release, maximal release and killing of targets by effectors in the absence of antibodies were determined in quadruplicate on each plate. For induction of maximal calcein-release Triton X-100 was added to the respective wells at a final concentration of 1%. After centrifugation for 2 min at 200×g the assay was incubated for 4 h at 37° C. in a humidified atmosphere with 5% CO₂. 100 μL cell culture supernatant were harvested from each well after an additional centrifugation for 5 min at 500×g, transferred to a black flat-bottom microplate, and the fluorescence of the released calcein was measured at 520 nm using a fluorescence plate reader (Infinite M Plex, Tecan Group, Mannedorf, Switzerland). On the basis of the measured counts, the specific cell lysis was calculated according to the following formula: [fluorescence (sample)−fluorescence (spontaneous)]/[fluorescence (maximum)−fluorescence (spontaneous)]×100%. Fluorescence (spontaneous) represents the fluorescent counts from target cells in the absence of effector cells and antibodies and fluorescence (maximum) represents the total cell lysis induced by the addition of Triton X-100. Mean lysis values and SD were plotted using GraphPad Prism software.

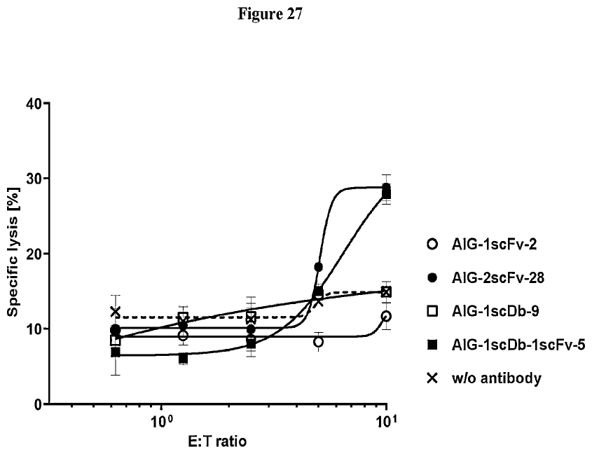

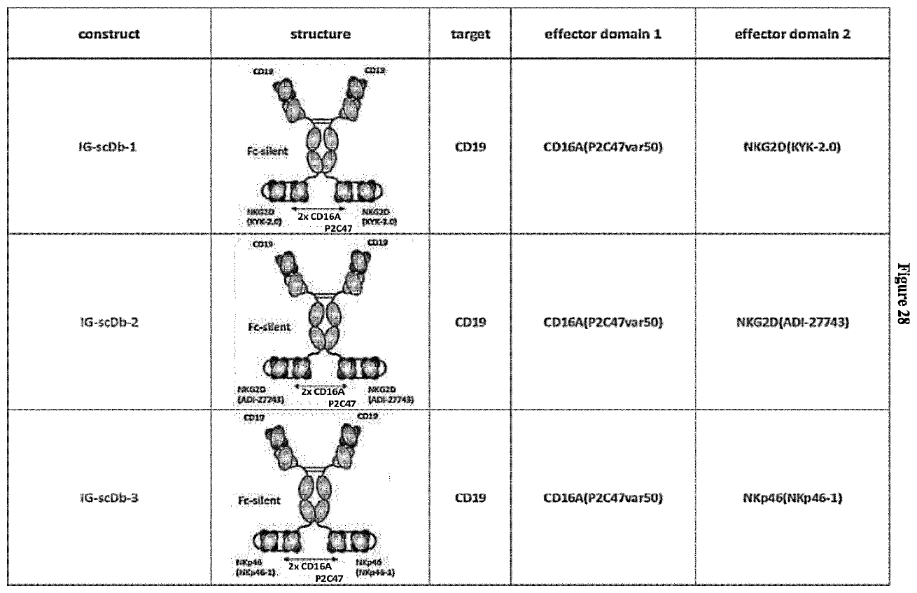

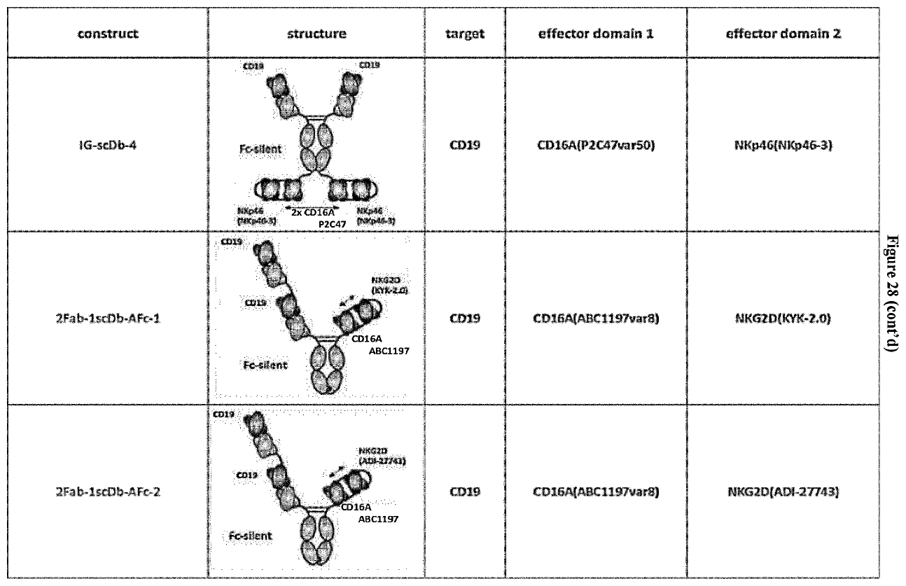

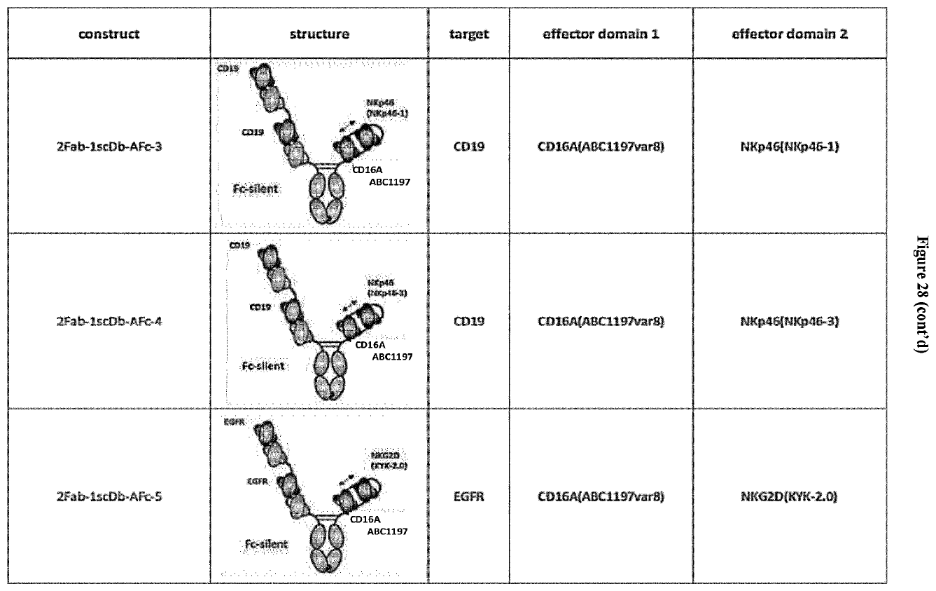

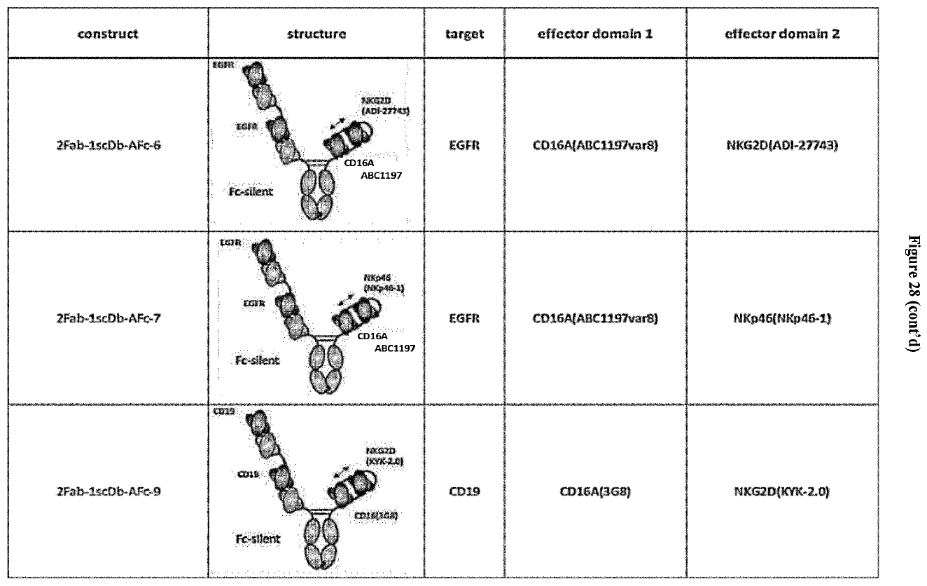

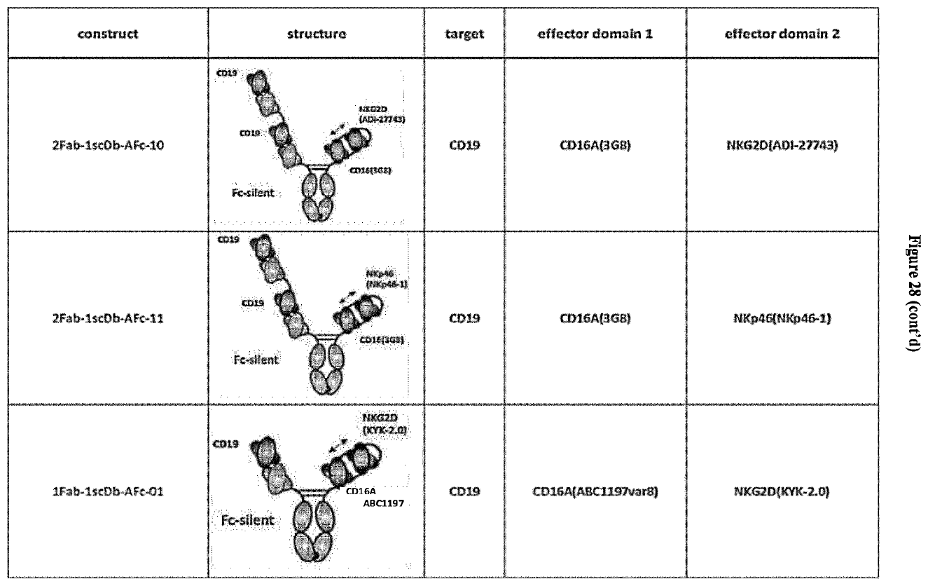

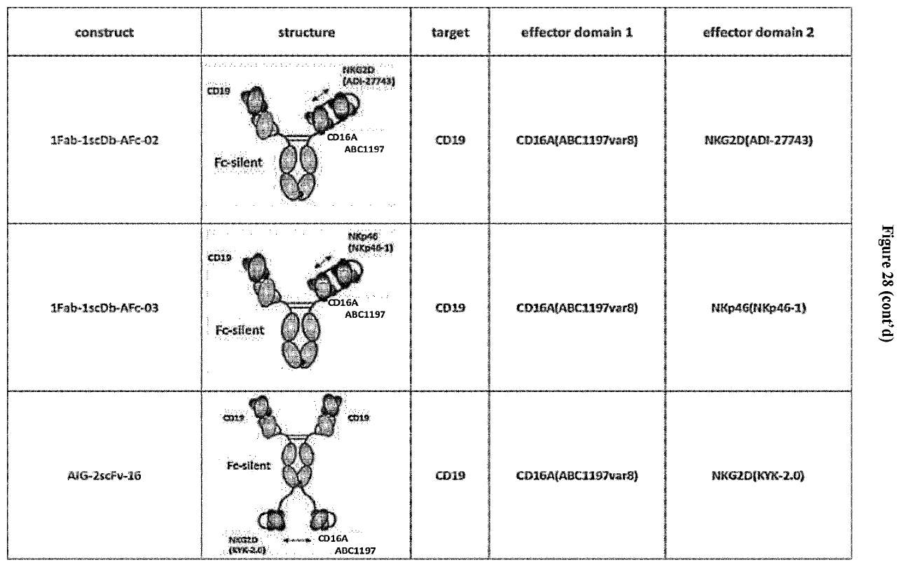

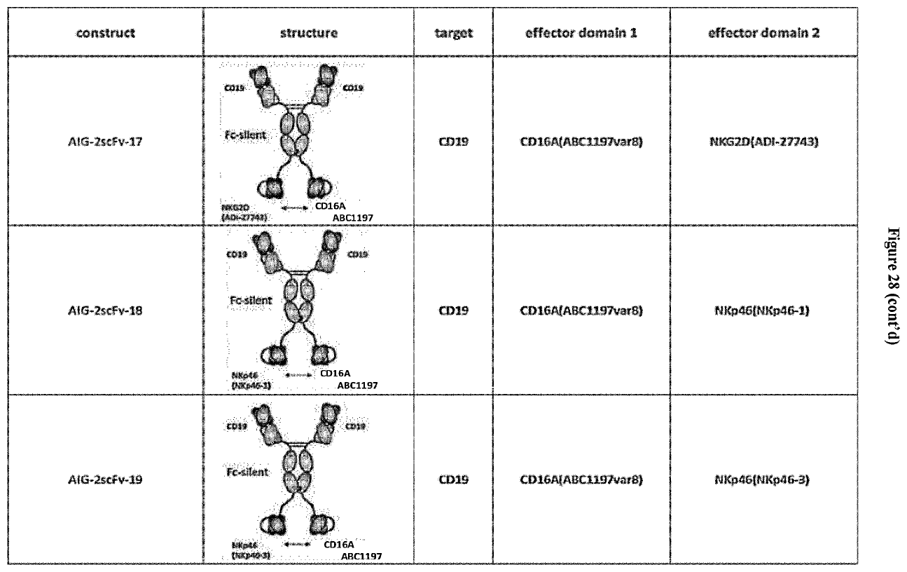

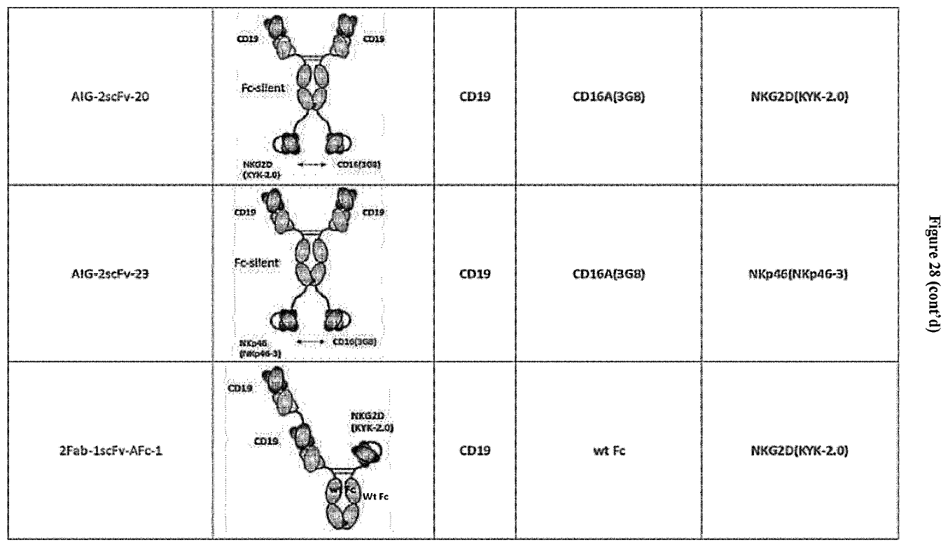

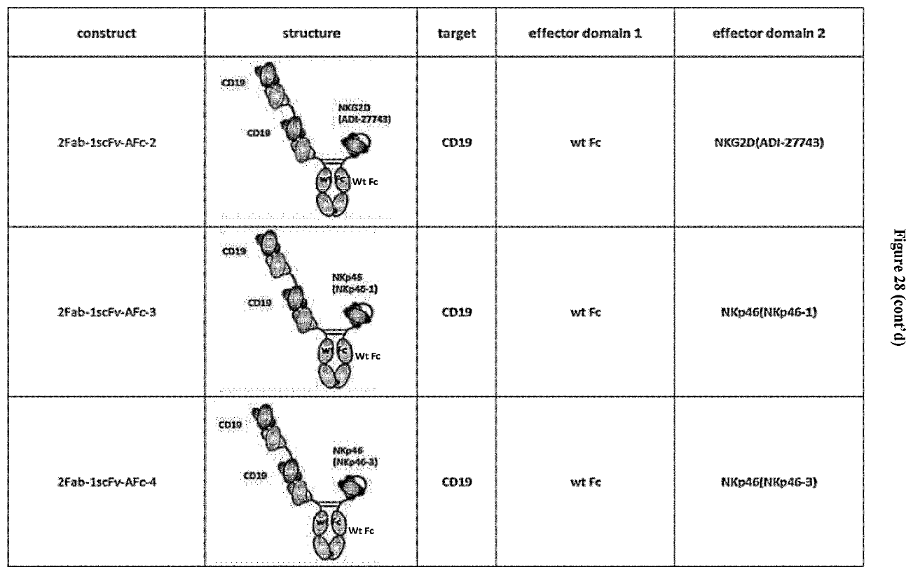

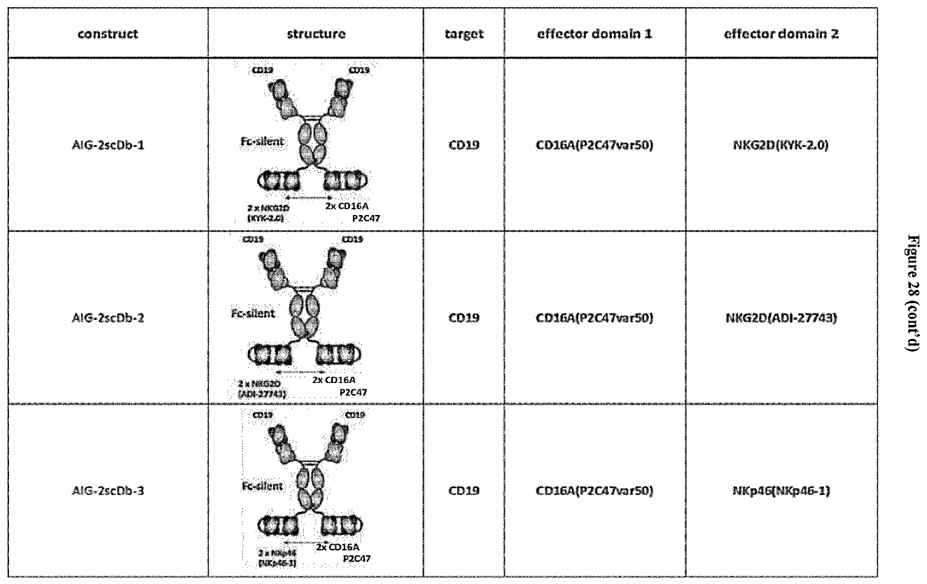

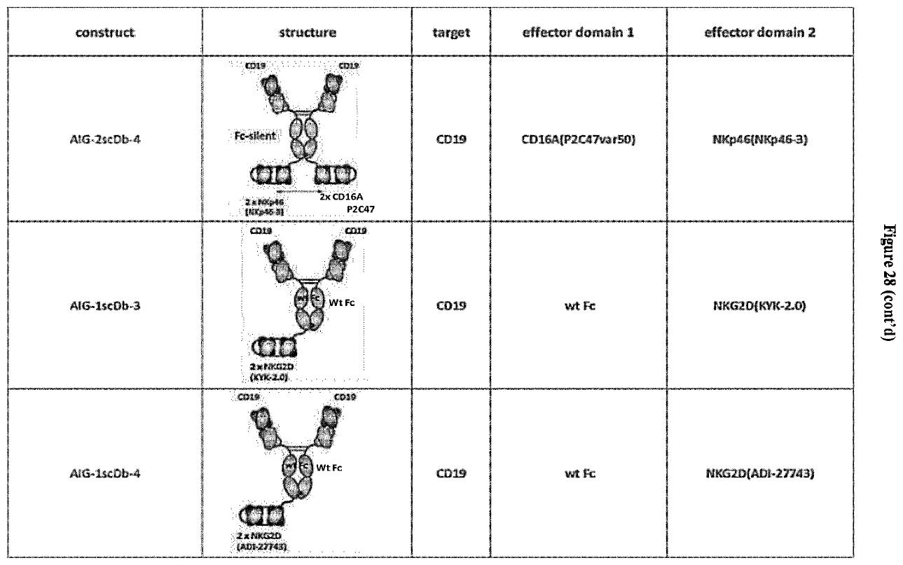

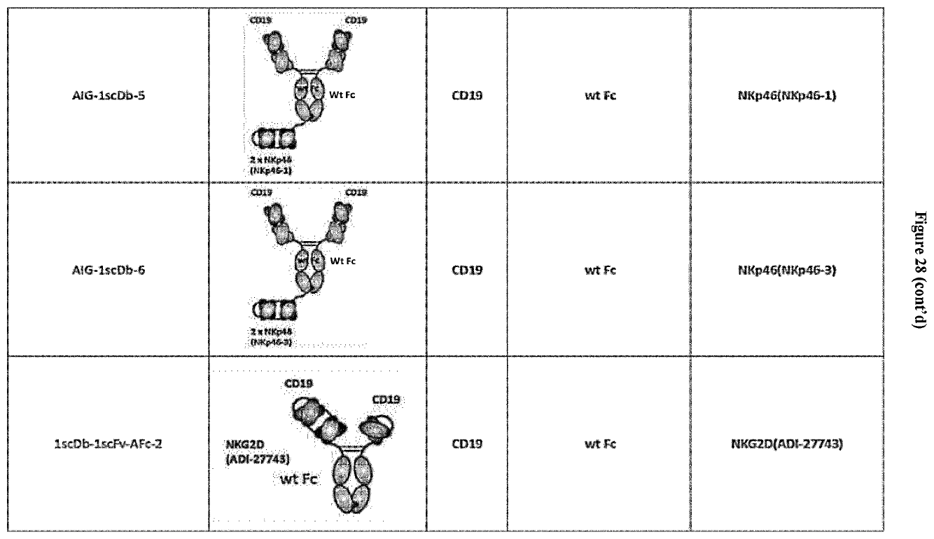

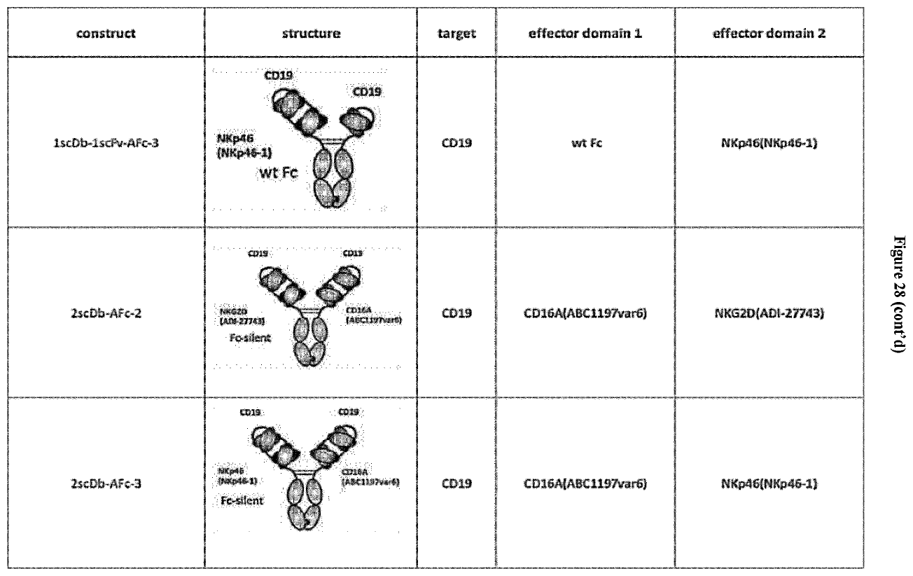

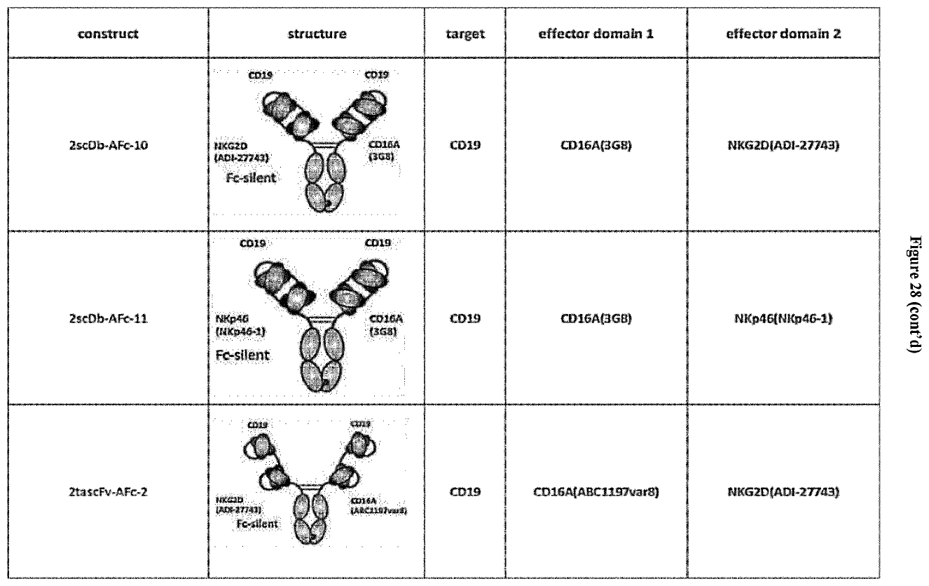

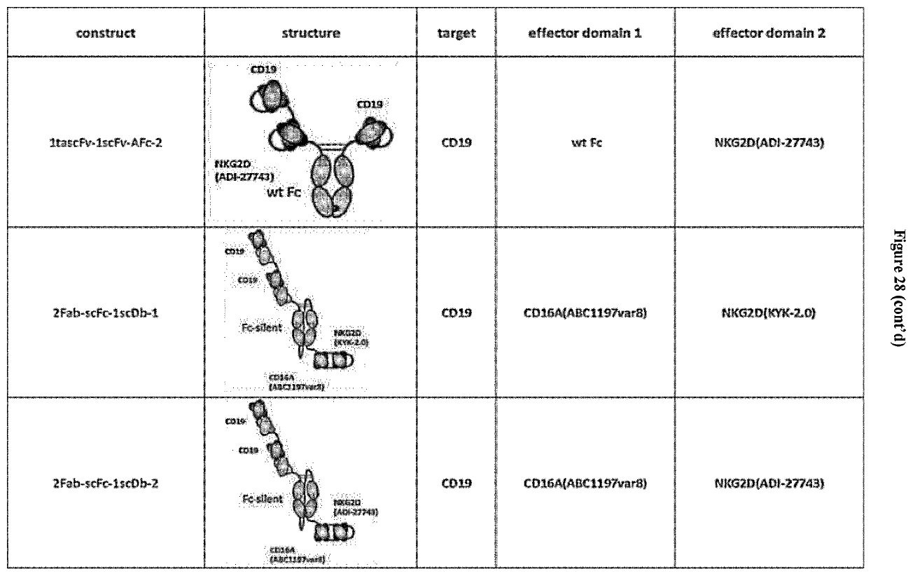

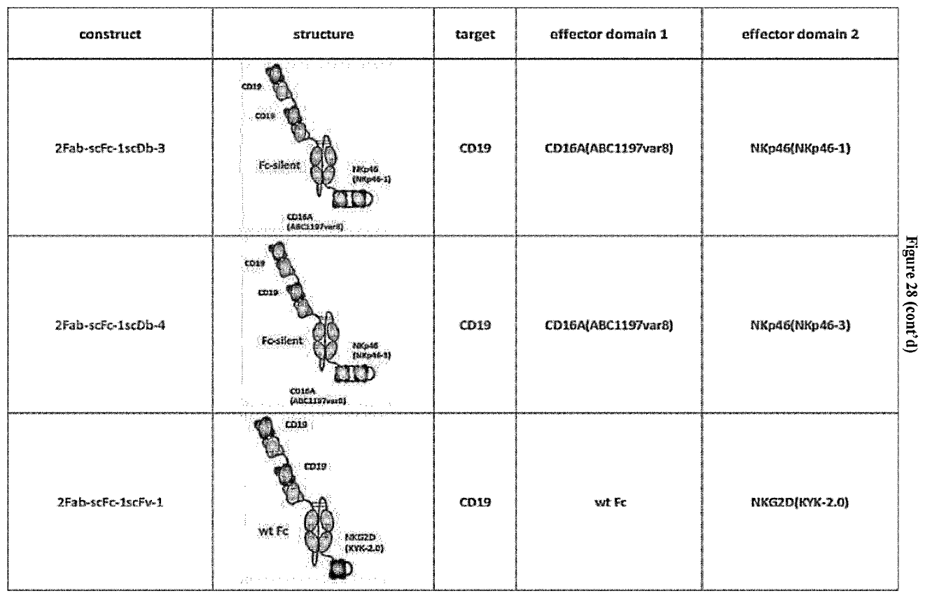

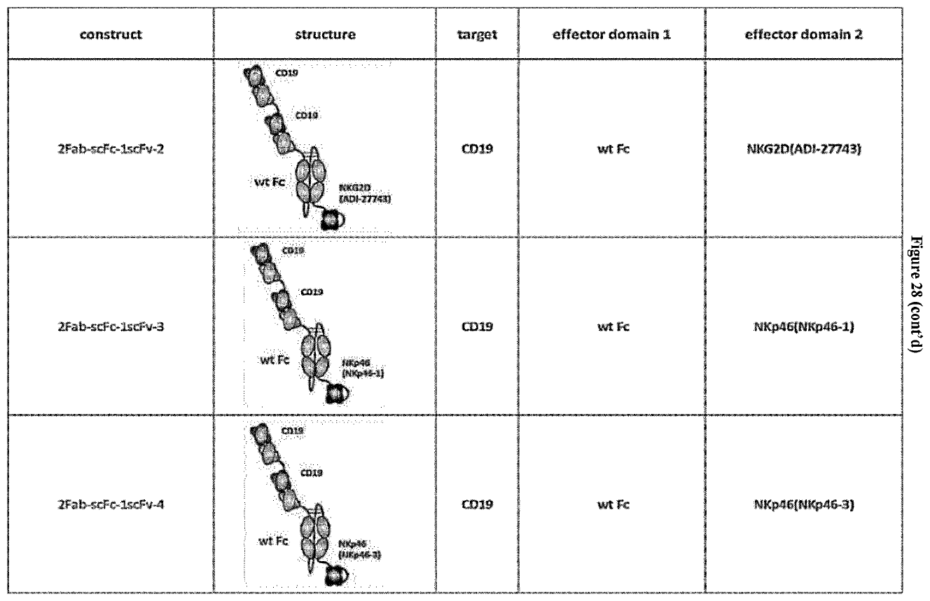

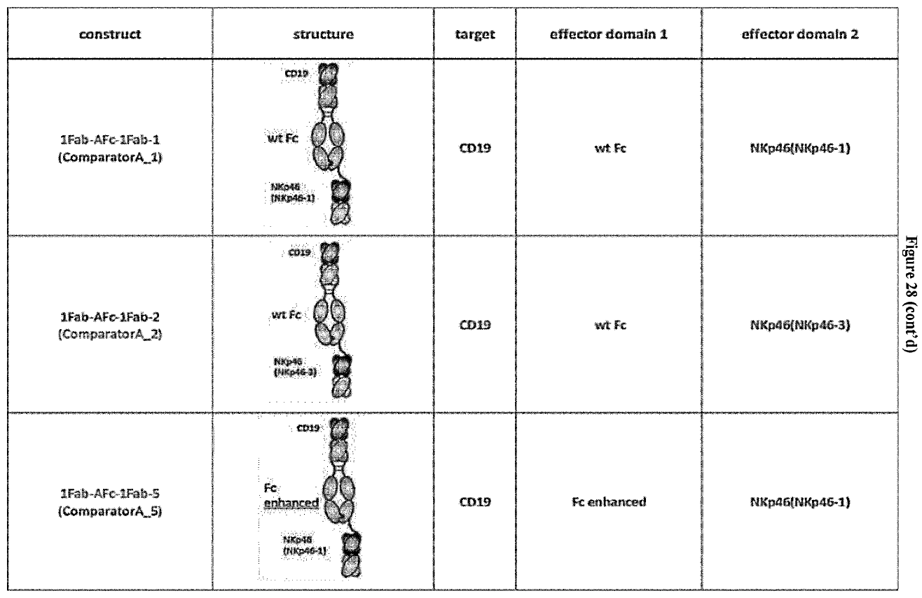

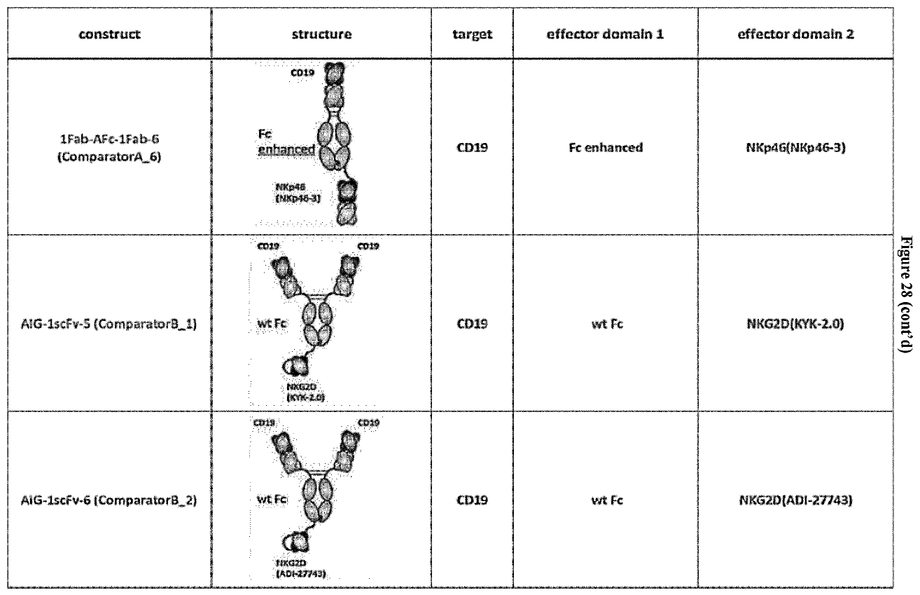

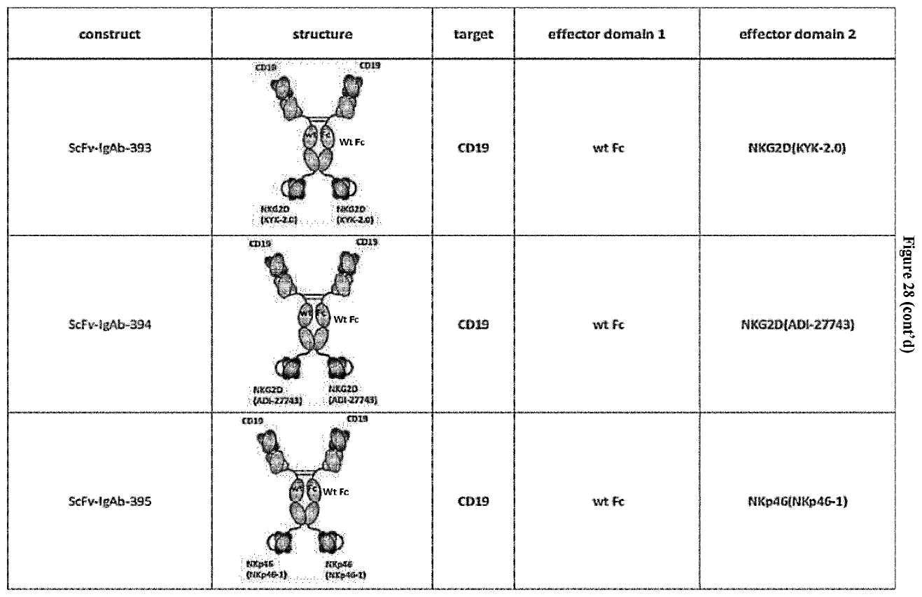

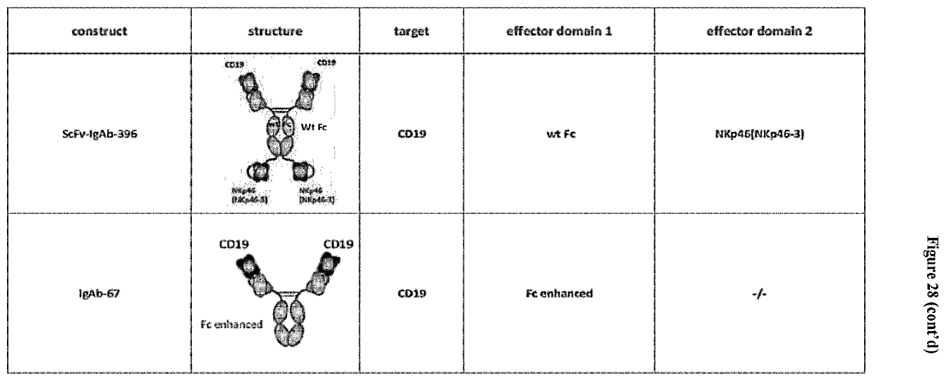

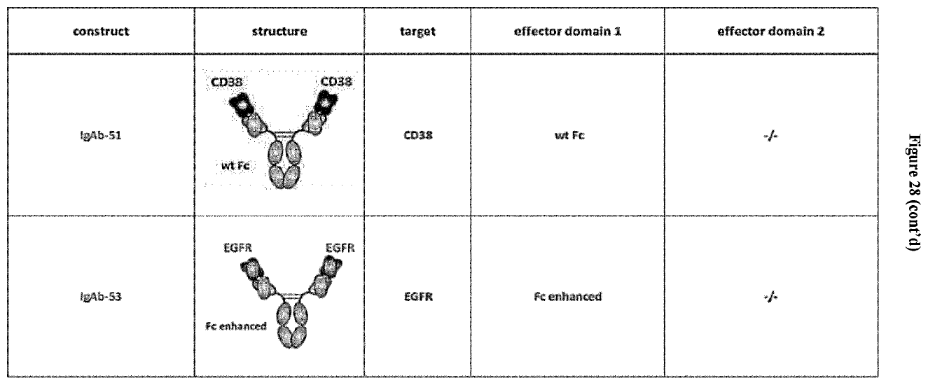

The results of the 4 h cytotoxicity assay presented in FIG. 27 clearly demonstrate, that trispecific of HER2/CD16A/CD89 constructs with either one anti-CD16A and one anti-CD89 Fv domain (AIG-2scFv-28) or two anti-CD16A and one anti-CD89 Fv domains (AIG-1scDb-1scFv-5) induce lysis of HER2⁺ target cells by neutrophils in an E:T ratio-dependent manner, whereas the corresponding bispecific HER2/CD16A constructs with either one anti-CD16A domain (AIG-1scFv-2) or two anti-CD16A domains (AIG-1scDb-9) induced no or only minimal target cell lysis that was comparable to the activity of neutrophils alone in the absence of antibodies.

Sequence Listing

| SEQ | |
|---|---|
| ID NO | Sequence |
| 1 | VH CD16A-1: |
| | QVQLVQSGAEVKKPGASVKVSCKASGYTFTSYYMHWVRQAPGQCLEWMGAIEPTYGSTSYAQKFQGRVTMTRDTSTSTVYMELS |
| | SLRSEDTAVYYCARGSAYYYDFADYWGQGTLVTVSS |
| 2 | VH CD16A-2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSS |
| 3 | VH CD16A-3: |
| | QVQLVQSGAEVKKPGASVKVSCKASGYTFTNYYMQWVRQAPGQGLEWMGIINPSGGVTSYAQKFQGRVTMTRDTSTSTVYMELS |
| | SLRSEDTAVYYCARGSAYYYDFADYWGQGTLVTVSS |
| 4 | VH CD16A-4: |
| | QVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSS |
| 5 | VL CD16A-1: |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVL |
| 6 | VL CD16A-3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGGGTKLTVL |
| 7 | VL CD16A-2/-4: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVL |
| 8 | VH CD16 (3G8): |
| | QVTLKESGPGILQPSQTLSLTCSFSGFSLRTSGMGVGWIRQPSGKGLEWLAHIWWDDDKRYNPALKSRLTISKDTSSNQVFLKI |
| | ASVDTADTATYYCAQINPAWFAYWGQGTLVTVSA |
| 9 | VL CD16 (3G8): |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELK |
| 10 | VH EGFR-1: |
| | QVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSS |
| 11 | VH EGFR-2: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSS |
| 12 | VL EGFR-1/-2: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVL |
| 13 | VH CD19 (MOR208): |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSS |
| 14 | VL CD19 (MOR208): |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIK |
| 15 | VH NKG2D -1: |
| | QVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSS |
| 16 | VH NKG2D -2: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSS |
| 17 | VL NKG2D -1/-2: |
| | QSALTQPASVSGSPGQSITISCSGSSSNIGNNAVNWYQQLPGKAPKLLIYYDDLLPSGVSDRFSGSKSGTSAFLAISGLQSEDE |
| | ADYYCAAWDDSLNGPVFGGGTKLTVL |
| 18 | VH NKG2D -3: |
| | QVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSS |
| 19 | VH NKG2D -4: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSS |
| 20 | VL NKG2D -3/-4: |
| | DIQMTQSPSTLSASVGDRVTITCRASQSISSWLAWYQQKPGKAPKLLIYKASSLESGVPSRFSGSGSGTEFTLTISSLQPDDFA |
| | TYYCQQYNSYPTFGGGTKVEIK |
| 21 | VH NKp46 -1: |
| | QVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSS |
| 22 | VH NKp46 -2: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSS |
| 23 | VL NKp46 -1/-2: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIK |
| 24 | VH NKp46 -3: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSS |
| 25 | VL NKp46 -3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELK |
| 26 | HCDR1 CD16A-1: |
| | SYYMH |
| 27 | HCDR2 CD16A-1: |
| | AIEPTYGSTSYAQKFQG |
| 28 | HCDR3 CD16A-1: |
| | GSAYYYDFADY |
| 29 | LCDR1 CD16A-1: |
| | GGHNIGSKNVH |
| 30 | LCDR2 CD16A-1: |
| | QDNKRPS |
| 31 | LCDR3 CD16A-1: |
| | QVWDNYNVL |
| 32 | HCDR1 CD16A -2/-3/-4: |
| | NYYMQ |
| 33 | HCDR2 CD16A -2/-3/-4: |
| | IINPSGGVTSYAQKFQG |
| 34 | HCDR3 CD16A -2/-3/-4: |
| | GSAYYYDFADY |
| 35 | LCDR1 CD16A -2/-3/-4: |
| | GGNNIGSKSVH |
| 36 | LCDR2 CD16A -2/-3/-4: |
| | QDKKRPS |
| 37 | LCDR3 CD16A -2/-3/-4: |
| | QVWDDYIVL |
| 38 | HCDR1 CD16 (3G8): |
| | TSGMGVG |
| 39 | HCDR2 CD16 (3G8): |
| | HIWWDDDKRYNPALKS |
| 40 | HCDR3 CD16 (3G8): |
| | INPAWFAY |
| 41 | LCDR1 CD16 (3G8): |
| | KASQSVDFDGDSFMN |
| 42 | LCDR2 CD16 (3G8): |
| | TTSNLES |
| 43 | LCDR3 CD16 (3G8): |
| | QQSNEDPYT |
| 44 | HCDR1 EGFR-1/-2: |
| | SGSYYWS |
| 45 | HCDR2 EGFR-1/-2: |
| | YIYYSGSTNYNPSLKS |
| 46 | HCDR3 EGFR-1/-2: |
| | NPISIPAFDI |
| 47 | LCDR1 EGFR-1/-2: |
| | GGNNIGSKSVH |
| 48 | LCDR2 EGFR-1/-2: |
| | YDSDRPS |
| 49 | LCDR3 EGFR-1/-2: |
| | QVWDTSSDHVL |
| 50 | HCDR1 CD19 (MOR208): |
| | SYVMH |
| 51 | HCDR2 CD19 (MOR208): |
| | YINPYNDGTKYNEKFQG |
| 52 | HCDR3 CD19 (MOR208): |
| | GTYYYGTRVFDY |
| 53 | LCDR1 CD19 (MOR208): |
| | RSSKSLQNVNGNTYLY |
| 54 | LCDR2 CD19 (MOR208): |
| | RMSNLNS |
| 55 | LCDR3 CD19 (MOR208): |
| | MQHLEYPIT |
| 56 | HCDR1 NKG2D -1/-2: |
| | SYGMH |
| 57 | HCDR2 NKG2D -1/-2: |
| | FIRYDGSNKYYADSVKG |
| 58 | HCDR3 NKG2D -1/-2: |
| | DRGLGDGTYFDY |
| 59 | LCDR1 NKG2D -1/-2: |
| | SGSSSNIGNNAVN |
| 60 | LCDR2 NKG2D -1/-2: |
| | YDDLLPS |
| 61 | LCDR3 NKG2D -1/-2: |
| | AAWDDSLNGPV |
| 62 | HCDR1 NKG2D -3/-4: |
| | GYYWS |
| 63 | HCDR2 NKG2D -3/-4: |
| | EIDHSGSTNYNPSLKS |
| 64 | HCDR3 NKG2D -3/-4: |
| | ARGPWSFDP |
| 65 | LCDR1 NKG2D -3/-4: |
| | RASQSISSWLA |
| 66 | LCDR2 NKG2D -3/-4: |
| | KASSLES |
| 67 | LCDR3 NKG2D- 3/-4: |
| | QQYNSYPT |
| 68 | HCDR1 Nkp46 -1/-2: |
| | DYVIN |
| 69 | HCDR2 Nkp46 -1/-2: |
| | EIYPGSGTNYYNEKFKA |
| 70 | HCDR3 Nkp46 -1/-2: |
| | RGRYGLYAMDY |
| 71 | LCDR1 Nkp46 -1/-2: |
| | RASQDISNYLN |
| 72 | LCDR2 Nkp46 -1/-2: |
| | YTSRLHS |
| 73 | LCDR3 Nkp46 -1/-2: |
| | QQGNTRPWT |
| 74 | HCDR1 Nkp46 -3: |
| | EYTMH |
| 75 | HCDR2 Nkp46 -3: |
| | GISPNIGGTSYNQKFKG |
| 76 | HCDR3 Nkp46 -3: |
| | RGGSFDY |
| 77 | LCDR1 Nkp46 -3: |
| | RASQSISDYLH |
| 78 | LCDR2 Nkp46 -3: |
| | YASQSIS |
| 79 | LCDR3 Nkp46 -3: |
| | QNGHSFPLT |
| 80 | Linker: |
| | GGSGGS |
| 81 | Linker: |
| | GGSGGSGGS |
| 82 | Linker: |
| | GGSGGSGGSGGSGGSGGS |
| 83 | Linker: |
| | GGSGGSGGSGGSGGSGGSGGS |
| 84 | Connector: |
| | GGGGS |
| 85 | Connector: |
| | GGGGSGGGGS |
| 86 | Connector: |
| | GGGGSGGGGSGGGGSGGGGS |
| 87 | Connector: |
| | GGGGSGGGGSGGGGSGGGGSGGGGSGGGGS |
| 88 | hinge: |
| | EPKSCDKTHTCPPCP |
| 89 | upper.hinge: |
| | EPKSCDKTHT |
| 90 | middle.hinge: |
| | DKTHTCPPCP |
| 91 | Human IgG1 CH1, CH2 and CH3 heavy chain constant domain: |
| | ASTKGPSVFPLAPSSKSTSGGTAALGCLVKDYFPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNVFSCSVMHEALHNHYTQKSLSLSPG |
| 92 | Human IgG1 CH1, CH2 and CH3 heavy chain constant domain with silencing mutation-1: |
| | ASTKGPSVFPLAPSSKSTSGGTAALGCLVKDYFPEPVTVSWNSGALTSGVHTFPAVLQSSGLYSLSSVVTVPSSSLGTQTYICN |
| | VNHKPSNTKVDKKVEPKSCDKTHTCPPCPAPEFEGGPSVFLFPPKPKDTLMISRTPEVTCVVVAVSHEDPEVKFNWYVDGVEVH |
| | NAKTKPREEQYGSTYRVVSVLTVLHQDWLNGKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNVFSCSVMHEALHNHYTQKSLSLSPG |
| 93 | Human IgG1 CH1, CH2 and CH3 heavy chain constant domain with enhancing mutation-1: |
| | ASTKGPSVFPLAPSSKSTSGGTAALGCLVKDYFPEPVTVSWNSGALTSGVHTFPAVLQSSGLYSLSSVVTVPSSSLGTQTYICN |
| | VNHKPSNTKVDKKVEPKSCDKTHTCPPCPAPELLGGPDVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPEEKTISKAKGQPREPQVYTLPPSREEMTKNQVSLTCLV |
| | KGFYPSDIAVEWESNGQPENNYKTTPPVLDSDGSFFLYSKLTVDKSRWQQGNVFSCSVMHEALHNHYTQKSLSLSPG |
| 94 | Human lambda light chain constant domain: |
| | GQPKAAPSVTLFPPSSEELQANKATLVCLISDFYPGAVTVAWKADSSPVKAGVETTTPSKQSNNKYAASSYLSLTPEQWKSHRS |
| | YSCQVTHEGSTVEKTVAPTECS |
| 95 | Human Kappa light chain constant domain: |
| | RTVAAPSVFIFPPSDEQLKSGTASVVCLLNNFYPREAKVQWKVDNALQSGNSQESVTEQDSKDSTYSLSSTLTLSKADYEKHKV |
| | YACEVTHQGLSSPVTKSFNRGEC |
| 96 | CH1 heavy chain constant domain: |
| | ASTKGPSVFPLAPSSKSTSGGTAALGCLVKDYFPEPVTVSWNSGALTSGVHTFPAVLQSSGLYSLSSVVTVPSSSLGTQTYICN |
| | VNHKPSNTKVDKKV |
| 97 | CH2-CH3 heavy chain constant domain: |
| | APELLGGPSVFLFPPKPKDTLMISRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSREEMTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| 98 | CH2-CH3 heavy chain constant domain with silencing mutation-1: |
| | APEFEGGPSVFLFPPKPKDTLMISRTPEVTCVVVAVSHEDPEVKFNWYVDGVEVHNAKTKPREEQYGSTYRVVSVLTVLHQDWL |
| | NGKEYKCKVSNKALPAPIEKTISKAKGQPREPQVYTLPPSREEMTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| 99 | CH2-CH3 heavy chain constant domain with enhancing mutation-1: |
| | APELLGGPDVFLFPPKPKDTLMISRTPEVTCVVVDVSHEDPEVKFNWYVDGVEVHNAKTKPREEQYNSTYRVVSVLTVLHQDWL |
| | NGKEYKCKVSNKALPAPEEKTISKAKGQPREPQVYTLPPSREEMTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| 100 | Hole chain_CH2-CH3 heavy chain constant domain: |
| | APELLGGPSVFLFPPKPKDTLMISRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSREEMTKNQVSLSCAVKGFYPSDIAVEWESNGQPENNYKTTPPVL |
| | DSDGSFFLVSKLTVDKSRWQQGNVFSCSVMHEALHNHYTQKSLSLSPG |
| 101 | Knob chain_CH2-CH3 heavy chain constant domain: |
| | APELLGGPSVFLFPPKPKDTLMISRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSREEMTKNQVSLWCLVKGFYPSDIAVEWESNGQPENNYKTTPPVL |
| | DSDGSFFLYSKLTVDKSRWQQGNVFSCSVMHEALHNHYTQKSLSLSPG |
| 102 | Hole chain_CH2-CH3 heavy chain constant domain with silencing mutation-1: |
| | APEFEGGPSVFLFPPKPKDTLMISRTPEVTCVVVAVSHEDPEVKFNWYVDGVEVHNAKTKPREEQYGSTYRVVSVLTVLHQDWL |
| | NGKEYKCKVSNKALPAPIEKTISKAKGQPREPQVYTLPPSREEMTKNQVSLSCAVKGFYPSDIAVEWESNGQPENNYKTTPPVL |
| | DSDGSFFLVSKLTVDKSRWQQGNVFSCSVMHEALHNHYTQKSLSLSPG |
| 103 | Knob chain_CH2-CH3 heavy chain constant domain with silencing mutation-1: |
| | APEFEGGPSVFLFPPKPKDTLMISRTPEVTCVVVAVSHEDPEVKFNWYVDGVEVHNAKTKPREEQYGSTYRVVSVLTVLHQDWL |
| | NGKEYKCKVSNKALPAPIEKTISKAKGQPREPQVYTLPPSREEMTKNQVSLWCLVKGFYPSDIAVEWESNGQPENNYKTTPPVL |
| | DSDGSFFLYSKLTVDKSRWQQGNVFSCSVMHEALHNHYTQKSLSLSPG |
| 104 | Hole chain_CH2-CH3 heavy chain constant domain with enhancing mutation-1: |
| | APELLGGPDVFLFPPKPKDTLMISRTPEVTCVVVDVSHEDPEVKFNWYVDGVEVHNAKTKPREEQYNSTYRVVSVLTVLHQDWL |
| | NGKEYKCKVSNKALPAPEEKTISKAKGQPREPQVYTLPPSREEMTKNQVSLSCAVKGFYPSDIAVEWESNGQPENNYKTTPPVL |
| | DSDGSFFLVSKLTVDKSRWQQGNVFSCSVMHEALHNHYTQKSLSLSPG |
| 105 | Knob chain_CH2-CH3 heavy chain constant domain with enhancing mutation-1: |
| | APELLGGPDVFLFPPKPKDTLMISRTPEVTCVVVDVSHEDPEVKFNWYVDGVEVHNAKTKPREEQYNSTYRVVSVLTVLHQDWL |
| | NGKEYKCKVSNKALPAPEEKTISKAKGQPREPQVYTLPPSREEMTKNQVSLWCLVKGFYPSDIAVEWESNGQPENNYKTTPPVL |
| | DSDGSFFLYSKLTVDKSRWQQGNVFSCSVMHEALHNHYTQKSLSLSPG |
| 106 | single chain Fc: CH2-CH3 heavy chain constant domain: |
| | APELLGGPSVFLFPPKPKDTLMISRTPEVTCVVVDVSHEDPEVKFNWYVDGVEVHNAKTKPCEEQYNSTYRCVSVLTVLHQDWL |
| | NGKEYKCKVSNKALPAPIEKTISKAKGQPREPQVYTLPPSREEMTKNQVSLTCLVKGFYPSDIAVEWESNGQPENNYKTTPPVL |
| | DSDGSFFLYSKLTVDKSRWQQGNVFSCSVMHEALHNHYTQKSLSLSPGKGGGGSGGGGSGGGGSGGGGSGGGGSGGGGSDKTHT |
| | CPPCPAPELLGGPSVFLFPPKPKDTLMISRTPEVTCVVVDVSHEDPEVKFNWYVDGVEVHNAKTKPCEEQYNSTYRCVSVLTVL |
| | HQDWLNGKEYKCKVSNKALPAPIEKTISKAKGQPREPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK |
| 107 | single chain Fc: CH2-CH3 heavy chain constant domain with silencing mutation-1: |
| | APEFEGGPSVFLFPPKPKDTLMISRTPEVTCVVVAVSHEDPEVKFNWYVDGVEVHNAKTKPCEEQYGSTYRCVSVLTVLHQDWL |
| | NGKEYKCKVSNKALPAPIEKTISKAKGQPREPQVYTLPPSREEMTKNQVSLTCLVKGFYPSDIAVEWESNGQPENNYKTTPPVL |
| | DSDGSFFLYSKLTVDKSRWQQGNVFSCSVMHEALHNHYTQKSLSLSPGKGGGGSGGGGSGGGGSGGGGSGGGGSGGGGSDKTHT |
| | CPPCPAPEFEGGPSVFLFPPKPKDTLMISRTPEVTCVVVAVSHEDPEVKFNWYVDGVEVHNAKTKPCEEQYGSTYRCVSVLTVL |
| | HQDWLNGKEYKCKVSNKALPAPIEKTISKAKGQPREPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK |
| 108 | BisscDb-1: CD16A-2 × CD19 (MOR208) |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSSYELTQPLSVSVALGQTARITCGGNNIG |
| | SKSVHWYQQKPGQAPVLVIYQDKKRPSGIPERFSGSNSGNTATLTISRAQAGDEADYYCQVWDDYIVLFGCGTKLTVL |
| 109 | BisscDb-2: CD16A-2 × EGFR-1 |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGCGTKLTVL |
| 110 | BisscDb-3: NKG2D-2 × CD19 (MOR208) |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSQSALTQPASVSGSPGQSITISCSGSSS |
| | NIGNNAVNWYQQLPGKAPKLLIYYDDLLPSGVSDRFSGSKSGTSAFLAISGLQSEDEADYYCAAWDDSLNGPVFGGGTKLTVL |
| 111 | BisscDb-4: NKG2D-4 × CD19 (MOR208) |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSDIVMTQSPATLSLSPGERATLSCRSSKSLQNVNGNTYLYWFQ |
| | QKPGQSPQLLIYRMSNLNSGVPDRFSGSGSGTEFTLTISSLEPEDFAVYYCMQHLEYPITFGAGTKLEIKGGSGGSGGSGGSGG |
| | SGGSGGSEVQLVESGGGLVKPGGSLKLSCAASGYTFTSYVMHWVRQAPGKGLEWIGYINPYNDGTKYNEKFQGRVTISSDKSIS |
| | TAYMELSSLRSEDTAMYYCARGTYYYGTRVFDYWGQGTLVTVSSGGSGGSGGSDIQMTQSPSTLSASVGDRVTITCRASQSISS |
| | WLAWYQQKPGKAPKLLIYKASSLESGVPSRFSGSGSGTEFTLTISSLQPDDFATYYCQQYNSYPTFGGGTKVEIK |
| 112 | BisscDb-5: NKp46-2 × CD19 (MOR208) |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSDIQMTQTTSSLSASLGDRVTISCRASQD |
| | ISNYLNWYQQKPDGTVKLLIYYTSRLHSGVPSRFSGSGSGTDYSLTINNLEQEDIATYFCQQGNTRPWTFGGGTKLEIK |
| 113 | BisscDb-6: NKp46-3 × CD19 (MOR208) |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSDIVMTQSPATLSLSPGERATLSCRSSKSLQNVNGNTYLYWFQQ |
| | KPGQSPQLLIYRMSNLNSGVPDRFSGSGSGTEFTLTISSLEPEDFAVYYCMQHLEYPITFGAGTKLEIKGGSGGSGGSGGSGGS |
| | GGSGGSEVQLVESGGGLVKPGGSLKLSCAASGYTFTSYVMHWVRQAPGKGLEWIGYINPYNDGTKYNEKFQGRVTISSDKSIST |
| | AYMELSSLRSEDTAMYYCARGTYYYGTRVFDYWGQGTLVTVSSGGSGGSGGSDIVMTQSPATLSVTPGDRVSLSCRASQSISDY |
| | LHWYQQKSHESPRLLIKYASQSISGIPSRFSGSGSGSDFTLSINSVEPEDVGVYYCQNGHSFPLTFGAGTKLELK |
| 114 | BisscDb-7: NKG2D-2x EGFR-1 |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSQPVLTQPPSVSVAPGKTARITCGGNNIGSKSVHWYQQK |
| | PGQAPVLVIYYDSDRPSGIPERFSGSNSGNTATLTISRVEAGDEADYYCQVWDTSSDHVLFGGGTKLTVLGGSGGSGGSGGSGG |
| | SGGSGGSQVQLQESGPGLVKPSETLSLTCTVSGGSVSSGSYYWSWIRQPPGKGLEWIGYIYYSGSTNYNPSLKSRVTISVDTSK |
| | NQFSLKLSSVTAADTAVYYCARNPISIPAFDIWGQGTMVTVSSGGSGGSGGSQSALTQPASVSGSPGQSITISCSGSSSNIGNN |
| | AVNWYQQLPGKAPKLLIYYDDLLPSGVSDRFSGSKSGTSAFLAISGLQSEDEADYYCAAWDDSLNGPVFGGGTKLTVL |
| 115 | BisscDb-8: NKG2D-4 × EGFR-1 |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSQPVLTQPPSVSVAPGKTARITCGGNNIGSKSVHWYQQKPGQA |
| | PVLVIYYDSDRPSGIPERFSGSNSGNTATLTISRVEAGDEADYYCQVWDTSSDHVLFGGGTKLTVLGGSGGSGGSGGSGGSGGS |
| | GGSQVQLQESGPGLVKPSETLSLTCTVSGGSVSSGSYYWSWIRQPPGKGLEWIGYIYYSGSTNYNPSLKSRVTISVDTSKNQFS |
| | LKLSSVTAADTAVYYCARNPISIPAFDIWGQGTMVTVSSGGSGGSGGSDIQMTQSPSTLSASVGDRVTITCRASQSISSWLAWY |
| | QQKPGKAPKLLIYKASSLESGVPSRFSGSGSGTEFTLTISSLQPDDFATYYCQQYNSYPTFGGGTKVEIK |
| 116 | BisscDb-9: NKp46-2 × EGFR-1 |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSDIQMTQTTSSLSASLGDRVTISCRASQDISNYL |
| | NWYQQKPDGTVKLLIYYTSRLHSGVPSRFSGSGSGTDYSLTINNLEQEDIATYFCQQGNTRPWTFGGGTKLEIK |
| 117 | BisscDb-10: NKp46-3 × EGFR-1 |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSQPVLTQPPSVSVAPGKTARITCGGNNIGSKSVHWYQQKPGQAP |
| | VLVIYYDSDRPSGIPERFSGSNSGNTATLTISRVEAGDEADYYCQVWDTSSDHVLFGGGTKLTVLGGSGGSGGSGGSGGSGGSG |
| | GSQVQLQESGPGLVKPSETLSLTCTVSGGSVSSGSYYWSWIRQPPGKGLEWIGYIYYSGSTNYNPSLKSRVTISVDTSKNQFSL |
| | KLSSVTAADTAVYYCARNPISIPAFDIWGQGTMVTVSSGGSGGSGGSDIVMTQSPATLSVTPGDRVSLSCRASQSISDYLHWYQ |
| | QKSHESPRLLIKYASQSISGIPSRFSGSGSGSDFTLSINSVEPEDVGVYYCQNGHSFPLTFGAGTKLELK |
| 118 | BisscDb-11: CD16A-4 × NKG2D -1 |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLVESGGGLVKPGGSLRLSCAASGFTFSSYGMHWVRQAPGKGLEWVAFIRYDGS |
| | NKYYADSVKGRFTISRDNSKNTLYLQMNSLRAEDTAVYYCAKDRGLGDGTYFDYWGQGTTVTVSSGGSGGSGGSGGSGGSGGSQ |
| | SALTQPASVSGSPGQSITISCSGSSSNIGNNAVNWYQQLPGKAPKLLIYYDDLLPSGVSDRFSGSKSGTSAFLAISGLQSEDEA |
| | DYYCAAWDDSLNGPVFGGGTKLTVLGGSGGSQVQLVQSGAEVKKPGASVKVSCKASGYTFTNYYMQWVRQAPGQCLEWMGIINP |
| | SGGVTSYAQKFQGRVTMTRDTSTSTVYMELSSLRSEDTAVYYCARGSAYYYDFADYWGQGTLVTVSS |
| 119 | BisscDb-12: CD16A-4 × NKG2D-3 |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLQQWGAGLLKPSETLSLTCAVYGGSFSGYYWSWIRQPPGKGLEWIGEIDHSGS |
| | TNYNPSLKSRVTISVDTSKNQFSLKLSSVTAADTAVYYCARARGPWSFDPWGQGTLVTVSSGGSGGSGGSGGSGGSGGSDIQMT |
| | QSPSTLSASVGDRVTITCRASQSISSWLAWYQQKPGKAPKLLIYKASSLESGVPSRFSGSGSGTEFTLTISSLQPDDFATYYCQ |
| | QYNSYPTFGGGTKVEIKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSS |
| 120 | BisscDb-13: CD16A-4 × NKp46-1 |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLQQSGPELVKPGASVKMSCKASGYTFTDYVINWGKQRSGQGLEWIGEIYPGSG |
| | TNYYNEKFKAKATLTADKSSNIAYMQLSSLTSEDSAVYFCARRGRYGLYAMDYWGQGTSVTVSSGGSGGSGGSGGSGGSGGSDI |
| | QMTQTTSSLSASLGDRVTISCRASQDISNYLNWYQQKPDGTVKLLIYYTSRLHSGVPSRFSGSGSGTDYSLTINNLEQEDIATY |
| | FCQQGNTRPWTFGGGTKLEIKGGSGGSQVQLVQSGAEVKKPGASVKVSCKASGYTFTNYYMQWVRQAPGQCLEWMGIINPSGGV |
| | TSYAQKFQGRVTMTRDTSTSTVYMELSSLRSEDTAVYYCARGSAYYYDFADYWGQGTLVTVSS |
| 121 | BisscDb-14: CD16A-4 × NKp46-3 |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSEVQLQQSGPELVKPGASVKISCKTSGYTFTEYTMHWVKQSHGKSLEWIGGISPNIG |
| | GTSYNQKFKGKATLTVDKSSSTAYMELRSLTSEDSAVYYCARRGGSFDYWGQGTTLTVSSGGSGGSGGSGGSGGSGGSDIVMTQ |
| | SPATLSVTPGDRVSLSCRASQSISDYLHWYQQKSHESPRLLIKYASQSISGIPSRFSGSGSGSDFTLSINSVEPEDVGVYYCQN |
| | GHSFPLTFGAGTKLELKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSS |
| 122 | BisscDb-15: CD16A (3G8) × CD19 (MOR208) |
| | QVTLKESGPGILQPSQTLSLTCSFSGFSLRTSGMGVGWIRQPSGKGLEWLAHIWWDDDKRYNPALKSRLTISKDTSSNQVFLKI |
| | ASVDTADTATYYCAQINPAWFAYWGQGTLVTVSAGGSGGSGGSDIVMTQSPATLSLSPGERATLSCRSSKSLQNVNGNTYLYWF |
| | QQKPGQSPQLLIYRMSNLNSGVPDRFSGSGSGTEFTLTISSLEPEDFAVYYCMQHLEYPITFGAGTKLEIKGGSGGSGGSGGSG |
| | GSGGSGGSEVQLVESGGGLVKPGGSLKLSCAASGYTFTSYVMHWVRQAPGKGLEWIGYINPYNDGTKYNEKFQGRVTISSDKSI |
| | STAYMELSSLRSEDTAMYYCARGTYYYGTRVFDYWGQGTLVTVSSGGSGGSGGSDIVLTQSPASLAVSLGQRATISCKASQSVD |
| | FDGDSFMNWYQQKPGQPPKLLIYTTSNLESGIPARFSASGSGTDFTLNIHPVEEEDTATYYCQQSNEDPYTFGGGTKLELK |
| 123 | BisscDb-16: CD16A (3G8) × EGFR-1 |
| | QVTLKESGPGILQPSQTLSLTCSFSGFSLRTSGMGVGWIRQPSGKGLEWLAHIWWDDDKRYNPALKSRLTISKDTSSNQVFLKI |
| | ASVDTADTATYYCAQINPAWFAYWGQGTLVTVSAGGSGGSGGSQPVLTQPPSVSVAPGKTARITCGGNNIGSKSVHWYQQKPGQ |
| | APVLVIYYDSDRPSGIPERFSGSNSGNTATLTISRVEAGDEADYYCQVWDTSSDHVLFGGGTKLTVLGGSGGSGGSGGSGGSGG |
| | SGGSQVQLQESGPGLVKPSETLSLTCTVSGGSVSSGSYYWSWIRQPPGKGLEWIGYIYYSGSTNYNPSLKSRVTISVDTSKNQF |
| | SLKLSSVTAADTAVYYCARNPISIPAFDIWGQGTMVTVSSGGSGGSGGSDIVLTQSPASLAVSLGQRATISCKASQSVDFDGDS |
| | FMNWYQQKPGQPPKLLIYTTSNLESGIPARFSASGSGTDFTLNIHPVEEEDTATYYCQQSNEDPYTFGGGTKLELK |
| 124 | BisscDb-17: CD16 (3G8) × NKG2D-1 |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSQVQLVESGGGLVKPGGSLRLSCAASGFTFSSYGMHWVRQAPGKGLEWVAFI |
| | RYDGSNKYYADSVKGRFTISRDNSKNTLYLQMNSLRAEDTAVYYCAKDRGLGDGTYFDYWGQGTTVTVSSGGSGGSGGSGGSGG |
| | SGGSQSALTQPASVSGSPGQSITISCSGSSSNIGNNAVNWYQQLPGKAPKLLIYYDDLLPSGVSDRFSGSKSGTSAFLAISGLQ |
| | SEDEADYYCAAWDDSLNGPVFGGGTKLTVLGGSGGSQVTLKESGPGILQPSQTLSLTCSFSGFSLRTSGMGVGWIRQPSGKGLE |
| | WLAHIWWDDDKRYNPALKSRLTISKDTSSNQVFLKIASVDTADTATYYCAQINPAWFAYWGQGTLVTVSA |
| 125 | BisscDb-18: CD16 (3G8) × NKG2D-2 |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSQVQLQQWGAGLLKPSETLSLTCAVYGGSFSGYYWSWIRQPPGKGLEWIGEI |
| | DHSGSTNYNPSLKSRVTISVDTSKNQFSLKLSSVTAADTAVYYCARARGPWSFDPWGQGTLVTVSSGGSGGSGGSGGSGGSGGS |
| | DIQMTQSPSTLSASVGDRVTITCRASQSISSWLAWYQQKPGKAPKLLIYKASSLESGVPSRFSGSGSGTEFTLTISSLQPDDFA |
| | TYYCQQYNSYPTFGGGTKVEIKGGSGGSQVTLKESGPGILQPSQTLSLTCSFSGFSLRTSGMGVGWIRQPSGKGLEWLAHIWWD |
| | DDKRYNPALKSRLTISKDTSSNQVFLKIASVDTADTATYYCAQINPAWFAYWGQGTLVTVSA |
| 126 | BisscDb-19: CD16 (3G8) × NKp46-1 |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSQVQLQQSGPELVKPGASVKMSCKASGYTFTDYVINWGKQRSGQGLEWIGEI |
| | YPGSGTNYYNEKFKAKATLTADKSSNIAYMQLSSLTSEDSAVYFCARRGRYGLYAMDYWGQGTSVTVSSGGSGGSGGSGGSGGS |
| | GGSDIQMTQTTSSLSASLGDRVTISCRASQDISNYLNWYQQKPDGTVKLLIYYTSRLHSGVPSRFSGSGSGTDYSLTINNLEQE |
| | DIATYFCQQGNTRPWTFGGGTKLEIKGGSGGSQVTLKESGPGILQPSQTLSLTCSFSGFSLRTSGMGVGWIRQPSGKGLEWLAH |
| | IWWDDDKRYNPALKSRLTISKDTSSNQVFLKIASVDTADTATYYCAQINPAWFAYWGQGTLVTVSA |
| 127 | BisscDb-20: CD16 (3G8) × NKp46-2 |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSEVQLQQSGPELVKPGASVKISCKTSGYTFTEYTMHWVKQSHGKSLEWIGGI |
| | SPNIGGTSYNQKFKGKATLTVDKSSSTAYMELRSLTSEDSAVYYCARRGGSFDYWGQGTTLTVSSGGSGGSGGSGGSGGSGGSD |
| | IVMTQSPATLSVTPGDRVSLSCRASQSISDYLHWYQQKSHESPRLLIKYASQSISGIPSRFSGSGSGSDFTLSINSVEPEDVGV |
| | YYCQNGHSFPLTFGAGTKLELKGGSGGSQVTLKESGPGILQPSQTLSLTCSFSGFSLRTSGMGVGWIRQPSGKGLEWLAHIWWD |
| | DDKRYNPALKSRLTISKDTSSNQVFLKIASVDTADTATYYCAQINPAWFAYWGQGTLVTVSA |
| 128 | BisscDb-21: CD16A-1 × NKG2D-1 |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVLGGSGGSQVQLVESGGGLVKPGGSLRLSCAASGFTFSSYGMHWVRQAPGKGLEWVAFIRYDGS |
| | NKYYADSVKGRFTISRDNSKNTLYLQMNSLRAEDTAVYYCAKDRGLGDGTYFDYWGQGTTVTVSSGGSGGSGGSGGSGGSGGSQ |
| | SALTQPASVSGSPGQSITISCSGSSSNIGNNAVNWYQQLPGKAPKLLIYYDDLLPSGVSDRFSGSKSGTSAFLAISGLQSEDEA |
| | DYYCAAWDDSLNGPVFGGGTKLTVLGGSGGSQVQLVQSGAEVKKPGASVKVSCKASGYTFTSYYMHWVRQAPGQCLEWMGAIEP |
| | TYGSTSYAQKFQGRVTMTRDTSTSTVYMELSSLRSEDTAVYYCARGSAYYYDFADYWGQGTLVTVSS |
| 129 | BisscDb-22: CD16A-1 × NKG2D-3 |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVLGGSGGSQVQLQQWGAGLLKPSETLSLTCAVYGGSFSGYYWSWIRQPPGKGLEWIGEIDHSGS |
| | TNYNPSLKSRVTISVDTSKNQFSLKLSSVTAADTAVYYCARARGPWSFDPWGQGTLVTVSSGGSGGSGGSGGSGGSGGSDIQMT |
| | QSPSTLSASVGDRVTITCRASQSISSWLAWYQQKPGKAPKLLIYKASSLESGVPSRFSGSGSGTEFTLTISSLQPDDFATYYCQ |
| | QYNSYPTFGGGTKVEIKGGSGGSQVQLVQSGAEVKKPGASVKVSCKASGYTFTSYYMHWVRQAPGQCLEWMGAIEPTYGSTSYA |
| | QKFQGRVTMTRDTSTSTVYMELSSLRSEDTAVYYCARGSAYYYDFADYWGQGTLVTVSS |
| 130 | BisscDb-23: CD16A-1 × NKp46-1 |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVLGGSGGSQVQLQQSGPELVKPGASVKMSCKASGYTFTDYVINWGKQRSGQGLEWIGEIYPGSG |
| | TNYYNEKFKAKATLTADKSSNIAYMQLSSLTSEDSAVYFCARRGRYGLYAMDYWGQGTSVTVSSGGSGGSGGSGGSGGSGGSDI |
| | QMTQTTSSLSASLGDRVTISCRASQDISNYLNWYQQKPDGTVKLLIYYTSRLHSGVPSRFSGSGSGTDYSLTINNLEQEDIATY |
| | FCQQGNTRPWTFGGGTKLEIKGGSGGSQVQLVQSGAEVKKPGASVKVSCKASGYTFTSYYMHWVRQAPGQCLEWMGAIEPTYGS |
| | TSYAQKFQGRVTMTRDTSTSTVYMELSSLRSEDTAVYYCARGSAYYYDFADYWGQGTLVTVSS |
| 131 | BisscDb-24: CD16A-1 × NKp46-3 |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVLGGSGGSEVQLQQSGPELVKPGASVKISCKTSGYTFTEYTMHWVKQSHGKSLEWIGGISPNIG |
| | GTSYNQKFKGKATLTVDKSSSTAYMELRSLTSEDSAVYYCARRGGSFDYWGQGTTLTVSSGGSGGSGGSGGSGGSGGSDIVMTQ |
| | SPATLSVTPGDRVSLSCRASQSISDYLHWYQQKSHESPRLLIKYASQSISGIPSRFSGSGSGSDFTLSINSVEPEDVGVYYCQN |
| | GHSFPLTFGAGTKLELKGGSGGSQVQLVQSGAEVKKPGASVKVSCKASGYTFTSYYMHWVRQAPGQCLEWMGAIEPTYGSTSYA |
| | QKFQGRVTMTRDTSTSTVYMELSSLRSEDTAVYYCARGSAYYYDFADYWGQGTLVTVSS |
| 132 | scDb-1: CD16A-1 |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVLGGSGGSQVQLVQSGAEVKKPGASVKVSCKASGYTFTSYYMHWVRQAPGQCLEWMGAIEPTYG |
| | STSYAQKFQGRVTMTRDTSTSTVYMELSSLRSEDTAVYYCARGSAYYYDFADYWGQGTLVTVSSGGSGGSGGSGGSGGSGGSSY |
| | ELTQPLSVSVALGQTARITCGGHNIGSKNVHWYQQKPGQAPVLVIYQDNKRPSGIPERFSGSNSGNTATLTISRAQAGDEADYY |
| | CQVWDNYNVLFGCGTKLTVLGGSGGSQVQLVQSGAEVKKPGASVKVSCKASGYTFTSYYMHWVRQAPGQCLEWMGAIEPTYGST |
| | SYAQKFQGRVTMTRDTSTSTVYMELSSLRSEDTAVYYCARGSAYYYDFADYWGQGTLVTVSS |
| 133 | scDb-2: NKG2D-1 |
| | QSALTQPASVSGSPGQSITISCSGSSSNIGNNAVNWYQQLPGKAPKLLIYYDDLLPSGVSDRFSGSKSGTSAFLAISGLQSEDE |
| | ADYYCAAWDDSLNGPVFGGGTKLTVLGGSGGSQVQLVESGGGLVKPGGSLRLSCAASGFTFSSYGMHWVRQAPGKGLEWVAFIR |
| | YDGSNKYYADSVKGRFTISRDNSKNTLYLQMNSLRAEDTAVYYCAKDRGLGDGTYFDYWGQGTTVTVSSGGSGGSGGSGGSGGS |
| | GGSQSALTQPASVSGSPGQSITISCSGSSSNIGNNAVNWYQQLPGKAPKLLIYYDDLLPSGVSDRFSGSKSGTSAFLAISGLQS |
| | EDEADYYCAAWDDSLNGPVFGGGTKLTVLGGSGGSQVQLVESGGGLVKPGGSLRLSCAASGFTFSSYGMHWVRQAPGKGLEWVA |
| | FIRYDGSNKYYADSVKGRFTISRDNSKNTLYLQMNSLRAEDTAVYYCAKDRGLGDGTYFDYWGQGTTVTVSS |
| 134 | scDb-3: NKG2D-3 |
| | DIQMTQSPSTLSASVGDRVTITCRASQSISSWLAWYQQKPGKAPKLLIYKASSLESGVPSRFSGSGSGTEFTLTISSLQPDDFA |
| | TYYCQQYNSYPTFGGGTKVEIKGGSGGSQVQLQQWGAGLLKPSETLSLTCAVYGGSFSGYYWSWIRQPPGKGLEWIGEIDHSGS |
| | TNYNPSLKSRVTISVDTSKNQFSLKLSSVTAADTAVYYCARARGPWSFDPWGQGTLVTVSSGGSGGSGGSGGSGGSGGSDIQMT |
| | QSPSTLSASVGDRVTITCRASQSISSWLAWYQQKPGKAPKLLIYKASSLESGVPSRFSGSGSGTEFTLTISSLQPDDFATYYCQ |
| | QYNSYPTFGGGTKVEIKGGSGGSQVQLQQWGAGLLKPSETLSLTCAVYGGSFSGYYWSWIRQPPGKGLEWIGEIDHSGSTNYNP |
| | SLKSRVTISVDTSKNQFSLKLSSVTAADTAVYYCARARGPWSFDPWGQGTLVTVSS |
| 135 | scDb-4: NKp46-1 |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKGGSGGSQVQLQQSGPELVKPGASVKMSCKASGYTFTDYVINWGKQRSGQGLEWIGEIYPGS |
| | GTNYYNEKFKAKATLTADKSSNIAYMQLSSLTSEDSAVYFCARRGRYGLYAMDYWGQGTSVTVSSGGSGGSGGSGGSGGSGGSD |
| | IQMTQTTSSLSASLGDRVTISCRASQDISNYLNWYQQKPDGTVKLLIYYTSRLHSGVPSRFSGSGSGTDYSLTINNLEQEDIAT |
| | YFCQQGNTRPWTFGGGTKLEIKGGSGGSQVQLQQSGPELVKPGASVKMSCKASGYTFTDYVINWGKQRSGQGLEWIGEIYPGSG |
| | TNYYNEKFKAKATLTADKSSNIAYMQLSSLTSEDSAVYFCARRGRYGLYAMDYWGQGTSVTVSS |
| 136 | scDb-5: NKp46-3 |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKGGSGGSEVQLQQSGPELVKPGASVKISCKTSGYTFTEYTMHWVKQSHGKSLEWIGGISPNI |
| | GGTSYNQKFKGKATLTVDKSSSTAYMELRSLTSEDSAVYYCARRGGSFDYWGQGTTLTVSSGGSGGSGGSGGSGGSGGSDIVMT |
| | QSPATLSVTPGDRVSLSCRASQSISDYLHWYQQKSHESPRLLIKYASQSISGIPSRFSGSGSGSDFTLSINSVEPEDVGVYYCQ |
| | NGHSFPLTFGAGTKLELKGGSGGSEVQLQQSGPELVKPGASVKISCKTSGYTFTEYTMHWVKQSHGKSLEWIGGISPNIGGTSY |
| | NQKFKGKATLTVDKSSSTAYMELRSLTSEDSAVYYCARRGGSFDYWGQGTTLTVSS |
| 137 | scDb-6: CD19 (MOR208) |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | K |
| 138 | scDb-7: EGFR-2 |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSQPVLTQPPSVSVAPGKTARITCGGNNIGSKSVH |
| | WYQQKPGQAPVLVIYYDSDRPSGIPERFSGSNSGNTATLTISRVEAGDEADYYCQVWDTSSDHVLFGGGTKLTVL |
| 139 | ta-scFv-1: CD19 (MOR208) × CD16A-4 |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSSYELTQPLSVSVALGQTARITCGGNNIGSKSVHWYQQKPGQAPVLVIYQDKKRPSGIPERFSGSNSGNTATLTISRAçAG |
| | DEADYYCQVWDDYIVLFGCGTKLTVLGGSGGSGGSGGSGGSGGSGGSQVQLVQSGAEVKKPGASVKVSCKASGYTFTNYYMQWV |
| | RQAPGQCLEWMGIINPSGGVTSYAQKFQGRVTMTRDTSTSTVYMELSSLRSEDTAVYYCARGSAYYYDFADYWGQGTLVTVSS |
| 140 | ta-scFv-2: CD19 (MOR208) × NKG2D-1 |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSQSALTQPASVSGSPGQSITISCSGSSSNIGNNAVNWYQQLPGKAPKLLIYYDDLLPSGVSDRFSGSKSGTSAFLAISGLQ |
| | SEDEADYYCAAWDDSLNGPVFGGGTKLTVLGGSGGSGGSGGSGGSGGSGGSQVQLVESGGGLVKPGGSLRLSCAASGFTFSSYG |
| | MHWVRQAPGKGLEWVAFIRYDGSNKYYADSVKGRFTISRDNSKNTLYLQMNSLRAEDTAVYYCAKDRGLGDGTYFDYWGQGTTV |
| | TVSS |
| 141 | ta-scFv-3: CD19 (MOR208) × NKG2D-3 |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIQMTQSPSTLSASVGDRVTITCRASQSISSWLAWYQQKPGKAPKLLIYKASSLESGVPSRFSGSGSGTEFTLTISSLQP |
| | DDFATYYCQQYNSYPTFGGGTKVEIKGGSGGSGGSGGSGGSGGSGGSQVQLQQWGAGLLKPSETLSLTCAVYGGSFSGYYWSWI |
| | RQPPGKGLEWIGEIDHSGSTNYNPSLKSRVTISVDTSKNQFSLKLSSVTAADTAVYYCARARGPWSFDPWGQGTLVTVSS |
| 142 | ta-scFv-4: CD19 (MOR208) × NKp46-1 |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIQMTQTTSSLSASLGDRVTISCRASQDISNYLNWYQQKPDGTVKLLIYYTSRLHSGVPSRFSGSGSGTDYSLTINNLEQ |
| | EDIATYFCQQGNTRPWTFGGGTKLEIKGGSGGSGGSGGSGGSGGSGGSQVQLQQSGPELVKPGASVKMSCKASGYTFTDYVINW |
| | GKQRSGQGLEWIGEIYPGSGTNYYNEKFKAKATLTADKSSNIAYMQLSSLTSEDSAVYFCARRGRYGLYAMDYWGQGTSVTVSS |
| 143 | ta-scFv-5: CD19 (MOR208) × NKp46-3 |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIVMTQSPATLSVTPGDRVSLSCRASQSISDYLHWYQQKSHESPRLLIKYASQSISGIPSRFSGSGSGSDFTLSINSVEP |
| | EDVGVYYCQNGHSFPLTFGAGTKLELKGGSGGSGGSGGSGGSGGSGGSEVQLQQSGPELVKPGASVKISCKTSGYTFTEYTMHW |
| | VKQSHGKSLEWIGGISPNIGGTSYNQKFKGKATLTVDKSSSTAYMELRSLTSEDSAVYYCARRGGSFDYWGQGTTLTVSS |
| 144 | ta-scFv-6: EGFR-2 × CD16A-4 |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSS |
| | YELTQPLSVSVALGQTARITCGGNNIGSKSVHWYQQKPGQAPVLVIYQDKKRPSGIPERFSGSNSGNTATLTISRAQAGDEADY |
| | YCQVWDDYIVLFGCGTKLTVLGGSGGSGGSGGSGGSGGSGGSQVQLVQSGAEVKKPGASVKVSCKASGYTFTNYYMQWVRQAPG |
| | QCLEWMGIINPSGGVTSYAQKFQGRVTMTRDTSTSTVYMELSSLRSEDTAVYYCARGSAYYYDFADYWGQGTLVTVSS |
| 145 | ta-scFv-7: EGFR-2 × NKG2D-1 |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSQ |
| | SALTQPASVSGSPGQSITISCSGSSSNIGNNAVNWYQQLPGKAPKLLIYYDDLLPSGVSDRFSGSKSGTSAFLAISGLQSEDEA |
| | DYYCAAWDDSLNGPVFGGGTKLTVLGGSGGSGGSGGSGGSGGSGGSQVQLVESGGGLVKPGGSLRLSCAASGFTFSSYGMHWVR |
| | QAPGKGLEWVAFIRYDGSNKYYADSVKGRFTISRDNSKNTLYLQMNSLRAEDTAVYYCAKDRGLGDGTYFDYWGQGTTVTVSS |
| 146 | ta-scFv-8: EGFR-2 × NKG2D-3 |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSD |
| | IQMTQSPSTLSASVGDRVTITCRASQSISSWLAWYQQKPGKAPKLLIYKASSLESGVPSRFSGSGSGTEFTLTISSLQPDDFAT |
| | YYCQQYNSYPTFGGGTKVEIKGGSGGSGGSGGSGGSGGSGGSQVQLQQWGAGLLKPSETLSLTCAVYGGSFSGYYWSWIRQPPG |
| | KGLEWIGEIDHSGSTNYNPSLKSRVTISVDTSKNQFSLKLSSVTAADTAVYYCARARGPWSFDPWGQGTLVTVSS |
| 147 | ta-scFv-9: EGFR-2 × NKp46-1 |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSD |
| | IQMTQTTSSLSASLGDRVTISCRASQDISNYLNWYQQKPDGTVKLLIYYTSRLHSGVPSRFSGSGSGTDYSLTINNLEQEDIAT |
| | YFCQQGNTRPWTFGGGTKLEIKGGSGGSGGSGGSGGSGGSGGSQVQLQQSGPELVKPGASVKMSCKASGYTFTDYVINWGKQRS |
| | GQGLEWIGEIYPGSGTNYYNEKFKAKATLTADKSSNIAYMQLSSLTSEDSAVYFCARRGRYGLYAMDYWGQGTSVTVSS |
| 148 | ta-scFv-10: EGFR-2 × NKp46-3 |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSD |
| | IVMTQSPATLSVTPGDRVSLSCRASQSISDYLHWYQQKSHESPRLLIKYASQSISGIPSRFSGSGSGSDFTLSINSVEPEDVGV |
| | YYCQNGHSFPLTFGAGTKLELKGGSGGSGGSGGSGGSGGSGGSEVQLQQSGPELVKPGASVKISCKTSGYTFTEYTMHWVKQSH |
| | GKSLEWIGGISPNIGGTSYNQKFKGKATLTVDKSSSTAYMELRSLTSEDSAVYYCARRGGSFDYWGQGTTLTVSS |
| 149 | scFv-1: CD16A-4 |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSGGSGGSGGSGGSGGSQVQLVQSGAEVKKPGASVKVSCKASGYTFTNYYMQWVRQAP |
| | GQCLEWMGIINPSGGVTSYAQKFQGRVTMTRDTSTSTVYMELSSLRSEDTAVYYCARGSAYYYDFADYWGQGTLVTVSS |
| 150 | scFv-2: NKG2D-1 |
| | QVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSGGSGGSGGSQSALTQPASVSGSPGQSITISCSGSSSNI |
| | GNNAVNWYQQLPGKAPKLLIYYDDLLPSGVSDRFSGSKSGTSAFLAISGLQSEDEADYYCAAWDDSLNGPVFGGGTKLTVL |
| 151 | scFv-3: NKG2D-2 |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSGGSGGSGGSQSALTQPASVSGSPGQSITISCSGSSSNI |
| | GNNAVNWYQQLPGKAPKLLIYYDDLLPSGVSDRFSGSKSGTSAFLAISGLQSEDEADYYCAAWDDSLNGPVFGGGTKLTVL |
| 152 | scFv-4: NKG2D-3 |
| | QVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSGGSGGSGGSDIQMTQSPSTLSASVGDRVTITCRASQSISSWL |
| | AWYQQKPGKAPKLLIYKASSLESGVPSRFSGSGSGTEFTLTISSLQPDDFATYYCQQYNSYPTFGGGTKVEIK |
| 153 | scFv-5: NKG2D-4 |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSGGSGGSGGSDIQMTQSPSTLSASVGDRVTITCRASQSISSWL |
| | AWYQQKPGKAPKLLIYKASSLESGVPSRFSGSGSGTEFTLTISSLQPDDFATYYCQQYNSYPTFGGGTKVEIK |
| 154 | scFv-6: NKp46-1 |
| | QVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSGGSGGSGGSDIQMTQTTSSLSASLGDRVTISCRASQDIS |
| | NYLNWYQQKPDGTVKLLIYYTSRLHSGVPSRFSGSGSGTDYSLTINNLEQEDIATYFCQQGNTRPWTFGGGTKLEIK |
| 155 | scFv-7: NKp46-2 |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSGGSGGSGGSDIQMTQTTSSLSASLGDRVTISCRASQDIS |
| | NYLNWYQQKPDGTVKLLIYYTSRLHSGVPSRFSGSGSGTDYSLTINNLEQEDIATYFCQQGNTRPWTFGGGTKLEIK |
| 156 | scFv-8: NKp46-3 |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSGGSGGSGGSDIVMTQSPATLSVTPGDRVSLSCRASQSISDYLH |
| | WYQQKSHESPRLLIKYASQSISGIPSRFSGSGSGSDFTLSINSVEPEDVGVYYCQNGHSFPLTFGAGTKLELK |
| 157 | scFv-9: CD16 (3G8) |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSGGSGGSGGSGGSGGSQVTLKESGPGILQPSQTLSLTCSFSGFSLRTSGMGV |
| | GWIRQPSGKGLEWLAHIWWDDDKRYNPALKSRLTISKDTSSNQVFLKIASVDTADTATYYCAQINPAWFAYWGQGTLVTVSA |
| 158 | scFv-10: CD19 (MOR208) |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKGGSGGSGGSGGSGGSGGSGGSEVQLVESGGGLVKPGGSLKLSCAASGYTFTSYVMH |
| | WVRQAPGKGLEWIGYINPYNDGTKYNEKFQGRVTISSDKSISTAYMELSSLRSEDTAMYYCARGTYYYGTRVFDYWGQGTLVTV |
| | SS |
| 159 | scFv-11: CD19 (MOR208) |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIK |
| 160 | scFv-12: EGFR-1 |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGGSGGSGGSGGSGGSGGSGGSQVQLQESGPGLVKPSETLSLTCTVSGGSVSSGSYYWSWI |
| | RQPPGKGLEWIGYIYYSGSTNYNPSLKSRVTISVDTSKNQFSLKLSSVTAADTAVYYCARNPISIPAFDIWGQGTMVTVSS |
| 161 | 2Fab-scFc-1scDb-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPCEEQYGSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPEFEGGPSVFLFPPKPKDTLMISRTPEVTCVVVAV |
| | SHEDPEVKFNWYVDGVEVHNAKTKPCEEQYGSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSSYELTQPLSVSVALGQTARITCGGNNIGSKSVHWYQQKPGQAPVLVIYQDKKRPSGIPERFS |
| | GSNSGNTATLTISRAQAGDEADYYCQVWDDYIVLFGCGTKLTVLGGSGGSQVQLVESGGGLVKPGGSLRLSCAASGFTFSSYGM |
| | HWVRQAPGKGLEWVAFIRYDGSNKYYADSVKGRFTISRDNSKNTLYLQMNSLRAEDTAVYYCAKDRGLGDGTYFDYWGQGTTVT |
| | VSSGGSGGSGGSGGSGGSGGSQSALTQPASVSGSPGQSITISCSGSSSNIGNNAVNWYQQLPGKAPKLLIYYDDLLPSGVSDRF |
| | SGSKSGTSAFLAISGLQSEDEADYYCAAWDDSLNGPVFGGGTKLTVLGGSGGSQVQLVQSGAEVKKPGASVKVSCKASGYTFTN |
| | YYMQWVRQAPGQCLEWMGIINPSGGVTSYAQKFQGRVTMTRDTSTSTVYMELSSLRSEDTAVYYCARGSAYYYDFADYWGQGTL |
| | VTVSS |
| 162 | 2Fab-scFc-1scDb-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 163 | 2Fab-scFc-1scDb-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPCEEQYGSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPEFEGGPSVFLFPPKPKDTLMISRTPEVTCVVVAV |
| | SHEDPEVKFNWYVDGVEVHNAKTKPCEEQYGSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSSYELTQPLSVSVALGQTARITCGGNNIGSKSVHWYQQKPGQAPVLVIYQDKKRPSGIPERFS |
| | GSNSGNTATLTISRAQAGDEADYYCQVWDDYIVLFGCGTKLTVLGGSGGSQVQLQQWGAGLLKPSETLSLTCAVYGGSFSGYYW |
| | SWIRQPPGKGLEWIGEIDHSGSTNYNPSLKSRVTISVDTSKNQFSLKLSSVTAADTAVYYCARARGPWSFDPWGQGTLVTVSSG |
| | GSGGSGGSGGSGGSGGSDIQMTQSPSTLSASVGDRVTITCRASQSISSWLAWYQQKPGKAPKLLIYKASSLESGVPSRFSGSGS |
| | GTEFTLTISSLQPDDFATYYCQQYNSYPTFGGGTKVEIKGGSGGSQVQLVQSGAEVKKPGASVKVSCKASGYTFTNYYMQWVRQ |
| | APGQCLEWMGIINPSGGVTSYAQKFQGRVTMTRDTSTSTVYMELSSLRSEDTAVYYCARGSAYYYDFADYWGQGTLVTVSS |
| 164 | 2Fab-scFc-1scDb-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 165 | 2Fab-scFc-1scDb-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPCEEQYGSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPEFEGGPSVFLFPPKPKDTLMISRTPEVTCVVVAV |
| | SHEDPEVKFNWYVDGVEVHNAKTKPCEEQYGSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSSYELTQPLSVSVALGQTARITCGGNNIGSKSVHWYQQKPGQAPVLVIYQDKKRPSGIPERFS |
| | GSNSGNTATLTISRAQAGDEADYYCQVWDDYIVLFGCGTKLTVLGGSGGSQVQLQQSGPELVKPGASVKMSCKASGYTFTDYVI |
| | NWGKQRSGQGLEWIGEIYPGSGTNYYNEKFKAKATLTADKSSNIAYMQLSSLTSEDSAVYFCARRGRYGLYAMDYWGQGTSVTV |
| | SSGGSGGSGGSGGSGGSGGSDIQMTQTTSSLSASLGDRVTISCRASQDISNYLNWYQQKPDGTVKLLIYYTSRLHSGVPSRFSG |
| | SGSGTDYSLTINNLEQEDIATYFCQQGNTRPWTFGGGTKLEIKGGSGGSQVQLVQSGAEVKKPGASVKVSCKASGYTFTNYYMQ |
| | WVRQAPGQCLEWMGIINPSGGVTSYAQKFQGRVTMTRDTSTSTVYMELSSLRSEDTAVYYCARGSAYYYDFADYWGQGTLVTVS |
| | S |
| 166 | 2Fab-scFc-1scDb-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 167 | 2Fab-scFc-1scDb-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPCEEQYGSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPEFEGGPSVFLFPPKPKDTLMISRTPEVTCVVVAV |
| | SHEDPEVKFNWYVDGVEVHNAKTKPCEEQYGSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSSYELTQPLSVSVALGQTARITCGGNNIGSKSVHWYQQKPGQAPVLVIYQDKKRPSGIPERFS |
| | GSNSGNTATLTISRAQAGDEADYYCQVWDDYIVLFGCGTKLTVLGGSGGSEVQLQQSGPELVKPGASVKISCKTSGYTFTEYTM |
| | HWVKQSHGKSLEWIGGISPNIGGTSYNQKFKGKATLTVDKSSSTAYMELRSLTSEDSAVYYCARRGGSFDYWGQGTTLTVSSGG |
| | SGGSGGSGGSGGSGGSDIVMTQSPATLSVTPGDRVSLSCRASQSISDYLHWYQQKSHESPRLLIKYASQSISGIPSRFSGSGSG |
| | SDFTLSINSVEPEDVGVYYCQNGHSFPLTFGAGTKLELKGGSGGSQVQLVQSGAEVKKPGASVKVSCKASGYTFTNYYMQWVRQ |
| | APGQCLEWMGIINPSGGVTSYAQKFQGRVTMTRDTSTSTVYMELSSLRSEDTAVYYCARGSAYYYDFADYWGQGTLVTVSS |
| 168 | 2Fab-scFc-1scDb-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 169 | 2Fab-scFc-1scFv-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPCEEQYNSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPELLGGPSVFLFPPKPKDTLMISRTPEVTCVVVDV |
| | SHEDPEVKFNWYVDGVEVHNAKTKPCEEQYNSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSQVQLVESGGGLVKPGGSLRLSCAASGFTFSSYGMHWVRQAPGKGLEWVAFIRYDGSNKYYAD |
| | SVKGRFTISRDNSKNTLYLQMNSLRAEDTAVYYCAKDRGLGDGTYFDYWGQGTTVTVSSGGSGGSGGSGGSGGSGGSQSALTQP |
| | ASVSGSPGQSITISCSGSSSNIGNNAVNWYQQLPGKAPKLLIYYDDLLPSGVSDRFSGSKSGTSAFLAISGLQSEDEADYYCAA |
| | WDDSLNGPVFGGGTKLTVL |
| 170 | 2Fab-scFc-1scFv-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 171 | 2Fab-scFc-1scFv-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPCEEQYNSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPELLGGPSVFLFPPKPKDTLMISRTPEVTCVVVDV |
| | SHEDPEVKFNWYVDGVEVHNAKTKPCEEQYNSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSQVQLQQWGAGLLKPSETLSLTCAVYGGSFSGYYWSWIRQPPGKGLEWIGEIDHSGSTNYNPS |
| | LKSRVTISVDTSKNQFSLKLSSVTAADTAVYYCARARGPWSFDPWGQGTLVTVSSGGSGGSGGSGGSGGSGGSDIQMTQSPSTL |
| | SASVGDRVTITCRASQSISSWLAWYQQKPGKAPKLLIYKASSLESGVPSRFSGSGSGTEFTLTISSLQPDDFATYYCQQYNSYP |
| | TFGGGTKVEIKS |
| 172 | 2Fab-scFc-1scFv-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 173 | 2Fab-scFc-1scFv-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPCEEQYNSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPELLGGPSVFLFPPKPKDTLMISRTPEVTCVVVDV |
| | SHEDPEVKFNWYVDGVEVHNAKTKPCEEQYNSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSQVQLQQSGPELVKPGASVKMSCKASGYTFTDYVINWGKQRSGQGLEWIGEIYPGSGTNYYNE |
| | KFKAKATLTADKSSNIAYMQLSSLTSEDSAVYFCARRGRYGLYAMDYWGQGTSVTVSSGGSGGSGGSGGSGGSGGSDIQMTQTT |
| | SSLSASLGDRVTISCRASQDISNYLNWYQQKPDGTVKLLIYYTSRLHSGVPSRFSGSGSGTDYSLTINNLEQEDIATYFCQQGN |
| | TRPWTFGGGTKLEIK |
| 174 | 2Fab-scFc-1scFv-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 175 | 2Fab-scFc-1scFv-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPCEEQYNSTYRCVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEALHNHYTQK |
| | SLSLSPGKGGGGSGGGGSGGGGSGGGGSGGGGSGGGGSDKTHTCPPCPAPELLGGPSVFLFPPKPKDTLMISRTPEVTCVVVDV |
| | SHEDPEVKFNWYVDGVEVHNAKTKPCEEQYNSTYRCVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYSKLTVDKSRWQQGNVFSCSVMHEALHN |
| | HYTQKSLSLSPGGGGGSGGGGSEVQLQQSGPELVKPGASVKISCKTSGYTFTEYTMHWVKQSHGKSLEWIGGISPNIGGTSYNQ |
| | KFKGKATLTVDKSSSTAYMELRSLTSEDSAVYYCARRGGSFDYWGQGTTLTVSSGGSGGSGGSGGSGGSGGSDIVMTQSPATLS |
| | VTPGDRVSLSCRASQSISDYLHWYQQKSHESPRLLIKYASQSISGIPSRFSGSGSGSDFTLSINSVEPEDVGVYYCQNGHSFPL |
| | TFGAGTKLELK |
| 176 | 2Fab-scFc-1scFv-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 177 | 2Fab-1scDb-AFc-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 178 | 2Fab-1scDb-AFc-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 179 | 2Fab-1scDb-AFc-1 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLVESGGGLVKPGGSLRLSCAASGFTFSSYGMHWVRQAPGKGLEWVAFIRYDGS |
| | NKYYADSVKGRFTISRDNSKNTLYLQMNSLRAEDTAVYYCAKDRGLGDGTYFDYWGQGTTVTVSSGGSGGSGGSGGSGGSGGSQ |
| | SALTQPASVSGSPGQSITISCSGSSSNIGNNAVNWYQQLPGKAPKLLIYYDDLLPSGVSDRFSGSKSGTSAFLAISGLQSEDEA |
| | DYYCAAWDDSLNGPVFGGGTKLTVLGGSGGSQVQLVQSGAEVKKPGASVKVSCKASGYTFTNYYMQWVRQAPGQCLEWMGIINP |
| | SGGVTSYAQKFQGRVTMTRDTSTSTVYMELSSLRSEDTAVYYCARGSAYYYDFADYWGQGTLVTVSSDKTHTCPPCPAPEFEGG |
| | PSVFLFPPKPKDTLMISRTPEVTCVVVAVSHEDPEVKFNWYVDGVEVHNAKTKPREEQYGSTYRVVSVLTVLHQDWLNGKEYKC |
| | KVSNKALPAPIEKTISKAKGQPREPQVYTLPPSREEMTKNQVSLSCAVKGFYPSDIAVEWESNGQPENNYKTTPPVLDSDGSFF |
| | LVSKLTVDKSRWQQGNVFSCSVMHEALHNHYTQKSLSLSPG |
| 180 | 2Fab-1scDb-AFc-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 181 | 2Fab-1scDb-AFc-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 182 | 2Fab-1scDb-AFc-2 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLQQWGAGLLKPSETLSLTCAVYGGSFSGYYWSWIRQPPGKGLEWIGEIDHSGS |
| | TNYNPSLKSRVTISVDTSKNQFSLKLSSVTAADTAVYYCARARGPWSFDPWGQGTLVTVSSGGSGGSGGSGGSGGSGGSDIQMT |
| | QSPSTLSASVGDRVTITCRASQSISSWLAWYQQKPGKAPKLLIYKASSLESGVPSRFSGSGSGTEFTLTISSLQPDDFATYYCQ |
| | QYNSYPTFGGGTKVEIKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSSDKTHTCPPCPAPEFEGGPSVFLFPP |
| | KPKDTLMISRTPEVTCVVVAVSHEDPEVKFNWYVDGVEVHNAKTKPREEQYGSTYRVVSVLTVLHQDWLNGKEYKCKVSNKALP |
| | APIEKTISKAKGQPREPQVYTLPPSREEMTKNQVSLSCAVKGFYPSDIAVEWESNGQPENNYKTTPPVLDSDGSFFLVSKLTVD |
| | KSRWQQGNVFSCSVMHEALHNHYTQKSLSLSPG |
| 183 | 2Fab-1scDb-AFc-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 184 | 2Fab-1scDb-AFc-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 185 | 2Fab-1scDb-AFc-3 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLQQSGPELVKPGASVKMSCKASGYTFTDYVINWGKQRSGQGLEWIGEIYPGSG |
| | TNYYNEKFKAKATLTADKSSNIAYMQLSSLTSEDSAVYFCARRGRYGLYAMDYWGQGTSVTVSSGGSGGSGGSGGSGGSGGSDI |
| | QMTQTTSSLSASLGDRVTISCRASQDISNYLNWYQQKPDGTVKLLIYYTSRLHSGVPSRFSGSGSGTDYSLTINNLEQEDIATY |
| | FCQQGNTRPWTFGGGTKLEIKGGSGGSQVQLVQSGAEVKKPGASVKVSCKASGYTFTNYYMQWVRQAPGQCLEWMGIINPSGGV |
| | TSYAQKFQGRVTMTRDTSTSTVYMELSSLRSEDTAVYYCARGSAYYYDFADYWGQGTLVTVSSDKTHTCPPCPAPEFEGGPSVF |
| | LFPPKPKDTLMISRTPEVTCVVVAVSHEDPEVKFNWYVDGVEVHNAKTKPREEQYGSTYRVVSVLTVLHQDWLNGKEYKCKVSN |
| | KALPAPIEKTISKAKGQPREPQVYTLPPSREEMTKNQVSLSCAVKGFYPSDIAVEWESNGQPENNYKTTPPVLDSDGSFFLVSK |
| | LTVDKSRWQQGNVFSCSVMHEALHNHYTQKSLSLSPG |
| 186 | 2Fab-1scDb-AFc-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 187 | 2Fab-1scDb-AFc-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 188 | 2Fab-1scDb-AFc-4 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSEVQLQQSGPELVKPGASVKISCKTSGYTFTEYTMHWVKQSHGKSLEWIGGISPNIG |
| | GTSYNQKFKGKATLTVDKSSSTAYMELRSLTSEDSAVYYCARRGGSFDYWGQGTTLTVSSGGSGGSGGSGGSGGSGGSDIVMTQ |
| | SPATLSVTPGDRVSLSCRASQSISDYLHWYQQKSHESPRLLIKYASQSISGIPSRFSGSGSGSDFTLSINSVEPEDVGVYYCQN |
| | GHSFPLTFGAGTKLELKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSSDKTHTCPPCPAPEFEGGPSVFLFPP |
| | KPKDTLMISRTPEVTCVVVAVSHEDPEVKFNWYVDGVEVHNAKTKPREEQYGSTYRVVSVLTVLHQDWLNGKEYKCKVSNKALP |
| | APIEKTISKAKGQPREPQVYTLPPSREEMTKNQVSLSCAVKGFYPSDIAVEWESNGQPENNYKTTPPVLDSDGSFFLVSKLTVD |
| | KSRWQQGNVFSCSVMHEALHNHYTQKSLSLSPG |
| 189 | 2Fab-1scDb-AFc-5 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTQVQLQESGPGLVKPSETLSLTCTV |
| | SGGSVSSGSYYWSWIRQPPGKGLEWIGYIYYSGSTNYNPSLKSRVTISVDTSKNQFSLKLSSVTAADTAVYYCARNPISIPAFD |
| | IWGQGTMVTVSSASTKGPSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPEFEGGPSVFLFPPKPKDTLMISRTPEVTCVVVAVSHEDPEV |
| | KFNWYVDGVEVHNAKTKPREEQYGSTYRVVSVLTVLHQDWLNGKEYKCKVSNKALPAPIEKTISKAKGQPREPQVYTLPPSREE |
| | MTKNQVSLWCLVKGFYPSDIAVEWESNGQPENNYKTTPPVLDSDGSFFLYSKLTVDKSRWQQGNVFSCSVMHEALHNHYTQKSL |
| | SLSPG |
| 190 | 2Fab-1scDb-AFc-5 Chain2: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECS |
| 191 | 2Fab-1scDb-AFc-5 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLVESGGGLVKPGGSLRLSCAASGFTFSSYGMHWVRQAPGKGLEWVAFIRYDGS |
| | NKYYADSVKGRFTISRDNSKNTLYLQMNSLRAEDTAVYYCAKDRGLGDGTYFDYWGQGTTVTVSSGGSGGSGGSGGSGGSGGSQ |
| | SALTQPASVSGSPGQSITISCSGSSSNIGNNAVNWYQQLPGKAPKLLIYYDDLLPSGVSDRFSGSKSGTSAFLAISGLQSEDEA |
| | DYYCAAWDDSLNGPVFGGGTKLTVLGGSGGSQVQLVQSGAEVKKPGASVKVSCKASGYTFTNYYMQWVRQAPGQCLEWMGIINP |
| | SGGVTSYAQKFQGRVTMTRDTSTSTVYMELSSLRSEDTAVYYCARGSAYYYDFADYWGQGTLVTVSSDKTHTCPPCPAPEFEGG |
| | PSVFLFPPKPKDTLMISRTPEVTCVVVAVSHEDPEVKFNWYVDGVEVHNAKTKPREEQYGSTYRVVSVLTVLHQDWLNGKEYKC |
| | KVSNKALPAPIEKTISKAKGQPREPQVYTLPPSREEMTKNQVSLSCAVKGFYPSDIAVEWESNGQPENNYKTTPPVLDSDGSFF |
| | LVSKLTVDKSRWQQGNVFSCSVMHEALHNHYTQKSLSLSPG |
| 192 | 2Fab-1scDb-AFc-6 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTQVQLQESGPGLVKPSETLSLTCTV |
| | SGGSVSSGSYYWSWIRQPPGKGLEWIGYIYYSGSTNYNPSLKSRVTISVDTSKNQFSLKLSSVTAADTAVYYCARNPISIPAFD |
| | IWGQGTMVTVSSASTKGPSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPEFEGGPSVFLFPPKPKDTLMISRTPEVTCVVVAVSHEDPEV |
| | KFNWYVDGVEVHNAKTKPREEQYGSTYRVVSVLTVLHQDWLNGKEYKCKVSNKALPAPIEKTISKAKGQPREPQVYTLPPSREE |
| | MTKNQVSLWCLVKGFYPSDIAVEWESNGQPENNYKTTPPVLDSDGSFFLYSKLTVDKSRWQQGNVFSCSVMHEALHNHYTQKSL |
| | SLSPG |
| 193 | 2Fab-1scDb-AFc-6 Chain2: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECS |
| 194 | 2Fab-1scDb-AFc-6 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLQQWGAGLLKPSETLSLTCAVYGGSFSGYYWSWIRQPPGKGLEWIGEIDHSGS |
| | TNYNPSLKSRVTISVDTSKNQFSLKLSSVTAADTAVYYCARARGPWSFDPWGQGTLVTVSSGGSGGSGGSGGSGGSGGSDIQMT |
| | QSPSTLSASVGDRVTITCRASQSISSWLAWYQQKPGKAPKLLIYKASSLESGVPSRFSGSGSGTEFTLTISSLQPDDFATYYCQ |
| | QYNSYPTFGGGTKVEIKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSSDKTHTCPPCPAPEFEGGPSVFLFPP |
| | KPKDTLMISRTPEVTCVVVAVSHEDPEVKFNWYVDGVEVHNAKTKPREEQYGSTYRVVSVLTVLHQDWLNGKEYKCKVSNKALP |
| | APIEKTISKAKGQPREPQVYTLPPSREEMTKNQVSLSCAVKGFYPSDIAVEWESNGQPENNYKTTPPVLDSDGSFFLVSKLTVD |
| | KSRWQQGNVFSCSVMHEALHNHYTQKSLSLSPG |
| 195 | 2Fab-1scDb-AFc-7 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTQVQLQESGPGLVKPSETLSLTCTV |
| | SGGSVSSGSYYWSWIRQPPGKGLEWIGYIYYSGSTNYNPSLKSRVTISVDTSKNQFSLKLSSVTAADTAVYYCARNPISIPAFD |
| | IWGQGTMVTVSSASTKGPSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPEFEGGPSVFLFPPKPKDTLMISRTPEVTCVVVAVSHEDPEV |
| | KFNWYVDGVEVHNAKTKPREEQYGSTYRVVSVLTVLHQDWLNGKEYKCKVSNKALPAPIEKTISKAKGQPREPQVYTLPPSREE |
| | MTKNQVSLWCLVKGFYPSDIAVEWESNGQPENNYKTTPPVLDSDGSFFLYSKLTVDKSRWQQGNVFSCSVMHEALHNHYTQKSL |
| | SLSPG |
| 196 | 2Fab-1scDb-AFc-7 Chain2: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECS |
| 197 | 2Fab-1scDb-AFc-7 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLQQSGPELVKPGASVKMSCKASGYTFTDYVINWGKQRSGQGLEWIGEIYPGSG |
| | TNYYNEKFKAKATLTADKSSNIAYMQLSSLTSEDSAVYFCARRGRYGLYAMDYWGQGTSVTVSSGGSGGSGGSGGSGGSGGSDI |
| | QMTQTTSSLSASLGDRVTISCRASQDISNYLNWYQQKPDGTVKLLIYYTSRLHSGVPSRFSGSGSGTDYSLTINNLEQEDIATY |
| | FCQQGNTRPWTFGGGTKLEIKGGSGGSQVQLVQSGAEVKKPGASVKVSCKASGYTFTNYYMQWVRQAPGQCLEWMGIINPSGGV |
| | TSYAQKFQGRVTMTRDTSTSTVYMELSSLRSEDTAVYYCARGSAYYYDFADYWGQGTLVTVSSDKTHTCPPCPAPEFEGGPSVF |
| | LFPPKPKDTLMISRTPEVTCVVVAVSHEDPEVKFNWYVDGVEVHNAKTKPREEQYGSTYRVVSVLTVLHQDWLNGKEYKCKVSN |
| | KALPAPIEKTISKAKGQPREPQVYTLPPSREEMTKNQVSLSCAVKGFYPSDIAVEWESNGQPENNYKTTPPVLDSDGSFFLVSK |
| | LTVDKSRWQQGNVFSCSVMHEALHNHYTQKSLSLSPG |
| 198 | 2Fab-1scDb-AFc-8 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTQVQLQESGPGLVKPSETLSLTCTV |
| | SGGSVSSGSYYWSWIRQPPGKGLEWIGYIYYSGSTNYNPSLKSRVTISVDTSKNQFSLKLSSVTAADTAVYYCARNPISIPAFD |
| | IWGQGTMVTVSSASTKGPSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPEFEGGPSVFLFPPKPKDTLMISRTPEVTCVVVAVSHEDPEV |
| | KFNWYVDGVEVHNAKTKPREEQYGSTYRVVSVLTVLHQDWLNGKEYKCKVSNKALPAPIEKTISKAKGQPREPQVYTLPPSREE |
| | MTKNQVSLWCLVKGFYPSDIAVEWESNGQPENNYKTTPPVLDSDGSFFLYSKLTVDKSRWQQGNVFSCSVMHEALHNHYTQKSL |
| | SLSPG |
| 199 | 2Fab-1scDb-AFc-8 Chain2: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECS |
| 200 | 2Fab-1scDb-AFc-8 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSEVQLQQSGPELVKPGASVKISCKTSGYTFTEYTMHWVKQSHGKSLEWIGGISPNIG |
| | GTSYNQKFKGKATLTVDKSSSTAYMELRSLTSEDSAVYYCARRGGSFDYWGQGTTLTVSSGGSGGSGGSGGSGGSGGSDIVMTQ |
| | SPATLSVTPGDRVSLSCRASQSISDYLHWYQQKSHESPRLLIKYASQSISGIPSRFSGSGSGSDFTLSINSVEPEDVGVYYCQN |
| | GHSFPLTFGAGTKLELKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSSDKTHTCPPCPAPEFEGGPSVFLFPP |
| | KPKDTLMISRTPEVTCVVVAVSHEDPEVKFNWYVDGVEVHNAKTKPREEQYGSTYRVVSVLTVLHQDWLNGKEYKCKVSNKALP |
| | APIEKTISKAKGQPREPQVYTLPPSREEMTKNQVSLSCAVKGFYPSDIAVEWESNGQPENNYKTTPPVLDSDGSFFLVSKLTVD |
| | KSRWQQGNVFSCSVMHEALHNHYTQKSLSLSPG |
| 201 | 2Fab-1scFv-AFc-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 202 | 2Fab-1scFv-AFc-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 203 | 2Fab-1scFv-AFc-1 Chain3: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSGGSGGSGGSQSALTQPASVSGSPGQSITISCSGSSSNI |
| | GNNAVNWYQQLPGKAPKLLIYYDDLLPSGVSDRFSGSKSGTSAFLAISGLQSEDEADYYCAAWDDSLNGPVFGGGTKLTVLDKT |
| | HTCPPCPAPELLGGPSVFLFPPKPKDTLMISRTPEVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQKSLSLSPG |
| 204 | 2Fab-1scFv-AFc-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 205 | 2Fab-1scFv-AFc-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 206 | 2Fab-1scFv-AFc-2 Chain3: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSGGSGGSGGSDIQMTQSPSTLSASVGDRVTITCRASQSISSWL |
| | AWYQQKPGKAPKLLIYKASSLESGVPSRFSGSGSGTEFTLTISSLQPDDFATYYCQQYNSYPTFGGGTKVEIKDKTHTCPPCPA |
| | PELLGGPSVFLFPPKPKDTLMISRTPEVTCVVVDVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNHYTQKSLSLSPG |
| 207 | 2Fab-1scFv-AFc-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 208 | 2Fab-1scFv-AFc-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 209 | 2Fab-1scFv-AFc-3 Chain3: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSGGSGGSGGSDIQMTQTTSSLSASLGDRVTISCRASQDIS |
| | NYLNWYQQKPDGTVKLLIYYTSRLHSGVPSRFSGSGSGTDYSLTINNLEQEDIATYFCQQGNTRPWTFGGGTKLEIKDKTHTCP |
| | PCPAPELLGGPSVFLFPPKPKDTLMISRTPEVTCVVVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQVSLSCAVKGFYPSDIAVEWESNGQPENNYKTTP |
| | PVLDSDGSFFLVSKLTVDKSRWQQGNVFSCSVMHEALHNHYTQKSLSLSPG |
| 210 | 2Fab-1scFv-AFc-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 211 | 2Fab-1scFv-AFc-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 212 | 2Fab-1scFv-AFc-4 Chain3: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSGGSGGSGGSDIVMTQSPATLSVTPGDRVSLSCRASQSISDYLH |
| | WYQQKSHESPRLLIKYASQSISGIPSRFSGSGSGSDFTLSINSVEPEDVGVYYCQNGHSFPLTFGAGTKLELKDKTHTCPPCPA |
| | PELLGGPSVFLFPPKPKDTLMISRTPEVTCVVVDVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNHYTQKSLSLSPG |
| 213 | 2Fab-1scDb-AFc-9 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 214 | 2Fab-1scDb-AFc-9 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 215 | 2Fab-1scDb-AFc-9 Chain3: |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSQVQLVESGGGLVKPGGSLRLSCAASGFTFSSYGMHWVRQAPGKGLEWVAFI |
| | RYDGSNKYYADSVKGRFTISRDNSKNTLYLQMNSLRAEDTAVYYCAKDRGLGDGTYFDYWGQGTTVTVSSGGSGGSGGSGGSGG |
| | SGGSQSALTQPASVSGSPGQSITISCSGSSSNIGNNAVNWYQQLPGKAPKLLIYYDDLLPSGVSDRFSGSKSGTSAFLAISGLQ |
| | SEDEADYYCAAWDDSLNGPVFGGGTKLTVLGGSGGSQVTLKESGPGILQPSQTLSLTCSFSGFSLRTSGMGVGWIRQPSGKGLE |
| | WLAHIWWDDDKRYNPALKSRLTISKDTSSNQVFLKIASVDTADTATYYCAQINPAWFAYWGQGTLVTVSADKTHTCPPCPAPEF |
| | EGGPSVFLFPPKPKDTLMISRTPEVTCVVVAVSHEDPEVKFNWYVDGVEVHNAKTKPREEQYGSTYRVVSVLTVLHQDWLNGKE |
| | YKCKVSNKALPAPIEKTISKAKGQPREPQVYTLPPSREEMTKNQVSLSCAVKGFYPSDIAVEWESNGQPENNYKTTPPVLDSDG |
| | SFFLVSKLTVDKSRWQQGNVFSCSVMHEALHNHYTQKSLSLSPG |
| 216 | 2Fab-1scDb-AFc-10 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 217 | 2Fab-1scDb-AFc-10 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 218 | 2Fab-1scDb-AFc-10 Chain3: |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSQVQLQQWGAGLLKPSETLSLTCAVYGGSFSGYYWSWIRQPPGKGLEWIGEI |
| | DHSGSTNYNPSLKSRVTISVDTSKNQFSLKLSSVTAADTAVYYCARARGPWSFDPWGQGTLVTVSSGGSGGSGGSGGSGGSGGS |
| | DIQMTQSPSTLSASVGDRVTITCRASQSISSWLAWYQQKPGKAPKLLIYKASSLESGVPSRFSGSGSGTEFTLTISSLQPDDFA |
| | TYYCQQYNSYPTFGGGTKVEIKGGSGGSQVTLKESGPGILQPSQTLSLTCSFSGFSLRTSGMGVGWIRQPSGKGLEWLAHIWWD |
| | DDKRYNPALKSRLTISKDTSSNQVFLKIASVDTADTATYYCAQINPAWFAYWGQGTLVTVSADKTHTCPPCPAPEFEGGPSVFL |
| | FPPKPKDTLMISRTPEVTCVVVAVSHEDPEVKFNWYVDGVEVHNAKTKPREEQYGSTYRVVSVLTVLHQDWLNGKEYKCKVSNK |
| | ALPAPIEKTISKAKGQPREPQVYTLPPSREEMTKNQVSLSCAVKGFYPSDIAVEWESNGQPENNYKTTPPVLDSDGSFFLVSKL |
| | TVDKSRWQQGNVFSCSVMHEALHNHYTQKSLSLSPG |
| 219 | 2Fab-1scDb-AFc-11 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 220 | 2Fab-1scDb-AFc-11 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 221 | 2Fab-1scDb-AFc-11 Chain3: |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSQVQLQQSGPELVKPGASVKMSCKASGYTFTDYVINWGKQRSGQGLEWIGEI |
| | YPGSGTNYYNEKFKAKATLTADKSSNIAYMQLSSLTSEDSAVYFCARRGRYGLYAMDYWGQGTSVTVSSGGSGGSGGSGGSGGS |
| | GGSDIQMTQTTSSLSASLGDRVTISCRASQDISNYLNWYQQKPDGTVKLLIYYTSRLHSGVPSRFSGSGSGTDYSLTINNLEQE |
| | DIATYFCQQGNTRPWTFGGGTKLEIKGGSGGSQVTLKESGPGILQPSQTLSLTCSFSGFSLRTSGMGVGWIRQPSGKGLEWLAH |
| | IWWDDDKRYNPALKSRLTISKDTSSNQVFLKIASVDTADTATYYCAQINPAWFAYWGQGTLVTVSADKTHTCPPCPAPEFEGGP |
| | SVFLFPPKPKDTLMISRTPEVTCVVVAVSHEDPEVKFNWYVDGVEVHNAKTKPREEQYGSTYRVVSVLTVLHQDWLNGKEYKCK |
| | VSNKALPAPIEKTISKAKGQPREPQVYTLPPSREEMTKNQVSLSCAVKGFYPSDIAVEWESNGQPENNYKTTPPVLDSDGSFFL |
| | VSKLTVDKSRWQQGNVFSCSVMHEALHNHYTQKSLSLSPG |
| 222 | 2Fab-1scDb-AFc-12 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTEVQLVESGGGLVKPGGSLKLSCA |
| | ASGYTFTSYVMHWVRQAPGKGLEWIGYINPYNDGTKYNEKFQGRVTISSDKSISTAYMELSSLRSEDTAMYYCARGTYYYGTRV |
| | FDYWGQGTLVTVSSASTKGPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEFEGGPSVFLFPPKPKDTLMISRTPEVTCVVVAVSHEDP |
| | EVKFNWYVDGVEVHNAKTKPREEQYGSTYRVVSVLTVLHQDWLNGKEYKCKVSNKALPAPIEKTISKAKGQPREPQVYTLPPSR |
| | EEMTKNQVSLWCLVKGFYPSDIAVEWESNGQPENNYKTTPPVLDSDGSFFLYSKLTVDKSRWQQGNVFSCSVMHEALHNHYTQK |
| | SLSLSPG |
| 223 | 2Fab-1scDb-AFc-12 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 224 | 2Fab-1scDb-AFc-12 Chain3: |
| | DIVLTQSPASLAVSLGQRATISCKASQSVDFDGDSFMNWYQQKPGQPPKLLIYTTSNLESGIPARFSASGSGTDFTLNIHPVEE |
| | EDTATYYCQQSNEDPYTFGGGTKLELKGGSGGSEVQLQQSGPELVKPGASVKISCKTSGYTFTEYTMHWVKQSHGKSLEWIGGI |
| | SPNIGGTSYNQKFKGKATLTVDKSSSTAYMELRSLTSEDSAVYYCARRGGSFDYWGQGTTLTVSSGGSGGSGGSGGSGGSGGSD |
| | IVMTQSPATLSVTPGDRVSLSCRASQSISDYLHWYQQKSHESPRLLIKYASQSISGIPSRFSGSGSGSDFTLSINSVEPEDVGV |
| | YYCQNGHSFPLTFGAGTKLELKGGSGGSQVTLKESGPGILQPSQTLSLTCSFSGFSLRTSGMGVGWIRQPSGKGLEWLAHIWWD |
| | DDKRYNPALKSRLTISKDTSSNQVFLKIASVDTADTATYYCAQINPAWFAYWGQGTLVTVSADKTHTCPPCPAPEFEGGPSVFL |
| | FPPKPKDTLMISRTPEVTCVVVAVSHEDPEVKFNWYVDGVEVHNAKTKPREEQYGSTYRVVSVLTVLHQDWLNGKEYKCKVSNK |
| | ALPAPIEKTISKAKGQPREPQVYTLPPSREEMTKNQVSLSCAVKGFYPSDIAVEWESNGQPENNYKTTPPVLDSDGSFFLVSKL |
| | TVDKSRWQQGNVFSCSVMHEALHNHYTQKSLSLSPG |
| 225 | 1Fab-1scDb-AFc-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPG |
| 226 | 1Fab-1scDb-AFc-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 227 | 1Fab-1scDb-AFc-1 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLVESGGGLVKPGGSLRLSCAASGFTFSSYGMHWVRQAPGKGLEWVAFIRYDGS |
| | NKYYADSVKGRFTISRDNSKNTLYLQMNSLRAEDTAVYYCAKDRGLGDGTYFDYWGQGTTVTVSSGGSGGSGGSGGSGGSGGSQ |
| | SALTQPASVSGSPGQSITISCSGSSSNIGNNAVNWYQQLPGKAPKLLIYYDDLLPSGVSDRFSGSKSGTSAFLAISGLQSEDEA |
| | DYYCAAWDDSLNGPVFGGGTKLTVLGGSGGSQVQLVQSGAEVKKPGASVKVSCKASGYTFTNYYMQWVRQAPGQCLEWMGIINP |
| | SGGVTSYAQKFQGRVTMTRDTSTSTVYMELSSLRSEDTAVYYCARGSAYYYDFADYWGQGTLVTVSSDKTHTCPPCPAPEFEGG |
| | PSVFLFPPKPKDTLMISRTPEVTCVVVAVSHEDPEVKFNWYVDGVEVHNAKTKPREEQYGSTYRVVSVLTVLHQDWLNGKEYKC |
| | KVSNKALPAPIEKTISKAKGQPREPQVYTLPPSREEMTKNQVSLSCAVKGFYPSDIAVEWESNGQPENNYKTTPPVLDSDGSFF |
| | LVSKLTVDKSRWQQGNVFSCSVMHEALHNHYTQKSLSLSPG |
| 228 | 1Fab-1scDb-AFc-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPG |
| 229 | 1Fab-1scDb-AFc-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 230 | 1Fab-1scDb-AFc-2 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSQVQLQQWGAGLLKPSETLSLTCAVYGGSFSGYYWSWIRQPPGKGLEWIGEIDHSGS |
| | TNYNPSLKSRVTISVDTSKNQFSLKLSSVTAADTAVYYCARARGPWSFDPWGQGTLVTVSSGGSGGSGGSGGSGGSGGSDIQMT |
| | QSPSTLSASVGDRVTITCRASQSISSWLAWYQQKPGKAPKLLIYKASSLESGVPSRFSGSGSGTEFTLTISSLQPDDFATYYCQ |
| | QYNSYPTFGGGTKVEIKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSSDKTHTCPPCPAPEFEGGPSVFLFPP |
| | KPKDTLMISRTPEVTCVVVAVSHEDPEVKFNWYVDGVEVHNAKTKPREEQYGSTYRVVSVLTVLHQDWLNGKEYKCKVSNKALP |
| | APIEKTISKAKGQPREPQVYTLPPSREEMTKNQVSLSCAVKGFYPSDIAVEWESNGQPENNYKTTPPVLDSDGSFFLVSKLTVD |
| | KSRWQQGNVFSCSVMHEALHNHYTQKSLSLSPG |
| 231 | 1Fab-1scDb-AFc-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPG |
| 232 | 1Fab-1scDb-AFc-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 233 | 1Fab-1scDb-AFc-3 Chain3: |
| | SYELTQPLSVSVALGQTARITCGGNNIGSKSVHWYQQKPGQAPVLVIYQDKKRPSGIPERFSGSNSGNTATLTISRAçAGDEAD |
| | YYCQVWDDYIVLFGCGTKLTVLGGSGGSQVQLQQSGPELVKPGASVKMSCKASGYTFTDYVINWGKQRSGQGLEWIGEIYPGSG |
| | TNYYNEKFKAKATLTADKSSNIAYMQLSSLTSEDSAVYFCARRGRYGLYAMDYWGQGTSVTVSSGGSGGSGGSGGSGGSGGSDI |
| | QMTQTTSSLSASLGDRVTISCRASQDISNYLNWYQQKPDGTVKLLIYYTSRLHSGVPSRFSGSGSGTDYSLTINNLEQEDIATY |
| | FCQQGNTRPWTFGGGTKLEIKGGSGGSQVQLVQSGAEVKKPGASVKVSCKASGYTFTNYYMQWVRQAPGQCLEWMGIINPSGGV |
| | TSYAQKFQGRVTMTRDTSTSTVYMELSSLRSEDTAVYYCARGSAYYYDFADYWGQGTLVTVSSDKTHTCPPCPAPEFEGGPSVF |
| | LFPPKPKDTLMISRTPEVTCVVVAVSHEDPEVKFNWYVDGVEVHNAKTKPREEQYGSTYRVVSVLTVLHQDWLNGKEYKCKVSN |
| | KALPAPIEKTISKAKGQPREPQVYTLPPSREEMTKNQVSLSCAVKGFYPSDIAVEWESNGQPENNYKTTPPVLDSDGSFFLVSK |
| | LTVDKSRWQQGNVFSCSVMHEALHNHYTQKSLSLSPG |
| 234 | 1Fab-1scDb-AFc-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPG |
| 235 | 1 Fab-1scDb-AFc-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 236 | 1Fab-1scDb-AFc-4 Chain3: |
| | SYELTQPLSVSVALGQTARITCGGNNIGSKSVHWYQQKPGQAPVLVIYQDKKRPSGIPERFSGSNSGNTATLTISRAQAGDEAD |
| | YYCQVWDDYIVLFGCGTKLTVLGGSGGSEVQLQQSGPELVKPGASVKISCKTSGYTFTEYTMHWVKQSHGKSLEWIGGISPNIG |
| | GTSYNQKFKGKATLTVDKSSSTAYMELRSLTSEDSAVYYCARRGGSFDYWGQGTTLTVSSGGSGGSGGSGGSGGSGGSDIVMTQ |
| | SPATLSVTPGDRVSLSCRASQSISDYLHWYQQKSHESPRLLIKYASQSISGIPSRFSGSGSGSDFTLSINSVEPEDVGVYYCQN |
| | GHSFPLTFGAGTKLELKGGSGGSQVQLVQSGAEVKKPGASVKVSCKASGYTFTNYYMQWVRQAPGQCLEWMGIINPSGGVTSYA |
| | QKFQGRVTMTRDTSTSTVYMELSSLRSEDTAVYYCARGSAYYYDFADYWGQGTLVTVSSDKTHTCPPCPAPEFEGGPSVFLFPP |
| | KPKDTLMISRTPEVTCVVVAVSHEDPEVKFNWYVDGVEVHNAKTKPREEQYGSTYRVVSVLTVLHQDWLNGKEYKCKVSNKALP |
| | APIEKTISKAKGQPREPQVYTLPPSREEMTKNQVSLSCAVKGFYPSDIAVEWESNGQPENNYKTTPPVLDSDGSFFLVSKLTVD |
| | KSRWQQGNVFSCSVMHEALHNHYTQKSLSLSPG |
| 237 | 2scDb-AFc-1 Chain1: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSQSALTQPASVSGSPGQSITISCSGSSS |
| | NIGNNAVNWYQQLPGKAPKLLIYYDDLLPSGVSDRFSGSKSGTSAFLAISGLQSEDEADYYCAAWDDSLNGPVFGGGTKLTVLD |
| | KTHTCPPCPAPEFEGGPSVFLFPPKPKDTLMISRTPEVTCVVVAVSHEDPEVKFNWYVDGVEVHNAKTKPREEQYGSTYRVVSV |
| | LTVLHQDWLNGKEYKCKVSNKALPAPIEKTISKAKGQPREPQVYTLPPSREEMTKNQVSLWCLVKGFYPSDIAVEWESNGQPEN |
| | NYKTTPPVLDSDGSFFLYSKLTVDKSRWQQGNVFSCSVMHEALHNHYTQKSLSLSPG |
| 238 | 2scDb-AFc-1 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSSYELTQPLSVSVALGQTARITCGGNNIG |
| | SKSVHWYQQKPGQAPVLVIYQDKKRPSGIPERFSGSNSGNTATLTISRAQAGDEADYYCQVWDDYIVLFGCGTKLTVLDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 239 | 2scDb-AFc-2 Chain1: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSDIVMTQSPATLSLSPGERATLSCRSSKSLQNVNGNTYLYWFQ |
| | QKPGQSPQLLIYRMSNLNSGVPDRFSGSGSGTEFTLTISSLEPEDFAVYYCMQHLEYPITFGAGTKLEIKGGSGGSGGSGGSGG |
| | SGGSGGSEVQLVESGGGLVKPGGSLKLSCAASGYTFTSYVMHWVRQAPGKGLEWIGYINPYNDGTKYNEKFQGRVTISSDKSIS |
| | TAYMELSSLRSEDTAMYYCARGTYYYGTRVFDYWGQGTLVTVSSGGSGGSGGSDIQMTQSPSTLSASVGDRVTITCRASQSISS |
| | WLAWYQQKPGKAPKLLIYKASSLESGVPSRFSGSGSGTEFTLTISSLQPDDFATYYCQQYNSYPTFGGGTKVEIKDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 240 | 2scDb-AFc-2 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSSYELTQPLSVSVALGQTARITCGGNNIG |
| | SKSVHWYQQKPGQAPVLVIYQDKKRPSGIPERFSGSNSGNTATLTISRAQAGDEADYYCQVWDDYIVLFGCGTKLTVLDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 241 | 2scDb-AFc-3 Chain1: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSDIQMTQTTSSLSASLGDRVTISCRASQD |
| | ISNYLNWYQQKPDGTVKLLIYYTSRLHSGVPSRFSGSGSGTDYSLTINNLEQEDIATYFCQQGNTRPWTFGGGTKLEIKDKTHT |
| | CPPCPAPEFEGGPSVFLFPPKPKDTLMISRTPEVTCVVVAVSHEDPEVKFNWYVDGVEVHNAKTKPREEQYGSTYRVVSVLTVL |
| | HQDWLNGKEYKCKVSNKALPAPIEKTISKAKGQPREPQVYTLPPSREEMTKNQVSLWCLVKGFYPSDIAVEWESNGQPENNYKT |
| | TPPVLDSDGSFFLYSKLTVDKSRWQQGNVFSCSVMHEALHNHYTQKSLSLSPG |
| 242 | 2scDb-AFc-3 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSSYELTQPLSVSVALGQTARITCGGNNIG |
| | SKSVHWYQQKPGQAPVLVIYQDKKRPSGIPERFSGSNSGNTATLTISRAQAGDEADYYCQVWDDYIVLFGCGTKLTVLDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 243 | 2scDb-AFc-4 Chain1: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSDIVMTQSPATLSLSPGERATLSCRSSKSLQNVNGNTYLYWFQQ |
| | KPGQSPQLLIYRMSNLNSGVPDRFSGSGSGTEFTLTISSLEPEDFAVYYCMQHLEYPITFGAGTKLEIKGGSGGSGGSGGSGGS |
| | GGSGGSEVQLVESGGGLVKPGGSLKLSCAASGYTFTSYVMHWVRQAPGKGLEWIGYINPYNDGTKYNEKFQGRVTISSDKSIST |
| | AYMELSSLRSEDTAMYYCARGTYYYGTRVFDYWGQGTLVTVSSGGSGGSGGSDIVMTQSPATLSVTPGDRVSLSCRASQSISDY |
| | LHWYQQKSHESPRLLIKYASQSISGIPSRFSGSGSGSDFTLSINSVEPEDVGVYYCQNGHSFPLTFGAGTKLELKDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 244 | 2scDb-AFc-4 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSSYELTQPLSVSVALGQTARITCGGNNIG |
| | SKSVHWYQQKPGQAPVLVIYQDKKRPSGIPERFSGSNSGNTATLTISRAQAGDEADYYCQVWDDYIVLFGCGTKLTVLDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 245 | 2scDb-AFc-5 Chain1: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSQPVLTQPPSVSVAPGKTARITCGGNNIGSKSVHWYQQK |
| | PGQAPVLVIYYDSDRPSGIPERFSGSNSGNTATLTISRVEAGDEADYYCQVWDTSSDHVLFGGGTKLTVLGGSGGSGGSGGSGG |
| | SGGSGGSQVQLQESGPGLVKPSETLSLTCTVSGGSVSSGSYYWSWIRQPPGKGLEWIGYIYYSGSTNYNPSLKSRVTISVDTSK |
| | NQFSLKLSSVTAADTAVYYCARNPISIPAFDIWGQGTMVTVSSGGSGGSGGSQSALTQPASVSGSPGQSITISCSGSSSNIGNN |
| | AVNWYQQLPGKAPKLLIYYDDLLPSGVSDRFSGSKSGTSAFLAISGLQSEDEADYYCAAWDDSLNGPVFGGGTKLTVLDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLWCLVKGFYPSDIAVEWESNGQPENNYKTT |
| | PPVLDSDGSFFLYSKLTVDKSRWQQGNVFSCSVMHEALHNHYTQKSLSLSPG |
| 246 | 2scDb-AFc-5 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGCGTKLTVLDKTHTCPPCPA |
| | PEFEGGPSVFLFPPKPKDTLMISRTPEVTCVVVAVSHEDPEVKFNWYVDGVEVHNAKTKPREEQYGSTYRVVSVLTVLHQDWLN |
| | GKEYKCKVSNKALPAPIEKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNHYTQKSLSLSPG |
| 247 | 2scDb-AFc-6 Chain1: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSQPVLTQPPSVSVAPGKTARITCGGNNIGSKSVHWYQQKPGQA |
| | PVLVIYYDSDRPSGIPERFSGSNSGNTATLTISRVEAGDEADYYCQVWDTSSDHVLFGGGTKLTVLGGSGGSGGSGGSGGSGGS |
| | GGSQVQLQESGPGLVKPSETLSLTCTVSGGSVSSGSYYWSWIRQPPGKGLEWIGYIYYSGSTNYNPSLKSRVTISVDTSKNQFS |
| | LKLSSVTAADTAVYYCARNPISIPAFDIWGQGTMVTVSSGGSGGSGGSDIQMTQSPSTLSASVGDRVTITCRASQSISSWLAWY |
| | QQKPGKAPKLLIYKASSLESGVPSRFSGSGSGTEFTLTISSLQPDDFATYYCQQYNSYPTFGGGTKVEIKDKTHTCPPCPAPEF |
| | EGGPSVFLFPPKPKDTLMISRTPEVTCVVVAVSHEDPEVKFNWYVDGVEVHNAKTKPREEQYGSTYRVVSVLTVLHQDWLNGKE |
| | YKCKVSNKALPAPIEKTISKAKGQPREPQVYTLPPSREEMTKNQVSLWCLVKGFYPSDIAVEWESNGQPENNYKTTPPVLDSDG |
| | SFFLYSKLTVDKSRWQQGNVFSCSVMHEALHNHYTQKSLSLSPG |
| 248 | 2scDb-AFc-6 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGCGTKLTVLDKTHTCPPCPA |
| | PEFEGGPSVFLFPPKPKDTLMISRTPEVTCVVVAVSHEDPEVKFNWYVDGVEVHNAKTKPREEQYGSTYRVVSVLTVLHQDWLN |
| | GKEYKCKVSNKALPAPIEKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNHYTQKSLSLSPG |
| 249 | 2scDb-AFc-7 Chain1: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSDIQMTQTTSSLSASLGDRVTISCRASQDISNYL |
| | NWYQQKPDGTVKLLIYYTSRLHSGVPSRFSGSGSGTDYSLTINNLEQEDIATYFCQQGNTRPWTFGGGTKLEIKDKTHTCPPCP |
| | APEFEGGPSVFLFPPKPKDTLMISRTPEVTCVVVAVSHEDPEVKFNWYVDGVEVHNAKTKPREEQYGSTYRVVSVLTVLHQDWL |
| | NGKEYKCKVSNKALPAPIEKTISKAKGQPREPQVYTLPPSREEMTKNQVSLWCLVKGFYPSDIAVEWESNGQPENNYKTTPPVL |
| | DSDGSFFLYSKLTVDKSRWQQGNVFSCSVMHEALHNHYTQKSLSLSPG |
| 250 | 2scDb-AFc-7 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGCGTKLTVLDKTHTCPPCPA |
| | PEFEGGPSVFLFPPKPKDTLMISRTPEVTCVVVAVSHEDPEVKFNWYVDGVEVHNAKTKPREEQYGSTYRVVSVLTVLHQDWLN |
| | GKEYKCKVSNKALPAPIEKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNHYTQKSLSLSPG |
| 251 | 2scDb-AFc-8 Chain1: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSQPVLTQPPSVSVAPGKTARITCGGNNIGSKSVHWYQQKPGQAP |
| | VLVIYYDSDRPSGIPERFSGSNSGNTATLTISRVEAGDEADYYCQVWDTSSDHVLFGGGTKLTVLGGSGGSGGSGGSGGSGGSG |
| | GSQVQLQESGPGLVKPSETLSLTCTVSGGSVSSGSYYWSWIRQPPGKGLEWIGYIYYSGSTNYNPSLKSRVTISVDTSKNQFSL |
| | KLSSVTAADTAVYYCARNPISIPAFDIWGQGTMVTVSSGGSGGSGGSDIVMTQSPATLSVTPGDRVSLSCRASQSISDYLHWYQ |
| | QKSHESPRLLIKYASQSISGIPSRFSGSGSGSDFTLSINSVEPEDVGVYYCQNGHSFPLTFGAGTKLELKDKTHTCPPCPAPEF |
| | EGGPSVFLFPPKPKDTLMISRTPEVTCVVVAVSHEDPEVKFNWYVDGVEVHNAKTKPREEQYGSTYRVVSVLTVLHQDWLNGKE |
| | YKCKVSNKALPAPIEKTISKAKGQPREPQVYTLPPSREEMTKNQVSLWCLVKGFYPSDIAVEWESNGQPENNYKTTPPVLDSDG |
| | SFFLYSKLTVDKSRWQQGNVFSCSVMHEALHNHYTQKSLSLSPG |
| 252 | 2scDb-AFc-8 Chain2: |
| | EVQLVQSGAEVKKPGASVKVSCKASGYTFTNYYMQWVRQAPGQCLEWMGIINPSGGVTSYAQKFQGRVTMTRDTSTSTVYMELS |
| | SLRSEDTAVYYCARGSAYYYDFADYWGQGTLVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGCGTKLTVLDKTHTCPPCPA |
| | PEFEGGPSVFLFPPKPKDTLMISRTPEVTCVVVAVSHEDPEVKFNWYVDGVEVHNAKTKPREEQYGSTYRVVSVLTVLHQDWLN |
| | GKEYKCKVSNKALPAPIEKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNHYTQKSLSLSPG |
| 253 | 1scDb-1scFv-AFc-1 Chain1: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSQSALTQPASVSGSPGQSITISCSGSSS |
| | NIGNNAVNWYQQLPGKAPKLLIYYDDLLPSGVSDRFSGSKSGTSAFLAISGLQSEDEADYYCAAWDDSLNGPVFGGGTKLTVLD |
| | KTHTCPPCPAPELLGGPSVFLFPPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLWCLVKGFYPSDIAVEWESNGQPEN |
| | NYKTTPPVLDSDGSFFLYSKLTVDKSRWQQGNVFSCSVMHEALHNHYTQKSLSLSPG |
| 254 | 1scDb-1scFv-AFc-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKGGSGGSGGSGGSGGSGGSGGSEVQLVESGGGLVKPGGSLKLSCAASGYTFTSYVMH |
| | WVRQAPGKGLEWIGYINPYNDGTKYNEKFQGRVTISSDKSISTAYMELSSLRSEDTAMYYCARGTYYYGTRVFDYWGQGTLVTV |
| | SSDKTHTCPPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSREEMTKNQVSLSCAVKGFYPSDIAVEWESNGQ |
| | PENNYKTTPPVLDSDGSFFLVSKLTVDKSRWQQGNVFSCSVMHEALHNHYTQKSLSLSPG |
| 255 | 1scDb-1scFv-AFc-2 Chain1: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSDIVMTQSPATLSLSPGERATLSCRSSKSLQNVNGNTYLYWFQ |
| | QKPGQSPQLLIYRMSNLNSGVPDRFSGSGSGTEFTLTISSLEPEDFAVYYCMQHLEYPITFGAGTKLEIKGGSGGSGGSGGSGG |
| | SGGSGGSEVQLVESGGGLVKPGGSLKLSCAASGYTFTSYVMHWVRQAPGKGLEWIGYINPYNDGTKYNEKFQGRVTISSDKSIS |
| | TAYMELSSLRSEDTAMYYCARGTYYYGTRVFDYWGQGTLVTVSSGGSGGSGGSDIQMTQSPSTLSASVGDRVTITCRASQSISS |
| | WLAWYQQKPGKAPKLLIYKASSLESGVPSRFSGSGSGTEFTLTISSLQPDDFATYYCQQYNSYPTFGGGTKVEIKDKTHTCPPC |
| | PAPELLGGPSVFLFPPKPKDTLMISRTPEVTCVVVDVSHEDPEVKFNWYVDGVEVHNAKTKPREEQYNSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 256 | 1scDb-1scFv-AFc-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKGGSGGSGGSGGSGGSGGSGGSEVQLVESGGGLVKPGGSLKLSCAASGYTFTSYVMH |
| | WVRQAPGKGLEWIGYINPYNDGTKYNEKFQGRVTISSDKSISTAYMELSSLRSEDTAMYYCARGTYYYGTRVFDYWGQGTLVTV |
| | SSDKTHTCPPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSREEMTKNQVSLSCAVKGFYPSDIAVEWESNGQ |
| | PENNYKTTPPVLDSDGSFFLVSKLTVDKSRWQQGNVFSCSVMHEALHNHYTQKSLSLSPG |
| 257 | 1scDb-1scFv-AFc-3 Chain1: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSDIQMTQTTSSLSASLGDRVTISCRASQD |
| | ISNYLNWYQQKPDGTVKLLIYYTSRLHSGVPSRFSGSGSGTDYSLTINNLEQEDIATYFCQQGNTRPWTFGGGTKLEIKDKTHT |
| | CPPCPAPELLGGPSVFLFPPKPKDTLMISRTPEVTCVVVDVSHEDPEVKFNWYVDGVEVHNAKTKPREEQYNSTYRVVSVLTVL |
| | HQDWLNGKEYKCKVSNKALPAPIEKTISKAKGQPREPQVYTLPPSREEMTKNQVSLWCLVKGFYPSDIAVEWESNGQPENNYKT |
| | TPPVLDSDGSFFLYSKLTVDKSRWQQGNVFSCSVMHEALHNHYTQKSLSLSPG |
| 258 | 1scDb-1scFv-AFc-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKGGSGGSGGSGGSGGSGGSGGSEVQLVESGGGLVKPGGSLKLSCAASGYTFTSYVMH |
| | WVRQAPGKGLEWIGYINPYNDGTKYNEKFQGRVTISSDKSISTAYMELSSLRSEDTAMYYCARGTYYYGTRVFDYWGQGTLVTV |
| | SSDKTHTCPPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSREEMTKNQVSLSCAVKGFYPSDIAVEWESNGQ |
| | PENNYKTTPPVLDSDGSFFLVSKLTVDKSRWQQGNVFSCSVMHEALHNHYTQKSLSLSPG |
| 259 | 1scDb-1scFv-AFc-4 Chain1: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSDIVMTQSPATLSLSPGERATLSCRSSKSLQNVNGNTYLYWFQQ |
| | KPGQSPQLLIYRMSNLNSGVPDRFSGSGSGTEFTLTISSLEPEDFAVYYCMQHLEYPITFGAGTKLEIKGGSGGSGGSGGSGGS |
| | GGSGGSEVQLVESGGGLVKPGGSLKLSCAASGYTFTSYVMHWVRQAPGKGLEWIGYINPYNDGTKYNEKFQGRVTISSDKSIST |
| | AYMELSSLRSEDTAMYYCARGTYYYGTRVFDYWGQGTLVTVSSGGSGGSGGSDIVMTQSPATLSVTPGDRVSLSCRASQSISDY |
| | LHWYQQKSHESPRLLIKYASQSISGIPSRFSGSGSGSDFTLSINSVEPEDVGVYYCQNGHSFPLTFGAGTKLELKDKTHTCPPC |
| | PAPELLGGPSVFLFPPKPKDTLMISRTPEVTCVVVDVSHEDPEVKFNWYVDGVEVHNAKTKPREEQYNSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 260 | 1scDb-1scFv-AFc-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKGGSGGSGGSGGSGGSGGSGGSEVQLVESGGGLVKPGGSLKLSCAASGYTFTSYVMH |
| | WVRQAPGKGLEWIGYINPYNDGTKYNEKFQGRVTISSDKSISTAYMELSSLRSEDTAMYYCARGTYYYGTRVFDYWGQGTLVTV |
| | SSDKTHTCPPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSREEMTKNQVSLSCAVKGFYPSDIAVEWESNGQ |
| | PENNYKTTPPVLDSDGSFFLVSKLTVDKSRWQQGNVFSCSVMHEALHNHYTQKSLSLSPG |
| 261 | 2scDb-AFc-9 Chain1: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSQSALTQPASVSGSPGQSITISCSGSSS |
| | NIGNNAVNWYQQLPGKAPKLLIYYDDLLPSGVSDRFSGSKSGTSAFLAISGLQSEDEADYYCAAWDDSLNGPVFGGGTKLTVLD |
| | KTHTCPPCPAPEFEGGPSVFLFPPKPKDTLMISRTPEVTCVVVAVSHEDPEVKFNWYVDGVEVHNAKTKPREEQYGSTYRVVSV |
| | LTVLHQDWLNGKEYKCKVSNKALPAPIEKTISKAKGQPREPQVYTLPPSREEMTKNQVSLWCLVKGFYPSDIAVEWESNGQPEN |
| | NYKTTPPVLDSDGSFFLYSKLTVDKSRWQQGNVFSCSVMHEALHNHYTQKSLSLSPG |
| 262 | 2scDb-AFc-9 Chain2: |
| | QVTLKESGPGILQPSQTLSLTCSFSGFSLRTSGMGVGWIRQPSGKGLEWLAHIWWDDDKRYNPALKSRLTISKDTSSNQVFLKI |
| | ASVDTADTATYYCAQINPAWFAYWGQGTLVTVSAGGSGGSGGSDIVMTQSPATLSLSPGERATLSCRSSKSLQNVNGNTYLYWF |
| | QQKPGQSPQLLIYRMSNLNSGVPDRFSGSGSGTEFTLTISSLEPEDFAVYYCMQHLEYPITFGAGTKLEIKGGSGGSGGSGGSG |
| | GSGGSGGSEVQLVESGGGLVKPGGSLKLSCAASGYTFTSYVMHWVRQAPGKGLEWIGYINPYNDGTKYNEKFQGRVTISSDKSI |
| | STAYMELSSLRSEDTAMYYCARGTYYYGTRVFDYWGQGTLVTVSSGGSGGSGGSDIVLTQSPASLAVSLGQRATISCKASQSVD |
| | FDGDSFMNWYQQKPGQPPKLLIYTTSNLESGIPARFSASGSGTDFTLNIHPVEEEDTATYYCQQSNEDPYTFGGGTKLELKDKT |
| | HTCPPCPAPEFEGGPSVFLFPPKPKDTLMISRTPEVTCVVVAVSHEDPEVKFNWYVDGVEVHNAKTKPREEQYGSTYRVVSVLT |
| | VLHQDWLNGKEYKCKVSNKALPAPIEKTISKAKGQPREPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQKSLSLSPG |
| 263 | 2scDb-AFc-10 Chain1: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSGGSGGSGGSDIVMTQSPATLSLSPGERATLSCRSSKSLQNVNGNTYLYWFQ |
| | QKPGQSPQLLIYRMSNLNSGVPDRFSGSGSGTEFTLTISSLEPEDFAVYYCMQHLEYPITFGAGTKLEIKGGSGGSGGSGGSGG |
| | SGGSGGSEVQLVESGGGLVKPGGSLKLSCAASGYTFTSYVMHWVRQAPGKGLEWIGYINPYNDGTKYNEKFQGRVTISSDKSIS |
| | TAYMELSSLRSEDTAMYYCARGTYYYGTRVFDYWGQGTLVTVSSGGSGGSGGSDIQMTQSPSTLSASVGDRVTITCRASQSISS |
| | WLAWYQQKPGKAPKLLIYKASSLESGVPSRFSGSGSGTEFTLTISSLQPDDFATYYCQQYNSYPTFGGGTKVEIKDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 264 | 2scDb-AFc-10 Chain2: |
| | QVTLKESGPGILQPSQTLSLTCSFSGFSLRTSGMGVGWIRQPSGKGLEWLAHIWWDDDKRYNPALKSRLTISKDTSSNQVFLKI |
| | ASVDTADTATYYCAQINPAWFAYWGQGTLVTVSAGGSGGSGGSDIVMTQSPATLSLSPGERATLSCRSSKSLQNVNGNTYLYWF |
| | QQKPGQSPQLLIYRMSNLNSGVPDRFSGSGSGTEFTLTISSLEPEDFAVYYCMQHLEYPITFGAGTKLEIKGGSGGSGGSGGSG |
| | GSGGSGGSEVQLVESGGGLVKPGGSLKLSCAASGYTFTSYVMHWVRQAPGKGLEWIGYINPYNDGTKYNEKFQGRVTISSDKSI |
| | STAYMELSSLRSEDTAMYYCARGTYYYGTRVFDYWGQGTLVTVSSGGSGGSGGSDIVLTQSPASLAVSLGQRATISCKASQSVD |
| | FDGDSFMNWYQQKPGQPPKLLIYTTSNLESGIPARFSASGSGTDFTLNIHPVEEEDTATYYCQQSNEDPYTFGGGTKLELKDKT |
| | HTCPPCPAPEFEGGPSVFLFPPKPKDTLMISRTPEVTCVVVAVSHEDPEVKFNWYVDGVEVHNAKTKPREEQYGSTYRVVSVLT |
| | VLHQDWLNGKEYKCKVSNKALPAPIEKTISKAKGQPREPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQKSLSLSPG |
| 265 | 2scDb-AFc-11 Chain1: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSGGSGGSGGSDIVMTQSPATLSLSPGERATLSCRSSKSLQNVNGNTYLY |
| | WFQQKPGQSPQLLIYRMSNLNSGVPDRFSGSGSGTEFTLTISSLEPEDFAVYYCMQHLEYPITFGAGTKLEIKGGSGGSGGSGG |
| | SGGSGGSGGSEVQLVESGGGLVKPGGSLKLSCAASGYTFTSYVMHWVRQAPGKGLEWIGYINPYNDGTKYNEKFQGRVTISSDK |
| | SISTAYMELSSLRSEDTAMYYCARGTYYYGTRVFDYWGQGTLVTVSSGGSGGSGGSDIQMTQTTSSLSASLGDRVTISCRASQD |
| | ISNYLNWYQQKPDGTVKLLIYYTSRLHSGVPSRFSGSGSGTDYSLTINNLEQEDIATYFCQQGNTRPWTFGGGTKLEIKDKTHT |
| | CPPCPAPEFEGGPSVFLFPPKPKDTLMISRTPEVTCVVVAVSHEDPEVKFNWYVDGVEVHNAKTKPREEQYGSTYRVVSVLTVL |
| | HQDWLNGKEYKCKVSNKALPAPIEKTISKAKGQPREPQVYTLPPSREEMTKNQVSLWCLVKGFYPSDIAVEWESNGQPENNYKT |
| | TPPVLDSDGSFFLYSKLTVDKSRWQQGNVFSCSVMHEALHNHYTQKSLSLSPG |
| 266 | 2scDb-AFc-11 Chain2: |
| | QVTLKESGPGILQPSQTLSLTCSFSGFSLRTSGMGVGWIRQPSGKGLEWLAHIWWDDDKRYNPALKSRLTISKDTSSNQVFLKI |
| | ASVDTADTATYYCAQINPAWFAYWGQGTLVTVSAGGSGGSGGSDIVMTQSPATLSLSPGERATLSCRSSKSLQNVNGNTYLYWF |
| | QQKPGQSPQLLIYRMSNLNSGVPDRFSGSGSGTEFTLTISSLEPEDFAVYYCMQHLEYPITFGAGTKLEIKGGSGGSGGSGGSG |
| | GSGGSGGSEVQLVESGGGLVKPGGSLKLSCAASGYTFTSYVMHWVRQAPGKGLEWIGYINPYNDGTKYNEKFQGRVTISSDKSI |
| | STAYMELSSLRSEDTAMYYCARGTYYYGTRVFDYWGQGTLVTVSSGGSGGSGGSDIVLTQSPASLAVSLGQRATISCKASQSVD |
| | FDGDSFMNWYQQKPGQPPKLLIYTTSNLESGIPARFSASGSGTDFTLNIHPVEEEDTATYYCQQSNEDPYTFGGGTKLELKDKT |
| | HTCPPCPAPEFEGGPSVFLFPPKPKDTLMISRTPEVTCVVVAVSHEDPEVKFNWYVDGVEVHNAKTKPREEQYGSTYRVVSVLT |
| | VLHQDWLNGKEYKCKVSNKALPAPIEKTISKAKGQPREPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQKSLSLSPG |
| 267 | 2scDb-AFc-12 Chain1: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSGGSGGSGGSDIVMTQSPATLSLSPGERATLSCRSSKSLQNVNGNTYLYWFQQ |
| | KPGQSPQLLIYRMSNLNSGVPDRFSGSGSGTEFTLTISSLEPEDFAVYYCMQHLEYPITFGAGTKLEIKGGSGGSGGSGGSGGS |
| | GGSGGSEVQLVESGGGLVKPGGSLKLSCAASGYTFTSYVMHWVRQAPGKGLEWIGYINPYNDGTKYNEKFQGRVTISSDKSIST |
| | AYMELSSLRSEDTAMYYCARGTYYYGTRVFDYWGQGTLVTVSSGGSGGSGGSDIVMTQSPATLSVTPGDRVSLSCRASQSISDY |
| | LHWYQQKSHESPRLLIKYASQSISGIPSRFSGSGSGSDFTLSINSVEPEDVGVYYCQNGHSFPLTFGAGTKLELKDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 268 | 2scDb-AFc-12 Chain2: |
| | QVTLKESGPGILQPSQTLSLTCSFSGFSLRTSGMGVGWIRQPSGKGLEWLAHIWWDDDKRYNPALKSRLTISKDTSSNQVFLKI |
| | ASVDTADTATYYCAQINPAWFAYWGQGTLVTVSAGGSGGSGGSDIVMTQSPATLSLSPGERATLSCRSSKSLQNVNGNTYLYWF |
| | QQKPGQSPQLLIYRMSNLNSGVPDRFSGSGSGTEFTLTISSLEPEDFAVYYCMQHLEYPITFGAGTKLEIKGGSGGSGGSGGSG |
| | GSGGSGGSEVQLVESGGGLVKPGGSLKLSCAASGYTFTSYVMHWVRQAPGKGLEWIGYINPYNDGTKYNEKFQGRVTISSDKSI |
| | STAYMELSSLRSEDTAMYYCARGTYYYGTRVFDYWGQGTLVTVSSGGSGGSGGSDIVLTQSPASLAVSLGQRATISCKASQSVD |
| | FDGDSFMNWYQQKPGQPPKLLIYTTSNLESGIPARFSASGSGTDFTLNIHPVEEEDTATYYCQQSNEDPYTFGGGTKLELKDKT |
| | HTCPPCPAPEFEGGPSVFLFPPKPKDTLMISRTPEVTCVVVAVSHEDPEVKFNWYVDGVEVHNAKTKPREEQYGSTYRVVSVLT |
| | VLHQDWLNGKEYKCKVSNKALPAPIEKTISKAKGQPREPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQKSLSLSPG |
| 269 | 2tascFv-AFc-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSQSALTQPASVSGSPGQSITISCSGSSSNIGNNAVNWYQQLPGKAPKLLIYYDDLLPSGVSDRFSGSKSGTSAFLAISGLQ |
| | SEDEADYYCAAWDDSLNGPVFGGGTKLTVLGGSGGSGGSGGSGGSGGSGGSQVQLVESGGGLVKPGGSLRLSCAASGFTFSSYG |
| | MHWVRQAPGKGLEWVAFIRYDGSNKYYADSVKGRFTISRDNSKNTLYLQMNSLRAEDTAVYYCAKDRGLGDGTYFDYWGQGTTV |
| | TVSSDKTHTCPPCPAPEFEGGPSVFLFPPKPKDTLMISRTPEVTCVVVAVSHEDPEVKFNWYVDGVEVHNAKTKPREEQYGSTY |
| | RVVSVLTVLHQDWLNGKEYKCKVSNKALPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKSLSLSPG |
| 270 | 2tascFv-AFc-1 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSSYELTQPLSVSVALGQTARITCGGNNIGSKSVHWYQQKPGQAPVLVIYQDKKRPSGIPERFSGSNSGNTATLTISRAQAG |
| | DEADYYCQVWDDYIVLFGCGTKLTVLGGSGGSGGSGGSGGSGGSGGSQVQLVQSGAEVKKPGASVKVSCKASGYTFTNYYMQWV |
| | RQAPGQCLEWMGIINPSGGVTSYAQKFQGRVTMTRDTSTSTVYMELSSLRSEDTAVYYCARGSAYYYDFADYWGQGTLVTVSSD |
| | KTHTCPPCPAPEFEGGPSVFLFPPKPKDTLMISRTPEVTCVVVAVSHEDPEVKFNWYVDGVEVHNAKTKPREEQYGSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 271 | 2tascFv-AFc-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIQMTQSPSTLSASVGDRVTITCRASQSISSWLAWYQQKPGKAPKLLIYKASSLESGVPSRFSGSGSGTEFTLTISSLQP |
| | DDFATYYCQQYNSYPTFGGGTKVEIKGGSGGSGGSGGSGGSGGSGGSQVQLQQWGAGLLKPSETLSLTCAVYGGSFSGYYWSWI |
| | RQPPGKGLEWIGEIDHSGSTNYNPSLKSRVTISVDTSKNQFSLKLSSVTAADTAVYYCARARGPWSFDPWGQGTLVTVSSDKTH |
| | TCPPCPAPEFEGGPSVFLFPPKPKDTLMISRTPEVTCVVVAVSHEDPEVKFNWYVDGVEVHNAKTKPREEQYGSTYRVVSVLTV |
| | LHQDWLNGKEYKCKVSNKALPAPIEKTISKAKGQPREPQVYTLPPSREEMTKNQVSLWCLVKGFYPSDIAVEWESNGQPENNYK |
| | TTPPVLDSDGSFFLYSKLTVDKSRWQQGNVFSCSVMHEALHNHYTQKSLSLSPG |
| 272 | 2tascFv-AFc-2 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSSYELTQPLSVSVALGQTARITCGGNNIGSKSVHWYQQKPGQAPVLVIYQDKKRPSGIPERFSGSNSGNTATLTISRAçAG |
| | DEADYYCQVWDDYIVLFGCGTKLTVLGGSGGSGGSGGSGGSGGSGGSQVQLVQSGAEVKKPGASVKVSCKASGYTFTNYYMQWV |
| | RQAPGQCLEWMGIINPSGGVTSYAQKFQGRVTMTRDTSTSTVYMELSSLRSEDTAVYYCARGSAYYYDFADYWGQGTLVTVSSD |
| | KTHTCPPCPAPEFEGGPSVFLFPPKPKDTLMISRTPEVTCVVVAVSHEDPEVKFNWYVDGVEVHNAKTKPREEQYGSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 273 | 2tascFv-AFc-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIQMTQTTSSLSASLGDRVTISCRASQDISNYLNWYQQKPDGTVKLLIYYTSRLHSGVPSRFSGSGSGTDYSLTINNLEQ |
| | EDIATYFCQQGNTRPWTFGGGTKLEIKGGSGGSGGSGGSGGSGGSGGSQVQLQQSGPELVKPGASVKMSCKASGYTFTDYVINW |
| | GKQRSGQGLEWIGEIYPGSGTNYYNEKFKAKATLTADKSSNIAYMQLSSLTSEDSAVYFCARRGRYGLYAMDYWGQGTSVTVSS |
| | DKTHTCPPCPAPEFEGGPSVFLFPPKPKDTLMISRTPEVTCVVVAVSHEDPEVKFNWYVDGVEVHNAKTKPREEQYGSTYRVVS |
| | VLTVLHQDWLNGKEYKCKVSNKALPAPIEKTISKAKGQPREPQVYTLPPSREEMTKNQVSLWCLVKGFYPSDIAVEWESNGQPE |
| | NNYKTTPPVLDSDGSFFLYSKLTVDKSRWQQGNVFSCSVMHEALHNHYTQKSLSLSPG |
| 274 | 2tascFv-AFc-3 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSSYELTQPLSVSVALGQTARITCGGNNIGSKSVHWYQQKPGQAPVLVIYQDKKRPSGIPERFSGSNSGNTATLTISRAçAG |
| | DEADYYCQVWDDYIVLFGCGTKLTVLGGSGGSGGSGGSGGSGGSGGSQVQLVQSGAEVKKPGASVKVSCKASGYTFTNYYMQWV |
| | RQAPGQCLEWMGIINPSGGVTSYAQKFQGRVTMTRDTSTSTVYMELSSLRSEDTAVYYCARGSAYYYDFADYWGQGTLVTVSSD |
| | KTHTCPPCPAPEFEGGPSVFLFPPKPKDTLMISRTPEVTCVVVAVSHEDPEVKFNWYVDGVEVHNAKTKPREEQYGSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 275 | 2tascFv-AFc-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIVMTQSPATLSVTPGDRVSLSCRASQSISDYLHWYQQKSHESPRLLIKYASQSISGIPSRFSGSGSGSDFTLSINSVEP |
| | EDVGVYYCQNGHSFPLTFGAGTKLELKGGSGGSGGSGGSGGSGGSGGSEVQLQQSGPELVKPGASVKISCKTSGYTFTEYTMHW |
| | VKQSHGKSLEWIGGISPNIGGTSYNQKFKGKATLTVDKSSSTAYMELRSLTSEDSAVYYCARRGGSFDYWGQGTTLTVSSDKTH |
| | TCPPCPAPEFEGGPSVFLFPPKPKDTLMISRTPEVTCVVVAVSHEDPEVKFNWYVDGVEVHNAKTKPREEQYGSTYRVVSVLTV |
| | LHQDWLNGKEYKCKVSNKALPAPIEKTISKAKGQPREPQVYTLPPSREEMTKNQVSLWCLVKGFYPSDIAVEWESNGQPENNYK |
| | TTPPVLDSDGSFFLYSKLTVDKSRWQQGNVFSCSVMHEALHNHYTQKSLSLSPG |
| 276 | 2tascFv-AFc-4 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSSYELTQPLSVSVALGQTARITCGGNNIGSKSVHWYQQKPGQAPVLVIYQDKKRPSGIPERFSGSNSGNTATLTISRAQAG |
| | DEADYYCQVWDDYIVLFGCGTKLTVLGGSGGSGGSGGSGGSGGSGGSQVQLVQSGAEVKKPGASVKVSCKASGYTFTNYYMQWV |
| | RQAPGQCLEWMGIINPSGGVTSYAQKFQGRVTMTRDTSTSTVYMELSSLRSEDTAVYYCARGSAYYYDFADYWGQGTLVTVSSD |
| | KTHTCPPCPAPEFEGGPSVFLFPPKPKDTLMISRTPEVTCVVVAVSHEDPEVKFNWYVDGVEVHNAKTKPREEQYGSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 277 | 2tascFv-AFc-5 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSQ |
| | SALTQPASVSGSPGQSITISCSGSSSNIGNNAVNWYQQLPGKAPKLLIYYDDLLPSGVSDRFSGSKSGTSAFLAISGLQSEDEA |
| | DYYCAAWDDSLNGPVFGGGTKLTVLGGSGGSGGSGGSGGSGGSGGSQVQLVESGGGLVKPGGSLRLSCAASGFTFSSYGMHWVR |
| | QAPGKGLEWVAFIRYDGSNKYYADSVKGRFTISRDNSKNTLYLQMNSLRAEDTAVYYCAKDRGLGDGTYFDYWGQGTTVTVSSD |
| | KTHTCPPCPAPEFEGGPSVFLFPPKPKDTLMISRTPEVTCVVVAVSHEDPEVKFNWYVDGVEVHNAKTKPREEQYGSTYRVVSV |
| | LTVLHQDWLNGKEYKCKVSNKALPAPIEKTISKAKGQPREPQVYTLPPSREEMTKNQVSLWCLVKGFYPSDIAVEWESNGQPEN |
| | NYKTTPPVLDSDGSFFLYSKLTVDKSRWQQGNVFSCSVMHEALHNHYTQKSLSLSPG |
| 278 | 2tascFv-AFc-5 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSS |
| | YELTQPLSVSVALGQTARITCGGNNIGSKSVHWYQQKPGQAPVLVIYQDKKRPSGIPERFSGSNSGNTATLTISRAQAGDEADY |
| | YCQVWDDYIVLFGCGTKLTVLGGSGGSGGSGGSGGSGGSGGSQVQLVQSGAEVKKPGASVKVSCKASGYTFTNYYMQWVRQAPG |
| | QCLEWMGIINPSGGVTSYAQKFQGRVTMTRDTSTSTVYMELSSLRSEDTAVYYCARGSAYYYDFADYWGQGTLVTVSSDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 279 | 2tascFv-AFc-6 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSD |
| | IQMTQSPSTLSASVGDRVTITCRASQSISSWLAWYQQKPGKAPKLLIYKASSLESGVPSRFSGSGSGTEFTLTISSLQPDDFAT |
| | YYCQQYNSYPTFGGGTKVEIKGGSGGSGGSGGSGGSGGSGGSQVQLQQWGAGLLKPSETLSLTCAVYGGSFSGYYWSWIRQPPG |
| | KGLEWIGEIDHSGSTNYNPSLKSRVTISVDTSKNQFSLKLSSVTAADTAVYYCARARGPWSFDPWGQGTLVTVSSDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 280 | 2tascFv-AFc-6 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKI |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSS |
| | YELTQPLSVSVALGQTARITCGGNNIGSKSVHWYQQKPGQAPVLVIYQDKKRPSGIPERFSGSNSGNTATLTISRAQAGDEADY |
| | YCQVWDDYIVLFGCGTKLTVLGGSGGSGGSGGSGGSGGSGGSQVQLVQSGAEVKKPGASVKVSCKASGYTFTNYYMQWVRQAPG |
| | QCLEWMGIINPSGGVTSYAQKFQGRVTMTRDTSTSTVYMELSSLRSEDTAVYYCARGSAYYYDFADYWGQGTLVTVSSDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 281 | 2tascFv-AFc-7 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSD |
| | IQMTQTTSSLSASLGDRVTISCRASQDISNYLNWYQQKPDGTVKLLIYYTSRLHSGVPSRFSGSGSGTDYSLTINNLEQEDIAT |
| | YFCQQGNTRPWTFGGGTKLEIKGGSGGSGGSGGSGGSGGSGGSQVQLQQSGPELVKPGASVKMSCKASGYTFTDYVINWGKQRS |
| | GQGLEWIGEIYPGSGTNYYNEKFKAKATLTADKSSNIAYMQLSSLTSEDSAVYFCARRGRYGLYAMDYWGQGTSVTVSSDKTHT |
| | CPPCPAPEFEGGPSVFLFPPKPKDTLMISRTPEVTCVVVAVSHEDPEVKFNWYVDGVEVHNAKTKPREEQYGSTYRVVSVLTVL |
| | HQDWLNGKEYKCKVSNKALPAPIEKTISKAKGQPREPQVYTLPPSREEMTKNQVSLWCLVKGFYPSDIAVEWESNGQPENNYKT |
| | TPPVLDSDGSFFLYSKLTVDKSRWQQGNVFSCSVMHEALHNHYTQKSLSLSPG |
| 282 | 2tascFv-AFc-7 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSS |
| | YELTQPLSVSVALGQTARITCGGNNIGSKSVHWYQQKPGQAPVLVIYQDKKRPSGIPERFSGSNSGNTATLTISRAQAGDEADY |
| | YCQVWDDYIVLFGCGTKLTVLGGSGGSGGSGGSGGSGGSGGSQVQLVQSGAEVKKPGASVKVSCKASGYTFTNYYMQWVRQAPG |
| | QCLEWMGIINPSGGVTSYAQKFQGRVTMTRDTSTSTVYMELSSLRSEDTAVYYCARGSAYYYDFADYWGQGTLVTVSSDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 283 | 2tascFv-AFc-8 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSD |
| | IVMTQSPATLSVTPGDRVSLSCRASQSISDYLHWYQQKSHESPRLLIKYASQSISGIPSRFSGSGSGSDFTLSINSVEPEDVGV |
| | YYCQNGHSFPLTFGAGTKLELKGGSGGSGGSGGSGGSGGSGGSEVQLQQSGPELVKPGASVKISCKTSGYTFTEYTMHWVKQSH |
| | GKSLEWIGGISPNIGGTSYNQKFKGKATLTVDKSSSTAYMELRSLTSEDSAVYYCARRGGSFDYWGQGTTLTVSSDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 284 | 2tascFv-AFc-8 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKI |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGSS |
| | YELTQPLSVSVALGQTARITCGGNNIGSKSVHWYQQKPGQAPVLVIYQDKKRPSGIPERFSGSNSGNTATLTISRAQAGDEADY |
| | YCQVWDDYIVLFGCGTKLTVLGGSGGSGGSGGSGGSGGSGGSQVQLVQSGAEVKKPGASVKVSCKASGYTFTNYYMQWVRQAPG |
| | QCLEWMGIINPSGGVTSYAQKFQGRVTMTRDTSTSTVYMELSSLRSEDTAVYYCARGSAYYYDFADYWGQGTLVTVSSDKTHTC |
| | PPCPAPEFEGGPSVFLFPPKPKDTLMISRTPEVTCVVVAVSHEDPEVKFNWYVDGVEVHNAKTKPREEQYGSTYRVVSVLTVLH |
| | QDWLNGKEYKCKVSNKALPAPIEKTISKAKGQPREPQVYTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPG |
| 285 | 1tascFv-1scFv-AFc-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSQSALTQPASVSGSPGQSITISCSGSSSNIGNNAVNWYQQLPGKAPKLLIYYDDLLPSGVSDRFSGSKSGTSAFLAISGLQ |
| | SEDEADYYCAAWDDSLNGPVFGGGTKLTVLGGSGGSGGSGGSGGSGGSGGSQVQLVESGGGLVKPGGSLRLSCAASGFTFSSYG |
| | MHWVRQAPGKGLEWVAFIRYDGSNKYYADSVKGRFTISRDNSKNTLYLQMNSLRAEDTAVYYCAKDRGLGDGTYFDYWGQGTTV |
| | TVSSDKTHTCPPCPAPELLGGPSVFLFPPKPKDTLMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEALHNHYTQKSLSLSPG |
| 286 | 1tascFv-1scFv-AFc-1 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKD |
| | KTHTCPPCPAPELLGGPSVFLFPPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 287 | 1tascFv-1scFv-AFc-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIQMTQSPSTLSASVGDRVTITCRASQSISSWLAWYQQKPGKAPKLLIYKASSLESGVPSRFSGSGSGTEFTLTISSLQP |
| | DDFATYYCQQYNSYPTFGGGTKVEIKGGSGGSGGSGGSGGSGGSGGSQVQLQQWGAGLLKPSETLSLTCAVYGGSFSGYYWSWI |
| | RQPPGKGLEWIGEIDHSGSTNYNPSLKSRVTISVDTSKNQFSLKLSSVTAADTAVYYCARARGPWSFDPWGQGTLVTVSSDKTH |
| | TCPPCPAPELLGGPSVFLFPPKPKDTLMISRTPEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMTKNQVSLWCLVKGFYPSDIAVEWESNGQPENNYK |
| | TTPPVLDSDGSFFLYSKLTVDKSRWQQGNVFSCSVMHEALHNHYTQKSLSLSPG |
| 288 | 1tascFv-1scFv-AFc-2 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKD |
| | KTHTCPPCPAPELLGGPSVFLFPPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 289 | 1tascFv-1scFv-AFc-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIQMTQTTSSLSASLGDRVTISCRASQDISNYLNWYQQKPDGTVKLLIYYTSRLHSGVPSRFSGSGSGTDYSLTINNLEQ |
| | EDIATYFCQQGNTRPWTFGGGTKLEIKGGSGGSGGSGGSGGSGGSGGSQVQLQQSGPELVKPGASVKMSCKASGYTFTDYVINW |
| | GKQRSGQGLEWIGEIYPGSGTNYYNEKFKAKATLTADKSSNIAYMQLSSLTSEDSAVYFCARRGRYGLYAMDYWGQGTSVTVSS |
| | DKTHTCPPCPAPELLGGPSVFLFPPKPKDTLMISRTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSREEMTKNQVSLWCLVKGFYPSDIAVEWESNGQPE |
| | NNYKTTPPVLDSDGSFFLYSKLTVDKSRWQQGNVFSCSVMHEALHNHYTQKSLSLSPG |
| 290 | 1tascFv-1scFv-AFc-3 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKD |
| | KTHTCPPCPAPELLGGPSVFLFPPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 291 | 1tascFv-1scFv-AFc-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSDIVMTQSPATLSVTPGDRVSLSCRASQSISDYLHWYQQKSHESPRLLIKYASQSISGIPSRFSGSGSGSDFTLSINSVEP |
| | EDVGVYYCQNGHSFPLTFGAGTKLELKGGSGGSGGSGGSGGSGGSGGSEVQLQQSGPELVKPGASVKISCKTSGYTFTEYTMHW |
| | VKQSHGKSLEWIGGISPNIGGTSYNQKFKGKATLTVDKSSSTAYMELRSLTSEDSAVYYCARRGGSFDYWGQGTTLTVSSDKTH |
| | TCPPCPAPELLGGPSVFLFPPKPKDTLMISRTPEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMTKNQVSLWCLVKGFYPSDIAVEWESNGQPENNYK |
| | TTPPVLDSDGSFFLYSKLTVDKSRWQQGNVFSCSVMHEALHNHYTQKSLSLSPG |
| 292 | 1tascFv-1scFv-AFc-4 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKD |
| | KTHTCPPCPAPELLGGPSVFLFPPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLSCAVKGFYPSDIAVEWESNGQPEN |
| | NYKTTPPVLDSDGSFFLVSKLTVDKSRWQQGNVFSCSVMHEALHNHYTQKSLSLSPG |
| 293 | 1scDb-2Fab-AFc-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPEFEGGPSVFLFPPKPKDTLMISRTPEVTCVVVAVSHEDPEVKFNWYVDGVEVHNAKTKPREEQYGSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 294 | 1scDb-2Fab-AFc-1 Chain2: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTQVQLVQSGAEVKKPGASVKVSCK |
| | ASGYTFTNYYMQWVRQAPGQGLEWMGIINPSGGVTSYAQKFQGRVTMTRDTSTSTVYMELSSLRSEDTAVYYCARGSAYYYDFA |
| | DYWGQGTLVTVSSASTKGPSVFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPEFEGGPSVFLFPPKPKDTLMISRTPEVTCVVVAVSHEDPE |
| | VKFNWYVDGVEVHNAKTKPREEQYGSTYRVVSVLTVLHQDWLNGKEYKCKVSNKALPAPIEKTISKAKGQPREPQVYTLPPSRE |
| | EMTKNQVSLSCAVKGFYPSDIAVEWESNGQPENNYKTTPPVLDSDGSFFLVSKLTVDKSRWQQGNVFSCSVMHEALHNHYTQKS |
| | LSLSPG |
| 295 | 1scDb-2Fab-AFc-1 Chain3: |
| | QSALTQPASVSGSPGQSITISCSGSSSNIGNNAVNWYQQLPGKAPKLLIYYDDLLPSGVSDRFSGSKSGTSAFLAISGLQSEDE |
| | ADYYCAAWDDSLNGPVFGGGTKLTVLGQPKAAPSVTLFPPSSEELQANKATLVCLISDFYPGAVTVAWKADSSPVKAGVETTTP |
| | SKQSNNKYAASSYLSLTPEQWKSHRSYSCQVTHEGSTVEKTVAPTECSGGGGSSYELTQPLSVSVALGQTARITCGGNNIGSKS |
| | VHWYQQKPGQAPVLVIYQDKKRPSGIPERFSGSNSGNTATLTISRAQAGDEADYYCQVWDDYIVLFGGGTKLTVLGQPKAAPSV |
| | TLFPPSSEELQANKATLVCLISDFYPGAVTVAWKADSSPVKAGVETTTPSKQSNNKYAASSYLSLTPEQWKSHRSYSCQVTHEG |
| | STVEKTVAPTECS |
| 296 | 1scDb-2Fab-AFc-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPEFEGGPSVFLFPPKPKDTLMISRTPEVTCVVVAVSHEDPEVKFNWYVDGVEVHNAKTKPREEQYGSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 297 | 1scDb-2Fab-AFc-2 Chain2: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTQVQLVQSGAEVKKPGASVKVSCKASGY |
| | TFTNYYMQWVRQAPGQGLEWMGIINPSGGVTSYAQKFQGRVTMTRDTSTSTVYMELSSLRSEDTAVYYCARGSAYYYDFADYWG |
| | QGTLVTVSSASTKGPSVFPLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPEFEGGPSVFLFPPKPKDTLMISRTPEVTCVVVAVSHEDPEVKFN |
| | WYVDGVEVHNAKTKPREEQYGSTYRVVSVLTVLHQDWLNGKEYKCKVSNKALPAPIEKTISKAKGQPREPQVYTLPPSREEMTK |
| | NQVSLSCAVKGFYPSDIAVEWESNGQPENNYKTTPPVLDSDGSFFLVSKLTVDKSRWQQGNVFSCSVMHEALHNHYTQKSLSLS |
| | PG |
| 298 | 1scDb-2Fab-AFc-2 Chain3: |
| | DIQMTQSPSTLSASVGDRVTITCRASQSISSWLAWYQQKPGKAPKLLIYKASSLESGVPSRFSGSGSGTEFTLTISSLQPDDFA |
| | TYYCQQYNSYPTFGGGTKVEIKRTVAAPSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGECGGGGSSYELTQPLSVSVALGQTARITCGGNNIGSKSVHW |
| | YQQKPGQAPVLVIYQDKKRPSGIPERFSGSNSGNTATLTISRAQAGDEADYYCQVWDDYIVLFGGGTKLTVLGQPKAAPSVTLF |
| | PPSSEELQANKATLVCLISDFYPGAVTVAWKADSSPVKAGVETTTPSKQSNNKYAASSYLSLTPEQWKSHRSYSCQVTHEGSTV |
| | EKTVAPTECS |
| 299 | 1scDb-2Fab-AFc-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPEFEGGPSVFLFPPKPKDTLMISRTPEVTCVVVAVSHEDPEVKFNWYVDGVEVHNAKTKPREEQYGSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 300 | 1scDb-2Fab-AFc-3 Chain2: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTQVQLVQSGAEVKKPGASVKVSCKA |
| | SGYTFTNYYMQWVRQAPGQGLEWMGIINPSGGVTSYAQKFQGRVTMTRDTSTSTVYMELSSLRSEDTAVYYCARGSAYYYDFAD |
| | YWGQGTLVTVSSASTKGPSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPEFEGGPSVFLFPPKPKDTLMISRTPEVTCVVVAVSHEDPEV |
| | KFNWYVDGVEVHNAKTKPREEQYGSTYRVVSVLTVLHQDWLNGKEYKCKVSNKALPAPIEKTISKAKGQPREPQVYTLPPSREE |
| | MTKNQVSLSCAVKGFYPSDIAVEWESNGQPENNYKTTPPVLDSDGSFFLVSKLTVDKSRWQQGNVFSCSVMHEALHNHYTQKSL |
| | SLSPG |
| 301 | 1scDb-2Fab-AFc-3 Chain3: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGECGGGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGGGTKLTVLGQPKAAPSVTL |
| | FPPSSEELQANKATLVCLISDFYPGAVTVAWKADSSPVKAGVETTTPSKQSNNKYAASSYLSLTPEQWKSHRSYSCQVTHEGST |
| | VEKTVAPTECS |
| 302 | 1scDb-2Fab-AFc-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPEFEGGPSVFLFPPKPKDTLMISRTPEVTCVVVAVSHEDPEVKFNWYVDGVEVHNAKTKPREEQYGSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 303 | 1scDb-2Fab-AFc-4 Chain2: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKKVEPKSCDKTHTQVQLVQSGAEVKKPGASVKVSCKASGYT |
| | FTNYYMQWVRQAPGQGLEWMGIINPSGGVTSYAQKFQGRVTMTRDTSTSTVYMELSSLRSEDTAVYYCARGSAYYYDFADYWGQ |
| | GTLVTVSSASTKGPSVFPLAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPEFEGGPSVFLFPPKPKDTLMISRTPEVTCVVVAVSHEDPEVKFNW |
| | YVDGVEVHNAKTKPREEQYGSTYRVVSVLTVLHQDWLNGKEYKCKVSNKALPAPIEKTISKAKGQPREPQVYTLPPSREEMTKN |
| | QVSLSCAVKGFYPSDIAVEWESNGQPENNYKTTPPVLDSDGSFFLVSKLTVDKSRWQQGNVFSCSVMHEALHNHYTQKSLSLSP |
| | G |
| 304 | 1scDb-2Fab-AFc-4 Chain3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGECGGGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGGGTKLTVLGQPKAAPSVTL |
| | FPPSSEELQANKATLVCLISDFYPGAVTVAWKADSSPVKAGVETTTPSKQSNNKYAASSYLSLTPEQWKSHRSYSCQVTHEGST |
| | VEKTVAPTECS |
| 305 | 1scDb-2Fab-AFc-5 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSQPVLTQPPSVSVAPGKTARITCGGNNIGSKSVH |
| | WYQQKPGQAPVLVIYYDSDRPSGIPERFSGSNSGNTATLTISRVEAGDEADYYCQVWDTSSDHVLFGGGTKLTVLDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 306 | 1scDb-2Fab-AFc-5 Chain2: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTQVQLVQSGAEVKKPGASVKVSCK |
| | ASGYTFTNYYMQWVRQAPGQGLEWMGIINPSGGVTSYAQKFQGRVTMTRDTSTSTVYMELSSLRSEDTAVYYCARGSAYYYDFA |
| | DYWGQGTLVTVSSASTKGPSVFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPEFEGGPSVFLFPPKPKDTLMISRTPEVTCVVVAVSHEDPE |
| | VKFNWYVDGVEVHNAKTKPREEQYGSTYRVVSVLTVLHQDWLNGKEYKCKVSNKALPAPIEKTISKAKGQPREPQVYTLPPSRE |
| | EMTKNQVSLSCAVKGFYPSDIAVEWESNGQPENNYKTTPPVLDSDGSFFLVSKLTVDKSRWQQGNVFSCSVMHEALHNHYTQKS |
| | LSLSPG |
| 307 | 1scDb-2Fab-AFc-5 Chain3: |
| | QSALTQPASVSGSPGQSITISCSGSSSNIGNNAVNWYQQLPGKAPKLLIYYDDLLPSGVSDRFSGSKSGTSAFLAISGLQSEDE |
| | ADYYCAAWDDSLNGPVFGGGTKLTVLGQPKAAPSVTLFPPSSEELQANKATLVCLISDFYPGAVTVAWKADSSPVKAGVETTTP |
| | SKQSNNKYAASSYLSLTPEQWKSHRSYSCQVTHEGSTVEKTVAPTECSGGGGSSYELTQPLSVSVALGQTARITCGGNNIGSKS |
| | VHWYQQKPGQAPVLVIYQDKKRPSGIPERFSGSNSGNTATLTISRAQAGDEADYYCQVWDDYIVLFGGGTKLTVLGQPKAAPSV |
| | TLFPPSSEELQANKATLVCLISDFYPGAVTVAWKADSSPVKAGVETTTPSKQSNNKYAASSYLSLTPEQWKSHRSYSCQVTHEG |
| | STVEKTVAPTECS |
| 308 | 1scDb-2Fab-AFc-6 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSQPVLTQPPSVSVAPGKTARITCGGNNIGSKSVH |
| | WYQQKPGQAPVLVIYYDSDRPSGIPERFSGSNSGNTATLTISRVEAGDEADYYCQVWDTSSDHVLFGGGTKLTVLDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 309 | 1scDb-2Fab-AFc-6 Chain2: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTQVQLVQSGAEVKKPGASVKVSCKASGY |
| | TFTNYYMQWVRQAPGQGLEWMGIINPSGGVTSYAQKFQGRVTMTRDTSTSTVYMELSSLRSEDTAVYYCARGSAYYYDFADYWG |
| | QGTLVTVSSASTKGPSVFPLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPEFEGGPSVFLFPPKPKDTLMISRTPEVTCVVVAVSHEDPEVKFN |
| | WYVDGVEVHNAKTKPREEQYGSTYRVVSVLTVLHQDWLNGKEYKCKVSNKALPAPIEKTISKAKGQPREPQVYTLPPSREEMTK |
| | NQVSLSCAVKGFYPSDIAVEWESNGQPENNYKTTPPVLDSDGSFFLVSKLTVDKSRWQQGNVFSCSVMHEALHNHYTQKSLSLS |
| | PG |
| 310 | 1scDb-2Fab-AFc-6 Chain3: |
| | DIQMTQSPSTLSASVGDRVTITCRASQSISSWLAWYQQKPGKAPKLLIYKASSLESGVPSRFSGSGSGTEFTLTISSLQPDDFA |
| | TYYCQQYNSYPTFGGGTKVEIKRTVAAPSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGECGGGGSSYELTQPLSVSVALGQTARITCGGNNIGSKSVHW |
| | YQQKPGQAPVLVIYQDKKRPSGIPERFSGSNSGNTATLTISRAQAGDEADYYCQVWDDYIVLFGGGTKLTVLGQPKAAPSVTLF |
| | PPSSEELQANKATLVCLISDFYPGAVTVAWKADSSPVKAGVETTTPSKQSNNKYAASSYLSLTPEQWKSHRSYSCQVTHEGSTV |
| | EKTVAPTECS |
| 311 | 1scDb-2Fab-AFc-7 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSQPVLTQPPSVSVAPGKTARITCGGNNIGSKSVH |
| | WYQQKPGQAPVLVIYYDSDRPSGIPERFSGSNSGNTATLTISRVEAGDEADYYCQVWDTSSDHVLFGGGTKLTVLDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 312 | 1scDb-2Fab-AFc-7 Chain2: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTQVQLVQSGAEVKKPGASVKVSCKA |
| | SGYTFTNYYMQWVRQAPGQGLEWMGIINPSGGVTSYAQKFQGRVTMTRDTSTSTVYMELSSLRSEDTAVYYCARGSAYYYDFAD |
| | YWGQGTLVTVSSASTKGPSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPEFEGGPSVFLFPPKPKDTLMISRTPEVTCVVVAVSHEDPEV |
| | KFNWYVDGVEVHNAKTKPREEQYGSTYRVVSVLTVLHQDWLNGKEYKCKVSNKALPAPIEKTISKAKGQPREPQVYTLPPSREE |
| | MTKNQVSLSCAVKGFYPSDIAVEWESNGQPENNYKTTPPVLDSDGSFFLVSKLTVDKSRWQQGNVFSCSVMHEALHNHYTQKSL |
| | SLSPG |
| 313 | 1scDb-2Fab-AFc-7 Chain3: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGECGGGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGGGTKLTVLGQPKAAPSVTL |
| | FPPSSEELQANKATLVCLISDFYPGAVTVAWKADSSPVKAGVETTTPSKQSNNKYAASSYLSLTPEQWKSHRSYSCQVTHEGST |
| | VEKTVAPTECS |
| 314 | 1scDb-2Fab-AFc-8 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSQPVLTQPPSVSVAPGKTARITCGGNNIGSKSVHWYQQKP |
| | GQAPVLVIYYDSDRPSGIPERFSGSNSGNTATLTISRVEAGDEADYYCQVWDTSSDHVLFGGGTKLTVLGGSGGSGGSGGSGGS |
| | GGSGGSQVQLQESGPGLVKPSETLSLTCTVSGGSVSSGSYYWSWIRQPPGKGLEWIGYIYYSGSTNYNPSLKSRVTISVDTSKN |
| | QFSLKLSSVTAADTAVYYCARNPISIPAFDIWGQGTMVTVSSGGSGGSGGSQPVLTQPPSVSVAPGKTARITCGGNNIGSKSVH |
| | WYQQKPGQAPVLVIYYDSDRPSGIPERFSGSNSGNTATLTISRVEAGDEADYYCQVWDTSSDHVLFGGGTKLTVLDKTHTCPPC |
| | PAPEFEGGPSVFLFPPKPKDTLMISRTPEVTCVVVAVSHEDPEVKFNWYVDGVEVHNAKTKPREEQYGSTYRVVSVLTVLHQDW |
| | LNGKEYKCKVSNKALPAPIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPG |
| 315 | 1scDb-2Fab-AFc-8 Chain2: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKKVEPKSCDKTHTQVQLVQSGAEVKKPGASVKVSCKASGYT |
| | FTNYYMQWVRQAPGQGLEWMGIINPSGGVTSYAQKFQGRVTMTRDTSTSTVYMELSSLRSEDTAVYYCARGSAYYYDFADYWGQ |
| | GTLVTVSSASTKGPSVFPLAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPEFEGGPSVFLFPPKPKDTLMISRTPEVTCVVVAVSHEDPEVKFNW |
| | YVDGVEVHNAKTKPREEQYGSTYRVVSVLTVLHQDWLNGKEYKCKVSNKALPAPIEKTISKAKGQPREPQVYTLPPSREEMTKN |
| | QVSLSCAVKGFYPSDIAVEWESNGQPENNYKTTPPVLDSDGSFFLVSKLTVDKSRWQQGNVFSCSVMHEALHNHYTQKSLSLSP |
| | G |
| 316 | 1scDb-2Fab-AFc-8 Chain3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGECGGGGSSYELTQPLSVSVALGQTARITCGGNNIGSKSVH |
| | WYQQKPGQAPVLVIYQDKKRPSGIPERFSGSNSGNTATLTISRAQAGDEADYYCQVWDDYIVLFGGGTKLTVLGQPKAAPSVTL |
| | FPPSSEELQANKATLVCLISDFYPGAVTVAWKADSSPVKAGVETTTPSKQSNNKYAASSYLSLTPEQWKSHRSYSCQVTHEGST |
| | VEKTVAPTECS |
| 317 | 1scDb-1Fab-AFc-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPELLGGPSVFLFPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 318 | 1scDb-1Fab-AFc-1 Chain2: |
| | EVQLVESGGGLVKPGGSLRLSCAASGFTFSSYGMHWVRQAPGKGLEWVAFIRYDGSNKYYADSVKGRFTISRDNSKNTLYLQMN |
| | SLRAEDTAVYYCAKDRGLGDGTYFDYWGQGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPG |
| 319 | 1scDb-1Fab-AFc-1 Chain3: |
| | QSALTQPASVSGSPGQSITISCSGSSSNIGNNAVNWYQQLPGKAPKLLIYYDDLLPSGVSDRFSGSKSGTSAFLAISGLQSEDE |
| | ADYYCAAWDDSLNGPVFGGGTKLTVLGQPKAAPSVTLFPPSSEELQANKATLVCLISDFYPGAVTVAWKADSSPVKAGVETTTP |
| | SKQSNNKYAASSYLSLTPEQWKSHRSYSCQVTHEGSTVEKTVAPTECS |
| 320 | 1scDb-1Fab-AFc-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPELLGGPSVFLFPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 321 | 1scDb-1Fab-AFc-2 Chain2: |
| | EVQLQQWGAGLLKPSETLSLTCAVYGGSFSGYYWSWIRQPPGKGLEWIGEIDHSGSTNYNPSLKSRVTISVDTSKNQFSLKLSS |
| | VTAADTAVYYCARARGPWSFDPWGQGTLVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYNSTYRVVSVLTVLHQDWLNGKEYKCKVSNKALPAPIEKTI |
| | SKAKGQPREPQVYTLPPSREEMTKNQVSLSCAVKGFYPSDIAVEWESNGQPENNYKTTPPVLDSDGSFFLVSKLTVDKSRWQQG |
| | NVFSCSVMHEALHNHYTQKSLSLSPG |
| 322 | 1scDb-1Fab-AFc-2 Chain3: |
| | DIQMTQSPSTLSASVGDRVTITCRASQSISSWLAWYQQKPGKAPKLLIYKASSLESGVPSRFSGSGSGTEFTLTISSLQPDDFA |
| | TYYCQQYNSYPTFGGGTKVEIKRTVAAPSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC |
| 323 | 1scDb-1Fab-AFc-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPELLGGPSVFLFPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 324 | 1scDb-1Fab-AFc-3 Chain2: |
| | EVQLQQSGPELVKPGASVKMSCKASGYTFTDYVINWGKQRSGQGLEWIGEIYPGSGTNYYNEKFKAKATLTADKSSNIAYMQLS |
| | SLTSEDSAVYFCARRGRYGLYAMDYWGQGTSVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPG |
| 325 | 1scDb-1Fab-AFc-3 Chain3: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 326 | 1scDb-1Fab-AFc-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSDIVMTQSPATLSLSPGERATLSCRSSKSLQNVNGNTYL |
| | YWFQQKPGQSPQLLIYRMSNLNSGVPDRFSGSGSGTEFTLTISSLEPEDFAVYYCMQHLEYPITFGAGTKLEIKGGSGGSGGSG |
| | GSGGSGGSGGSEVQLVESGGGLVKPGGSLKLSCAASGYTFTSYVMHWVRQAPGKGLEWIGYINPYNDGTKYNEKFQGRVTISSD |
| | KSISTAYMELSSLRSEDTAMYYCARGTYYYGTRVFDYWGQGTLVTVSSGGSGGSGGSDIVMTQSPATLSLSPGERATLSCRSSK |
| | SLQNVNGNTYLYWFQQKPGQSPQLLIYRMSNLNSGVPDRFSGSGSGTEFTLTISSLEPEDFAVYYCMQHLEYPITFGAGTKLEI |
| | KDKTHTCPPCPAPELLGGPSVFLFPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNHYTQKSLSLSPG |
| 327 | 1scDb-1Fab-AFc-4 Chain2: |
| | EVQLQQSGPELVKPGASVKISCKTSGYTFTEYTMHWVKQSHGKSLEWIGGISPNIGGTSYNQKFKGKATLTVDKSSSTAYMELR |
| | SLTSEDSAVYYCARRGGSFDYWGQGTTLTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWLNGKEYKCKVSNKALPAPIEKTIS |
| | KAKGQPREPQVYTLPPSREEMTKNQVSLSCAVKGFYPSDIAVEWESNGQPENNYKTTPPVLDSDGSFFLVSKLTVDKSRWQQGN |
| | VFSCSVMHEALHNHYTQKSLSLSPG |
| 328 | 1scDb-1Fab-AFc-4 Chain3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 329 | AIG-2scFv-16 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLVESGGGLVKPGGSLRLSCAA |
| | SGFTFSSYGMHWVRQAPGKGLEWVAFIRYDGSNKYYADSVKGRFTISRDNSKNTLYLQMNSLRAEDTAVYYCAKDRGLGDGTYF |
| | DYWGQGTTVTVSSGGSGGSGGSGGSGGSGGSQSALTQPASVSGSPGQSITISCSGSSSNIGNNAVNWYQQLPGKAPKLLIYYDD |
| | LLPSGVSDRFSGSKSGTSAFLAISGLQSEDEADYYCAAWDDSLNGPVFGGGTKLTVL |
| 330 | AIG-2scFv-16 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 331 | AIG-2scFv-16 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSSYELTQPLSVSVALGQTARITCGG |
| | NNIGSKSVHWYQQKPGQAPVLVIYQDKKRPSGIPERFSGSNSGNTATLTISRAQAGDEADYYCQVWDDYIVLFGCGTKLTVLGG |
| | SGGSGGSGGSGGSGGSGGSQVQLVQSGAEVKKPGASVKVSCKASGYTFTNYYMQWVRQAPGQCLEWMGIINPSGGVTSYAQKFQ |
| | GRVTMTRDTSTSTVYMELSSLRSEDTAVYYCARGSAYYYDFADYWGQGTLVTVSS |
| 332 | AIG-2scFv-17 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLQQWGAGLLKPSETLSLTCAV |
| | YGGSFSGYYWSWIRQPPGKGLEWIGEIDHSGSTNYNPSLKSRVTISVDTSKNQFSLKLSSVTAADTAVYYCARARGPWSFDPWG |
| | QGTLVTVSSGGSGGSGGSGGSGGSGGSDIQMTQSPSTLSASVGDRVTITCRASQSISSWLAWYQQKPGKAPKLLIYKASSLESG |
| | VPSRFSGSGSGTEFTLTISSLQPDDFATYYCQQYNSYPTFGGGTKVEIK |
| 333 | AIG-2scFv-17 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 334 | AIG-2scFv-17 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSSYELTQPLSVSVALGQTARITCGG |
| | NNIGSKSVHWYQQKPGQAPVLVIYQDKKRPSGIPERFSGSNSGNTATLTISRAQAGDEADYYCQVWDDYIVLFGCGTKLTVLGG |
| | SGGSGGSGGSGGSGGSGGSQVQLVQSGAEVKKPGASVKVSCKASGYTFTNYYMQWVRQAPGQCLEWMGIINPSGGVTSYAQKFQ |
| | GRVTMTRDTSTSTVYMELSSLRSEDTAVYYCARGSAYYYDFADYWGQGTLVTVSS |
| 335 | AIG-2scFv-18 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLQQSGPELVKPGASVKMSCKA |
| | SGYTFTDYVINWGKQRSGQGLEWIGEIYPGSGTNYYNEKFKAKATLTADKSSNIAYMQLSSLTSEDSAVYFCARRGRYGLYAMD |
| | YWGQGTSVTVSSGGSGGSGGSGGSGGSGGSDIQMTQTTSSLSASLGDRVTISCRASQDISNYLNWYQQKPDGTVKLLIYYTSRL |
| | HSGVPSRFSGSGSGTDYSLTINNLEQEDIATYFCQQGNTRPWTFGGGTKLEIK |
| 336 | AIG-2scFv-18 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 337 | AIG-2scFv-18 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSSYELTQPLSVSVALGQTARITCGG |
| | NNIGSKSVHWYQQKPGQAPVLVIYQDKKRPSGIPERFSGSNSGNTATLTISRAQAGDEADYYCQVWDDYIVLFGCGTKLTVLGG |
| | SGGSGGSGGSGGSGGSGGSQVQLVQSGAEVKKPGASVKVSCKASGYTFTNYYMQWVRQAPGQCLEWMGIINPSGGVTSYAQKFQ |
| | GRVTMTRDTSTSTVYMELSSLRSEDTAVYYCARGSAYYYDFADYWGQGTLVTVSS |
| 338 | AIG-2scFv-19 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSEVQLQQSGPELVKPGASVKISCKT |
| | SGYTFTEYTMHWVKQSHGKSLEWIGGISPNIGGTSYNQKFKGKATLTVDKSSSTAYMELRSLTSEDSAVYYCARRGGSFDYWGQ |
| | GTTLTVSSGGSGGSGGSGGSGGSGGSDIVMTQSPATLSVTPGDRVSLSCRASQSISDYLHWYQQKSHESPRLLIKYASQSISGI |
| | PSRFSGSGSGSDFTLSINSVEPEDVGVYYCQNGHSFPLTFGAGTKLELK |
| 339 | AIG-2scFv-19 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 340 | AIG-2scFv-19 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSSYELTQPLSVSVALGQTARITCGG |
| | NNIGSKSVHWYQQKPGQAPVLVIYQDKKRPSGIPERFSGSNSGNTATLTISRAQAGDEADYYCQVWDDYIVLFGCGTKLTVLGG |
| | SGGSGGSGGSGGSGGSGGSQVQLVQSGAEVKKPGASVKVSCKASGYTFTNYYMQWVRQAPGQCLEWMGIINPSGGVTSYAQKFQ |
| | GRVTMTRDTSTSTVYMELSSLRSEDTAVYYCARGSAYYYDFADYWGQGTLVTVSS |
| 341 | AIG-2scFv-20 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLVESGGGLVKPGGSLRLSCAA |
| | SGFTFSSYGMHWVRQAPGKGLEWVAFIRYDGSNKYYADSVKGRFTISRDNSKNTLYLQMNSLRAEDTAVYYCAKDRGLGDGTYF |
| | DYWGQGTTVTVSSGGSGGSGGSGGSGGSGGSQSALTQPASVSGSPGQSITISCSGSSSNIGNNAVNWYQQLPGKAPKLLIYYDD |
| | LLPSGVSDRFSGSKSGTSAFLAISGLQSEDEADYYCAAWDDSLNGPVFGGGTKLTVL |
| 342 | AIG-2scFv-20 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 343 | AIG-2scFv-20 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSDIVLTQSPASLAVSLGQRATISCK |
| | ASQSVDFDGDSFMNWYQQKPGQPPKLLIYTTSNLESGIPARFSASGSGTDFTLNIHPVEEEDTATYYCQQSNEDPYTFGGGTKL |
| | ELKGGSGGSGGSGGSGGSGGSGGSQVTLKESGPGILQPSQTLSLTCSFSGFSLRTSGMGVGWIRQPSGKGLEWLAHIWWDDDKR |
| | YNPALKSRLTISKDTSSNQVFLKIASVDTADTATYYCAQINPAWFAYWGQGTLVTVSA |
| 344 | AIG-2scFv-21 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLQQWGAGLLKPSETLSLTCAV |
| | YGGSFSGYYWSWIRQPPGKGLEWIGEIDHSGSTNYNPSLKSRVTISVDTSKNQFSLKLSSVTAADTAVYYCARARGPWSFDPWG |
| | QGTLVTVSSGGSGGSGGSGGSGGSGGSDIQMTQSPSTLSASVGDRVTITCRASQSISSWLAWYQQKPGKAPKLLIYKASSLESG |
| | VPSRFSGSGSGTEFTLTISSLQPDDFATYYCQQYNSYPTFGGGTKVEIK |
| 345 | AIG-2scFv-21 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 346 | AIG-2scFv-21 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSDIVLTQSPASLAVSLGQRATISCK |
| | ASQSVDFDGDSFMNWYQQKPGQPPKLLIYTTSNLESGIPARFSASGSGTDFTLNIHPVEEEDTATYYCQQSNEDPYTFGGGTKL |
| | ELKGGSGGSGGSGGSGGSGGSGGSQVTLKESGPGILQPSQTLSLTCSFSGFSLRTSGMGVGWIRQPSGKGLEWLAHIWWDDDKR |
| | YNPALKSRLTISKDTSSNQVFLKIASVDTADTATYYCAQINPAWFAYWGQGTLVTVSA |
| 347 | AIG-2scFv-22 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLQQSGPELVKPGASVKMSCKA |
| | SGYTFTDYVINWGKQRSGQGLEWIGEIYPGSGTNYYNEKFKAKATLTADKSSNIAYMQLSSLTSEDSAVYFCARRGRYGLYAMD |
| | YWGQGTSVTVSSGGSGGSGGSGGSGGSGGSDIQMTQTTSSLSASLGDRVTISCRASQDISNYLNWYQQKPDGTVKLLIYYTSRL |
| | HSGVPSRFSGSGSGTDYSLTINNLEQEDIATYFCQQGNTRPWTFGGGTKLEIK |
| 348 | AIG-2scFv-22 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 349 | AIG-2scFv-22 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSDIVLTQSPASLAVSLGQRATISCK |
| | ASQSVDFDGDSFMNWYQQKPGQPPKLLIYTTSNLESGIPARFSASGSGTDFTLNIHPVEEEDTATYYCQQSNEDPYTFGGGTKL |
| | ELKGGSGGSGGSGGSGGSGGSGGSQVTLKESGPGILQPSQTLSLTCSFSGFSLRTSGMGVGWIRQPSGKGLEWLAHIWWDDDKR |
| | YNPALKSRLTISKDTSSNQVFLKIASVDTADTATYYCAQINPAWFAYWGQGTLVTVSA |
| 350 | AIG-2scFv-23 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSEVQLQQSGPELVKPGASVKISCKT |
| | SGYTFTEYTMHWVKQSHGKSLEWIGGISPNIGGTSYNQKFKGKATLTVDKSSSTAYMELRSLTSEDSAVYYCARRGGSFDYWGQ |
| | GTTLTVSSGGSGGSGGSGGSGGSGGSDIVMTQSPATLSVTPGDRVSLSCRASQSISDYLHWYQQKSHESPRLLIKYASQSISGI |
| | PSRFSGSGSGSDFTLSINSVEPEDVGVYYCQNGHSFPLTFGAGTKLELK |
| 351 | AIG-2scFv-23 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 352 | AIG-2scFv-23 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSGGGGSGGGGSGGGGSGGGGSDIVLTQSPASLAVSLGQRATISCK |
| | ASQSVDFDGDSFMNWYQQKPGQPPKLLIYTTSNLESGIPARFSASGSGTDFTLNIHPVEEEDTATYYCQQSNEDPYTFGGGTKL |
| | ELKGGSGGSGGSGGSGGSGGSGGSQVTLKESGPGILQPSQTLSLTCSFSGFSLRTSGMGVGWIRQPSGKGLEWLAHIWWDDDKR |
| | YNPALKSRLTISKDTSSNQVFLKIASVDTADTATYYCAQINPAWFAYWGQGTLVTVSA |
| 353 | IG-scDb-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSQVQLVESGGGLVKPGG |
| | SLRLSCAASGFTFSSYGMHWVRQAPGKGLEWVAFIRYDGSNKYYADSVKGRFTISRDNSKNTLYLQMNSLRAEDTAVYYCAKDR |
| | GLGDGTYFDYWGQGTTVTVSSGGSGGSGGSGGSGGSGGSQSALTQPASVSGSPGQSITISCSGSSSNIGNNAVNWYQQLPGKAP |
| | KLLIYYDDLLPSGVSDRFSGSKSGTSAFLAISGLQSEDEADYYCAAWDDSLNGPVFGGGTKLTVLGGSGGSQVQLVQSGAEVKK |
| | PGASVKVSCKASGYTFTSYYMHWVRQAPGQCLEWMGAIEPTYGSTSYAQKFQGRVTMTRDTSTSTVYMELSSLRSEDTAVYYCA |
| | RGSAYYYDFADYWGQGTLVTVSS |
| 354 | IG-scDb-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 355 | IG-scDb-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSQVQLQQWGAGLLKPSE |
| | TLSLTCAVYGGSFSGYYWSWIRQPPGKGLEWIGEIDHSGSTNYNPSLKSRVTISVDTSKNQFSLKLSSVTAADTAVYYCARARG |
| | PWSFDPWGQGTLVTVSSGGSGGSGGSGGSGGSGGSDIQMTQSPSTLSASVGDRVTITCRASQSISSWLAWYQQKPGKAPKLLIY |
| | KASSLESGVPSRFSGSGSGTEFTLTISSLQPDDFATYYCQQYNSYPTFGGGTKVEIKGGSGGSQVQLVQSGAEVKKPGASVKVS |
| | CKASGYTFTSYYMHWVRQAPGQCLEWMGAIEPTYGSTSYAQKFQGRVTMTRDTSTSTVYMELSSLRSEDTAVYYCARGSAYYYD |
| | FADYWGQGTLVTVSS |
| 356 | IG-scDb-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 357 | IG-scDb-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSQVQLQQSGPELVKPGA |
| | SVKMSCKASGYTFTDYVINWGKQRSGQGLEWIGEIYPGSGTNYYNEKFKAKATLTADKSSNIAYMQLSSLTSEDSAVYFCARRG |
| | RYGLYAMDYWGQGTSVTVSSGGSGGSGGSGGSGGSGGSDIQMTQTTSSLSASLGDRVTISCRASQDISNYLNWYQQKPDGTVKL |
| | LIYYTSRLHSGVPSRFSGSGSGTDYSLTINNLEQEDIATYFCQQGNTRPWTFGGGTKLEIKGGSGGSQVQLVQSGAEVKKPGAS |
| | VKVSCKASGYTFTSYYMHWVRQAPGQCLEWMGAIEPTYGSTSYAQKFQGRVTMTRDTSTSTVYMELSSLRSEDTAVYYCARGSA |
| | YYYDFADYWGQGTLVTVSS |
| 358 | IG-scDb-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 359 | IG-scDb-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSEVQLQQSGPELVKPGA |
| | SVKISCKTSGYTFTEYTMHWVKQSHGKSLEWIGGISPNIGGTSYNQKFKGKATLTVDKSSSTAYMELRSLTSEDSAVYYCARRG |
| | GSFDYWGQGTTLTVSSGGSGGSGGSGGSGGSGGSDIVMTQSPATLSVTPGDRVSLSCRASQSISDYLHWYQQKSHESPRLLIKY |
| | ASQSISGIPSRFSGSGSGSDFTLSINSVEPEDVGVYYCQNGHSFPLTFGAGTKLELKGGSGGSQVQLVQSGAEVKKPGASVKVS |
| | CKASGYTFTSYYMHWVRQAPGQCLEWMGAIEPTYGSTSYAQKFQGRVTMTRDTSTSTVYMELSSLRSEDTAVYYCARGSAYYYD |
| | FADYWGQGTLVTVSS |
| 360 | IG-scDb-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 361 | IG-scDb-5 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIVLTQSPASLAVSLGQRATISCKASQSVDFDGDSFMNWYQQKP |
| | GQPPKLLIYTTSNLESGIPARFSASGSGTDFTLNIHPVEEEDTATYYCQQSNEDPYTFGGGTKLELKGGSGGSQVQLVESGGGL |
| | VKPGGSLRLSCAASGFTFSSYGMHWVRQAPGKGLEWVAFIRYDGSNKYYADSVKGRFTISRDNSKNTLYLQMNSLRAEDTAVYY |
| | CAKDRGLGDGTYFDYWGQGTTVTVSSGGSGGSGGSGGSGGSGGSQSALTQPASVSGSPGQSITISCSGSSSNIGNNAVNWYQQL |
| | PGKAPKLLIYYDDLLPSGVSDRFSGSKSGTSAFLAISGLQSEDEADYYCAAWDDSLNGPVFGGGTKLTVLGGSGGSQVTLKESG |
| | PGILQPSQTLSLTCSFSGFSLRTSGMGVGWIRQPSGKGLEWLAHIWWDDDKRYNPALKSRLTISKDTSSNQVFLKIASVDTADT |
| | ATYYCAQINPAWFAYWGQGTLVTVSA |
| 362 | IG-scDb-5 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 363 | IG-scDb-6 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIVLTQSPASLAVSLGQRATISCKASQSVDFDGDSFMNWYQQKP |
| | GQPPKLLIYTTSNLESGIPARFSASGSGTDFTLNIHPVEEEDTATYYCQQSNEDPYTFGGGTKLELKGGSGGSQVQLQQWGAGL |
| | LKPSETLSLTCAVYGGSFSGYYWSWIRQPPGKGLEWIGEIDHSGSTNYNPSLKSRVTISVDTSKNQFSLKLSSVTAADTAVYYC |
| | ARARGPWSFDPWGQGTLVTVSSGGSGGSGGSGGSGGSGGSDIQMTQSPSTLSASVGDRVTITCRASQSISSWLAWYQQKPGKAP |
| | KLLIYKASSLESGVPSRFSGSGSGTEFTLTISSLQPDDFATYYCQQYNSYPTFGGGTKVEIKGGSGGSQVTLKESGPGILQPSQ |
| | TLSLTCSFSGFSLRTSGMGVGWIRQPSGKGLEWLAHIWWDDDKRYNPALKSRLTISKDTSSNQVFLKIASVDTADTATYYCAQI |
| | NPAWFAYWGQGTLVTVSA |
| 364 | IG-scDb-6 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 365 | IG-scDb-7 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIVLTQSPASLAVSLGQRATISCKASQSVDFDGDSFMNWYQQKP |
| | GQPPKLLIYTTSNLESGIPARFSASGSGTDFTLNIHPVEEEDTATYYCQQSNEDPYTFGGGTKLELKGGSGGSQVQLQQSGPEL |
| | VKPGASVKMSCKASGYTFTDYVINWGKQRSGQGLEWIGEIYPGSGTNYYNEKFKAKATLTADKSSNIAYMQLSSLTSEDSAVYF |
| | CARRGRYGLYAMDYWGQGTSVTVSSGGSGGSGGSGGSGGSGGSDIQMTQTTSSLSASLGDRVTISCRASQDISNYLNWYQQKPD |
| | GTVKLLIYYTSRLHSGVPSRFSGSGSGTDYSLTINNLEQEDIATYFCQQGNTRPWTFGGGTKLEIKGGSGGSQVTLKESGPGIL |
| | QPSQTLSLTCSFSGFSLRTSGMGVGWIRQPSGKGLEWLAHIWWDDDKRYNPALKSRLTISKDTSSNQVFLKIASVDTADTATYY |
| | CAQINPAWFAYWGQGTLVTVSA |
| 366 | IG-scDb-7 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 367 | IG-scDb-8 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIVLTQSPASLAVSLGQRATISCKASQSVDFDGDSFMNWYQQKP |
| | GQPPKLLIYTTSNLESGIPARFSASGSGTDFTLNIHPVEEEDTATYYCQQSNEDPYTFGGGTKLELKGGSGGSEVQLQQSGPEL |
| | VKPGASVKISCKTSGYTFTEYTMHWVKQSHGKSLEWIGGISPNIGGTSYNQKFKGKATLTVDKSSSTAYMELRSLTSEDSAVYY |
| | CARRGGSFDYWGQGTTLTVSSGGSGGSGGSGGSGGSGGSDIVMTQSPATLSVTPGDRVSLSCRASQSISDYLHWYQQKSHESPR |
| | LLIKYASQSISGIPSRFSGSGSGSDFTLSINSVEPEDVGVYYCQNGHSFPLTFGAGTKLELKGGSGGSQVTLKESGPGILQPSQ |
| | TLSLTCSFSGFSLRTSGMGVGWIRQPSGKGLEWLAHIWWDDDKRYNPALKSRLTISKDTSSNQVFLKIASVDTADTATYYCAQI |
| | NPAWFAYWGQGTLVTVSA |
| 368 | IG-scDb-8 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 369 | AIG-2scDb-1 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSQSALTQPASVSGSPGQSITISCSGSSSNIGNNAVNWYQQLPGKA |
| | PKLLIYYDDLLPSGVSDRFSGSKSGTSAFLAISGLQSEDEADYYCAAWDDSLNGPVFGGGTKLTVLGGSGGSQVQLVESGGGLV |
| | KPGGSLRLSCAASGFTFSSYGMHWVRQAPGKGLEWVAFIRYDGSNKYYADSVKGRFTISRDNSKNTLYLQMNSLRAEDTAVYYC |
| | AKDRGLGDGTYFDYWGQGTTVTVSSGGSGGSGGSGGSGGSGGSQSALTQPASVSGSPGQSITISCSGSSSNIGNNAVNWYQQLP |
| | GKAPKLLIYYDDLLPSGVSDRFSGSKSGTSAFLAISGLQSEDEADYYCAAWDDSLNGPVFGGGTKLTVLGGSGGSQVQLVESGG |
| | GLVKPGGSLRLSCAASGFTFSSYGMHWVRQAPGKGLEWVAFIRYDGSNKYYADSVKGRFTISRDNSKNTLYLQMNSLRAEDTAV |
| | YYCAKDRGLGDGTYFDYWGQGTTVTVSS |
| 370 | AIG-2scDb-1 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 371 | AIG-2scDb-1 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSQVQLVQSGAEVKKPGA |
| | SVKVSCKASGYTFTSYYMHWVRQAPGQCLEWMGAIEPTYGSTSYAQKFQGRVTMTRDTSTSTVYMELSSLRSEDTAVYYCARGS |
| | AYYYDFADYWGQGTLVTVSSGGSGGSGGSGGSGGSGGSSYELTQPLSVSVALGQTARITCGGHNIGSKNVHWYQQKPGQAPVLV |
| | IYQDNKRPSGIPERFSGSNSGNTATLTISRAQAGDEADYYCQVWDNYNVLFGCGTKLTVLGGSGGSQVQLVQSGAEVKKPGASV |
| | KVSCKASGYTFTSYYMHWVRQAPGQCLEWMGAIEPTYGSTSYAQKFQGRVTMTRDTSTSTVYMELSSLRSEDTAVYYCARGSAY |
| | YYDFADYWGQGTLVTVSS |
| 372 | AIG-2scDb-2 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIQMTQSPSTLSASVGDRVTITCRASQSISSWLAWYQQKPGKAP |
| | KLLIYKASSLESGVPSRFSGSGSGTEFTLTISSLQPDDFATYYCQQYNSYPTFGGGTKVEIKGGSGGSQVQLQQWGAGLLKPSE |
| | TLSLTCAVYGGSFSGYYWSWIRQPPGKGLEWIGEIDHSGSTNYNPSLKSRVTISVDTSKNQFSLKLSSVTAADTAVYYCARARG |
| | PWSFDPWGQGTLVTVSSGGSGGSGGSGGSGGSGGSDIQMTQSPSTLSASVGDRVTITCRASQSISSWLAWYQQKPGKAPKLLIY |
| | KASSLESGVPSRFSGSGSGTEFTLTISSLQPDDFATYYCQQYNSYPTFGGGTKVEIKGGSGGSQVQLQQWGAGLLKPSETLSLT |
| | CAVYGGSFSGYYWSWIRQPPGKGLEWIGEIDHSGSTNYNPSLKSRVTISVDTSKNQFSLKLSSVTAADTAVYYCARARGPWSFD |
| | PWGQGTLVTVSS |
| 373 | AIG-2scDb-2 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 374 | AIG-2scDb-2 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSQVQLVQSGAEVKKPGA |
| | SVKVSCKASGYTFTSYYMHWVRQAPGQCLEWMGAIEPTYGSTSYAQKFQGRVTMTRDTSTSTVYMELSSLRSEDTAVYYCARGS |
| | AYYYDFADYWGQGTLVTVSSGGSGGSGGSGGSGGSGGSSYELTQPLSVSVALGQTARITCGGHNIGSKNVHWYQQKPGQAPVLV |
| | IYQDNKRPSGIPERFSGSNSGNTATLTISRAQAGDEADYYCQVWDNYNVLFGCGTKLTVLGGSGGSQVQLVQSGAEVKKPGASV |
| | KVSCKASGYTFTSYYMHWVRQAPGQCLEWMGAIEPTYGSTSYAQKFQGRVTMTRDTSTSTVYMELSSLRSEDTAVYYCARGSAY |
| | YYDFADYWGQGTLVTVSS |
| 375 | AIG-2scDb-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIQMTQTTSSLSASLGDRVTISCRASQDISNYLNWYQQKPDGTV |
| | KLLIYYTSRLHSGVPSRFSGSGSGTDYSLTINNLEQEDIATYFCQQGNTRPWTFGGGTKLEIKGGSGGSQVQLQQSGPELVKPG |
| | ASVKMSCKASGYTFTDYVINWGKQRSGQGLEWIGEIYPGSGTNYYNEKFKAKATLTADKSSNIAYMQLSSLTSEDSAVYFCARR |
| | GRYGLYAMDYWGQGTSVTVSSGGSGGSGGSGGSGGSGGSDIQMTQTTSSLSASLGDRVTISCRASQDISNYLNWYQQKPDGTVK |
| | LLIYYTSRLHSGVPSRFSGSGSGTDYSLTINNLEQEDIATYFCQQGNTRPWTFGGGTKLEIKGGSGGSQVQLQQSGPELVKPGA |
| | SVKMSCKASGYTFTDYVINWGKQRSGQGLEWIGEIYPGSGTNYYNEKFKAKATLTADKSSNIAYMQLSSLTSEDSAVYFCARRG |
| | RYGLYAMDYWGQGTSVTVSS |
| 376 | AIG-2scDb-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 377 | AIG-2scDb-3 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSQVQLVQSGAEVKKPGA |
| | SVKVSCKASGYTFTSYYMHWVRQAPGQCLEWMGAIEPTYGSTSYAQKFQGRVTMTRDTSTSTVYMELSSLRSEDTAVYYCARGS |
| | AYYYDFADYWGQGTLVTVSSGGSGGSGGSGGSGGSGGSSYELTQPLSVSVALGQTARITCGGHNIGSKNVHWYQQKPGQAPVLV |
| | IYQDNKRPSGIPERFSGSNSGNTATLTISRAQAGDEADYYCQVWDNYNVLFGCGTKLTVLGGSGGSQVQLVQSGAEVKKPGASV |
| | KVSCKASGYTFTSYYMHWVRQAPGQCLEWMGAIEPTYGSTSYAQKFQGRVTMTRDTSTSTVYMELSSLRSEDTAVYYCARGSAY |
| | YYDFADYWGQGTLVTVSS |
| 378 | AIG-2scDb-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIVMTQSPATLSVTPGDRVSLSCRASQSISDYLHWYQQKSHESP |
| | RLLIKYASQSISGIPSRFSGSGSGSDFTLSINSVEPEDVGVYYCQNGHSFPLTFGAGTKLELKGGSGGSEVQLQQSGPELVKPG |
| | ASVKISCKTSGYTFTEYTMHWVKQSHGKSLEWIGGISPNIGGTSYNQKFKGKATLTVDKSSSTAYMELRSLTSEDSAVYYCARR |
| | GGSFDYWGQGTTLTVSSGGSGGSGGSGGSGGSGGSDIVMTQSPATLSVTPGDRVSLSCRASQSISDYLHWYQQKSHESPRLLIK |
| | YASQSISGIPSRFSGSGSGSDFTLSINSVEPEDVGVYYCQNGHSFPLTFGAGTKLELKGGSGGSEVQLQQSGPELVKPGASVKI |
| | SCKTSGYTFTEYTMHWVKQSHGKSLEWIGGISPNIGGTSYNQKFKGKATLTVDKSSSTAYMELRSLTSEDSAVYYCARRGGSFD |
| | YWGQGTTLTVSS |
| 379 | AIG-2scDb-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 380 | AIG-2scDb-4 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPEFEGGPSVFLFPPKPK |
| | DTLMISRTPEVTCVVVAVSHEDPEVKFNWYVDGVEVHNAKTKPREEQYGSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGGGGGSGGGGSSYELTQPLSVSVALGQTARITCGGHNIGSKNVHWYQQKPGQAPV |
| | LVIYQDNKRPSGIPERFSGSNSGNTATLTISRAQAGDEADYYCQVWDNYNVLFGCGTKLTVLGGSGGSQVQLVQSGAEVKKPGA |
| | SVKVSCKASGYTFTSYYMHWVRQAPGQCLEWMGAIEPTYGSTSYAQKFQGRVTMTRDTSTSTVYMELSSLRSEDTAVYYCARGS |
| | AYYYDFADYWGQGTLVTVSSGGSGGSGGSGGSGGSGGSSYELTQPLSVSVALGQTARITCGGHNIGSKNVHWYQQKPGQAPVLV |
| | IYQDNKRPSGIPERFSGSNSGNTATLTISRAQAGDEADYYCQVWDNYNVLFGCGTKLTVLGGSGGSQVQLVQSGAEVKKPGASV |
| | KVSCKASGYTFTSYYMHWVRQAPGQCLEWMGAIEPTYGSTSYAQKFQGRVTMTRDTSTSTVYMELSSLRSEDTAVYYCARGSAY |
| | YYDFADYWGQGTLVTVSS |
| 381 | AIG-1scDb-3 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSQSALTQPASVSGSPGQSITISCSGSSSNIGNNAVNWYQQLPGKA |
| | PKLLIYYDDLLPSGVSDRFSGSKSGTSAFLAISGLQSEDEADYYCAAWDDSLNGPVFGGGTKLTVLGGSGGSQVQLVESGGGLV |
| | KPGGSLRLSCAASGFTFSSYGMHWVRQAPGKGLEWVAFIRYDGSNKYYADSVKGRFTISRDNSKNTLYLQMNSLRAEDTAVYYC |
| | AKDRGLGDGTYFDYWGQGTTVTVSSGGSGGSGGSGGSGGSGGSQSALTQPASVSGSPGQSITISCSGSSSNIGNNAVNWYQQLP |
| | GKAPKLLIYYDDLLPSGVSDRFSGSKSGTSAFLAISGLQSEDEADYYCAAWDDSLNGPVFGGGTKLTVLGGSGGSQVQLVESGG |
| | GLVKPGGSLRLSCAASGFTFSSYGMHWVRQAPGKGLEWVAFIRYDGSNKYYADSVKGRFTISRDNSKNTLYLQMNSLRAEDTAV |
| | YYCAKDRGLGDGTYFDYWGQGTTVTVSS |
| 382 | AIG-1scDb-3 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 383 | AIG-1scDb-3 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPG |
| 384 | AIG-1scDb-4 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIQMTQSPSTLSASVGDRVTITCRASQSISSWLAWYQQKPGKAP |
| | KLLIYKASSLESGVPSRFSGSGSGTEFTLTISSLQPDDFATYYCQQYNSYPTFGGGTKVEIKGGSGGSQVQLQQWGAGLLKPSE |
| | TLSLTCAVYGGSFSGYYWSWIRQPPGKGLEWIGEIDHSGSTNYNPSLKSRVTISVDTSKNQFSLKLSSVTAADTAVYYCARARG |
| | PWSFDPWGQGTLVTVSSGGSGGSGGSGGSGGSGGSDIQMTQSPSTLSASVGDRVTITCRASQSISSWLAWYQQKPGKAPKLLIY |
| | KASSLESGVPSRFSGSGSGTEFTLTISSLQPDDFATYYCQQYNSYPTFGGGTKVEIKGGSGGSQVQLQQWGAGLLKPSETLSLT |
| | CAVYGGSFSGYYWSWIRQPPGKGLEWIGEIDHSGSTNYNPSLKSRVTISVDTSKNQFSLKLSSVTAADTAVYYCARARGPWSFD |
| | PWGQGTLVTVSS |
| 385 | AIG-1scDb-4 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 386 | AIG-1scDb-4 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPG |
| 387 | AIG-1scDb-5 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIQMTQTTSSLSASLGDRVTISCRASQDISNYLNWYQQKPDGTV |
| | KLLIYYTSRLHSGVPSRFSGSGSGTDYSLTINNLEQEDIATYFCQQGNTRPWTFGGGTKLEIKGGSGGSQVQLQQSGPELVKPG |
| | ASVKMSCKASGYTFTDYVINWGKQRSGQGLEWIGEIYPGSGTNYYNEKFKAKATLTADKSSNIAYMQLSSLTSEDSAVYFCARR |
| | GRYGLYAMDYWGQGTSVTVSSGGSGGSGGSGGSGGSGGSDIQMTQTTSSLSASLGDRVTISCRASQDISNYLNWYQQKPDGTVK |
| | LLIYYTSRLHSGVPSRFSGSGSGTDYSLTINNLEQEDIATYFCQQGNTRPWTFGGGTKLEIKGGSGGSQVQLQQSGPELVKPGA |
| | SVKMSCKASGYTFTDYVINWGKQRSGQGLEWIGEIYPGSGTNYYNEKFKAKATLTADKSSNIAYMQLSSLTSEDSAVYFCARRG |
| | RYGLYAMDYWGQGTSVTVSS |
| 388 | AIG-1scDb-5 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 389 | AIG-1scDb-5 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPG |
| 390 | AIG-1scDb-6 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSDIVMTQSPATLSVTPGDRVSLSCRASQSISDYLHWYQQKSHESP |
| | RLLIKYASQSISGIPSRFSGSGSGSDFTLSINSVEPEDVGVYYCQNGHSFPLTFGAGTKLELKGGSGGSEVQLQQSGPELVKPG |
| | ASVKISCKTSGYTFTEYTMHWVKQSHGKSLEWIGGISPNIGGTSYNQKFKGKATLTVDKSSSTAYMELRSLTSEDSAVYYCARR |
| | GGSFDYWGQGTTLTVSSGGSGGSGGSGGSGGSGGSDIVMTQSPATLSVTPGDRVSLSCRASQSISDYLHWYQQKSHESPRLLIK |
| | YASQSISGIPSRFSGSGSGSDFTLSINSVEPEDVGVYYCQNGHSFPLTFGAGTKLELKGGSGGSEVQLQQSGPELVKPGASVKI |
| | SCKTSGYTFTEYTMHWVKQSHGKSLEWIGGISPNIGGTSYNQKFKGKATLTVDKSSSTAYMELRSLTSEDSAVYYCARRGGSFD |
| | YWGQGTTLTVSS |
| 391 | AIG-1scDb-6 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 392 | AIG-1scDb-6 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPG |
| 393 | 1Fab-AFc-1Fab-1 Chain1: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGECDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWLNGKEYKCKVSNKALPAPIEKTIS |
| | KAKGQPREPQVYTLPPSREEMTKNQVSLWCLVKGFYPSDIAVEWESNGQPENNYKTTPPVLDSDGSFFLYSKLTVDKSRWQQGN |
| | VFSCSVMHEALHNHYTQKSLSLSPGK |
| 394 | 1Fab-AFc-1Fab-1 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGSTGSQVQLQQSGPELVKPGASVKMSCKASGYTFTDYVINWGKQRSGQGLEWIGE |
| | IYPGSGTNYYNEKFKAKATLTADKSSNIAYMQLSSLTSEDSAVYFCARRGRYGLYAMDYWGQGTSVTVSSRTVAAPSVFIFPPS |
| | DEQLKSGTASVVCLLNNFYPREAKVQWKVDNALQSGNSQESVTEQDSKDSTYSLSSTLTLSKADYEKHKVYACEVTHQGLSSPV |
| | TKSFNRGEC |
| 395 | 1Fab-AFc-1Fab-1 Chain3: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 396 | 1Fab-AFc-1Fab-2 Chain1: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGECDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWLNGKEYKCKVSNKALPAPIEKTIS |
| | KAKGQPREPQVYTLPPSREEMTKNQVSLWCLVKGFYPSDIAVEWESNGQPENNYKTTPPVLDSDGSFFLYSKLTVDKSRWQQGN |
| | VFSCSVMHEALHNHYTQKSLSLSPGK |
| 397 | 1Fab-AFc-1Fab-2 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGSTGSEVQLQQSGPELVKPGASVKISCKTSGYTFTEYTMHWVKQSHGKSLEWIGG |
| | ISPNIGGTSYNQKFKGKATLTVDKSSSTAYMELRSLTSEDSAVYYCARRGGSFDYWGQGTTLTVSSRTVAAPSVFIFPPSDEQL |
| | KSGTASVVCLLNNFYPREAKVQWKVDNALQSGNSQESVTEQDSKDSTYSLSSTLTLSKADYEKHKVYACEVTHQGLSSPVTKSF |
| | NRGEC |
| 398 | 1Fab-AFc-1Fab-2 Chain3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 399 | 1Fab-AFc-1Fab-3 Chain1: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECSDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVSNKALPAPIEKTISKAKGQ |
| | PREPQVYTLPPSREEMTKNQVSLWCLVKGFYPSDIAVEWESNGQPENNYKTTPPVLDSDGSFFLYSKLTVDKSRWQQGNVFSCS |
| | VMHEALHNHYTQKSLSLSPGK |
| 400 | 1Fab-AFc-1Fab-3 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGSTGSQVQLQQSGPELVKPGASVKMSCKASGYTFTDYVINWGKQRSGQGLEWIGEI |
| | YPGSGTNYYNEKFKAKATLTADKSSNIAYMQLSSLTSEDSAVYFCARRGRYGLYAMDYWGQGTSVTVSSRTVAAPSVFIFPPSD |
| | EQLKSGTASVVCLLNNFYPREAKVQWKVDNALQSGNSQESVTEQDSKDSTYSLSSTLTLSKADYEKHKVYACEVTHQGLSSPVT |
| | KSFNRGEC |
| 401 | 1Fab-AFc-1Fab-3 Chain3: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 402 | 1Fab-AFc-1Fab-4 Chain1: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECSDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVSNKALPAPIEKTISKAKGQ |
| | PREPQVYTLPPSREEMTKNQVSLWCLVKGFYPSDIAVEWESNGQPENNYKTTPPVLDSDGSFFLYSKLTVDKSRWQQGNVFSCS |
| | VMHEALHNHYTQKSLSLSPGK |
| 403 | 1Fab-AFc-1Fab-4 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGSTGSEVQLQQSGPELVKPGASVKISCKTSGYTFTEYTMHWVKQSHGKSLEWIGGI |
| | SPNIGGTSYNQKFKGKATLTVDKSSSTAYMELRSLTSEDSAVYYCARRGGSFDYWGQGTTLTVSSRTVAAPSVFIFPPSDEQLK |
| | SGTASVVCLLNNFYPREAKVQWKVDNALQSGNSQESVTEQDSKDSTYSLSSTLTLSKADYEKHKVYACEVTHQGLSSPVTKSFN |
| | RGEC |
| 404 | 1Fab-AFc-1Fab-4 Chain3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 405 | 1Fab-AFc-1Fab-5 Chain1: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGECDKTHTCPPCPAPELLGGPDVFLFPPKPKDTLMI |
| | SRTPEVTCVVVDVSHEDPEVKFNWYVDGVEVHNAKTKPREEQYNSTYRVVSVLTVLHQDWLNGKEYKCKVSNKALPAPEEKTIS |
| | KAKGQPREPQVYTLPPSREEMTKNQVSLWCLVKGFYPSDIAVEWESNGQPENNYKTTPPVLDSDGSFFLYSKLTVDKSRWQQGN |
| | VFSCSVMHEALHNHYTQKSLSLSPGK |
| 406 | 1Fab-AFc-1Fab-5 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPDVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPE |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGSTGSQVQLQQSGPELVKPGASVKMSCKASGYTFTDYVINWGKQRSGQGLEWIGE |
| | IYPGSGTNYYNEKFKAKATLTADKSSNIAYMQLSSLTSEDSAVYFCARRGRYGLYAMDYWGQGTSVTVSSRTVAAPSVFIFPPS |
| | DEQLKSGTASVVCLLNNFYPREAKVQWKVDNALQSGNSQESVTEQDSKDSTYSLSSTLTLSKADYEKHKVYACEVTHQGLSSPV |
| | TKSFNRGEC |
| 407 | 1Fab-AFc-1Fab-5 Chain3: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 408 | 1Fab-AFc-1Fab-6 Chain1: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGECDKTHTCPPCPAPELLGGPDVFLFPPKPKDTLMI |
| | SRTPEVTCVVVDVSHEDPEVKFNWYVDGVEVHNAKTKPREEQYNSTYRVVSVLTVLHQDWLNGKEYKCKVSNKALPAPEEKTIS |
| | KAKGQPREPQVYTLPPSREEMTKNQVSLWCLVKGFYPSDIAVEWESNGQPENNYKTTPPVLDSDGSFFLYSKLTVDKSRWQQGN |
| | VFSCSVMHEALHNHYTQKSLSLSPGK |
| 409 | 1Fab-AFc-1Fab-6 Chain2: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPDVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPE |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPGSTGSEVQLQQSGPELVKPGASVKISCKTSGYTFTEYTMHWVKQSHGKSLEWIGG |
| | ISPNIGGTSYNQKFKGKATLTVDKSSSTAYMELRSLTSEDSAVYYCARRGGSFDYWGQGTTLTVSSRTVAAPSVFIFPPSDEQL |
| | KSGTASVVCLLNNFYPREAKVQWKVDNALQSGNSQESVTEQDSKDSTYSLSSTLTLSKADYEKHKVYACEVTHQGLSSPVTKSF |
| | NRGEC |
| 410 | 1Fab-AFc-1Fab-6 Chain3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 411 | 1Fab-AFc-1Fab-7 Chain1: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECSDKTHTCPPCPAPELLGGPDVFLFPPKPKDTLMISRTPE |
| | VTCVVVDVSHEDPEVKFNWYVDGVEVHNAKTKPREEQYNSTYRVVSVLTVLHQDWLNGKEYKCKVSNKALPAPEEKTISKAKGQ |
| | PREPQVYTLPPSREEMTKNQVSLWCLVKGFYPSDIAVEWESNGQPENNYKTTPPVLDSDGSFFLYSKLTVDKSRWQQGNVFSCS |
| | VMHEALHNHYTQKSLSLSPGK |
| 412 | 1Fab-AFc-1Fab-7 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPDVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPEE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGSTGSQVQLQQSGPELVKPGASVKMSCKASGYTFTDYVINWGKQRSGQGLEWIGEI |
| | YPGSGTNYYNEKFKAKATLTADKSSNIAYMQLSSLTSEDSAVYFCARRGRYGLYAMDYWGQGTSVTVSSRTVAAPSVFIFPPSD |
| | EQLKSGTASVVCLLNNFYPREAKVQWKVDNALQSGNSQESVTEQDSKDSTYSLSSTLTLSKADYEKHKVYACEVTHQGLSSPVT |
| | KSFNRGEC |
| 413 | 1Fab-AFc-1Fab-7 Chain3: |
| | DIQMTQTTSSLSASLGDRVTISCRASQDISNYLNWYQQKPDGTVKLLIYYTSRLHSGVPSRFSGSGSGTDYSLTINNLEQEDIA |
| | TYFCQQGNTRPWTFGGGTKLEIKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 414 | 1Fab-AFc-1Fab-8 Chain1: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECSDKTHTCPPCPAPELLGGPDVFLFPPKPKDTLMISRTPE |
| | VTCVVVDVSHEDPEVKFNWYVDGVEVHNAKTKPREEQYNSTYRVVSVLTVLHQDWLNGKEYKCKVSNKALPAPEEKTISKAKGQ |
| | PREPQVYTLPPSREEMTKNQVSLWCLVKGFYPSDIAVEWESNGQPENNYKTTPPVLDSDGSFFLYSKLTVDKSRWQQGNVFSCS |
| | VMHEALHNHYTQKSLSLSPGK |
| 415 | 1Fab-AFc-1Fab-8 Chain2: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPDVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPEE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGSTGSEVQLQQSGPELVKPGASVKISCKTSGYTFTEYTMHWVKQSHGKSLEWIGGI |
| | SPNIGGTSYNQKFKGKATLTVDKSSSTAYMELRSLTSEDSAVYYCARRGGSFDYWGQGTTLTVSSRTVAAPSVFIFPPSDEQLK |
| | SGTASVVCLLNNFYPREAKVQWKVDNALQSGNSQESVTEQDSKDSTYSLSSTLTLSKADYEKHKVYACEVTHQGLSSPVTKSFN |
| | RGEC |
| 416 | 1Fab-AFc-1Fab-8 Chain3: |
| | DIVMTQSPATLSVTPGDRVSLSCRASQSISDYLHWYQQKSHESPRLLIKYASQSISGIPSRFSGSGSGSDFTLSINSVEPEDVG |
| | VYYCQNGHSFPLTFGAGTKLELKASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTH |
| 417 | AIG-1scFv-5 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLVESGGGLVKPGGSLRLSCAA |
| | SGFTFSSYGMHWVRQAPGKGLEWVAFIRYDGSNKYYADSVKGRFTISRDNSKNTLYLQMNSLRAEDTAVYYCAKDRGLGDGTYF |
| | DYWGQGTTVTVSSGGSGGSGGSGGSGGSGGSQSALTQPASVSGSPGQSITISCSGSSSNIGNNAVNWYQQLPGKAPKLLIYYDD |
| | LLPSGVSDRFSGSKSGTSAFLAISGLQSEDEADYYCAAWDDSLNGPVFGGGTKLTVL |
| 418 | AIG-1scFv-5 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 419 | AIG-1scFv-5 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPG |
| 420 | AIG-1scFv-6 Chain1: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGGGGGSGGGGSGGGGSGGGGSGGGGSGGGGSQVQLQQWGAGLLKPSETLSLTCAV |
| | YGGSFSGYYWSWIRQPPGKGLEWIGEIDHSGSTNYNPSLKSRVTISVDTSKNQFSLKLSSVTAADTAVYYCARARGPWSFDPWG |
| | QGTLVTVSSGGSGGSGGSGGSGGSGGSDIQMTQSPSTLSASVGDRVTITCRASQSISSWLAWYQQKPGKAPKLLIYKASSLESG |
| | VPSRFSGSGSGTEFTLTISSLQPDDFATYYCQQYNSYPTFGGGTKVEIK |
| 421 | AIG-1scFv-6 Chain2: |
| | DIVMTQSPATLSLSPGERATLSCRSSKSLQNVNGNTYLYWFQQKPGQSPQLLIYRMSNLNSGVPDRFSGSGSGTEFTLTISSLE |
| | PEDFAVYYCMQHLEYPITFGAGTKLEIKRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 422 | AIG-1scFv-6 Chain3: |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLDSDGSFFLVSKLTVDKSR |
| | WQQGNVFSCSVMHEALHNHYTQKSLSLSPG |
| 423 | AIG-1scFv-7 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSQVQLVESGGGLVKPGGSLRLSCAAS |
| | GFTFSSYGMHWVRQAPGKGLEWVAFIRYDGSNKYYADSVKGRFTISRDNSKNTLYLQMNSLRAEDTAVYYCAKDRGLGDGTYFD |
| | YWGQGTTVTVSSGGSGGSGGSGGSGGSGGSQSALTQPASVSGSPGQSITISCSGSSSNIGNNAVNWYQQLPGKAPKLLIYYDDL |
| | LPSGVSDRFSGSKSGTSAFLAISGLQSEDEADYYCAAWDDSLNGPVFGGGTKLTVL |
| 424 | AIG-1scFv-7 Chain2: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECS |
| 425 | AIG-1scFv-7 Chain3: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPG |
| 426 | AIG-1scFv-8 Chain1: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSQVQLQQWGAGLLKPSETLSLTCAVY |
| | GGSFSGYYWSWIRQPPGKGLEWIGEIDHSGSTNYNPSLKSRVTISVDTSKNQFSLKLSSVTAADTAVYYCARARGPWSFDPWGQ |
| | GTLVTVSSGGSGGSGGSGGSGGSGGSDIQMTQSPSTLSASVGDRVTITCRASQSISSWLAWYQQKPGKAPKLLIYKASSLESGV |
| | PSRFSGSGSGTEFTLTISSLQPDDFATYYCQQYNSYPTFGGGTKVEIK |
| 427 | AIG-1scFv-8 Chain2: |
| | QPVLTQPPSVSVAPGKTARITCGGNNIGSKSVHWYQQKPGQAPVLVIYYDSDRPSGIPERFSGSNSGNTATLTISRVEAGDEAD |
| | YYCQVWDTSSDHVLFGGGTKLTVLGQPKAAPSVTLFPPSSEELQANKATLVCLISDFYPGAVTVAWKADSSPVKAGVETTTPSK |
| | QSNNKYAASSYLSLTPEQWKSHRSYSCQVTHEGSTVEKTVAPTECS |
| 428 | AIG-1scFv-8 Chain3: |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPG |
| 429 | IgAb_51 Chain1: |
| | EVQLLESGGGLVQPGGSLRLSCAVSGFTFNSFAMSWVRQAPGKGLEWVSAISGSGGGTYYADSVKGRFTISRDNSKNTLYLQMN |
| | SLRAEDTAVYFCAKDKILWFGEPVFDYWGQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKRVEPKSCDKTHTCPPCPAPELLGGPSVFLFPPKP |
| | KDTLMISRTPEVTCVVVDVSHEDPEVKFNWYVDGVEVHNAKTKPREEQYNSTYRVVSVLTVLHQDWLNGKEYKCKVSNKALPAP |
| | IEKTISKAKGQPREPQVYTLPPSREEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| 430 | IgAb_51 Chain2: |
| | EIVLTQSPATLSLSPGERATLSCRASQSVSSYLAWYQQKPGQAPRLLIYDASNRATGIPARFSGSGSGTDFTLTISSLEPEDFA |
| | VYYCQQRSNWPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 431 | AIG-2scFv-7 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSQVQLVESGGGLVKPGGSLRLSCAAS |
| | GFTFSSYGMHWVRQAPGKGLEWVAFIRYDGSNKYYADSVKGRFTISRDNSKNTLYLQMNSLRAEDTAVYYCAKDRGLGDGTYFD |
| | YWGQGTTVTVSSGGSGGSGGSGGSGGSGGSQSALTQPASVSGSPGQSITISCSGSSSNIGNNAVNWYQQLPGKAPKLLIYYDDL |
| | LPSGVSDRFSGSKSGTSAFLAISGLQSEDEADYYCAAWDDSLNGPVFGGGTKLTVL |
| 432 | AIG-2scFv-7 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 433 | AIG-2scFv-7 Chain3: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSSYELTQPLSVSVALGQTARITCGGN |
| | NIGSKSVHWYQQKPGQAPVLVIYQDKKRPSGIPERFSGSNSGNTATLTISRAQAGDEADYYCQVWDDYIVLFGCGTKLTVLGGS |
| | GGSGGSGGSGGSGGSGGSEVQLVQSGAEVKKPGASVKVSCKASGYTFTNYYMQWVRQAPGQCLEWMGIINPSGGVTSYAQKFQG |
| | RVTMTRDTSTSTVYMELSSLRSEDTAVYYCARGSAYYYDFADYWGQGTLVTVSS |
| 434 | AIG-2scFv-8 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSQVQLVESGGGLVKPGGSLRLSCAAS |
| | GFTFSSYGMHWVRQAPGKGLEWVAFIRYDGSNKYYADSVKGRFTISRDNSKNTLYLQMNSLRAEDTAVYYCAKDRGLGDGTYFD |
| | YWGQGTTVTVSSGGSGGSGGSGGSGGSGGSQSALTQPASVSGSPGQSITISCSGSSSNIGNNAVNWYQQLPGKAPKLLIYYDDL |
| | LPSGVSDRFSGSKSGTSAFLAISGLQSEDEADYYCAAWDDSLNGPVFGGGTKLTVL |
| 435 | AIG-2scFv-8 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 436 | AIG-2scFv-8 Chain3: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSSYELTQPLSVSVALGQTARITCGGH |
| | NIGSKNVHWYQQKPGQAPVLVIYQDNKRPSGIPERFSGSNSGNTATLTISRAQAGDEADYYCQVWDNYNVLFGCGTKLTVLGGS |
| | GGSGGSGGSGGSGGSGGSEVQLVQSGAEVKKPGASVKVSCKASGYTFTSYYMHWVRQAPGQCLEWMGAIEPTYGSTSYAQKFQG |
| | RVTMTRDTSTSTVYMELSSLRSEDTAVYYCARGSAYYYDFADYWGQGTLVTVSS |
| 437 | AIG-2scFv-10 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSQSALTQPASVSGSPGQSITISCSGS |
| | SSNIGNNAVNWYQQLPGKAPKLLIYYDDLLPSGVSDRFSGSKSGTSAFLAISGLQSEDEADYYCAAWDDSLNGPVFGGGTKLTV |
| | LGGSGGSGGSGGSGGSGGSGGSQVQLVESGGGLVKPGGSLRLSCAASGFTFSSYGMHWVRQAPGKGLEWVAFIRYDGSNKYYAD |
| | SVKGRFTISRDNSKNTLYLQMNSLRAEDTAVYYCAKDRGLGDGTYFDYWGQGTTVTVSS |
| 438 | AIG-2scFv-10 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 439 | AIG-2scFv-10 Chain3: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSSYELTQPLSVSVALGQTARITCGGH |
| | NIGSKNVHWYQQKPGQAPVLVIYQDNKRPSGIPERFSGSNSGNTATLTISRAQAGDEADYYCQVWDNYNVLFGCGTKLTVLGGS |
| | GGSGGSGGSGGSGGSGGSEVQLVQSGAEVKKPGASVKVSCKASGYTFTSYYMHWVRQAPGQCLEWMGAIEPTYGSTSYAQKFQG |
| | RVTMTRDTSTSTVYMELSSLRSEDTAVYYCARGSAYYYDFADYWGQGTLVTVSS |
| 440 | AIG-2scFv-14 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSQSALTQPASVSGSPGQSITISCSGS |
| | SSNIGNNAVNWYQQLPGKAPKLLIYYDDLLPSGVSDRFSGSKSGTSAFLAISGLQSEDEADYYCAAWDDSLNGPVFGGGTKLTV |
| | LGGSGGSGGSGGSGGSGGSGGSQVQLVESGGGLVKPGGSLRLSCAASGFTFSSYGMHWVRQAPGKGLEWVAFIRYDGSNKYYAD |
| | SVKGRFTISRDNSKNTLYLQMNSLRAEDTAVYYCAKDRGLGDGTYFDYWGQGTTVTVSS |
| 441 | AIG-2scFv-14 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 442 | AIG-2scFv-14 Chain3: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSQSALTQPASVSGSPGQSITISCSGS |
| | SSNIGNNAVNWYQQLPGKAPKLLIYYDDLLPSGVSDRFSGSKSGTSAFLAISGLQSEDEADYYCAAWDDSLNGPVFGGGTKLTV |
| | LGGSGGSGGSGGSGGSGGSGGSQVQLVESGGGLVKPGGSLRLSCAASGFTFSSYGMHWVRQAPGKGLEWVAFIRYDGSNKYYAD |
| | SVKGRFTISRDNSKNTLYLQMNSLRAEDTAVYYCAKDRGLGDGTYFDYWGQGTTVTVSS |
| 443 | AIG-2scFv-15 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSDIQMTQSPSTLSASVGDRVTITCSA |
| | SSRVGYMHWYQQKPGKAPKLLIYDTSKLASGVPSRFSGSGSGTEFTLTISSLQPDDFATYYCFQGSGYPFTFGGGTKVEIKGGS |
| | GGSGGSGGSGGSGGSGGSQVTLRESGPALVKPTQTLTLTCTFSGFSLSTAGMSVGWIRQPPGKALEWLADIWWDDKKHYNPSLK |
| | DRLTISKDTSKNQVVLKVTNMDPADTATYYCARDMIFNFYFDVWGQGTTVTVSS |
| 444 | AIG-2scFv-15 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 445 | AIG-2scFv-15 Chain3: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSQSALTQPASVSGSPGQSITISCSGS |
| | SSNIGNNAVNWYQQLPGKAPKLLIYYDDLLPSGVSDRFSGSKSGTSAFLAISGLQSEDEADYYCAAWDDSLNGPVFGGGTKLTV |
| | LGGSGGSGGSGGSGGSGGSGGSQVQLVESGGGLVKPGGSLRLSCAASGFTFSSYGMHWVRQAPGKGLEWVAFIRYDGSNKYYAD |
| | SVKGRFTISRDNSKNTLYLQMNSLRAEDTAVYYCAKDRGLGDGTYFDYWGQGTTVTVSS |
| 446 | AIG-1scFv-4 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPG |
| 447 | AIG-1scFv-4 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 448 | AIG-1scFv-4 Chain3: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSQSALTQPASVSGSPGQSITISCSGS |
| | SSNIGNNAVNWYQQLPGKAPKLLIYYDDLLPSGVSDRFSGSKSGTSAFLAISGLQSEDEADYYCAAWDDSLNGPVFGGGTKLTV |
| | LGGSGGSGGSGGSGGSGGSGGSQVQLVESGGGLVKPGGSLRLSCAASGFTFSSYGMHWVRQAPGKGLEWVAFIRYDGSNKYYAD |
| | SVKGRFTISRDNSKNTLYLQMNSLRAEDTAVYYCAKDRGLGDGTYFDYWGQGTTVTVSS |
| 449 | Human CD16A |
| | MWQLLLPTALLLLVSAGMRTEDLPKAVVFLEPQWYRVLEKDSVTLKCQGAYSPEDNSTQWFHNESLISSQASSYFIDAATVDDS |
| | GEYRCQTNLSTLSDPVQLEVHIGWLLLQAPRWVFKEEDPIHLRCHSWKNTALHKVTYLQNGKGRKYFHHNSDFYIPKATLKDSG |
| | SYFCRGLFGSKNVSSETVNITITQGLAVSTISSFFPPGYQVSFCLVMVLLFAVDTGLYFSVKTNIRSSTRDWKDHKFKWRKDPQ |
| | DK |
| 450 | Human CD16B |
| | MWQLLLPTALLLLVSAGMRTEDLPKAVVFLEPQWYSVLEKDSVTLKCQGAYSPEDNSTQWFHNESLISSQASSYFIDAATVNDS |
| | GEYRCQTNLSTLSDPVQLEVHIGWLLLQAPRWVFKEEDPIHLRCHSWKNTALHKVTYLQNGKDRKYFHHNSDFHIPKATLKDSG |
| | SYFCRGLVGSKNVSSETVNITITQGLAVSTISSFSPPGYQVSFCLVMVLLFAVDTGLYFSVKTNI |
| 451 | Linker |
| | GGGS |
| 452 | IgG2 subtype hinge: |
| | ERKCCVECPPCP |
| 453 | IgG3 subtype hinge: |
| | ELKTPLDTTHTCPRCP |
| 454 | IgG3 subtype hinge: |
| | ELKTPLGDTTHTCPRCP |
| 455 | IgG4 subtype hinge: |
| | ESKYGPPCPSCP |
| 456 | VH CD89-1: |
| | QVQLVESGGGVVQPGRSLRLSCAASGFTFSSYVLHWVRQAPGKGLDWVAVISDDGRNKYFADSVKGRFTISRDNSKNTLYLQMN |
| | SLRAEDTAVYYCVREGYSGSWFDYWGQGTLVTVSS |
| 457 | VH CD89-2: |
| | EIQLQQTGPELVKPGASVKISCKASGYSFTDYIIFWVKQSHGKSLEWTGNINPYYGSTSYNLKFKGKATLTVDKSSSTAYMQLN |
| | SLTSEDSAVYYCVRGVYYYGSSYEAFPYWGQGTLVTVSA |
| 458 | VL CD89-1: |
| | AIQLTQSPSSLSASVGDRVTITCRASQGISSALAWYQQKPGKAPKLLIYGASSLEGGVPSRFSGSGSGTDFTLTISSLQPEDFA |
| | TYYCQQFNSYPFTFGPGTKVDIK |
| 459 | VL CD89-2: |
| | DIQLTQSPLTLSITIGQPASISCKSSQSLLDSDGKTYLNWLLQRPGQSPTRLIYLVSKLDSGVPDRFTGSGSGTDFTLKISRVE |
| | AEDLGIYYCWQGAHFPQTFGGGTKLEIK |
| 460 | HCDR1 CD89-1: |
| | SYVLH |
| 461 | HCDR2 CD89-1: |
| | VISDDGRNKYFADSVKG |
| 462 | HCDR3 CD89-1: |
| | EGYSGSWFDY |
| 463 | LCDR1 CD89-1: |
| | RASQGISSALA |
| 464 | LCDR2 CD89-1: |
| | GASSLEG |
| 465 | LCDR3 CD89-1: |
| | QQFNSYPFT |
| 466 | HCDR1 CD89-2: |
| | DYIIF |
| 467 | HCDR2 CD89-2: |
| | NINPYYGSTSYNLKFKG |
| 468 | HCDR3 CD89-2: |
| | GVYYYGSSYEAFPY |
| 469 | LCDR1 CD89-2: |
| | KSSQSLLDSDGKTYLN |
| 470 | LCDR2 CD89-2: |
| | LVSKLDS |
| 471 | LCDR3 CD89-2: |
| | WQGAHFPQT |
| 472 | BisscDb-25: CD16A-1 × CD89-1 |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVLGGSGGSQVQLVESGGGVVQPGRSLRLSCAASGFTFSSYVLHWVRQAPGKGLDWVAVISDDGR |
| | NKYFADSVKGRFTISRDNSKNTLYLQMNSLRAEDTAVYYCVREGYSGSWFDYWGQGTLVTVSSGGSGGSGGSGGSGGSGGSAIQ |
| | LTQSPSSLSASVGDRVTITCRASQGISSALAWYQQKPGKAPKLLIYGASSLEGGVPSRFSGSGSGTDFTLTISSLQPEDFATYY |
| | SYAQKFQGRVTMTRDTSTSTVYMELSSLRSEDTAVYYCARGSAYYYDFADYWGQGTLVTVSS |
| 473 | BisscDb-26: CD16A-1 × CD89-2 |
| | SYELTQPLSVSVALGQTARITCGGHNIGSKNVHWYQQKPGQAPVLVIYQDNKRPSGIPERFSGSNSGNTATLTISRAQAGDEAD |
| | YYCQVWDNYNVLFGCGTKLTVLGGSGGSEIQLQQTGPELVKPGASVKISCKASGYSFTDYIIFWVKQSHGKSLEWTGNINPYYG |
| | STSYNLKFKGKATLTVDKSSSTAYMQLNSLTSEDSAVYYCVRGVYYYGSSYEAFPYWGQGTLVTVSAGGSGGSGGSGGSGGSGG |
| | SDIQLTQSPLTLSITIGQPASISCKSSQSLLDSDGKTYLNWLLQRPGQSPTRLIYLVSKLDSGVPDRFTGSGSGTDFTLKISRV |
| | EAEDLGIYYCWQGAHFPQTFGGGTKLEIKGGSGGSQVQLVQSGAEVKKPGASVKVSCKASGYTFTSYYMHWVRQAPGQCLEWMG |
| | AIEPTYGSTSYAQKFQGRVTMTRDTSTSTVYMELSSLRSEDTAVYYCARGSAYYYDFADYWGQGTLVTVSS |
| 474 | BisscDb-27: CD89-1 × EGFR-1 |
| | QVQLVESGGGVVQPGRSLRLSCAASGFTFSSYVLHWVRQAPGKGLDWVAVISDDGRNKYFADSVKGRFTISRDNSKNTLYLQMN |
| | SLRAEDTAVYYCVREGYSGSWFDYWGQGTLVTVSSGGSGGSGGSQPVLTQPPSVSVAPGKTARITCGGNNIGSKSVHWYQQKPG |
| | QAPVLVIYYDSDRPSGIPERFSGSNSGNTATLTISRVEAGDEADYYCQVWDTSSDHVLFGGGTKLTVLGGSGGSGGSGGSGGSG |
| | GSGGSQVQLQESGPGLVKPSETLSLTCTVSGGSVSSGSYYWSWIRQPPGKGLEWIGYIYYSGSTNYNPSLKSRVTISVDTSKNQ |
| | FSLKLSSVTAADTAVYYCARNPISIPAFDIWGQGTMVTVSSGGSGGSGGSAIQLTQSPSSLSASVGDRVTITCRASQGISSALA |
| | WYQQKPGKAPKLLIYGASSLEGGVPSRFSGSGSGTDFTLTISSLQPEDFATYYCQQFNSYPFTFGPGTKVDIK |
| 475 | BisscDb-28: CD89-1 × CD19 (MOR208) |
| | QVQLVESGGGVVQPGRSLRLSCAASGFTFSSYVLHWVRQAPGKGLDWVAVISDDGRNKYFADSVKGRFTISRDNSKNTLYLQMN |
| | SLRAEDTAVYYCVREGYSGSWFDYWGQGTLVTVSSGGSGGSGGSDIVMTQSPATLSLSPGERATLSCRSSKSLQNVNGNTYLYW |
| | FQQKPGQSPQLLIYRMSNLNSGVPDRFSGSGSGTEFTLTISSLEPEDFAVYYCMQHLEYPITFGAGTKLEIKGGSGGSGGSGGS |
| | GGSGGSGGSEVQLVESGGGLVKPGGSLKLSCAASGYTFTSYVMHWVRQAPGKGLEWIGYINPYNDGTKYNEKFQGRVTISSDKS |
| | ISTAYMELSSLRSEDTAMYYCARGTYYYGTRVFDYWGQGTLVTVSSGGSGGSGGSAIQLTQSPSSLSASVGDRVTITCRASQGI |
| | SSALAWYQQKPGKAPKLLIYGASSLEGGVPSRFSGSGSGTDFTLTISSLQPEDFATYYCQQFNSYPFTFGPGTKVDIK |
| 476 | BisscDb-29: CD89-2 × EGFR-1 |
| | EIQLQQTGPELVKPGASVKISCKASGYSFTDYIIFWVKQSHGKSLEWTGNINPYYGSTSYNLKFKGKATLTVDKSSSTAYMQLN |
| | SLTSEDSAVYYCVRGVYYYGSSYEAFPYWGQGTLVTVSAGGSGGSGGSQPVLTQPPSVSVAPGKTARITCGGNNIGSKSVHWYQ |
| | QKPGQAPVLVIYYDSDRPSGIPERFSGSNSGNTATLTISRVEAGDEADYYCQVWDTSSDHVLFGGGTKLTVLGGSGGSGGSGGS |
| | GGSGGSGGSQVQLQESGPGLVKPSETLSLTCTVSGGSVSSGSYYWSWIRQPPGKGLEWIGYIYYSGSTNYNPSLKSRVTISVDT |
| | SKNQFSLKLSSVTAADTAVYYCARNPISIPAFDIWGQGTMVTVSSGGSGGSGGSDIQLTQSPLTLSITIGQPASISCKSSQSLL |
| | DSDGKTYLNWLLQRPGQSPTRLIYLVSKLDSGVPDRFTGSGSGTDFTLKISRVEAEDLGIYYCWQGAHFPQTFGGGTKLEIK |
| 477 | BisscDb-30: CD89-2 × CD19 (MOR208) |
| | EIQLQQTGPELVKPGASVKISCKASGYSFTDYIIFWVKQSHGKSLEWTGNINPYYGSTSYNLKFKGKATLTVDKSSSTAYMQLN |
| | SLTSEDSAVYYCVRGVYYYGSSYEAFPYWGQGTLVTVSAGGSGGSGGSDIVMTQSPATLSLSPGERATLSCRSSKSLQNVNGNT |
| | YLYWFQQKPGQSPQLLIYRMSNLNSGVPDRFSGSGSGTEFTLTISSLEPEDFAVYYCMQHLEYPITFGAGTKLEIKGGSGGSGG |
| | SGGSGGSGGSGGSEVQLVESGGGLVKPGGSLKLSCAASGYTFTSYVMHWVRQAPGKGLEWIGYINPYNDGTKYNEKFQGRVTIS |
| | SDKSISTAYMELSSLRSEDTAMYYCARGTYYYGTRVFDYWGQGTLVTVSSGGSGGSGGSDIQLTQSPLTLSITIGQPASISCKS |
| | SQSLLDSDGKTYLNWLLQRPGQSPTRLIYLVSKLDSGVPDRFTGSGSGTDFTLKISRVEAEDLGIYYCWQGAHFPQTFGGGTKL |
| | EIK |
| 478 | scDb-8: CD89-1 |
| | AIQLTQSPSSLSASVGDRVTITCRASQGISSALAWYQQKPGKAPKLLIYGASSLEGGVPSRFSGSGSGTDFTLTISSLQPEDFA |
| | TYYCQQFNSYPFTFGPGTKVDIKGGSGGSQVQLVESGGGVVQPGRSLRLSCAASGFTFSSYVLHWVRQAPGKGLDWVAVISDDG |
| | RNKYFADSVKGRFTISRDNSKNTLYLQMNSLRAEDTAVYYCVREGYSGSWFDYWGQGTLVTVSSGGSGGSGGSGGSGGSGGS |
| | AIQLTQSPSSLSASVGDRVTITCRASQGISSALAWYQQKPGKAPKLLIYGASSLEGGVPSRFSGSGSGTDFTLTISSLQPEDFA |
| | TYYCQQFNSYPFTFGPGTKVDIKGGSGGSQVQLVESGGGVVQPGRSLRLSCAASGFTFSSYVLHWVRQAPGKGLDWVAVISDDG |
| | RNKYFADSVKGRFTISRDNSKNTLYLQMNSLRAEDTAVYYCVREGYSGSWFDYWGQGTLVTVSS |
| 479 | scDb-9: CD89-2 |
| | DIQLTQSPLTLSITIGQPASISCKSSQSLLDSDGKTYLNWLLQRPGQSPTRLIYLVSKLDSGVPDRFTGSGSGTDFTLKISRVE |
| | AEDLGIYYCWQGAHFPQTFGGGTKLEIKGGSGGSEIQLQQTGPELVKPGASVKISCKASGYSFTDYIIFWVKQSHGKSLEWTGN |
| | INPYYGSTSYNLKFKGKATLTVDKSSSTAYMQLNSLTSEDSAVYYCVRGVYYYGSSYEAFPYWGQGTLVTVSAGGSGGSGGSGG |
| | SGGSGGSDIQLTQSPLTLSITIGQPASISCKSSQSLLDSDGKTYLNWLLQRPGQSPTRLIYLVSKLDSGVPDRFTGSGSGTDFT |
| | LKISRVEAEDLGIYYCWQGAHFPQTFGGGTKLEIKGGSGGSEIQLQQTGPELVKPGASVKISCKASGYSFTDYIIFWVKQSHGK |
| | SLEWTGNINPYYGSTSYNLKFKGKATLTVDKSSSTAYMQLNSLTSEDSAVYYCVRGVYYYGSSYEAFPYWGQGTLVTVSA |
| 480 | ta-scFv-11: CD19 (MOR208) × CD89-1 |
| | EVQLVESGGGLVKPGGSLKLSCAASGYTFTSYVMHWVRQAPGKGLEWIGYINPYNDGTKYNEKFQGRVTISSDKSISTAYMELS |
| | SLRSEDTAMYYCARGTYYYGTRVFDYWGQGTLVTVSSGGSGGSGGSGGSGGSGGSDIVMTQSPATLSLSPGERATLSCRSSKSL |
| | QNVNGNTYLYWFQQKPGQSPQLLIYRMSNLNSGVPDRFSGSGSGTEFTLTISSLEPEDFAVYYCMQHLEYPITFGAGTKLEIKG |
| | GGGSAIQLTQSPSSLSASVGDRVTITCRASQGISSALAWYQQKPGKAPKLLIYGASSLEGGVPSRFSGSGSGTDFTLTISSLQP |
| | EDFATYYCQQFNSYPFTFGPGTKVDIKGGSGGSGGSGGSGGSGGSGGSQVQLVESGGGVVQPGRSLRLSCAASGFTFSSYVLHW |
| | VRQAPGKGLDWVAVISDDGRNKYFADSVKGRFTISRDNSKNTLYLQMNSLRAEDTAVYYCVREGYSGSWFDYWGQGTLVTVSS |
| 481 | ta-scFv-12: EGFR-2 × CD89-1 |
| | EVQLQESGPGLVKPSETLSLTCTVSGGSVSSGSYYWSWIRQPPGKGLEWIGYIYYSGSTNYNPSLKSRVTISVDTSKNQFSLKL |
| | SSVTAADTAVYYCARNPISIPAFDIWGQGTMVTVSSGGSGGSGGSGGSGGSGGSQPVLTQPPSVSVAPGKTARITCGGNNIGSK |
| | SVHWYQQKPGQAPVLVIYYDSDRPSGIPERFSGSNSGNTATLTISRVEAGDEADYYCQVWDTSSDHVLFGGGTKLTVLGGGGS |
| | AIQLTQSPSSLSASVGDRVTITCRASQGISSALAWYQQKPGKAPKLLIYGASSLEGGVPSRFSGSGSGTDFTLTISSLQPEDFA |
| | TYYCQQFNSYPFTFGPGTKVDIKGGSGGSGGSGGSGGSGGSGGSQVQLVESGGGVVQPGRSLRLSCAASGFTFSSYVLHWVRQA |
| | PGKGLDWVAVISDDGRNKYFADSVKGRFTISRDNSKNTLYLQMNSLRAEDTAVYYCVREGYSGSWFDYWGQGTLVTVSS |
| 482 | scFv-13: CD89-1 |
| | AIQLTQSPSSLSASVGDRVTITCRASQGISSALAWYQQKPGKAPKLLIYGASSLEGGVPSRFSGSGSGTDFTLTISSLQPEDFA |
| | TYYCQQFNSYPFTFGPGTKVDIKGGSGGSGGSGGSGGSGGSGGSQVQLVESGGGVVQPGRSLRLSCAASGFTFSSYVLHWVRQA |
| | PGKGLDWVAVISDDGRNKYFADSVKGRFTISRDNSKNTLYLQMNSLRAEDTAVYYCVREGYSGSWFDYWGQGTLVTVSS |
| 483 | scFv-14: CD89-2 |
| | DIQLTQSPLTLSITIGQPASISCKSSQSLLDSDGKTYLNWLLQRPGQSPTRLIYLVSKLDSGVPDRFTGSGSGTDFTLKISRVE |
| | AEDLGIYYCWQGAHFPQTFGGGTKLEIKGGSGGSGGSGGSGGSGGSGGSEIQLQQTGPELVKPGASVKISCKASGYSFTDYIIF |
| | WVKQSHGKSLEWTGNINPYYGSTSYNLKFKGKATLTVDKSSSTAYMQLNSLTSEDSAVYYCVRGVYYYGSSYEAFPYWGQGTLV |
| | TVSA |
| 484 | AIG-1scFv-6 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPG |
| 485 | AIG-1scFv-6 Chain2: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSAIQLTQSPSSLSASVGDRVTITCRA |
| | SQGISSALAWYQQKPGKAPKLLIYGASSLEGGVPSRFSGSGSGTDFTLTISSLQPEDFATYYCQQFNSYPFTFGPGTKVDIKGG |
| | SGGSGGSGGSGGSGGSGGSQVQLVESGGGVVQPGRSLRLSCAASGFTFSSYVLHWVRQAPGKGLDWVAVISDDGRNKYFADSVK |
| | GRFTISRDNSKNTLYLQMNSLRAEDTAVYYCVREGYSGSWFDYWGQGTLVTVSS |
| 486 | AIG-1scFv-6 Chain3: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 487 | AIG-1scFv-7 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPG |
| 488 | AIG-1scFv-7 Chain2: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSDIQLTQSPLTLSITIGQPASISCKS |
| | SQSLLDSDGKTYLNWLLQRPGQSPTRLIYLVSKLDSGVPDRFTGSGSGTDFTLKISRVEAEDLGIYYCWQGAHFPQTFGGGTKL |
| | EIKGGSGGSGGSGGSGGSGGSGGSEIQLQQTGPELVKPGASVKISCKASGYSFTDYIIFWVKQSHGKSLEWTGNINPYYGSTSY |
| | NLKFKGKATLTVDKSSSTAYMQLNSLTSEDSAVYYCVRGVYYYGSSYEAFPYWGQGTLVTVSA |
| 489 | AIG-1scFv-7 Chain3: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 490 | AIG-2scFv-28 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSSYELTQPLSVSVALGQTARITCGGHNIGSKNVHWYQQKPGQAPVL |
| | VIYQDNKRPSGIPERFSGSNSGNTATLTISRAQAGDEADYYCQVWDNYNVLFGCGTKLTVLGGSGGSGGSGGSGGSGGSGGSQV |
| | QLVQSGAEVKKPGASVKVSCKASGYTFTSYYMHWVRQAPGQCLEWMGAIEPTYGSTSYAQKFQGRVTMTRDTSTSTVYMELSSL |
| | RSEDTAVYYCARGSAYYYDFADYWGQGTLVTVSS |
| 491 | AIG-2scFv-28 Chain2: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSAIQLTQSPSSLSASVGDRVTITCRA |
| | SQGISSALAWYQQKPGKAPKLLIYGASSLEGGVPSRFSGSGSGTDFTLTISSLQPEDFATYYCQQFNSYPFTFGPGTKVDIKGG |
| | SGGSGGSGGSGGSGGSGGSQVQLVESGGGVVQPGRSLRLSCAASGFTFSSYVLHWVRQAPGKGLDWVAVISDDGRNKYFADSVK |
| | GRFTISRDNSKNTLYLQMNSLRAEDTAVYYCVREGYSGSWFDYWGQGTLVTVSS |
| 492 | AIG-2scFv-28 Chain3: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 493 | AIG-2scFv-29 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSSYELTQPLSVSVALGQTARITCGGHNIGSKNVHWYQQKPGQAPVL |
| | VIYQDNKRPSGIPERFSGSNSGNTATLTISRAQAGDEADYYCQVWDNYNVLFGCGTKLTVLGGSGGSGGSGGSGGSGGSGGSQV |
| | QLVQSGAEVKKPGASVKVSCKASGYTFTSYYMHWVRQAPGQCLEWMGAIEPTYGSTSYAQKFQGRVTMTRDTSTSTVYMELSSL |
| | RSEDTAVYYCARGSAYYYDFADYWGQGTLVTVSS |
| 494 | AIG-2scFv-29 Chain2: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSDIQLTQSPLTLSITIGQPASISCKS |
| | SQSLLDSDGKTYLNWLLQRPGQSPTRLIYLVSKLDSGVPDRFTGSGSGTDFTLKISRVEAEDLGIYYCWQGAHFPQTFGGGTKL |
| | EIKGGSGGSGGSGGSGGSGGSGGSEIQLQQTGPELVKPGASVKISCKASGYSFTDYIIFWVKQSHGKSLEWTGNINPYYGSTSY |
| | NLKFKGKATLTVDKSSSTAYMQLNSLTSEDSAVYYCVRGVYYYGSSYEAFPYWGQGTLVTVSA |
| 495 | AIG-2scFv-29 Chain3: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 496 | scFv-IgAb-441 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLTCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSAIQLTQSPSSLSASVGDRVTITCRA |
| | SQGISSALAWYQQKPGKAPKLLIYGASSLEGGVPSRFSGSGSGTDFTLTISSLQPEDFATYYCQQFNSYPFTFGPGTKVDIKGG |
| | SGGSGGSGGSGGSGGSGGSQVQLVESGGGVVQPGRSLRLSCAASGFTFSSYVLHWVRQAPGKGLDWVAVISDDGRNKYFADSVK |
| | GRFTISRDNSKNTLYLQMNSLRAEDTAVYYCVREGYSGSWFDYWGQGTLVTVSS |
| 497 | scFv-IgAb-441 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 498 | scFv-IgAb-442 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLTCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSGGGGSGGGGSGGGGSGGGGSDIQLTQSPLTLSITIGQPASISCKS |
| | SQSLLDSDGKTYLNWLLQRPGQSPTRLIYLVSKLDSGVPDRFTGSGSGTDFTLKISRVEAEDLGIYYCWQGAHFPQTFGGGTKL |
| | EIKGGSGGSGGSGGSGGSGGSGGSEIQLQQTGPELVKPGASVKISCKASGYSFTDYIIFWVKQSHGKSLEWTGNINPYYGSTSY |
| | NLKFKGKATLTVDKSSSTAYMQLNSLTSEDSAVYYCVRGVYYYGSSYEAFPYWGQGTLVTVSA |
| 499 | scFv-IgAb-442 Chain2: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 500 | AIG-1scDb-1scFv-5 Chain1: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDSDGSFFLVSKLTVDKSRW |
| | QQGNVFSCSVMHEALHNHYTQKSLSLSPGGGGGSGGGGSGGGGSGGGGSGGGGSGGGGSAIQLTQSPSSLSASVGDRVTITCRA |
| | SQGISSALAWYQQKPGKAPKLLIYGASSLEGGVPSRFSGSGSGTDFTLTISSLQPEDFATYYCQQFNSYPFTFGPGTKVDIKGG |
| | SGGSGGSGGSGGSGGSGGSQVQLVESGGGVVQPGRSLRLSCAASGFTFSSYVLHWVRQAPGKGLDWVAVISDDGRNKYFADSVK |
| | GRFTISRDNSKNTLYLQMNSLRAEDTAVYYCVREGYSGSWFDYWGQGTLVTVSS |
| 501 | AIG-1scDb-1scFv-5 Chain2: |
| | EVQLVESGGGLVQPGGSLRLSCAASGFNIKDTYIHWVRQAPGKGLEWVARIYPTNGYTRYADSVKGRFTISADTSKNTAYLQMN |
| | SLRAEDTAVYYCSRWGGDGFYAMDYWGQGTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCDKTHTCPPCPAPEFEGGPSVFLFPPKPKD |
| | TLMISRTPEVTCVVVAVSHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDSDGSFFLYSKLTVDKSRW |
| | QQGNVFSCSVMHEALHNHYTQKSLSLSPGGGGGSGGGGSSYELTQPLSVSVALGQTARITCGGHNIGSKNVHWYQQKPGQAPVL |
| | VIYQDNKRPSGIPERFSGSNSGNTATLTISRAQAGDEADYYCQVWDNYNVLFGCGTKLTVLGGSGGSQVQLVQSGAEVKKPGAS |
| | VKVSCKASGYTFTSYYMHWVRQAPGQCLEWMGAIEPTYGSTSYAQKFQGRVTMTRDTSTSTVYMELSSLRSEDTAVYYCARGSA |
| | YYYDFADYWGQGTLVTVSSGGSGGSGGSGGSGGSGGSSYELTQPLSVSVALGQTARITCGGHNIGSKNVHWYQQKPGQAPVLVI |
| | YQDNKRPSGIPERFSGSNSGNTATLTISRAQAGDEADYYCQVWDNYNVLFGCGTKLTVLGGSGGSQVQLVQSGAEVKKPGASVK |
| | VSCKASGYTFTSYYMHWVRQAPGQCLEWMGAIEPTYGSTSYAQKFQGRVTMTRDTSTSTVYMELSSLRSEDTAVYYCARGSAYY |
| | YDFADYWGQGTLVTVSS |
| 502 | AIG-1scDb-1scFv-5 Chain3: |
| | DIQMTQSPSSLSASVGDRVTITCRASQDVNTAVAWYQQKPGKAPKLLIYSASFLYSGVPSRFSGSRSGTDFTLTISSLQPEDFA |
| | TYYCQQHYTTPPTFGQGTKVEIKRTVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |

1. A trispecific antibody construct comprising
(i) a first binding domain (A), which is capable of specifically binding to a first target (A′) that is CD16A on the surface of an immune effector cell;
(ii) a second binding domain (B), which is capable of specifically binding to a second target (B′) that is another antigen on the surface of an immune effector cell, wherein said antigen is selected from the group comprising CD56, NKG2A, NKG2D, NKp30, NKp44, NKp46, NKp80, DNAM-1, SLAMF7, OX40, CD47/SIRPα, CD89, CD96, CD137, CD160, TIGIT, nectin-4, PD-1, PD-L1, LAG-3, CTLA-4, TIM-3, KIR2DL1-5, KIR3DL1-3, KIR2DS1-5 and CD3; and
(iii) a third binding domain (C), which is capable of specifically binding to a third target (C′) that is an antigen on the surface of a target cell,
wherein the first binding domain (A) comprises an VH and a VL domain of an antibody.

2. The antibody construct of claim 1, wherein the first binding domain (A) binds to an epitope on CD16A which is C-terminal to the physiological Fcγ receptor binding domain, said epitope preferably comprises Y158 of SEQ ID NO: 449.

3. The antibody construct of claim 1 or 2, wherein the first binding domain (A) and the second binding domain (B) are positioned to each other in a way that simultaneous binding of two immune effector cells is reduced or preferably prevented.

4. The antibody construct of any one of the preceding claims, wherein the antibody construct binds to a target cell and one immune effector cell simultaneously.

5. The antibody construct of any one of the preceding claims, further comprising a fourth domain (D) comprising a half-life extension domain.

6. The antibody construct of claim 5, wherein said half-life extension domain comprises a CH2 domain, wherein the Fcγ receptor binding domain is silenced.

7. The antibody construct of claim 5 or 6, wherein said half-life extension domain comprises a CH3 domain.

8. The antibody construct of any one of claims 5 to 7, wherein the antibody construct comprise at least one hinge domain and CH3 domain fused to a CH2 domain in an amino to carboxyl order in the order hinge domain-CH2 domain-CH3 domain.

9. The antibody construct of any one of claims 5 to 8, wherein the antibody construct comprises at least two of the hinge domain-CH2 domain-CH3 domain elements.

10. The antibody construct of any one of the preceding claims, wherein the third binding domain (C) comprises an VH and a VL domain of an antibody.

11. The antibody construct of any one of the preceding claims, wherein the third binding domain (C) binds to an antigen on the surface of a target cell, which antigen is selected from the group consisting of CD19, CD20, CD22, CD30, CD33, CD52, CD70, CD74, CD79b, CD123, CLL1, BCMA, FCRH5, EGFR, EGFRvlll, HER2, and GD2.

12. The antibody construct of any one of the preceding claims, wherein the second binding domain (B) comprises an VH and a VL domain of an antibody.

13. The antibody construct of any one of the preceding claims, wherein the first binding domain (A) is fused to the C terminus of a first CH3 domain and the second binding domain (B) is fused to the C terminus of a second CH3 domain.

14. The antibody construct of claim 13, wherein the antibody construct is monovalent for the first binding domain (A) and monovalent for the second binding domain (B).

15. The antibody construct of any one of claims 1 to 12, wherein the first binding domain (A) is fused to the N-terminus of a first hinge and the second binding domain (B) is fused to the N-terminus of a second hinge.

16. The antibody construct of any one of claims 1 to 12, wherein the first binding domain (A) and the second binding domain (B) are fused to each other.

17. The antibody construct of claim 16, wherein the antibody construct is monovalent for the first binding domain (A) and monovalent for the second binding domain (B).

18. The antibody construct of claim 16, wherein the antibody construct is bivalent for the first binding domain (A) and bivalent for the second binding domain (B), wherein each of the first binding domains (A) is fused to a second binding domain (B).

19. The antibody construct of any one of claims 16 to 18, wherein the C terminus of the VL of the first binding domain (A) is fused to the N terminus of the VH of the second binding domain (B) and the C terminus of the VL of the second binding domain (B) is fused to the N terminus of the VH of the first binding domain (A).

20. The antibody construct of any one of claims 16 to 18, wherein the N terminus of the VL of the first binding domain (A) is fused to the C terminus of the VH of the second binding domain (B) and the N terminus of the VL of the second binding domain (B) is fused to the C terminus of the VH of the first binding domain (A).

21. The antibody construct of any one of claims 16 to 18, wherein the C terminus of the VL of the first binding domain (A) is fused to the N terminus of the VL of the second binding domain (B) and the C terminus of the VH of the first binding domain (A) is fused to the N terminus of the VH of the second binding domain (B).

22. The antibody construct of any one of claims 16 to 18, wherein the C terminus of the VL of the second binding domain (B) is fused to the N terminus of the VL of the first binding domain (A) and the C terminus of the VH of the second binding domain (B) is fused to the N terminus of the VH of the first binding domain (A).

23. The antibody construct of any one of claims 16 to 18, wherein the first binding domain (A) and the second binding domain (B) are fused to each other in form of a bi-scFv, double Fab, db or scDb.

24. The antibody construct of claim 23, wherein the first binding domain (A) and the second binding domain (B) are fused to each other in form of a db or scDb.

25. The antibody construct of claim 24, wherein the variable domains of the db or scDb are arranged in V_{L}-V_{H}-V_{L}-V_{H} order.

26. The antibody construct of any one of claims 15 to 25, wherein (a) the first binding domain (A) is fused N-terminally to a hinge domain and the second binding domain (B) is fused N-terminally to the first binding domain (A); or (b) the first binding domain (A) is fused C-terminally to a CH3 domain and the second binding domain (B) is fused C-terminally to the first binding domain.

27. The antibody construct of any one of claims 15 to 26, wherein the first binding domain (A) is fused N-terminally to a hinge domain and the second binding domain (B) is fused N-terminally to the first binding domain (A).

28. The antibody construct of any one of the preceding claims, wherein the binding site of the first binding domain (A) and the binding site of the second binding domain (B) are within a distance of about 25 nm or less, preferably about 20 nm or less, preferably about 15 nm or less, preferably about 10 nm or less.

29. The antibody construct of any one of the preceding claims, wherein the binding site of the first binding domain (A) and the binding site of the second binding domain (B) are in cis orientation.

30. The antibody construct of any one of the preceding claims, wherein the binding site of the first binding domain (A) and the binding site of the third binding domain (C) are in trans orientation.

31. The antibody construct of any one of the preceding claims, wherein the binding site of the second binding domain (B) and the binding site of the third binding domain (C) are in trans orientation.

32. The antibody construct of any one of the preceding claims, wherein the first binding domain (A) comprises:
(i) a VL region comprising CDR-L1, CDR-L2 and CDR-L3 selected from:
(a) CDR-L1 as depicted in SEQ ID NO: 29, a CDR-L2 as depicted in SEQ ID NO: 30, a CDR-L3 as depicted in SEQ ID NO: 31; and
(b) CDR-L1 as depicted in SEQ ID NO: 35, a CDR-L2 as depicted in SEQ ID NO: 36, a CDR-L3 as depicted in SEQ ID NO: 37;
(ii) a VH region comprising CDR-H1, CDR-H2 and CDR-H3 selected from:
(a) CDR-H1 as depicted in SEQ ID NO: 26, a CDR-H2 as depicted in SEQ ID NO: 27, a CDR-H3 as depicted in SEQ ID NO: 28; and
(b) a CDR-L1 as depicted in SEQ ID NO: 29, a CDR-L2 as depicted in SEQ ID NO: 30, a CDR-L3 as depicted in SEQ ID NO: 31.

33. The antibody construct of any one of the preceding claims, having an amino acid sequence selected from the group consisting of SEQ ID NOs: 161-162; 163-164; 165-166; 167-168; 177-179; 180-182; 183-185; 186-188; 189-191; 192-194; 195-197; 198-200; 225-227; 228-230; 231-233; 234-236 237-238, 239-240, 241-242, 243-244, 245-246, 247-248, 249-250, 251-252; 269-270; 271-272; 273-274; 275-276; 277-278; 279-280; 281-282; 283-284; 293-295; 296-298; 299-301; 302-304; 305-307; 308-310; 311-313; 314-316; 329-331; 332-334; 335-337; 338-340; 353-354; 355-356; 357-358; 359-360; 369-371; 372-374; 375-377; 378-380; 431-433; 434-436; 437-439, 490-492, 493-495, and 500-502.

34. The antibody construct of and one of the preceding claims, wherein the antibody construct induces a lower degree of fratricide as compared to a control construct selected from the group consisting of SEQ ID NOs: 393-395; 396-398; 399-401; 402-404; 405-407; 408-410; 411-413; 414-416; 417-419; 420-422; 423-425; and 426-428.

35. The antibody construct of any one of the preceding claims, wherein the antibody construct induces a lower degree of fratricide as compared to the anti-CD38 antibody of SEQ ID NOs: 429 and 430.

36. The antibody construct of any one of the preceding claims, wherein the antibody construct induces about 25% or less NK cell fratricide in a cytotoxicity assay.

37. A nucleic acid molecule comprising a sequence encoding an antibody construct of any one of claims 1 to 36.

38. A vector comprising a nucleic acid molecule of claim 37.

39. A host cell comprising a nucleic acid molecule of claim 37 or a vector of claim 38.

40. A method of producing an antibody construct of any one of claims 1 to 36, said method comprising culturing a host cell of claim 39 under conditions allowing the expression of the antibody construct of any one of claims 1 to 36 and recovering the produced antibody construct from the culture.

41. A pharmaceutical composition comprising an antibody construct of any one of claims 1 to 36, or produced of the method of claim 40.

42. An antibody construct of any one of claims 1 to 36 for use in therapy.

43. The antibody construct of any one of claims 1 to 36, or produced of the method of claim 40, for use in the prevention, treatment or amelioration of a disease selected from a proliferative disease, a tumorous disease, a viral disease or an immunological disorder.

44. A method of treatment or amelioration of a proliferative disease, a tumorous disease, a viral disease or an immunological disorder, comprising the step of administering to a subject in need thereof the antibody construct of any one of claims 1 to 36, or produced of the process of claim 40.

45. A kit comprising an antibody construct of any one of claims 1 to 36, or produced of the method of claim 40, a nucleic acid molecule of claim 37, a vector of claim 38, and/or a host cell of claim 39.

46. A method of simultaneously binding a target cell and an immune effector cell, comprising administering to a subject the antibody construct of any one of claims 1 to 36, wherein the antibody construct binds the tumor cell and a first immune effector cell but does not essentially bind a further immune effector cell.

47. The method of claim 46, wherein the first binding domain and the second binding domain bind to a first target (A′) and a second target (B′) that are on the same first immune effector cell.

48. The method of claim 46 or 47, wherein the method comprises target cell specific activation of the first immune effector cell.
